SEQUENCE LISTING (137 sequences)

SEQ ID NO: 1 - DNA, 1593 bp, Bemisia tabaci
  source 1..1593: mol_type = other DNA
atgaccaacgagatggagtcctcagaaaatcaagccgaaagccagactgaagatttgatacctaagccaaaagaaataaaatacaaaaatgccggcagatcgacattctcacagatcgtggctatgcttgtcttggcctgtttgctcgttgactttgggctggagctgattatacccaccatcgtgataggtgctcttcataaaaaccccgacgaagctctgaacttgaccgacgagcaagcatcgtggtttggaagcatcttgtacttcgcgcacccgatcggggccctgatctcagggttcctgcaggagctcctagggcggaagcgtagcctcctgctggtgaacatcccgatgctggtggcctggagcacgctgtacctggcaagctccgtctaccagctctatttcgtctcagccgcgctgggcctttgcatcggcttttgcgaggcccccctccactcgtacatcggagagatgagcgaaccccacgtgcgaggaaccctctcagccatgggcacggcctcttgcctcatgggtatgctcatcatgtacctcatcggctacctcgtccactggaggacggccgccttgatcagcagcttcgttccggtcatcacgttcctagccattgcccagattccagaatcgcccacttggttggtcatgaatggccgcgaaaaagaagcccaaaaagctttaggttggctgagaggctggctcaagccggaggaggttcaggaggaatttcagcgtttgctagagtacactgacactaagccaaagtcgaaacgatttcgcggctctcaaagagaaaaatatgagatggtccctacctctgaaaacggagtgccgcaaccgcttcgagaacatgatgaaagttattggaggaaaaaattccgggaaattaccaacaaaaaactgtatttacctctgcgtatggtatttatcgtgttcttttttggcacagcaactcagctcgcagctatgagaccgtttatggtcggtgttctcaaacaatttggacttactgttgacaactacttagttttggttttgatctcctttttctacttcgtgggcgcgatgatgaacgtgatcttcgtgcgacgattgggcaagcgacgactcactttgtactgccaagcattggccacgctctccgtcctgcttttaggcgtgtacctcgcctatctcacggggccaaccaaggcccgagccatcgactggatcccgattttgctcttcgtttctttgttctttgccagtggctccagcattgctctcatcgcttggcaactttgcgcggaagtttttcccgtcgaaggccgggggactgcacaaggcctggttgcagcctgggcgtacttagtgaattttgtgatgtcgaagtcctatttgtacttggaaaggctggtccagctcaaaggagtcttttacttttatggtgcactctcagcgttgggatttttctactattggcgctatctaccggagaccgagggcaagtcacttgatcagatcgaaacttacttcacggagaactgtgatgagaaggacaaattcacaaaacggaaaaataatcgtggttag

SEQ ID NO: 2 - protein, 530 aa, Bemisia tabaci
MTNEMESSENQAESQTEDLIPKPKEIKYKNAGRSTFSQIVAMLVLACLLVDFGLELIIPTIVIGALHKNPDEALNLTDEQASWFGSILYFAHPIGALISGFLQELLGRKRSLLLVNIPMLVAWSTLYLASSVYQLYFVSAALGLCIGFCEAPLHSYIGEMSEPHVRGTLSAMGTASCLMGMLIMYLIGYLVHWRTAALISSFVPVITFLAIAQIPESPTWLVMNGREKEAQKALGWLRGWLKPEEVQEEFQRLLEYTDTKPKSKRFRGSQREKYEMVPTSENGVPQPLREHDESYWRKKFREITNKKLYLPLRMVFIVFFFGTATQLAAMRPFMVGVLKQFGLTVDNYLVLVLISFFYFVGAMMNVIFVRRLGKRRLTLYCQALATLSVLLLGVYLAYLTGPTKARAIDWIPILLFVSLFFASGSSIALIAWQLCAEVFPVEGRGTAQGLVAAWAYLVNFVMSKSYLYLERLVQLKGVFYFYGALSALGFFYYWRYLPETEGKSLDQIETYFTENCDEKDKFTKRKNNRG

SEQ ID NO: 3 - DNA, 1593 bp, Bemisia tabaci
  source 1..1593: mol_type = other DNA
atgaccaagaagatggagtcctcagaaaatccagccgaaagccagactgaagacctgctacctaagtcaaaagaaataaaatacaaaaatgccggcagatcgacattctcacagatcgtggctatgcttgtcttggactggttgctcgttgacttcgggctggagctgatcatacccaccatcgtgataggtgctcttcataaaaaccctgccgaagctctaaacttgaccgacgagcaagcatcgtggtttggaagcatcttgtatttcgcgcacccgatcggggctctgatctcagggttcctgcaggagcacctggggcggaagcggagtctcctgctggtgaacatccccatgcaggtggcctggagcacgctgtacctggccagctcagtctaccagctctactttgtctcggccacgcttggcctttgcatcggcttttgcgaggccccgctccactcgtacatcggcgagatgagcgaaccgcacgtgcgaggcagcctctcggccatgggcacggcctcttgcctcatgggcatgctcatcatgtaccttatcggctacctcgcccactggaggacggccgccttgatcagcagcttctttccggtcattacgttcctcgccattgcccagattccagaatcacccacttggttggtcatgaatggccgcgaaaaagaagcccaaaaagctttgggttggctgagaggctggctcaagccggaggaggttcaggaggaatttcagcgtttgctagagtacactgacactaagccaaagtcgaatcgatttcgcggctctcaaagagagaaatatgagatggtcccaacctctgaaaacggagtgccgcaaccgcttcgagatgatagtgaaagttattggaggaaaaaattccgggaaattaccaacaaaaaactgtatttaccactgcgtatggtatttatcgtgttcgtttttagtattgcaactcagctcgccgctatgagaccgtttatggtcggtgttctcaaacaatttggacttactgttgacaactacttagttttggttttgatctcctttttctactttgtgggcgcaatgatgaacgtgatcttcgtgcgacgattggggaagcgacgactcactttgtactgccaagcaatcgccacgctctccgttctgcttttaggcgtgtacctcgcctacctcacggggccaaccaaggtccgagccattgactggatcccaatttcgctcttcgtctctgtgttcttcttcagtggctccagcattgctctcatcgcttggcaactttgcgcagaagtttttcccgtcgaaggccgggggactgcacaaggccttgttgcagcctgggcgtactcggtgaattttgtgatgtcaaagtcctatttgtacttggaaaggctggtccagctcaaaggagtcttttatttttatggtgcactctcagtactgggctttttctactattggcgctatctaccggagaccgagggcaagtcacttgatcagattgaaacttacttcacggagaactgcgatgagaaggaaaaattcacaaaacgtaaaaataaccgtagttag

SEQ ID NO: 4 - protein, 530 aa, Bemisia tabaci
MTKKMESSENPAESQTEDLLPKSKEIKYKNAGRSTFSQIVAMLVLDWLLVDFGLELIIPTIVIGALHKNPAEALNLTDEQASWFGSILYFAHPIGALISGFLQEHLGRKRSLLLVNIPMQVAWSTLYLASSVYQLYFVSATLGLCIGFCEAPLHSYIGEMSEPHVRGSLSAMGTASCLMGMLIMYLIGYLAHWRTAALISSFFPVITFLAIAQIPESPTWLVMNGREKEAQKALGWLRGWLKPEEVQEEFQRLLEYTDTKPKSNRFRGSQREKYEMVPTSENGVPQPLRDDSESYWRKKFREITNKKLYLPLRMVFIVFVFSIATQLAAMRPFMVGVLKQFGLTVDNYLVLVLISFFYFVGAMMNVIFVRRLGKRRLTLYCQAIATLSVLLLGVYLAYLTGPTKVRAIDWIPISLFVSVFFFSGSSIALIAWQLCAEVFPVEGRGTAQGLVAAWAYSVNFVMSKSYLYLERLVQLKGVFYFYGALSVLGFFYYWRYLPETEGKSLDQIETYFTENCDEKEKFTKRKNNRS

SEQ ID NO: 5 - DNA, 1500 bp, Bemisia tabaci
  source 1..1500: mol_type = other DNA
atgtttaaaatttcgagaagcatcttccggcagacgctggcagccttttgctgttccatcggtccgatgacagtgggcgcaatcgcgggttggtcagcatccgctttcccaaaaatccgaaacgacgaactcaactttcggctgactcttttccaagaggcatgggtcatcaacacctactatgtgggcatcatgatgggccctctgctggccggcatcgccatggacgccatcggcaggaaaactaccctcctcctcttcagcatcttctccgtcgccaactggacgttggtcatcctcgcttcgaatgagtacatgctctatatcgcgcgggttttctctggactctgggctggcagcgttttcaccgtctgccctgctttccttgctgaggtactacaaccccacgtccgaggcagcctgggctcttttttgatgagcatgtacttcttaggcaacctctacgaatacatcatcggcccctacgtgtcatactccacattcggcatcgcctcctgcatcccctgcctgatctttgccgtcgcgtttctcttcatccctgaatccccctactactacatcatgaaaaaccaacgcggaaaagcagaggcctccctcagctggctgcgaggcgacgtcgacgtcaaccaagaactcgacgccatcgagacgtacgccgtggcgttcatgagaaaccgagggtcattcaaagacgtcttcctcaacgagaactaccgcgcggcactcatcaacgtccaggccatctacttcctccagaagctctgcgggatgttcaccgtgctcgcctacctgaccgtcatcatccctccatacgtgggtcctttcacgtctgagaactgcacccttatcgtgggggtcgttctttggatctcgaccaccctcgcagcttccctcatggatcgaattggtcggaagaggctcctcgtaatctcgaacgttggcatcatcgttacgatgacaattactggagcttggtactatttggacagtacggatttggatctctcggagactacttatgtgccgtttttggggttggtgatctatgggattttcttctgtctcggcctggggcctattcctaccctttaccaaggggagattctgccctcgaacattaaggctcgcgcttgcacggttacaacgatgtgctcggcgtgggcctctattttgaacacgactctgttcgccatttgtatcaggtatattggcctgtacatcaacttcttcctgttcgcggccacctcagtgttcggattgtacttcgcgaagtaccatttcacagaaacgagcgggaagacgctacaagagattcaagaagaactcatgaaaagacggcatcgagggaaattcacggatgacaagaagtcccccgtaaatgcaaaaccaaccatttacacagttccgatgccaaatgcaacggagaaggttgaaaccaagaaacatttcgagaaggatgctaagtggaccgacgaatga

SEQ ID NO: 6 - protein, 499 aa, Bemisia tabaci
MFKISRSIFRQTLAAFCCSIGPMTVGAIAGWSASAFPKIRNDELNFRLTLFQEAWVINTYYVGIMMGPLLAGIAMDAIGRKTTLLLFSIFSVANWTLVILASNEYMLYIARVFSGLWAGSVFTVCPAFLAEVLQPHVRGSLGSFLMSMYFLGNLYEYIIGPYVSYSTFGIASCIPCLIFAVAFLFIPESPYYYIMKNQRGKAEASLSWLRGDVDVNQELDAIETYAVAFMRNRGSFKDVFLNENYRAALINVQAIYFLQKLCGMFTVLAYLTVIIPPYVGPFTSENCTLIVGVVLWISTTLAASLMDRIGRKRLLVISNVGIIVTMTITGAWYYLDSTDLDLSETTYVPFLGLVIYGIFFCLGLGPIPTLYQGEILPSNIKARACTVTTMCSAWASILNTTLFAICIRYIGLYINFFLFAATSVFGLYFAKYHFTETSGKTLQEIQEELMKRRHRGKFTDDKKSPVNAKPTIYTVPMPNATEKVETKKHFEKDAKWTDE

SEQ ID NO: 7 - DNA, 1500 bp, Bemisia tabaci
  source 1..1500: mol_type = other DNA
atgttcaaagtgtcgaaaagcatcctccggcagacgttggcagccttttgctgttccatcggtcccatgacagtgggcgcaatcgcgggttggtcagcgtccgcgttcccaaaaatccgaaacgacgaactcaactttcggctgactcttttccaagaggcatgggtcatcaacttctactacgtcggcatcatgatggggcctctgctggccggcatcgccatggacgccgtcggcaggaaaactaccctcctccttttcagcatcttctccatcgccaactggacgttggtcattctcgcttccaatgagtacatgctctacctcgcgcgggttttctctggactctgggctggcagcgttttcaccgtctgtcctgcttttcttgctgaggtattacaaccccacgtccgaggcagcctgggctcctttttgatgagcatgtacttcttaggcaacctctacgaatacatcatcggcccctacgtgtcatactccacattcggcatcgcctcctgcatcccctgcctgatcttcgccgttgcgttcctcttcatccccgaatccccctactactacatcatgaaaaaccaacgcggaaaagcagaggccgctctcagctggctgcgcggcgacgtcgacgtcaaccaagaactcgacgccatcgagacgtacgccgtgacgttcatgagaaaccgaggatcgttcaaagacgtcttcctcaacgagaactaccgcgcggcgctcatcaacgtccaggccatctacttcctccagaagctctgcgggatgttcaccgtgcttgcttacctaaccgtcatcatccctccacacgtaggccctctcacgtccgagacctgcacccttatcgtgggggttgttctttggatctcgaccaccctcgcggcttccctcatggatcgaattggtcggaaaaggctcctcgttgtctctaacgtcggcatcgtcgttacaatgacagttactggatcttggtactatttggacagtacggatttggatctctcggagactacttatgtaccatttttggggttgctgatatatgggattttcttttgtctcggcttggggcctatccctaccatttatcaaggggagattctgccctcgaatattaaggcgcgcgcttgcacggttacaacgatgtgctcggcatgggcctctattttgaacacgactctgttcgccatttgtatcaggtatattggcctgtacatcaatttcttcctgttcgcggctacctcagtgttcggattgtacttcgcgaagtaccatttcatagaaacgagcgggaaaacgttgcaggagattcaagaagaactcatgaaaagacggcatcgagggaaattcacagatgacaagaagtcccccgtaaatgcgaaaccaaccatttatacagttccgatgccaaatgcaacggagaaggttgaaaccaagaaacatttcgagaaggatgctaagtggaccgacgaatga

SEQ ID NO: 8 - protein, 499 aa, Bemisia tabaci
MFKVSKSILRQTLAAFCCSIGPMTVGAIAGWSASAFPKIRNDELNFRLTLFQEAWVINFYYVGIMMGPLLAGIAMDAVGRKTTLLLFSIFSIANWTLVILASNEYMLYLARVFSGLWAGSVFTVCPAFLAEVLQPHVRGSLGSFLMSMYFLGNLYEYIIGPYVSYSTFGIASCIPCLIFAVAFLFIPESPYYYIMKNQRGKAEAALSWLRGDVDVNQELDAIETYAVTFMRNRGSFKDVFLNENYRAALINVQAIYFLQKLCGMFTVLAYLTVIIPPHVGPLTSETCTLIVGVVLWISTTLAASLMDRIGRKRLLVVSNVGIVVTMTVTGSWYYLDSTDLDLSETTYVPFLGLLIYGIFFCLGLGPIPTIYQGEILPSNIKARACTVTTMCSAWASILNTTLFAICIRYIGLYINFFLFAATSVFGLYFAKYHFIETSGKTLQEIQEELMKRRHRGKFTDDKKSPVNAKPTIYTVPMPNATEKVETKKHFEKDAKWTDE

SEQ ID NO: 9 - DNA, 2412 bp, Bemisia tabaci
  source 1..2412: mol_type = other DNA
atgggcagtataggcaatgatcaacctgctactgggaaaatcatcgtggtcgccaataggttaccgtttgtcttgaaacgaaatgaagccgggaaactcattcggattgccagtgctggtggtttagtcacagctgttgcacctgtggtcgtacgcggtaaaggactgtgggttggatggccaggaattcaccttgtaaacttaagtgaacccattccagaatcggatccagcagatcaaacacctacagctggtcttttatcagaaaaagtggtggctgtgcacgtagagcctgctatttttgatgcctattacaatggttgttgtaatggcacattttggcctctattccactcaatgccggatagaactattttcagtgcggataattggaaagcgtatcaggcagtaaatttagaatttgcagagaaaacagtaaatgctttaagaaggctgatagagactgaagaagaaaatgctgggactcctttagtctggttacatgattaccatctcatgctggcagccaacacaattcgcaattctgcagatgacctaggattaaaatgtaagcttggatttttcttacatataccttttcctccgtgggatatatttcgtcttttcccatgggcagatgaagtgctacaaggaatgctgggatgtgatatggtaggcttccatattgaggactattgtctgaacttcgttgattgctgccagcgaagattagggtgccgtgtcgacaggaaaggtcttcttgtggagcatggaggtcgcacagtcagagttcgaccgttacccattggtataccctacgaaagattcgtcgagttggctaaggcagcaccaccggtaatcacaaacaaagatcaaaaaactgtgctaggtgtcgacagattggattataccaaaggattggtccaccgcctcaaatcttttgaagttctcttagaaaaacatccggaacatagagaaaaggtggtactgatgcagattgctgttccatcacgaacagatgtcaaagaatatcaggacctgaaggaagaaatggaccaactcgttggcagaatcaatggtcgattcaccacacctaattggtctcccataaaatacatttacggatgtgtgtcgcaggatgaattggcagctttttacagggaagcggcagtagccttggtcactcctttgagagacggtatgaatttagtagcaaaagagttcgttgcttgccaaatcaacacaccacctggagttttgattgtatccccctttgctggtgccggagagatgatgcatgaagctctcatttgtaatccatatgagatcaatgaagcagccgaggtcatacacagagctttaacgatgccggaagatgaaagaactttgcgtatgaactccttgcgtaagagggaaaaagtgcatgatgtcgattattggatgaggtcctttttaaaaggaatgggaactctcatctctgaagatggcgatgatgttcttccaacaacaatgcagcctgtgactctggaagacttcgatgaatacctttccaaatacataggaaacaccgacaaattggcgcttatgttggattatgatggtaccctggcccctattgctcctcatccagatcttgccattttacctccagaaaccaaacatgttcttgaaaggctttccaatatgcctgaagtatatatttcaataatctctggacggaatgtcaataatgtcaaagcaatggttggtatcgaaggtttgacatatgctggtaatcatggtctagaaatccttcatccagacggaagcagattcgtacatcctatgcctgttgaatttgaagacaaggtcagcgacctgcttaaggttcttcaggagcaggtttgcaaggaaggtgcatgggtggaaaataaaggggctcttttaactttccattatcgggaaacgccattagatgtcagacaagaaatggtcgctcaagcaaaacttctcattgaacaagctggtttcaagcctgcccaagctcattgcgcaatcgaagcaaagccgcctgtgcaatggaacaaaggaagagccagtatatatattctacgaacagcttttggtctagattggtcggaacgcatccgaatcatctatgcaggtgatgacaccacagatgaagacgccatggaagcattgaaaggaatggctgctaccttcagagtaacgcaatctcagatcatccgcaccgttgcagaaaggcgtttgccaagtacggactctgttttgaccatgttaaaatgggtagaaagacacttcggcaagagactccctcgaggaaacagctatggtaataatctttgtaagaaaggcgatgtgatgaaaatggagatgagcttcaaaacagatgatatctctccaaccagttaa

SEQ ID NO: 10 - protein, 803 aa, Bemisia tabaci
MGSIGNDQPATGKIIVVANRLPFVLKRNEAGKLIRIASAGGLVTAVAPVVVRGKGLWVGWPGIHLVNLSEPIPESDPADQTPTAGLLSEKVVAVHVEPAIFDAYYNGCCNGTFWPLFHSMPDRTIFSADNWKAYQAVNLEFAEKTVNALRRLIETEEENAGTPLVWLHDYHLMLAANTIRNSADDLGLKCKLGFFLHIPFPPWDIFRLFPWADEVLQGMLGCDMVGFHIEDYCLNFVDCCQRRLGCRVDRKGLLVEHGGRTVRVRPLPIGIPYERFVELAKAAPPVITNKDQKTVLGVDRLDYTKGLVHRLKSFEVLLEKHPEHREKVVLMQIAVPSRTDVKEYQDLKEEMDQLVGRINGRFTTPNWSPIKYIYGCVSQDELAAFYREAAVALVTPLRDGMNLVAKEFVACQINTPPGVLIVSPFAGAGEMMHEALICNPYEINEAAEVIHRALTMPEDERTLRMNSLRKREKVHDVDYWMRSFLKGMGTLISEDGDDVLPTTMQPVTLEDFDEYLSKYIGNTDKLALMLDYDGTLAPIAPHPDLAILPPETKHVLERLSNMPEVYISIISGRNVNNVKAMVGIEGLTYAGNHGLEILHPDGSRFVHPMPVEFEDKVSDLLKVLQEQVCKEGAWVENKGALLTFHYRETPLDVRQEMVAQAKLLIEQAGFKPAQAHCAIEAKPPVQWNKGRASIYILRTAFGLDWSERIRIIYAGDDTTDEDAMEALKGMAATFRVTQSQIIRTVAERRLPSTDSVLTMLKWVERHFGKRLPRGNSYGNNLCKKGDVMKMEMSFKTDDISPTS

SEQ ID NO: 11 - DNA, 2411 bp, Bemisia tabaci
  source 1..2411: mol_type = other DNA
atgggcagtataggcaatgatcaacctgctactgggaaaatcatcgtggtcgccaataggttaccgtttgtcttgaaacgaaatgaagccgggaaactcattcggattgccagtgctggtggtttagtcacagctgttgcacccgtggtcgtacgcggtaaaggactgtgggttggatggccaggaattcaccttgtaaacttaagtgaacccattccagaatcggatccagcagatcaaactcctacagctggtcttttatcagaaaaagtggtggctgtgcacgtagagcctgctatttttgatgcctattacaatggttgttgtaatggcacattttggcctctattccactcaatgccggatagaactattttcagtgcggataattggaaagcgtatcaggcagtaaatttagaatttgcagagaaaacagtgaatgctttaagaaggctgatagagactgaagaagaaaatgctgggactcctttagtctggttacatgattaccatcttatgctggcagccaacacaattcgcaattctgcagatgacctaggattaaaatgtaagcttggatttttcttacatataccttttcctccgtgggatatatttcgtcttttcccatgggcagatgaagtgctacaaggaatgctgggatgtgatatggtaggcttccatattgaggactattgtctgaacttcgttgattgctgccagcgaagattagggtgccgtgtcgacaggaaaggtcttcttgtggagcatggagggcgcacagtcagagttcgaccgttacccattggtataccctacgaaagattcgtcgagttggctaaggcagcaccaccggtaatcacaaacaaagatcaaaaaaccgtgctaggtgtcgacagattggattataccaaaggattagtccaccgcctcaaatcttttgaagttctcttagaaaaacatccggaacacagagaaaaggtggtactgatgcagattgctgttccatcacgaacagatgtcaaagaatatcaggacctgaaggaagaaatggaccaactcgttggcagaatcaatggtcgattcaccacacctaattggtctcccataaaatacatttacggatgtgtgtcgcaggatgaattggcagctttttacagggaagctgcagtagccttggtcactcctttgagagacggtatgaatttagtagcaaaagagttcgttgcttgccaaatcaacacaccacctggagttttgattgtatccccctttgctggtgccggagaaatgatgcatgaagctctcatttgtaatccatatgagatcaatgaagcagccgaggtcatacacagagctttaacgatgccggaagatgaaagaactttgcgaatgaactccttgcgtaagagggaaaaagtgcatgatgtcgattattggatgaggtcctttttaaaaggaatgggaactctcatctctgaagatggcgatgatgttcttccaacaacaatgcagcctgtgactctggaagacttcgatgaatacctttccaaatacataggaaacaccgacaaattggcgcttatgttggattatgatggtaccctggcccctattgctcctcatccagatcttgccattttacctccagaaaccaaacatgttcttgaaaggctttccaatatgcctgaagtatatatttcaataatctctggacggaatgtcaataatgttaaagcaatggttggtatcgaaggtttgacgtatgctggtaatcatggtctagaaatccttcatccagatggaagcagattcgtacatcctatgcctgttgaatttgaagacaaggtcagcgacctgcttaaggttcttcaggagcaggtttgcaaggaaggtgcatgggtggaaaataaaggggctcttttaactttccattatcgggaaacgccattagatgtcagacaagaaatggtcgctcaagcaaaacttctcatcgaacaagctggtttcaagcctgcccaagctcattgcgcaatcgaagcaaagccgcctgtgcaatggaacaaaggaagagccagtatatatattctacgaacagcttttggtctagattggtcggaacgcatccgaatcatctatgcaggtgatgacaccacagatgaagacgccatggaagcattgaaaggaatggctgctaccttcagagtaacgcaatctcagatcatccgcaccgttgcagaaaggcgtttgccaagtacagactctgttttgaccatgttaaaatgggtagaaagacacttcggcaagagacttccccgaggaaacagctatggtagtaatctgtgtaagaaaggcgatgtgatgaaaatggagatgagcttcaaacagatgatatctctccaaccagttaa

SEQ ID NO: 12 - protein, 803 aa, Bemisia tabaci
MGSIGNDQPATGKIIVVANRLPFVLKRNEAGKLIRIASAGGLVTAVAPVVVRGKGLWVGWPGIHLVNLSEPIPESDPADQTPTAGLLSEKVVAVHVEPAIFDAYYNGCCNGTFWPLFHSMPDRTIFSADNWKAYQAVNLEFAEKTVNALRRLIETEEENAGTPLVWLHDYHLMLAANTIRNSADDLGLKCKLGFFLHIPFPPWDIFRLFPWADEVLQGMLGCDMVGFHIEDYCLNFVDCCQRRLGCRVDRKGLLVEHGGRTVRVRPLPIGIPYERFVELAKAAPPVITNKDQKTVLGVDRLDYTKGLVHRLKSFEVLLEKHPEHREKVVLMQIAVPSRTDVKEYQDLKEEMDQLVGRINGRFTTPNWSPIKYIYGCVSQDELAAFYREAAVALVTPLRDGMNLVAKEFVACQINTPPGVLIVSPFAGAGEMMHEALICNPYEINEAAEVIHRALTMPEDERTLRMNSLRKREKVHDVDYWMRSFLKGMGTLISEDGDDVLPTTMQPVTLEDFDEYLSKYIGNTDKLALMLDYDGTLAPIAPHPDLAILPPETKHVLERLSNMPEVYISIISGRNVNNVKAMVGIEGLTYAGNHGLEILHPDGSRFVHPMPVEFEDKVSDLLKVLQEQVCKEGAWVENKGALLTFHYRETPLDVRQEMVAQAKLLIEQAGFKPAQAHCAIEAKPPVQWNKGRASIYILRTAFGLDWSERIRIIYAGDDTTDEDAMEALKGMAATFRVTQSQIIRTVAERRLPSTDSVLTMLKWVERHFGKRLPRGNSYGSNLCKKGDVMKMEMSFKTDDISPTS

SEQ ID NO: 13 - DNA, 1410 bp, Bemisia tabaci
  source 1..1410: mol_type = other DNA
atggctcccgtcactgccgagctgctcgatcagcactgctctgaagtcaaacccctaaaactggagaacaaatggaaggcggagcaggtggaaaaaatgctacaaagattcaagttggatggaaaaacaatgttaaaaatggcagaaatttttcaaagggaaatggaattaggtcttgcagataaaccatcgtctcttttaatggaaaatacatatgttcctgagttgcccaatggaacagaggaaggcaaatatttggcattggatttaggaggcaccaactttcgtgtcattcttgtggatttagaaaaaggaagaatcgtcaatgaaagagtgaaacactatcatatcagtgatgaactcagattgaattgtggcattaaactttttgactttctggcagaatgtatctctgaattcgtcattgaaaatggtcttgcagatcgagtattgccaatgggttttacattctctttcccaatgcatcagcatgcccttgatagtggaattttaaaagtatggacaaaatcgtttgcctgtcctggtgttgaagggaaagatgctgtcaagctcttgaatgaggctattcacaggcgtggagataaccatgttgaattgatttgtgtccttaatgataccacaggtacgctgatgcaaggtgctatgttggataaagacactgctattggattaatccttggaactggtacaaatgcttgttaccttgagcgggcagagagagttcaacactgggaagttgaaaggcatggagaaaaacaggttgtcatagacattgagtggggtgcatttggtgataatggctgcatcgattttgtcaaaactgaatttgatagaactgtcgatgaaaaatctttaataccaaaatcgtttacgtttgaaaagaaaattggtggaaaattcatgggagaacttgttcaagtagtcttatgcagtttgacaagggaaggacatctattcaatggcataccatcagataaacttcttcagcatggtcacttccgaaccagttttgtgtctggtattgaagaggactcgatcaatggcaccaaagggaacacggaaaaaattctagatgatttaggactagattatgacgatgatgatgtcaaaattgttcaatatatctgtcatttggtgtctcttcgggcagctcagtttgttgcaatctgtgtgtctcaaatattaatccgaatgagcaaaccccaggttacagtcgctgttgacggttctctgttcaaattccatccaagactcagaatatggatggaaaaatacacaaagctcctggctcccagtcatccgttcaagattgttctggttcaagacggaagtggaaaaggtgctgctttagtttctgccattgccaagagaataactgaaaaatactga

SEQ ID NO: 14 - protein, 469 aa, Bemisia tabaci
MAPVTAELLDQHCSEVKPLKLENKWKAEQVEKMLQRFKLDGKTMLKMAEIFQREMELGLADKPSSLLMENTYVPELPNGTEEGKYLALDLGGTNFRVILVDLEKGRIVNERVKHYHISDELRLNCGIKLFDFLAECISEFVIENGLADRVLPMGFTFSFPMHQHALDSGILKVWTKSFACPGVEGKDAVKLLNEAIHRRGDNHVELICVLNDTTGTLMQGAMLDKDTAIGLILGTGTNACYLERAERVQHWEVERHGEKQVVIDIEWGAFGDNGCIDFVKTEFDRTVDEKSLIPKSFTFEKKIGGKFMGELVQVVLCSLTREGHLFNGIPSDKLLQHGHFRTSFVSGIEEDSINGTKGNTEKILDDLGLDYDDDDVKIVQYICHLVSLRAAQFVAICVSQILIRMSKPQVTVAVDGSLFKFHPRLRIWMEKYTKLLAPSHPFKIVLVQDGSGKGAALVSAIAKRITEKY

SEQ ID NO: 15 - DNA, 1410 bp, Bemisia tabaci
  source 1..1410: mol_type = other DNA
atggctcccgtcactgccgagctgctcgatcagcactgctctgaagtcaaaccgctgaaactggagaataaatggaaggcagagcaggtagaaaaaatgcttcaaagattcaagttagatggaaaaacaatgttaaaaatggcagaaatttttcaaagggaaatggaattaggtcttgcggataaaccatcgtctcttttaatggaaaatacgtatgttcctgagttgcccaatggaacagaggaaggcaaatatttggcattggacttaggaggcaccaacttccgtgtcattcttgtggatttagaaaaaggaagaatcgtcaatgaaagagtgaaacactatcatataagtgatgaactcagattgaattgtggcatcaaactttttgactttctggcagaatgtatctctgaatttgtcattgaaaatggtcttgcagatagagtattgccaatgggttttacattttctttcccaatgcatcagcatgcccttgatagtggaattttaaaagtatggacaaaatcgtttgcctgtcctggcgttgaagggaaggatgctgtcaagctcttgaatgaggctattcacaggcgtggagataaccatgttgaattgatttgtgtccttaatgataccacaggtacactgatgcaaggtgctatgttggataaagatactgccattggattaatccttggaactggtacaaatgcctgttaccttgagcgggcagagagagttcaacactgggaagttgagaggcatggcgaaaagcaggttgtcatagacattgagtggggtgcgtttggtgataatggctgcatcgattttgtcaaaactgaatttgatagaactgtcgatgaaaaatctttaataccaaaatcgttcacgtttgagaagaaaattggtggaaaattcatgggagaacttgttcaagtaatcctatgcagtttgacaagggaaggacatctattcaatggcattccgtcagataaacttcttgagcatggtcacttccgaaccagttttgtgtccggtattgaagaggactccatcaatggcaccaaggggaacacggaaaaaattctagacgatctaggattagattatgacgatgatgatgtcaaaattgttcaatacatatgtcatttggtgtctcttcgcgcagctcagtttgttgcaatctgtgtgtctcaaatattaatccgaatgagcaaaccgcaggttacggttgctgttgacggttctctgttcaaattccatccaagactcagaatatggatggaaaaatacacaaagcttctggctcccagtcatccgttcaagattgttctggtccaagatggaagtggaaaaggtgctgctttggtttctgccattgccaagagaataactgaaaaatactga

SEQ ID NO: 16 - protein, 469 aa, Bemisia tabaci
MAPVTAELLDQHCSEVKPLKLENKWKAEQVEKMLQRFKLDGKTMLKMAEIFQREMELGLADKPSSLLMENTYVPELPNGTEEGKYLALDLGGTNFRVILVDLEKGRIVNERVKHYHISDELRLNCGIKLFDFLAECISEFVIENGLADRVLPMGFTFSFPMHQHALDSGILKVWTKSFACPGVEGKDAVKLLNEAIHRRGDNHVELICVLNDTTGTLMQGAMLDKDTAIGLILGTGTNACYLERAERVQHWEVERHGEKQVVIDIEWGAFGDNGCIDFVKTEFDRTVDEKSLIPKSFTFEKKIGGKFMGELVQVILCSLTREGHLFNGIPSDKLLEHGHFRTSFVSGIEEDSINGTKGNTEKILDDLGLDYDDDDVKIVQYICHLVSLRAAQFVAICVSQILIRMSKPQVTVAVDGSLFKFHPRLRIWMEKYTKLLAPSHPFKIVLVQDGSGKGAALVSAIAKRITEKY

SEQ ID NO: 17 - DNA, 1596 bp, Bemisia tabaci
  source 1..1596: mol_type = other DNA
atgacgtctcaaatcgccgatgaatttttactcggtcacgctgaaaaagctgtatcgccaactgacagccctgaccaacctcgattttatagaaaggtgcgaggccaccaacgagttccctcaggttcgcaagaattcaaagacctcaccaaacgtgacgctttggaacaaatgaaaaacgatattgaacggttagtttcaacggtgcctgaggcgcagcgagaagccacgaaaaaggagttggatgggttctcgtctctatttgaacgatacctccaagagaaaggtccatctctctcttggaatgatattcagaaacttcctgaaaatgcggtcagagactatgcatcattggaaaccccatcgacagaagatgcccggaacatgttggaaaaattggtagttgtcaagttaaatggtggtcttggaacatccatgggttgcaggggtccaaaatctgtaattcctgtccggagtgatcttacgttcctagatcttactgtccaacagattgaatacctaaacaaaacatacaaagttaatgtgcctttggttttaatgaactctttcaatactgatgccgatacacaaaaaataattcgcaagtacaaaggtgttcaagttgaaatctacacatttagtcaaagttgttttcccagaatagagaaagagtcgctcatgcctgttcccaaagactgtcatgttgaaaagaatttagaagcgtggtaccctcctggtcatggtgacttttatgagagcttcagtaactcagggcttctcaagaagtttatcaaacaggggcgaaaattctgctttctctccaatattgacaacttgggagccacagtggatctgaatatcttgaatatgctgttgaaatcaaattcaacatctgaatttttaatggaagtcaccgacaaaaccagggctgatgtgaagggaggtaccctcattcaatatgacaacaagctgagacttctggaaattgcacaagtacctaaagaacacgttgatgaatttaaatcagtgaaaactttcaaatttttcaatacaaacaatttgtgggtggacttagaagccattgaaagagttgtctcagagggtggactaaacatggaaattatcatcaatcataagtcactaggaaatggtacaaacgtaatccaattagaaacagcagttggtgccgccatgaaagctttcaatcacagtgtcggcttaaatgtaccgaggagtcgattcttacccgtcaagaaaacatcagatttaatgctagtcatgagtaacctgtatgatttaaaccatgggtcattgatcatgagcccgaagagaatgttcccgacaactcctctgatcaagcttggagacgcacactttgccaaagttaaagattttctcagtcgtttcgccaatatcccagacttgctggagttggaccatttaactgtctctggagatgttacattcggaagaggagtttcattcaagggcactgtcataatcatcgccaatcatggagacaggattgacatcccagcagaatcacttttagaaaacaaaattatctccggaaacttgagaattttggaccactag

SEQ ID NO: 18 - protein, 531 aa, Bemisia tabaci
MTSQIADEFLLGHAEKAVSPTDSPDQPRFYRKVRGHQRVPSGSQEFKDLTKRDALEQMKNDIERLVSTVPEAQREATKKELDGFSSLFERYLQEKGPSLSWNDIQKLPENAVRDYASLETPSTEDARNMLEKLVVVKLNGGLGTSMGCRGPKSVIPVRSDLTFLDLTVQQIEYLNKTYKVNVPLVLMNSFNTDADTQKIIRKYKGVQVEIYTFSQSCFPRIEKESLMPVPKDCHVEKNLEAWYPPGHGDFYESFSNSGLLKKFIKQGRKFCFLSNIDNLGATVDLNILNMLLKSNSTSEFLMEVTDKTRADVKGGTLIQYDNKLRLLEIAQVPKEHVDEFKSVKTFKFFNTNNLWVDLEAIERVVSEGGLNMEIIINHKSLGNGTNVIQLETAVGAAMKAFNHSVGLNVPRSRFLPVKKTSDLMLVMSNLYDLNHGSLIMSPKRMFPTTPLIKLGDAHFAKVKDFLSRFANIPDLLELDHLTVSGDVTFGRGVSFKGTVIIIANHGDRIDIPAESLLENKIISGNLRILDH

SEQ ID NO: 19 - DNA, 1596 bp, Bemisia tabaci
  source 1..1596: mol_type = other DNA
atgacatctcaaatcgccgatgaatttttactcggccacgctgaaaaagctgtannnccaactgacagccctgatcaacctcgattttatagaaaggtgcgaggccaccaacgagttccctcaggttcgcaagaattcaaagacctcaccaaacgtgacgctttggaacaaatgaaaaacgatatcgagcggttggtttcaacggtgcctgaggcgcagcgagaagccacgaaaaaggagttggatgggttctcgtctctatttgaacggtacctccaagagaaaggtccatctctctcttggaatgatattcagaaacttcctgaaaatgcggtnagagactatgcctcattggaaaccccatcgacagaagatgcccggaacatgttggaaaaattggtggtggtcaagttaaatggtggtcttggaacatccatgggttgcaggggtccaaaatctgtaattcctgtccggagtgatcttacgttcctagatcttactgtccaacagattgaatacctaaacaaaacatacaaagttaatgtgcctttggttttaatgaactctttcaacactgatgccgatacacagaaaataattcgcaagtacaaaggtgttcaagttgaaatctacacatttagtcaaagttgtttccccagaatagagaaagagtcgctcatgcctgttcccaaagactgtcatgttgaaaagaatttagaagcgtggtaccctcctggtcatggtgacttttatgagagcttcagtaactcagggcttctcaagaagttcataaaacaggggcgaaaattctgctttctctccaatattgacaacttgggagccacagtggatctgaatatcttgaatatgctgttgaaatcaaattcaacatctgaatttttaatggaagtcacagacaaaaccagggctgatgtgaagggaggtaccctcattcagtacgacaacaagctgagacttctggaaattgcacaagtacccaaagaacacgtcgatgaatttaaatcagtgaaaactttcaaatttttcaatacaaacaatttgtgggtggacctagaagccattgaaagagttgtctctgagggtggactaaacatggaaattatcatcaatcataagtcactaggaaatggtacgaacgtaatccaattagaaacagcagttggtgccgccatgaaagctttcaatcacagtgttggcttaaatgtaccgaggagtcgattcttacccgtcaagaaaacatccgacttaatgctagtcatgagtaacctgtatgatttaaaccatgggtcattgatcatgagcccgaagagaatgttcccgacaactcctcttatcaagcttggagatgcacactttgccaaagttaaagattttctcagtcgtttcgccaatatcccagacttgctggagttagaccatttaaccgtctctggagatgttacattcggaagaggagtctcattcaagggcactgtcataatcatcgccaatcatggagacaggattgacatcccagcagaatcacttttagaaaacaaaattatctccggaaacttgagaattttggaccactag

SEQ ID NO: 20 - protein, 531 aa, Bemisia tabaci
MTSQIADEFLLGHAEKAVXPTDSPDQPRFYRKVRGHQRVPSGSQEFKDLTKRDALEQMKNDIERLVSTVPEAQREATKKELDGFSSLFERYLQEKGPSLSWNDIQKLPENAVRDYASLETPSTEDARNMLEKLVVVKLNGGLGTSMGCRGPKSVIPVRSDLTFLDLTVQQIEYLNKTYKVNVPLVLMNSFNTDADTQKIIRKYKGVQVEIYTFSQSCFPRIEKESLMPVPKDCHVEKNLEAWYPPGHGDFYESFSNSGLLKKFIKQGRKFCFLSNIDNLGATVDLNILNMLLKSNSTSEFLMEVTDKTRADVKGGTLIQYDNKLRLLEIAQVPKEHVDEFKSVKTFKFFNTNNLWVDLEAIERVVSEGGLNMEIIINHKSLGNGTNVIQLETAVGAAMKAFNHSVGLNVPRSRFLPVKKTSDLMLVMSNLYDLNHGSLIMSPKRMFPTTPLIKLGDAHFAKVKDFLSRFANIPDLLELDHLTVSGDVTFGRGVSFKGTVIIIANHGDRIDIPAESLLENKIISGNLRILDH

SEQ ID NO: 21 - DNA, 4602 bp, Bemisia tabaci
  source 1..4602: mol_type = other DNA
atggatcactatttttgcaactcacctctgtgggatttaaatgtgacatggaacacaagtgatcccaatttcactccatgttttcaagcaacaattctgtcttgggtgccttgcctatttttgtggttttttgcaccgttttcagttcggagtctaatgaaacgccagacatggacagtcccttgggcttggctgaatacggcaaaatttctgttgaccttacttttagtatttttatcattagttgacctcttttactctgttatctcgtattttgatgggagaccattttatctagtggattttatcacccctctcctaaagactgtcacttttgctttagctgcttctcttctgattttgtataaactccgaggtgtcccctcctcagccttgttgttcttgttctggtttttgtcagtcatctgtggtgctccacgatataaatatgaattggaccgtgcttatcgtcagaatttgggcgaagcagttgttcctttcgtcagttacatggtatattatgccatagtacttggtttgttcatcctgaatttttttgccgatctcttacccatctcaccttcctattcaccgaaagaaaaaagttctccgtactcgcaagcttcctttccatcattggtgacattctcatggtttgatcctcttgcatggaaaggttatagacgccccattgtccttgaagacttgtggtctctcgcagaagatgactccgttaaaaaaatcgcacctaaattcaacaagcattgcaggaagcgatcgaataaaaatcctcagtctagaaacgttaaggaaggcacaaccaggactgcaaatggtattgagctgaagccctattctgacaaagtagcctccaaggaagtttacccgtccatttggttcccattaatcaaaactttttggccatctgttttgtttggatttagtcttaaactttgcaatgacattctcatttttgtgagccctcatatattgaaatcactgattagctttatgggtagtgaagaggaaatttggcatggctatttctatgcaggagggctatttattgctgcttcctttcagacactcttccttgctcattacttcaaccgcatgaggataacagggcttaggatacgagttgctctcacgtctgctatttatcgcaaggccttaaatatatccaatgcatctcggaaagagtcaacagttggcgaaattgtgaatcttatgtctgttgatgcgcaacgatttatggaactaatgcctcacctatctttgctttggtctgcaccattgcagatatctctggctgtctttttcctgtgggagattttaggaccatctgtcttgggaggtttagttgtgatgatcctagtaattttgatcaatattccaattgcaaggaaaagtgatgcattgaccatgaaacaaatgcgatacaaggatgaacgtatcaaattaatgaatgagattttatctggaatcaaagtgctgaagttgtatgcatgggaaacaagctttgaaaagcacatccagaagattcgaatgaaggaggtgaacacactaaaacaactcgcaatcctagggtcaacaactgccttcatctggtcttgtgcaccacttcttgtagcactgacaacatttggtgtttatttattcattgacgatgagcactcattagatgcagaaaagacttttgcaacagtggcgttgttcaatattctcaagttcccacttgtcatgttgccaaacatgatctccaatcttgtccagacaatggtttctgttagaagattaaataagtttatgaactctgatgaattggacccgaattgtgtgacacattctgcctctgaaaaggagcctctggtaatcgaaaaaggaaattttggatggggaacatcagtgcctgtcctacatgatatagatatcaagatttttgataagtctcttgtcgcagtcgttggaactgttggttgtggaaaatcttcactacttgcagcatttctaggtgaaatggatctcatcagcgggagagtgaacacaaagggttccattgcatatgttactcagcaagcatggattcaaaatgcaactcttcgtgaaaacattctatttgggaaggagtatgattacaaaacatacaataaggtcatcaaagcatgtgccttgaggagtgattttgcgttgttgccaggtggagaccaaacagagattggtgaaaaaggtataaatttaagtggaggacaaaagcaacgagtcagtttagctcgagctgtgtatagtgatgctgatgtatattttcttgatgatcctttaagtgcagtggatagtcatgtgggcaagcacatttttgagaatgtaatcggacctcagggacttcttaagaataaaacacgggtccttgtcactcatggattgacctatctaccagatgtggatgagatatatgtcttgaaaaatggaatgatatcagagaaaggaacctacaaacagctcattgacaagaaaggcgcttttgccgagttcattcaaacccacatgttggagagtgaaaatgatgataaaccagcagaaggaacgcaagacatatcgcgaccgtcctctccacttgtgtatcaaagagtccaagatagatcacgtccctcttctccagccttgttacgtaaaagaagtcatagtgccagtgaaagaggatccaccttgtccttacggcaattttcgtacgaagaaggatctaattttgtggagcaggatgatgacaggaggaacaagataattgacgaagaaatagccatgacaggaaatataaaatggtctgtcttctcccattacatgcgagcaatgggttggctctttgttttcctggcatttgcttttaccctacttttccaagtttgcgccgttgggtcaaatctttggctgtctatatggacgtctcgaaataatactctaccagatggatcacaggatccagaagaaagaaattggtttcttgaagtctacgccattctaggactttccgcaggtctgtgtgcaattgtagctgatcttgcaccaagaattgggtgtgtgaaagcaagtcgggctcttcatggttacctgttacatggtatcttgagagctcctctcacattcacagacacaacccctattggacgaatcctttcccgcttctcaaaagatgtagacactatggataataaattacccatggaaatatctgacactctctattgtgttatggaggtgataggggtcttgttcattatcacctatgccacacctccatttttatggtgcattgtgcctctagttatactctactacattcttcagaggttttacacggccacatcacgacagctacaaagattagagtccatttcgcggtctccaattttttcccacttcagtgaaactgttgcaggagctcaagttatcagagcgtatggggctgtaaaccgatttattcaagaatatgagaagagaatggatgccaaccaaatctgtttccatccaactgtgatatcaatcagatggctgtcagttcgattagaaactattggtaacttgataatattttgtgctgccttttttgctgttttagcgcgaggcagcttatcagctggtcttgtcggcttgtctgttagctacgcccttcaggtaactggagcattgaattggtttgttcgtatggcatcaagtgttgaaacagatgttgttgcaattgagcgtgtgaaagagtacactgaaatccaagaggaagcaaaatgggatgtgccagaaacattgcctccagctaagtggccccaaaaaggaaatattgtgtttgaaaatttcaaagttcgctacaggagtgaattggatttggttctgaaagggttgaatttctctgtcaatgaaagagaaaaggtcggcattgtgggacgcactggtgctggaaaatcctcattgacccttgctttattccgcatcattgaagcagcagaaggagccatctatatagatggtattgatatttctactcttggactccacacactcagatctagactgaccgttatcccacaagatccagttcttttctctgggagcttgaggatgaatctagatccgctaggagtctactctgatacaaagatatggcatgcattagagttgtctcacttgaaggagtttgtgaagtcccaagcagcaggtttgcagcatgaaatagctgaaggtggtgaaaacctcagtgttggtcaaaggcaactcatttgtttagcccgggcactcttacgaaaaactaaagtacttattttggacgaagcaacagcagctgttgatgtagagacagacgatcttatccaaaccaccatccgtcaggagttcagcgactgcacaatattgacgattgctcatcggttaaacacaatcatggattccgacagagttctggtattggatcatggtctaataatggagtacgactctccattaaaccttctcaagaatccagcgtcccttttccactcactcgttaaagatgcaggcttggatgtagaaaccattctctccacccaaagcatgaatggaagacaactggtggtctga

SEQ ID NO: 22 - protein, 1533 aa, Bemisia tabaci
MDHYFCNSPLWDLNVTWNTSDPNFTPCFQATILSWVPCLFLWFFAPFSVRSLMKRQTWTVPWAWLNTAKFLLTLLLVFLSLVDLFYSVISYFDGRPFYLVDFITPLLKTVTFALAASLLILYKLRGVPSSALLFLFWFLSVICGAPRYKYELDRAYRQNLGEAVVPFVSYMVYYAIVLGLFILNFFADLLPISPSYSPKEKSSPYSQASFPSLVTFSWFDPLAWKGYRRPIVLEDLWSLAEDDSVKKIAPKFNKHCRKRSNKNPQSRNVKEGTTRTANGIELKPYSDKVASKEVYPSIWFPLIKTFWPSVLFGFSLKLCNDILIFVSPHILKSLISFMGSEEEIWHGYFYAGGLFIAASFQTLFLAHYFNRMRITGLRIRVALTSAIYRKALNISNASRKESTVGEIVNLMSVDAQRFMELMPHLSLLWSAPLQISLAVFFLWEILGPSVLGGLVVMILVILINIPIARKSDALTMKQMRYKDERIKLMNEILSGIKVLKLYAWETSFEKHIQKIRMKEVNTLKQLAILGSTTAFIWSCAPLLVALTTFGVYLFIDDEHSLDAEKTFATVALFNILKFPLVMLPNMISNLVQTMVSVRRLNKFMNSDELDPNCVTHSASEKEPLVIEKGNFGWGTSVPVLHDIDIKIFDKSLVAVVGTVGCGKSSLLAAFLGEMDLISGRVNTKGSIAYVTQQAWIQNATLRENILFGKEYDYKTYNKVIKACALRSDFALLPGGDQTEIGEKGINLSGGQKQRVSLARAVYSDADVYFLDDPLSAVDSHVGKHIFENVIGPQGLLKNKTRVLVTHGLTYLPDVDEIYVLKNGMISEKGTYKQLIDKKGAFAEFIQTHMLESENDDKPAEGTQDISRPSSPLVYQRVQDRSRPSSPALLRKRSHSASERGSTLSLRQFSYEEGSNFVEQDDDRRNKIIDEEIAMTGNIKWSVFSHYMRAMGWLFVFLAFAFTLLFQVCAVGSNLWLSIWTSRNNTLPDGSQDPEERNWFLEVYAILGLSAGLCAIVADLAPRIGCVKASRALHGYLLHGILRAPLTFTDTTPIGRILSRFSKDVDTMDNKLPMEISDTLYCVMEVIGVLFIITYATPPFLWCIVPLVILYYILQRFYTATSRQLQRLESISRSPIFSHFSETVAGAQVIRAYGAVNRFIQEYEKRMDANQICFHPTVISIRWLSVRLETIGNLIIFCAAFFAVLARGSLSAGLVGLSVSYALQVTGALNWFVRMASSVETDVVAIERVKEYTEIQEEAKWDVPETLPPAKWPQKGNIVFENFKVRYRSELDLVLKGLNFSVNEREKVGIVGRTGAGKSSLTLALFRIIEAAEGAIYIDGIDISTLGLHTLRSRLTVIPQDPVLFSGSLRMNLDPLGVYSDTKIWHALELSHLKEFVKSQAAGLQHEIAEGGENLSVGQRQLICLARALLRKTKVLILDEATAAVDVETDDLIQTTIRQEFSDCTILTIAHRLNTIMDSDRVLVLDHGLIMEYDSPLNLLKNPASLFHSLVKDAGLDVETILSTQSMNGRQLVV

SEQ ID NO: 23 - DNA, 4602 bp, Bemisia tabaci
  source 1..4602: mol_type = other DNA
atggatcactatttttgcaactcacctctgtgggatttaaatgtgacatggaacaccagtgatcctaatttcactccatgtcttcaagcaacaattctgtcatgggtgccttgcctatttttgtggttttttgcaccattttcagttcggagtctaatgaaacgccagacatggacagtcccttgggcttggctgaatacggcaaaatttctgttgaccttacttttaatatttttatcattagttgacctctcttactctgttatctcgtattttgacgggagaccattttatctagtggattttatcacccctctcctaaagactgtcacttttgctttagctgctgctcttctgcttttgtataaacttcgaggtgtcccctcctcagccttgttgttcttgttttggtttttgtcagtcatctgtggtgctccacgatataaatatgaattggaccgtgcttatcgtcagaatttgggcgaagcagttgttcctttcgtcagctacatggtatattatgccatggtacttggtttgttcatcctaaatttttttgctgatctcttacccatctcaccttcctattcaccgaaagaaaaaagttctccgtactcgcaagcctcctttccatcattggtgacattctcatggtttgatcctcttgcatggaaaggttacagacgccccattgtcgttgaagacttgtggtctctcgcagaagatgactctgtaaaaaaaattgcacctaaattcaacaagcattgcaggaagcgatcaaataaacatcctcagtctagaaacgttaaggaaggcacaaccaggactgcaaatggtattgagctgaagccctattccgacaaagcagcctccaaggaagtttacccatccatttggttcccattaattaaaactttttggccatccgttttgtttggatttagtcttaaactttgcaatgacattctcatttttgtgagccctcatatattgaaatcactgattagctttatgcaaagtgaggaggaagtttggcatggctatttctatgcaggagggctatttatcgctgcttcctttcagacactcttccttgcccattacttcaaccgcatgaggataacagggcttaggatacgagttgctctcacgtctgctatttatcgtaaggctctaaatatatccaatgcatctcggaaagagtcaacagtcggcgaaattgtgaatcttatgtctgtcgatgcgcaacgatttatggaactaatgcctcacctatctttgctttggtctgcaccattgcagatttctctggcggtctttttcctgtgggagattttaggaccatctgtcttgggaggtttagttgtgatgatcctagtaattttgatcaatattccaattgcaagaaaaagtgatgcattgaccatgaaacaaatgcgatacaaggatgaacgtatcaaattaatgaatgagattttatctggaatcaaagtgctgaagttgtatgcatgggaaacaagctttgaaaagcacatccagaagattcgaatgaaggaggtgaacacactaaaacaactcgcaatcttagggtcaacaactgccttcatctggtcttgtgcaccacttcttgtagcactgacaacatttggtgtttatttattcattgacgatgagcactcattagatgcagaaaagacttttgcaacagtggcgttgttcaatattctcaagttcccacttgtcatgttgccgaacatgatctccaatcttgtccagacaatggtttctgttagaagattaaataagtttatgaactctgatgaattggacccgaattgtgtgacacattctgcctctgaaacggagcctctggtaatcgaaaaaggaaattttggatggggaacaacagtacctgtcctacatgatatagatatcaagatttttgataagtctcttgtcgcagtcgttggaactgttggttgtggaaaatcttcactacttgcagcatttctaggtgaaatggatctcattagcgggagagtcaacacaaagggttccattgcatatgttactcagcaagcatggattcaaaatgcaactcttcgtgaaaacattctatttgggaaggagtatgattacagaacatacaataaggtcatcaaagcatgtgctttaaggagtgattttgcattgttgccaggtggagaccaaacagaaattggtgaaaaaggtattaatttaagtggaggacaaaaacaacgagtcagtttagctcgagctgtgtatagtgatgctgatgtatattttcttgatgaccctttaagtgcagtggatagtcatgtgggcaagcacatttttgaaaatgtaattgggcctcagggacttcttaagaataaaacacgggtccttgtcactcatggattgacctatctaccagatgtggatgagatatatgtcttgaaaaatggaatgatatcagagaaaggaacctacaaacagctcattgacaaaaaaggcgcttttgccgagttcattcaaacccacatgttggagagtgaaaatgatgataaaccagcagaaggaacgcaagacatatcgcgaccgtcctctccacttgtttatcaaagagtccaagatagatcacgtccctcttctccagccctgttacgtaaaagaagtcatagtgccagtgaaagaggatccaccttgtccttacggcaattttcgtacgaagaaggatctaattttgtggagcaggatgatgacaggaggaacaagataattgaagaagaaatagccatgacaggaaatataaaatggtctgtcttctcccattacatgcgagcaatgggttggctctttgttttcctggcatttgcttttaccctacttttccaagtttgtgccgttgggtcaaatctttggctgtctatatggacgtctcgaaataatactctaccagatggatcacaggatccagaagaaagaaattggttcctggaagtctacgccattctaggactttccgcaggtttgtgtgcaattgtagctgatcttgcaccaagaattgggtgtgtaaaagcaagtcgggctcttcatggttacctgttacatggtatcttgagagctcctctcacattcacagacacaacccctactggacgaatcctttcccgcttctcgaaagatgtagacactatggataataaattaccaatggaaatatcggacactctctattgtgtcatggaggtgataggggtcttgttcattatcacctatgccacacctccatttttatggtgcattgtgcctttagttattctctactatattcttcagaggttttacacggccacatcacgacagttacaaagattagagtccatttcgcggtctccaattttttcccacttcagtgaaactgttgcaggagctcaagttatcagagcatatggagctgtaaaccggtttattcaagaatatgagaagagaatggatgccaaccaaatctgtttccatccaactgttatatcaatcagatggctgtcagttcgattagaaactatcggtaacttgataatattttgtgctgccttttttgctgttttagcgcgaggcagcttatcagctggtcttgtcggcttgtctgttagctacgcccttcaggtaactggagcattgaattggtttgtgcgtatggcatcaagtgttgaaacagatgttgttgcaattgagcgtgtgaaagagtacactgaaatccaagaggaagcaaaatgggatgtgccagaaacattgcctccagctgagtggccccaaaaaggaattattgtgtttgaaaatttcaaagttcgttacaggagtgaattggatttggttctgaaagggttgaatttctctgtcaatgaaagagaaaaggtcggcattgtgggacgcactggtgctggaaaatcctcattgacccttgctttgttccgcatcattgaagcagcagaaggagccatctacatagatggcattgatatttctactcttggactccacacactcagatctagactgaccgtcatcccacaagatccagttcttttctctgggagtctgaggatgaatctagatccgctaggagtctactctgatacaaagatatggcatgcattagagttgtctcacttgaaggagtttgttaagtcccaagcagcaggtttgcagcatgaaatagctgaaggtggtgaaaacctcagtgttggtcaaaggcaactgatttgtttagcccgggcattgttacgaaaaactaaagtacttattttggatgaagcaacagcagctgttgatgtagaaacagacgatcttatccaaaccaccatccgtcaggagttcagcgactgcacaatattgacgattgctcatcggttaaacacaattatggattccgacagagttctggtattggatcacggtctaatgatggagtacgactctccattgaaccttctcaagaatccagcgtcccttttccactcactcgttaaagatgcaggcttagatgtagaaacgattctctccacccaaagcatgaatggaagacaattggtggtctga

SEQ ID NO: 24 - protein, 1533 aa, Bemisia tabaci
MDHYFCNSPLWDLNVTWNTSDPNFTPCLQATILSWVPCLFLWFFAPFSVRSLMKRQTWTVPWAWLNTAKFLLTLLLIFLSLVDLSYSVISYFDGRPFYLVDFITPLLKTVTFALAAALLLLYKLRGVPSSALLFLFWFLSVICGAPRYKYELDRAYRQNLGEAVVPFVSYMVYYAMVLGLFILNFFADLLPISPSYSPKEKSSPYSQASFPSLVTFSWFDPLAWKGYRRPIVVEDLWSLAEDDSVKKIAPKFNKHCRKRSNKHPQSRNVKEGTTRTANGIELKPYSDKAASKEVYPSIWFPLIKTFWPSVLFGFSLKLCNDILIFVSPHILKSLISFMQSEEEVWHGYFYAGGLFIAASFQTLFLAHYFNRMRITGLRIRVALTSAIYRKALNISNASRKESTVGEIVNLMSVDAQRFMELMPHLSLLWSAPLQISLAVFFLWEILGPSVLGGLVVMILVILINIPIARKSDALTMKQMRYKDERIKLMNEILSGIKVLKLYAWETSFEKHIQKIRMKEVNTLKQLAILGSTTAFIWSCAPLLVALTTFGVYLFIDDEHSLDAEKTFATVALFNILKFPLVMLPNMISNLVQTMVSVRRLNKFMNSDELDPNCVTHSASETEPLVIEKGNFGWGTTVPVLHDIDIKIFDKSLVAVVGTVGCGKSSLLAAFLGEMDLISGRVNTKGSIAYVTQQAWIQNATLRENILFGKEYDYRTYNKVIKACALRSDFALLPGGDQTEIGEKGINLSGGQKQRVSLARAVYSDADVYFLDDPLSAVDSHVGKHIFENVIGPQGLLKNKTRVLVTHGLTYLPDVDEIYVLKNGMISEKGTYKQLIDKKGAFAEFIQTHMLESENDDKPAEGTQDISRPSSPLVYQRVQDRSRPSSPALLRKRSHSASERGSTLSLRQFSYEEGSNFVEQDDDRRNKIIEEEIAMTGNIKWSVFSHYMRAMGWLFVFLAFAFTLLFQVCAVGSNLWLSIWTSRNNTLPDGSQDPEERNWFLEVYAILGLSAGLCAIVADLAPRIGCVKASRALHGYLLHGILRAPLTFTDTTPTGRILSRFSKDVDTMDNKLPMEISDTLYCVMEVIGVLFIITYATPPFLWCIVPLVILYYILQRFYTATSRQLQRLESISRSPIFSHFSETVAGAQVIRAYGAVNRFIQEYEKRMDANQICFHPTVISIRWLSVRLETIGNLIIFCAAFFAVLARGSLSAGLVGLSVSYALQVTGALNWFVRMASSVETDVVAIERVKEYTEIQEEAKWDVPETLPPAEWPQKGIIVFENFKVRYRSELDLVLKGLNFSVNEREKVGIVGRTGAGKSSLTLALFRIIEAAEGAIYIDGIDISTLGLHTLRSRLTVIPQDPVLFSGSLRMNLDPLGVYSDTKIWHALELSHLKEFVKSQAAGLQHEIAEGGENLSVGQRQLICLARALLRKTKVLILDEATAAVDVETDDLIQTTIRQEFSDCTILTIAHRLNTIMDSDRVLVLDHGLMMEYDSPLNLLKNPASLFHSLVKDAGLDVETILSTQSMNGRQLVV

SEQ ID NO: 25 - DNA, 4962 bp, Bemisia tabaci
  source 1..4962: mol_type = other DNA
atggggtgcatcatgggtctttggagacaaatccgagttttgcactatagaaacacgtgcatccgtagacaccagtggctggcctctttgtttgagatcctcctgccgactctggcctttgcactagtggcttacaccggtgcacgaaatacattcgagcatattaccacgaatgaaacaatctggcccacgtacagtgacgatttcgtcactaaaaatggattttgggatttcaaagatgtaatcaatatcgtatacactcctcaaagcagagccacccaacgattggtcaatgagatggtgtcgattttacaggacaagaccaaagatacagatcggaaattcaacgtgtcaactgctgactcggaggaggagatagaggacattttccgtcgtaagttcttcaaccctttgagggacctcgcccccggtttcggaatccacttcactaacataaacccgaacacccccgaagagctaccaaccaatttggagtttgtcattcgccctgtgaccagcatgttagaaacagatcaattgttcatatcaactggtcttcaagtctggggtgtctatcctgcttacttgagctttggattcgtgggaatcaacttggcactaaacgaggctttcctgagattgactgcccgcgatgctggtgtctctgtaccccggatattaataaagcaattcccttcgcctcgaacaagtcaagatgttgcgatttttaaagtaaccgattttttgcctggctggtgcatgttcctcgtcactctcatttttttgttcattttgaagaacgtgatctccgagaaggaaactggagcaaaggaactgatgaagatgatgggtctgaaaggttggatgaactggcttggctggataatgggtgttcttatgacaagcatttttgctgttacaatttgtgtttttcttctcacggtttctatttttggccaacctccactcctggcctcagccactgtactttgggtttttttaatgttctacatttatgctgctattggctacgcattttgtctctgtccaatgttcaaaaggccgacgttggctatttgcattgccaacgtcttatggccaatgaccggcatagcttacaaattagaaataccggaagattcggacttcggattcctcgataaatatttatatttgatacctaacgtagctctacgtcgaggctgggatacattcagttactttgaagaaagaggtggcgcgcattggtcagacctcttcacacggcccagtagtgaacacaatttttctttggccgttgtttttggcagtttcatactgcaatgcttcatttttggcaccattgcctggtatctagatgctgtttggccgggtccttacggcattcccgagactgcgctctttttcctgaagaaatcatattggtggtcaaacgcaggaagtaaagagagggaggacgactgggatccagaaaaagtaaaaaattttgagcgcgtgccaccgaacttgaagcctgggataaaagttcgcaacttgagaaaatcgttcaaaaagttcgttgcagtgaatgatgtatccattaatttttatgagggtgaaatcacggctttgctgggccacaacggagctggaaaaactaccaccatgtctatgctcaccggcatgattccttccgacgccggccgagtgattgttgatggatacaacattttcgacaacatgtcgcagttccgtcaaaatttaggactttgtccgcaacataatttgttgttcagcactctgaccgtgatggagcatcttacatttttcgggctgttaaaagggttatctaaacaacaagctcatacagaggcgttgcatttgatggaattacagaatatcagcattaaatgtaatgatctaccagataagctctccggtggaatgaaaagaaagctctctctagctatagcactaattggaagtcctaaaatactgatgctagatgaaccgacatcagggatggatccagaatcaaggcgagagatgtggaacttactcttgagtattcgaggtgaacggacaattattttaacaactcatttcatggaagaggctgacgtcctaggggaccgtattgcaatcatggatcatggccaagttatgtgttttggaacctccattatgctgaaaaatttatatggtgctggttattatctgaacattttaaagaagagggatgccggacaaaatcgggatgaaatcactgcggcagttcatgaatacgtatcagaggcagaattgaaaagtgacgagggacctaagttaacgtacgtcataccgatagagtctgtcgatgcactcccagatatgtttgagcaactcgagtccttgaaagatgagcttggtatcgccagtatggcggttgcttgcaccactttagaggaagtttttctcaaggttggagagattgcagacggagaaagaggtgcagttgagaatggcaccgtcaatcaaagctttgaacttgacaactcagtgcataatgcatcatctgaggtgatttatcgagatgaaaaggattacacgaaaaagaggagtatggctcttttatggcaacaatttacatcattgatgaagagaaagctgatatattcctgcaggcgtcgaaaaatgtaccttatttttgggatcgtagttttgttggcgtcgctactcgccagtagtcttatgagcgggcagaaaaatgctgaagtccctggacaaccagctctaccgttgaaaccggttacgtacaggtcaatgcgggtgcaatatcgctcggcagactcgaatccttacacagctcagttggaggagcttgccaaaccacagacaactctaaagatctcctccgacgaggatctggatacatatctcctgagccaggcatacagggatatccgcgagtactactacaaaaatatgatggtgctgaacgtgaatagcacatccctcatagtttttcacaaccctgttgcacaacactctttacccatttgcgtaaatcttgcctataatcttatactaaaggcgacaacgggcagaacgatatctgctgtgaatcatcctgtgaaccttaaaagaagggatttctgtcacttaactagtgacgaaaaaatgaagttggtcatttcaaggatcttcatattgttccaattttttgggacgacgatgcttgtgctcagtggctctttcattgcacttccgatccaggagcgtctcaacaacgctaagcaactacagttgatgactggtgctccttccaccctttattggtttaccatgttcttcgttgattactcctactacatactcatttctgtactttccgtgctaatcttccttttcaaagaaaattatatcgcaggagaagctgcggtcctcttcttgattacggcgttagctgggctgagtaacacattgttctcttccggcgttgcatttttctacgaatcttacgctaacgcattttcctttcatatcaaaatgaatttaatactaggtacttacggtggagccatcatgtacgctttgatgacgttgaacggttttccacattcacagcttgtagatggcccgtggctggacttagctgatggagttctccggcttttacctcaatactcgtttaccttttcactgaccagtttcgtattagccgcctatcagacagatgtttgcagaacgtgttcggagtctgaatgtcaaaaaatgttattcatcagggacatggaggttcagaaaacgagtttgaaatacaatttaacttcactggctctctctggccttttccaactggcactaatcttccttatggaatcagagcgtgcgcgaaaacttcgcaattttgtgtttatatcaatcatcggatccgagctcaaggacaagaagcaaacagatgaagatccggatgttcaagaggaaagggagaaaattgatgcagcaaagaatcacccagagcacagatctgagtacgtgctattggtggatcggctggtgaagaagttctcgcgatcgtcggcgggggtgcgcggggtgtcgttcggggtgcgcgccggcgagtgcttcgggttgttgggcgtcaacggggccggcaagacgaccaccttccgcatgctcacaggcgacacgctcccaacggccggcgactgctctgtccactccgtccgactctccagagacgcccgccgctacttggcccagatcggctactgcccccaggtcgacggcatcaacgagtacctcacaggccaagaactcctacgccacttcgcgaatctgagaggagtgcacagatatcaaatagatcaggaggttgccaggtgggtaacaacaatgggcctctcggagtacgcgagcaaactgtctgggacgtactctggcggcaacaagcggaaactaagcaccgcgatggcgttgataggagagccaaacttggtcgtgctcgacgaaccgactagtggtgtggacccgatttcaagaaggaaactttgggacatactaatcgaagctcggaaaagaggtcaagcaatcgtgttcacctcccactcaatggacgaatgcgagactctctgtgatcgacttacgataatggcgggtggtaaactcatgtgcatcgggaacgtggagtacctaaaacagcgttacgcacaaggctacacaatccagctgaaactgagtggcgcgcctcaaacggaactaactgccgtcaagcgggcggtggaacaagcgtttgaaggggccatgctcaaagacgagcacgctgggtctctacgctaccacattactaccccgacggtagctctctctgcgctgtttaataaaatgcgagaaattaaaggatccatgaatgttgttgaagattatcaagtttgcaatacgacactggagcaggtgttcctggctttcgccagaagcgaagccaaaatagtcgagtcctaa

SEQ ID NO: 26 - protein, 1652 aa, Bemisia tabaci
MGCIMGLWRQIRVLHYRNTCIRRHQWLASLFEILLPTLAFALVAYTGARNTFEHITTNETIWPTYSDDFVTKNGFWDFKDVINIVYTPQSRATQRLVNEMVSILQDKTKDTDRKFNVSTADSEEEIEDIFRRKFFNPLRDLAPGFGIHFTNINPNTPEELPTNLEFVIRPVTSMLETDQLFISTGLQVWGVYPAYLSFGFVGINLALNEAFLRLTARDAGVSVPRILIKQFPSPRTSQDVAIFKVTDFLPGWCMFLVTLIFLFILKNVISEKETGAKELMKMMGLKGWMNWLGWIMGVLMTSIFAVTICVFLLTVSIFGQPPLLASATVLVFLMFYIYAAIGYAFCLCPMFKRPTLAICIANVLWPMTGIAYKLEIPEDSDFGFLDKYLYLIPNVALRRGWDTFSYFEERGGAHWSDLFTRPSSEHNFSLAVVFGSFILQCFIFGTIAWYLDAVWPGPYGIPETALFFLKKSYWWSNAGSKEREDDWDPEKVKNFERVPPNLKPGIKVRNLRKSFKKFVAVNDVSINFYEGEITALLGHNGAGKTTTMSMLTGMIPSDAGRVIVDGYNIFDNMSQFRQNLGLCPQHNLLFSTLTVMEHLTFFGLLKGLSKQQAHTEALHLMELQNISIKCNDLPDKLSGGMKRKLSLAIALIGSPKILMLDEPTSGMDPESRREMWNLLLSIRGERTIILTTHFMEEADVLGDRIAIMDHGQVMCFGTSIMLKNLYGAGYYLNILKKRDAGQNRDEITAAVHEYVSEAELKSDEGPKLTYVIPIESVDALPDMFEQLESLKDELGIASMAVACTTLEEVFLKVGEIADGERGAVENGTVNQSFELDNSVHNASSEVIYRDEKDYTKKRSMALLWQQFTSLMKRKLIYSCRRRKMYLIFGIVVLLASLLASSLMSGQKNAEVPGQPALPLKPVTYRSMRVQYRSADSNPYTAQLEELAKPQTTLKISSDEDLDTYLLSQAYRDIREYYYKNMMVLNVNSTSLIVFHNPVAQHSLPICVNLAYNLILKATTGRTISAVNHPVNLKRRDFCHLTSDEKMKLVISRIFILFQFFGTTMLVLSGSFIALPIQERLNNAKQLQLMTGAPSTLYWFTMFFVDYSYYILISVLSVLIFLFKENYIAGEAAVLFLITALAGLSNTLFSSGVAFFYESYANAFSFHIKMNLILGTYGGAIMYALMTLNGFPHSQLVDGPWLDLADGVLRLLPQYSFTFSLTSFVLAAYQTDVCRTCSESECQKMLFIRDMEVQKTSLKYNLTSLALSGLFQLALIFLMESERARKLRNFVFISIIGSELKDKKQTDEDPDVQEEREKIDAAKNHPEHRSEYVLLVDRLVKKFSRSSAGVRGVSFGVRAGECFGLLGVNGAGKTTTFRMLTGDTLPTAGDCSVHSVRLSRDARRYLAQIGYCPQVDGINEYLTGQELLRHFANLRGVHRYQIDQEVARWVTTMGLSEYASKLSGTYSGGNKRKLSTAMALIGEPNLVVLDEPTSGVDPISRRKLWDILIEARKRGQAIVFTSHSMDECETLCDRLTIMAGGKLMCIGNVEYLKQRYAQGYTIQLKLSGAPQTELTAVKRAVEQAFEGAMLKDEHAGSLRYHITTPTVALSALFNKMREIKGSMNVVEDYQVCNTTLEQVFLAFARSEAKIVES

SEQ ID NO: 27 - DNA, 4947 bp, Bemisia tabaci
  source 1..4947: mol_type = other DNA
atgggtctttggagacaaatccgagttttgcactacagaaacacgtgcatccgtagacaccagtggctggcctctctgtttgagatcctcctaccgactctggccttcactttattggcctactccggtgcaagaaatacattccaaactattaccaagaatgaaacaacctggcccacgtacagtgaaaattccgtcattgaaaatgctttttggaattgcgaagatgtaaccaatatcgtatacaccccacaaaacagagccactcaacgattgatcgatgagatgatgtcgattttacgggacaaaaccaaaaatacagatcggcaattcaacgtgtcaactgctgagtcggaggaggagtttgaggacatttttcgtcgtaagttctggatcccttatagggacctcatccccggttttggaatccacttcaccaacataaacccgaacacccccgaggagctaccgaccaatttggagtttgtcattcgccctgtgaccacctttttagagacagataaattgttctcatcaactggtctccaagtctggggtgtctattctgattacttgagctttggattcgagggaatcaacttggcactaaatgaggctttcctgagactgaccgctcgcgatgctaatgtcttcgtaccccggatattcatcaaggcattcccatcccctcgaacaagtcgagatgttgagatttttaaagtaaccgattttttgccaggctggtgtatgtttctcgtcactctcgtttttctgtttatattgaagaacgtgatttccgagaaggaaactggagcaaaagaactgatgaagatgatgggtctaaaaggctggatgaactggcttggctggataatgggtgttctcatgacaagcatttttgctgttacaatgtgtgttttacttctcaccgttcctatttatggtgaacctccgctcaaggcctcagccactctactttgggtttatttaatgctctacatttatgccgctattggctacgcattttgtctctgtccaatgttcaagaggccgacgttggctatttgcattgctaatatcatatggccaatgaccggcatagcttacaaattggaaataccggagggctcggacttcggattcctcgataaatatttatatttgatacctaacgtagctctacgtcgaggttgggatacattcagttacttcgaagaaagaggtggcgcgcattggtcagacctcttcacacagcccaatactgaacacaatttttctttggccgttatttttggctgtttcatactgcaatgcttcattcttggcaccatcgcctggtatctagatgctgtttggccgggtccttacggcattcccgagacagtgctctttttcttgaagagatcgtattggtgttcaagtgcacgaagtaaagcgcaagaggatgattgggatccagaaaaagtgaaaaattttgagcgcgtgcctccgaacttgaagcctgggataaaagttcgcaacttgagaaaatcgttcagaaagctcgttgctgtgaatgatgtatccattaatttttatgagggtgaaatcaccgctttgctgggccacaacggagctggaaaaactaccaccatgtcaatgctcaccggtatgattccctccgacgccggccgagtgattgttgacggatacaacattttcgacaacatgtcgcagttccgtcaaaatctaggactttgtccgcaacataatttgttgttcagcactctgactgtgatggagcatcttacattttttgggctgttaaaaggattatctaaacaacaagctcatacagaggcgttacatttgatggaattacaaaatatcagcagtaaaagcaatgatctaccagataagctctcaggtggaatgaaaaggaagctatctctagctatagcactaattggaagtcctaaaatactgatgctcgatgaaccgacatcagggatggatccagaatctaggcgagaaatgtggaacttactcttgagtattcgaggtgaacggacaattattttaacaactcatttcatggaagaggcggatgtactaggggacagaattgcaattatggatcatggtcaagttgtgtgttttggaacctccattatgctgaaaaatttatacggtgccggttattacctgaacattttaaagaagaaggacgccgaacaaaatcgggatgaaattactgcggcaattcatgaatacgtatcagaggcagaattgaaaagtgacgaggggcctaagttaacgtacgtcataccgatagagtctgtcgatgcactcccagatatgtttgagcaactcgagtccctgaaagaggagctcggcatcgccagtatggcgatcgcatgcaccacgttagaggaagtttttctcaaggttggagagattgcagacggagaaagaggtgcagttgagaatggcaccgtcaaccaaagctttgaacttaacgaatcagtgcataatgcgtcatctgaagagatttatcgagatgaaaaggattacaagaaagtgaggatggctcttttatggcagcaatttacatcattgatgaagagaaagctgatatattcctgcaggcgtcgcaaaatgtaccttatttttgggatcgttgttttgttggcgtcattactcgctagtagtcttattagcggccagaaaaatgctaaggtccctggacaacctgctttaccattgacaccggctacgtataggtcaatgcgggtgcaatatcgctcggcagactctagtccttacattgcccagttggaggagcttgctaaaccacagacaactgtcaagatctcctctgacgaggatatagatacatatcttttgcgccaagcaaacagagatatccgcgagtactactacaaaaatatgatggtgctggacgtgaacagcacatccctcctggttcttcacaaccctgttgcacaacactctttacccatttgcctaaatcttgcctataatctcatactaaaagcgacaacgggtcgaacgatatctgttgtaaatcatcctgtgaatctcagaaaaagggatttctgccacttgactagtgacgaaaaaatggagttggtcgtttcaaggatcttcatattgttccaattttttgggacgacgatgcttgtgctcagtggttctttcattgcacttccgatccaggagcgtctcaacaacgcgaagcagctacagttgatggctggtgctccttcctccctttattggtttacgatgttcttcgttgattactcctactacatactgatttctgtactttcaattttaatcttcattttcagagacaattttatcgcaggagaagctgcggtcctcttcttgattacggcgttagctgggctgagtaacacactgttctcttacgtcttggcatttttccatgaatcttacgccaatgcattttcatttcatatcaaagtgaatttaatactaggtacttacggtgcggccgttatgttcgctttgatgacgttgaacggttttccacatacacagcttgtacatggcccgtggctagacttagctgatggagttctccggcttttaccgcaatactcgttcaccttttcactgaccagtttcgtattggccacctatcagaaagatgtttgcagaacgtgttcttactctgaatgtcaaaaaatgttattcatcagggaaatggaggttcagaaaacgagtttgaaatacaatgtatcttcactggctctctctggccttgtccaactggcactcatcttcctcatggagtcagagcgtgcacgaaaacttcgcaattttgtgtttaaatcgataattggatccgagttccaggacaagaagcaaacagacgaagatccggatgttcaagaggaaaaagagagagttgatgcagtaaagaatcacccagagcacagatctgagtacgtgctattggtggatcggctggtgaagaagttctcacgatcgtcggcgggggtgcgcggagtgtcgtttggggtgcgcgccggtgagtgcttcgggctgttgggggtcaacggggccgggaagacgaccaccttccgcatgctgaccggcgacacgctcccaacagccggcgactgctctgtccactccgtccgactctccagggacgcccgtcgcttcttagcccagatcggctactgcccccaggtcgacggcatcaacgagtacctcactggacaagaactcctacgccacttcgcgaatctgagaggagtgcacagatatcaaatagatcaggaggttgccaggtgggtaactacaatgggcctctcggagtacgcgagcaaactgtctggcacgtactctggcggcaacaagcggaaactaagcaccgcgatggcgttgataggagagccaagcttggtggtgctcgacgaaccgaccagtggcgtggacccgatctcaagaaggaaactttgggacatactaatcgaagctcggaaaagaggtcaagcaatcgtgttcacctcccactcaatggacgaatgcgagactctctgcgatcggcttacgataatggcgggtggtaaactcatgtgtatcgggaacgtggagtacctgaaacagcgttacgcacaaggctacacgatccagctgaaactgagtggcgcgcctcagacggagctggacgccgtcaagcgggcggtggaacaagcatttgaaggggccatgctcaaagacgagcacgctgggtctctacgataccatattactaccccgacggtagctctttccgcgctgtttaataaaatgcgagaaattaaaggatccatgactgctgttgaagattatcaagtgtgcaatacgacactagagcaggtgtttctggcttttgccagaagcgaagctaaaatagttgagtcttaa

SEQ ID NO: 28 - protein, 1648 aa, Bemisia tabaci
MGLWRQIRVLHYRNTCIRRHQWLASLFEILLPTLAFTLLAYSGARNTFQTITKNETTWPTYSENSVIENAFWNCEDVTNIVYTPQNRATQRLIDEMMSILRDKTKNTDRQFNVSTAESEEEFEDIFRRKFWIPYRDLIPGFGIHFTNINPNTPEELPTNLEFVIRPVTTFLETDKLFSSTGLQVWGVYSDYLSFGFEGINLALNEAFLRLTARDANVFVPRIFIKAFPSPRTSRDVEIFKVTDFLPGWCMFLVTLVFLFILKNVISEKETGAKELMKMMGLKGWMNWLGWIMGVLMTSIFAVTMCVLLLTVPIYGEPPLKASATLLWVYLMLYIYAAIGYAFCLCPMFKRPTLAICIANIIWPMTGIAYKLEIPEGSDFGFLDKYLYLIPNVALRRGWDTFSYFEERGGAHWSDLFTQPNTEHNFSLAVIFGCFILQCFILGTIAWYLDAVWPGPYGIPETVLFFLKRSYWCSSARSKAQEDDWDPEKVKNFERVPPNLKPGIKVRNLRKSFRKLVAVNDVSINFYEGEITALLGHNGAGKTTTMSMLTGMIPSDAGRVIVDGYNIFDNMSQFRQNLGLCPQHNLLFSTLTVMEHLTFFGLLKGLSKQQAHTEALHLMELQNISSKSNDLPDKLSGGMKRKLSLAIALIGSPKILMLDEPTSGMDPESRREMWNLLLSIRGERTIILTTHFMEEADVLGDRIAIMDHGQVVCFGTSIMLKNLYGAGYYLNILKKKDAEQNRDEITAAIHEYVSEAELKSDEGPKLTYVIPIESVDALPDMFEQLESLKEELGIASMAIACTTLEEVFLKVGEIADGERGAVENGTVNQSFELNESVHNASSEEIYRDEKDYKKVRMALLWQQFTSLMKRKLIYSCRRRKMYLIFGIVVLLASLLASSLISGQKNAKVPGQPALPLTPATYRSMRVQYRSADSSPYIAQLEELAKPQTTVKISSDEDIDTYLLRQANRDIREYYYKNMMVLDVNSTSLLVLHNPVAQHSLPICLNLAYNLILKATTGRTISVVNHPVNLRKRDFCHLTSDEKMELVVSRIFILFQFFGTTMLVLSGSFIALPIQERLNNAKQLQLMAGAPSSLYWFTMFFVDYSYYILISVLSILIFIFRDNFIAGEAAVLFLITALAGLSNTLFSYVLAFFHESYANAFSFHIKVNLILGTYGAAVMFALMTLNGFPHTQLVHGPWLDLADGVLRLLPQYSFTFSLTSFVLATYQKDVCRTCSYSECQKMLFIREMEVQKTSLKYNVSSLALSGLVQLALIFLMESERARKLRNFVFKSIIGSEFQDKKQTDEDPDVQEEKERVDAVKNHPEHRSEYVLLVDRLVKKFSRSSAGVRGVSFGVRAGECFGLLGVNGAGKTTTFRMLTGDTLPTAGDCSVHSVRLSRDARRFLAQIGYCPQVDGINEYLTGQELLRHFANLRGVHRYQIDQEVARWVTTMGLSEYASKLSGTYSGGNKRKLSTAMALIGEPSLVVLDEPTSGVDPISRRKLWDILIEARKRGQAIVFTSHSMDECETLCDRLTIMAGGKLMCIGNVEYLKQRYAQGYTIQLKLSGAPQTELDAVKRAVEQAFEGAMLKDEHAGSLRYHITTPTVALSALFNKMREIKGSMTAVEDYQVCNTTLEQVFLAFARSEAKIVES

SEQ ID NO: 29 - DNA, 1608 bp, Bemisia tabaci
  source 1..1608: mol_type = other DNA
atgcgttcgataataccccgtaaatacgggccgttgacgactctcattattattttgacactcgcagccgccacacatagctttaaaattttaatattgtacccgacaccgtcgtttagtcatcaacgtccgattatggcattaactgagcggttaatcaaagaaggccatgaattgtttgtagtcacaccaaaccctgtgtcggatcttaaggatcacgtcaactacactcttgcagacctctctttcgcgtatggtcagcgcgaagacaaaaaaaatggcgtaccccaatatcagcggcagctttccaaatggaaaacagctacaatgaccagggcgatgatgaaacttatacagatccagcttcgaagccgggaattccttcactttcaaaagcatctggagtcggaaaaaatcaaattcgacgttgtgattgttcaatcttatctacttccatacctggccccaatagcaagcatcctagctacaaatgccccgataatagccgtatcatcattttcttcggacctcatcggagaagactttctgggcagtaaaatgcatctgtctttttcaccctctacttttggtcattacacggatgaaatgaatttatggcagaagattgaaaactggttttcccatacttacatcaaatccagcataaggagaacggtagaggattctgcgaagcaatttttccaggagatttatggttcgggaggtgagagactggtggatttttggtgggtaaacgtcagcattactctgctcacctccaattcgttgtattactatccgaggcttttgagcccaaatgttattcaaaccggacctctgcatattaaacctccgaagcagctaccacaggatttacaagactggttggacggggcggagaaaggagtgatctacttcagtcttggaagtaatttaaaaagcacatctttaccacaagacgtgctcaaaaacttcttgaaattcttccgagagttgccggttggctacagggtgctctggaaatgggaatctggcggggagattccaggacagtccgataacattttaacccaaaggtggatgccgcaagatagcgtacttgctcatccgaaagtgaaagtattcattacgcagggagggcttcagagtttccaagaagcagtacactacggggtccccacagttggaatcccgtggtttgtggatcaaaattgtgtaacagccaaaatggtcgatgcgaaagttggagtcaagttgtcgccgcaagatatccactcctttgaaaaaattaaagcaggcatcgaagcaatgttacacgatgaaaagtatctggagaacatgcgacgactttcggccatatcccgtgacttcacttctcaagccatggacaaagcggtcttctggatagagcacgttgctcgtcatggaggtgcatctcatttgagaccagccatcgctgattcgagtctcttcgaattcctctgcctggacattacatcagtgatcttattttcatgtttcattagtctctacggatttttctctattggcagagtcgtaataacgtatttatgtaaaaattttgggggaaaaataaaaggagcttga

SEQ ID NO: 30 - protein, 535 aa, Bemisia tabaci
MRSIIPRKYGPLTTLIIILTLAAATHSFKILILYPTPSFSHQRPIMALTERLIKEGHELFVVTPNPVSDLKDHVNYTLADLSFAYGQREDKKNGVPQYQRQLSKWKTATMTRAMMKLIQIQLRSREFLHFQKHLESEKIKFDVVIVQSYLLPYLAPIASILATNAPIIAVSSFSSDLIGEDFLGSKMHLSFSPSTFGHYTDEMNLWQKIENWFSHTYIKSSIRRTVEDSAKQFFQEIYGSGGERLVDFWWVNVSITLLTSNSLYYYPRLLSPNVIQTGPLHIKPPKQLPQDLQDWLDGAEKGVIYFSLGSNLKSTSLPQDVLKNFLKFFRELPVGYRVLWKWESGGEIPGQSDNILTQRWMPQDSVLAHPKVKVFITQGGLQSFQEAVHYGVPTVGIPWFVDQNCVTAKMVDAKVGVKLSPQDIHSFEKIKAGIEAMLHDEKYLENMRRLSAISRDFTSQAMDKAVFWIEHVARHGGASHLRPAIADSSLFEFLCLDITSVILFSCFISLYGFFSIGRVVITYLCKNFGGKIKGA

SEQ ID NO: 31 - DNA, 1608 bp, Bemisia tabaci
  source 1..1608: mol_type = other DNA
atgcgttcgataatactccgtaaatatggcccgttagcggctctcattactattttgacacttacagccaccacacatagctttaaaattttaatattgtatccgacaccgtcatttagtcatcagcgcccgattatggcattgtctgagcgattaatcaaagaaggccatgaattgtttgtcgtcacaccaaatcctgtgtcggatcttaaggatcacgtcaactacactcttgcagacctatcttttgcgtatgatcagcgtgaagacaaaaaaaatggcgtaccccaatatcagagacagatttccaagtggaaaacagctacaataaccagggcgatgatgaaaattatacaatttcagcttcgaagccaggaattccttcactttcaaaagcatctggagtcggagaaaatcaaattagacgtcgtcatcgttcaatcatatctacttccatacctggctccaatagcaagcattctagctacaaacgccccaattatagccgtatcatcattttcttcggacctcatcggggaagactttntgggcagtaatatgcatctntctttttcgccctctacatttggtcattacacagataaaatgaatttatggcagaagattgaaaactggatctcccatacttacatcaaatccagcataaggagaacagtagaggattctgcaaggcaatttttccaggagaattatggtcaaggaggtgagaaactcgtggatttttggtgggtaaacgtcagcattactctgctcacctccaattcgttgtattactatccgaggcttttgagcccaaatgttattcagaccggacctctgcatattaaacctccaaagcagctaccaaaggatttacaagactggttggacggggcggatagaggagtgatctacttcagtcttggaagtaatttaaaaagcacatctttaccacaagacgtgctcaaaaatttcttgaaattcttccgggagttgccggttggctacagggtgctctggaaatgggaatcaggtggaaaaattccaggacagtccgataatattttaacccaaaagtggatgccgcaagacagcgtacttgctcatccgaaagtgaaccttttcattacgcagggagggcttcagagtttccaagaagccgttcactatggagtccccacacttggcatcccgtggttcgtggatcaaaattgtgtcgcagctaaaatgatcgatgcaaaggttggagttaagttgtcgccgcaagatatccactcttatgaaaaaattaaggcaggcgtcgaagcagtgttatacgatgaaaactatctgaagaacatgcagcgactttcggccatatcccgtgacttcacttctaaagccatggacaaggctgttttctggatagagcacgtggctcgtcacggaggtgcatctcatttgagaccagccatcgctgatgcgaatctcttccagttcctctgcctggacattatatcagtgatctcattgtcctgttttattagtctctatggatttttctctcttggcagagtcgcaataacgtatgcatgtaaaaattttggcggaaaaataaaagaagcgtga

SEQ ID NO: 32 - protein, 535 aa, Bemisia tabaci
MRSIILRKYGPLAALITILTLTATTHSFKILILYPTPSFSHQRPIMALSERLIKEGHELFVVTPNPVSDLKDHVNYTLADLSFAYDQREDKKNGVPQYQRQISKWKTATITRAMMKIIQFQLRSQEFLHFQKHLESEKIKLDVVIVQSYLLPYLAPIASILATNAPIIAVSSFSSDLIGEDFXGSNMHLSFSPSTFGHYTDKMNLWQKIENWISHTYIKSSIRRTVEDSARQFFQENYGQGGEKLVDFWWVNVSITLLTSNSLYYYPRLLSPNVIQTGPLHIKPPKQLPKDLQDWLDGADRGVIYFSLGSNLKSTSLPQDVLKNFLKFFRELPVGYRVLWKWESGGKIPGQSDNILTQKWMPQDSVLAHPKVNLFITQGGLQSFQEAVHYGVPTLGIPWFVDQNCVAAKMIDAKVGVKLSPQDIHSYEKIKAGVEAVLYDENYLKNMQRLSAISRDFTSKAMDKAVFWIEHVARHGGASHLRPAIADANLFQFLCLDIISVISLSCFISLYGFFSLGRVAITYACKNFGGKIKEA

SEQ ID NO: 33 - DNA, 1638 bp, Bemisia tabaci
  source 1..1638: mol_type = other DNA
atgctcatattatatgaggaacaagatcatcatttaaacatggcttcgtctctgaaacttttgttggttttcataattttcctggcttcttgtgcgaatgcatttaagattttaatcttgtatcctacgccttcatacagtcatcaaatttcagtcatgaacattgctgaggggcttgtcaagaaaggtcatgaggtcttcttcgtcagtccaaatcccataccaggtctgggagagaattatacatttgtggatctatcgttttcttacaagtactttaaaaaagaggacaatgacgaggctgttaatttagagcagcagatttcaagatgggacttcgtgaaagtttttgtgccctttgcagccgtaccagaaaaacaattcataagccagccttttgtggaatttcagaggcgggtagccgcggagcagatcaagttcgacgtggccatcatagaattcgtgggcatcccctacacctgcggtatgacgcgacttctgggcaggctgaccaacagcaccatccccttgatctccatgtcctccgtagcgagcgacttctacgcagagcgaaatctaggcagcttccatcatctgtcttacgtgcccactatcttcgacggttattcgaacaagatgtccctctggcagaagatcgacaactggttcctgtttcactggactgtgctccagatggaggcggctctcacagaaaaggcaaagggattttatagggatacctatggagaggaaagcggacgcctggtggacggatgtttcaggaatttgagtttgtcaatgatatcgtccaatgcgttgtacttctatccgaggttacttggcccgaatgtcatcgagattgggcctgtgcatttgaagacaccagaaaaactaccagagaatctacaaaattggatggacggagcagagaaaggtgtgatttacttcagtctcggaagtaacatgaaaagcaaatctcttcctgcagttgtgctcgataattttctgaggttttttaaacaacttccgtcaggctaccgagtcctgtggaaatgggaaaaagatggtaatataccaggccagtctgataatatattgagccaaaaatggttaccacaacatagcgttctggcgcatcctaaaatgaaagtttttatcactcaaggaggcttgcagagttttcaagaggcagttcactacggggttcctacggtaggcataccatggtacggggaccagaatactgtcgtcgcgaaaatcttggatgcccaagtaggcgtgcgcattctcccgaaggagctgacgtcatatagcaagattgaatccgggattaatgctgtgctatttgatcgaaggtattcagaaaatatgaaaaaactttcacttatatctcatgacttcacctctaagtccatggaaaacgctgtcttttggatagaacacgtggcaaggcatggtggggcacctcacctcaggccgtcaacagccgattcttcctattttgaatttttatgtcttgatattttatctgtgattctagtcattattactctagttttgtatcttttgtattttatctgtaaatttacaattgcgctggtgtcaaattacttcagttctaaagaaaaaaagtcgtga

SEQ ID NO: 34 - protein, 545 aa, Bemisia tabaci
MLILYEEQDHHLNMASSLKLLLVFIIFLASCANAFKILILYPTPSYSHQISVMNIAEGLVKKGHEVFFVSPNPIPGLGENYTFVDLSFSYKYFKKEDNDEAVNLEQQISRWDFVKVFVPFAAVPEKQFISQPFVEFQRRVAAEQIKFDVAIIEFVGIPYTCGMTRLLGRLTNSTIPLISMSSVASDFYAERNLGSFHHLSYVPTIFDGYSNKMSLWQKIDNWFLFHWTVLQMEAALTEKAKGFYRDTYGEESGRLVDGCFRNLSLSMISSNALYFYPRLLGPNVIEIGPVHLKTPEKLPENLQNWMDGAEKGVIYFSLGSNMKSKSLPAVVLDNFLRFFKQLPSGYRVLWKWEKDGNIPGQSDNILSQKWLPQHSVLAHPKMKVFITQGGLQSFQEAVHYGVPTVGIPWYGDQNTVVAKILDAQVGVRILPKELTSYSKIESGINAVLFDRRYSENMKKLSLISHDFTSKSMENAVFWIEHVARHGGAPHLRPSTADSSYFEFLCLDILSVILVIITLVLYLLYFICKFTIALVSNYFSSKEKKS

SEQ ID NO: 35 - DNA, 1638 bp, Bemisia tabaci
  source 1..1638: mol_type = other DNA
atgttcatattatatgaggaacaagatcattgtttgaacatggcttcgtctctgaaacttttgttggtttccatatttttcctggcttcctgtgtaaatgcatataagattttaatcttgtatcctacgccttcatacagtcatcaaatttcagttatgaacattgctgaagggcttgtcaagaaaggtcatgaggtcttcttcgtcagtccaaattctatatcaggtctgagagagaattatacatacgtggatctatcgttttcctacaagtattttaaaaaagaggacgataacgaggctgtcaatttagagcagcagatttcaagatgggacttcgtgaaagtttttgtacccttcgcagccgtaccagaaaaacaattcataagccagcctttcgtggaatttcagaggcgggtagccgcggagcagattaacttcgacgtggccatcatagaattcgtgggcatcccctacacctgcggtatgacgcgacttctgggcaggctcactaacagcaccatccccttgatctccatgtcctccattgcgagcgacttctacgcagagcgaaatctaggcagcttccatcatctgtcttacgtgcccaccatcttcgacggttattcgaacaagatgtccctctggcagaagatcgacaactggttcctgtttcactggactgtgctccagatggaggcggccctcacagaaagggcaaagggattttatagggatacctacggagaggccgctgaaggcttggtggacggatgtttcaggaatttgagtttgtcagtgatatcgtccaatgcgttgtacttctatccgaggttacttggcccgaatgttattgagattggacctgtgcacttgaaaacacctgaaaaactcccagagaatctgcaaaattggatggacggagcagaaaaaggtgtgatttacttcagtcttggaagtaacatgaaaagcaaatctcttcctgcagttgtgctcgataatttcctgaggttttttaaacaacttccatcaggctaccgagtcctgtggaaatgggaaaaagatggtaatataccaggccagtctgataatatcttgagccaaaaatggttaccacaacatagcgttctggcgcatcctaaaacgaaagttttcatcactcaaggaggcttgcagagttttcaagaggcagttcactacggagttcctacggtaggcataccatggtacggtgaccagaatactatcgtcgcgaaaatcctggatgcccaagtaggcgtgcgtgttctcccgaaggaactaacgtcatacagtaaaattgaatccgggatcaatgctgtgctatttgatcgaagatattcggacaatatgaaaaaactttcacttatatctcatgacttcacctctaagtccttggaaaacgctatcttctggatagaacatgtggcaaggcatggtggggcacctcatctcaggccgtcaacagccgattcttcctattttgaattcctatgtcttgatattttatctgtgattctagtcattactactctagttttgtatcttttgtattttatctgtaaatttacaattgcactggtgtcaaattacttcagttctaaagaaaaaaagtcgtga

SEQ ID NO: 36 - protein, 545 aa, Bemisia tabaci
MFILYEEQDHCLNMASSLKLLLVSIFFLASCVNAYKILILYPTPSYSHQISVMNIAEGLVKKGHEVFFVSPNSISGLRENYTYVDLSFSYKYFKKEDDNEAVNLEQQISRWDFVKVFVPFAAVPEKQFISQPFVEFQRRVAAEQINFDVAIIEFVGIPYTCGMTRLLGRLTNSTIPLISMSSIASDFYAERNLGSFHHLSYVPTIFDGYSNKMSLWQKIDNWFLFHWTVLQMEAALTERAKGFYRDTYGEAAEGLVDGCFRNLSLSVISSNALYFYPRLLGPNVIEIGPVHLKTPEKLPENLQNWMDGAEKGVIYFSLGSNMKSKSLPAVVLDNFLRFFKQLPSGYRVLWKWEKDGNIPGQSDNILSQKWLPQHSVLAHPKTKVFITQGGLQSFQEAVHYGVPTVGIPWYGDQNTIVAKILDAQVGVRVLPKELTSYSKIESGINAVLFDRRYSDNMKKLSLISHDFTSKSLENAIFWIEHVARHGGAPHLRPSTADSSYFEFLCLDILSVILVITTLVLYLLYFICKFTIALVSNYFSSKEKKS

SEQ ID NO: 37 - DNA, 1613 bp, Bemisia tabaci
  source 1..1613: mol_type = other DNA
atgaaaacagcaaatttattagcaatagtttttacaagtttcagcacaactgttttttctcatagagtattgatactgcaaccgactccatcatatagtcatcaactccctgcgattggtttgacagaggctctggtgaataaaggtcacgacgtttttttcttgagcccagattcagtgccgggactgcagaagaattacactcacttcgatttttcatggctgtaccagtattttcacgagagccttacagaaaaaggcgttcatttacagcaacaattttcaaaccaacttacgtggataccatttttggaagtgtgggcagggatggtgcggcacatgtttgaaagtgacactttcaagcagtttcttaaacgcgtccaagatgagaagatatcattcgacgttgtgatcgttgaatctttcgtcattccgtacggggtaggcctggtccgtctcttgacaaaaaacaaacctgtgatctccctggccaccatgactggaggcgaatttttcaacgaggatgccatcgggaacataaaacatttatcatattctccagcaatgctaagcgccttcaacggtaaaatgagtttgtgggagcgtttggaaaattgggttacacatcattacatatcatcgaaactgcagaatgttgttgagaactccgccaaagcccattttcgagatacttatggtccagacggagaggctttcgtcgaagggtgctggggaaacattagtctcgccatgataacctcaaacgccctgtattactatccaaggcctatcactcccaatgttatcgaggttgggccgcttcatataaaaaattcgacaaaattaccgaagacattgcaagattggctagacggggcagaacgaggagtaatttatttcagtctcggcagtaatatgcgaagtgcaaacctaccgcgagatgcactgtcgaacttcttgagggttttcagagagctgccggaaggttaccgagtgctgtggaaatgggagacgatgccgaaatttcaaaaaatgaggagggggtcggaaaatcaaaagtaactaatatccttactcaaaagtggataccacagcaagaagttttagcgcatccgaaggtaaaattgttcatcacacaaggaggatgtcaaagcttccaagaaacagttcactacggagtaccattggtaggcgtcccttggtttggagaccaagaggtgaacgttgcaaagatggtggacgcaggaattggagttcggctccgacctcaagagttggattcttttgtgaaagtcaaacaagctatcgaagctgttttgttcgataaaaggtatgctgaaaatatgaagaaattatctgatctatcgcatgatttcacaagacgagctccagacgaagctgttttttgggttgaacacgtggcaaaacacggtggagccgctcatttaaggccgtatatagctgatacttcttattttagatacttttgcctcgacattatatcagttatcctaagcattggcgctgtaatattctacttcttctggaaaatacttaagtatacaacttctagtataccgatattgtcaaatgagaaaattaaaagctcataa

SEQ ID NO: 38 - protein, 537 aa, Bemisia tabaci
MKTANLLAIVFTSFSTTVFSHRVLILQPTPSYSHQLPAIGLTEALVNKGHDVFFLSPDSVPGLQKNYTHFDFSWLYQYFHESLTEKGVHLQQQFSNQLTWIPFLEVWAGMVRHMFESDTFKQFLKRVQDEKISFDVVIVESFVIPYGVGLVRLLTKNKPVISLATMTGGEFFNEDAIGNIKHLSYSPAMLSAFNGKMSLWERLENWVTHHYISSKLQNVVENSAKAHFRDTYGPDGEAFVEGCWGNISLAMITSNALYYYPRPITPNVIEVGPLHIKNSTKLPKTLQDWLDGAERGVIYFSLGSNMRSANLPRDALSNFLRVFRELPEGYRVLWKWEDDAEISKNEEGVGKSKVTNILTQKWIPQQEVLAHPKVKLFITQGGCQSFQETVHYGVPLVGVPWFGDQEVNVAKMVDAGIGVRLRPQELDSFVKVKQAIEAVLFDKRYAENMKKLSDLSHDFTRRAPDEAVFWVEHVAKHGGAAHLRPYIADTSYFRYFCLDIISVILSIGAVIFYFFWKILKYTTSSIPILSNEKIKSS

SEQ ID NO: 39 - DNA, 1611 bp, Bemisia tabaci
  source 1..1611: mol_type = other DNA
atgaaaaccgcaaatttgttagcgattttaatgacatgtttcagcacaactttttctcatagagtattaatacttcaaccgactccatcatatagtcatcagctcccagcgatgggtttgatagaggctctagtgaaaaaaggtcacgacgtttttttcttgagcccagattcagtgccgggactgcagaaaaattacactcacttcgatttttcatggctataccagtattttcacgagagcctcacagaaaaaggtgttcatttacaacaacaattttcaaatcaacttacctggataccatttttggaagtgtgggcaggaacggtgcggcacatgtttgaaagtgacactttcaagcagtttcttaaacgcgtccaagatgagaaaatatcattcgacgttgtgatcgttgagtcttttgtcattccgtacggggtaggcctggctcgtctcttgaccaaaaacagacctgtaatctccctggccaccatgactggtggcgaatttttcaacgaggatgccatcgggaacataaaacatttatcatattctccagcaatgctaagcgctttcaatggtaaaatgagtctatgggagcgtttggaaaattgggtcacgcatcactacatatcgtcgaaattgcagaatgttgttgagaattccgccaaagcctatttccgagatacttatggcccagatggtgaggctttcgtcgaagggtgctggggaaacattagtctcgccatgataacctcaaacgctctgtattactatccaaggcctatcactcccaatgttatcgaggttgggccgcttcatataaaaaattcgacaaaattaccaaagacattgcaagattggttagacggggcagaacgaggagtaatttatttcagtctcggcagtaacatgagaagtgcaaacctaccccgagaggcattgtcgaacttcttgagggttttcagagagctgccgcaaggttaccgagtgctgtggaaatgggaggacgatgccgaaattgcaaaaaatgaagagggagtcgggaaatcaaaagtagctaatattcttactcaaaaatggataccacagcaagaagttttagcgcatccaaaggtaaaattgttcatcacacaaggaggatgtcaaagcttccaagaaacagttcactacggagtaccattggtaggggtcccttggtttggagaccaagaggtgaacgttgcaaagatggtggacgcaggaattggagttcggctccgacctcaggagttggattcttttgcgaaagtcaaacaagctattaaagctgtattgttcgataaaagttatgctgaaaatatgaagaaattatctgatttatctcatgacttcacaagacgagctccagatgaagctgttttttgggtggagcatgtggcaaaacatggtggagctgctcatttgagaccttacatagctgatatttcttacttcacatacttctgtctcgatatcatatctattatcttaggaagcagcatagcaatattgtacgttctgtggactatatttagatatttaatttcattaatgccaacactgtcaagtaaaaaaattaaaaacttgtga

SEQ ID NO: 40 - protein, 536 aa, Bemisia tabaci
MKTANLLAILMTCFSTTFSHRVLILQPTPSYSHQLPAMGLIEALVKKGHDVFFLSPDSVPGLQKNYTHFDFSWLYQYFHESLTEKGVHLQQQFSNQLTWIPFLEVWAGTVRHMFESDTFKQFLKRVQDEKISFDVVIVESFVIPYGVGLARLLTKNRPVISLATMTGGEFFNEDAIGNIKHLSYSPAMLSAFNGKMSLWERLENWVTHHYISSKLQNVVENSAKAYFRDTYGPDGEAFVEGCWGNISLAMITSNALYYYPRPITPNVIEVGPLHIKNSTKLPKTLQDWLDGAERGVIYFSLGSNMRSANLPREALSNFLRVFRELPQGYRVLWKWEDDAEIAKNEEGVGKSKVANILTQKWIPQQEVLAHPKVKLFITQGGCQSFQETVHYGVPLVGVPWFGDQEVNVAKMVDAGIGVRLRPQELDSFAKVKQAIKAVLFDKSYAENMKKLSDLSHDFTRRAPDEAVFWVEHVAKHGGAAHLRPYIADISYFTYFCLDIISIILGSSIAILYVLWTIFRYLISLMPTLSSKKIKNL

SEQ ID NO: 41 - protein, 262 aa, Bemisia tabaci
MEDISSSGEEISMKAISKVIGVPDIRDGPTLTKCIVAEFVGTLLLVLIGCMSVAFVHQDNFVDVVKIAMAFGLIIASMVQAIGHVSGCHINPAVTCGLAVSGHVSIIKGMLYIVAQCLGAICGAIILNEITPKTGYTAAGNLGVTTLSTGVSDLQGVAIEALITFVLLLVVQSVCDGKRTDIKGSIGVAIGFAIACCHLAAIKYTGASMNPARSLGPAFVSGIWDKHWVYWAGPILGGVTASLLYAITFKAKKRSDESSYDF

SEQ ID NO: 42 - DNA, 789 bp, Bemisia tabaci
  source 1..789: mol_type = genomic DNA
atggaggacatatcatcttccggcgaagaaatcagcatgaaagcgatctcaaaagtgattggagtgcccgatattcgagatgggcccactctcacaaaatgcatagttgctgagttcgttgggactttgctgttagtgctcataggatgcatgtcggtagcatttgtccatcaggacaacttcgttgacgttgtgaaaattgccatggctttcgggctcatcatcgcctctatggtccaggcaataggtcacgttagtggttgtcacatcaatccggctgtaacttgtggactagctgtgtcgggacatgttagcataataaaaggtatgctgtacattgtcgcgcaatgccttggagccatctgtggagcaatcattttgaatgaaatcacgccaaaaacaggttacacggctgctggtaatctcggagtaacgacactgtctacaggagtttccgacctgcagggtgtggcgatagaggcactaatcacatttgtgctgcttttagttgtccagtccgtctgcgatgggaagcggaccgacatcaaaggatctatcggtgttgcgataggattcgcaattgcttgctgccatctcgccgcgatcaagtacaccggtgctagtatgaaccctgctagatcattaggccctgcattcgtcagtggaatttgggacaaacattgggtctactgggccggtccaatactcggtggagtcactgccagtctgctttacgccatcactttcaaagccaaaaaaagatcggatgagagctcctatgatttctga

SEQ ID NO: 43 - protein, 588 aa, Bemisia tabaci
MEKLQMKIAVLLFLVFPTAWCREAMEEQPQRDLAWWEKGVIYQIYPRSFKDSDGDGVGDLKGIAEKIDYLSKLGVEAVWISPIFRSPMADFGYDISDFRAIEPMFGTMEDFERLKRLFHKNGLKMILDFVPNHTSDEHDWFKKSVARVDPYTDYYNWVNGKMSENGTIQPPNNWLSYFGGSAWTWNEKRKQYYLHQFHPKQPDLNYRNPLVVQEMKDVLTYWMDKGVDGFRMDAVMTIMEDIKLRDEPLSGKTDVLPTDEEYLNHIYTRDQPGTYEIIKQFRKHLDDYSRKTHTVKFMATEAYSNLTSTMKYYGTKDNPGAHFTFNFETIEALTPQSNAADFKEVIEDWYAALPAGKWSNWVLGNHDKRRVGSRYGIDMLDGLHMLQMCLHGTSVTYAGDEIGMVDTFIRWDQTKDPPALNVGPERYQRFTRDPARTPFQWNASTSAGFSTNPKTWLPVNPDYWSHNIVTEKKKTSSHFKNYRRLLALKKSPVIQFGSVNVYTLSDWVLVITRTLKDHPTYIVILNLGTELEDTKGLRTLANLPEQIRLHTCSINCGYSPGTQLRTDEIQLRPKAGFVCIARDGLKAS

SEQ ID NO: 44 - DNA, 1695 bp, Bemisia tabaci
  source 1..1695: mol_type = genomic DNA
atggaagagcaaccgcaacgcgatctagcttggtgggagaaaggagttatctatcaaatctatccgcgatcattcaaagactcggatggcgacggagtcggcgatttgaaaggaattgcggagaaaattgattacctatcaaaactaggcgtcgaagcagtttggatttctccgatttttcgctccccgatggcagattttggttacgatatatcggatttcagagcgatcgagccaatgtttggcacaatggaagattttgaaaagctgaaaagattattccataaaaatggattaaaaatgattctggacttcgtgccaaaccataccagtgacgaacatgattggtttaagaaatcagtcgcaaaagttgatccgtacacagattattacaactgggtcaacgggaagatgagtgagaatggaaccatgcaaccaccgaataactggctttcatattttggtgggtcagcttggacttggaatgaaaaacgaaagcagtactatctgcatcagttccatccgaaacaaccagatttgaactataggaaccctctcgttgttcaagagatgaaggatgttttgacttattggatggacaaaggagtagatggattcaggatggacgctgtgatgaccatcatggaggacattaaactgagggacgaaccactctcaggcaaaacggacgtcctgccaactgatgaggaataccttaaccacatttataccagagaccaaccggggacctacgagataattaaacagtttcgaaaacatctcgatgattattccaggaaaactcacacggtcaagttcatggcgacagaggcatattcgaatttgaccagcacgatgaaatattacggcacaaaagataatcctggagcccactttacgttcaattttgagaccatcgaagcgctaaccccacaatcgaacgcggcagatttcaaagaggtcatcgaagactggtatgctgctctccctgccggcaagtggtctaattgggtgttgggaaaccacgacaagcggcgggtaggctcgcgctacgggatcgacatgttggacgggctgcacatgcttcagatgtgcctgcacggtacctcggtgacgtatgccggggacgagatcggaatggtggacacgttcatccgctgggatcaaaccaaagatccgcccgctctgaacgtcggccctgagaggtatcaaagattcacccgggaccccgccagaactccgttccagtggaatgcgtccacaagtgcaggtttttcgacgaatccaaaaacttggcttccagtcaatcctgattattggtcacacaatctagttacagaaaagaaaaaggccagtagccatttcaaaaattaccgaagattgttggctctgaaaaagtcgcccgttattcaatttggcagtgtaaatgtctacaccctatcagactgggtccttgtcataaccagaactctcaaagaccacccgacatacattgtcattctgaaccttggcacagaattagaggatacgaaagggttgagaactatcgcgaacttgccagaccaaatcaaactgcatacgtgcagcatcaactgtggctacacgcctggagctcaacttcggacggatgaaatccagcttcgaccgaaggccggctttgtgtgcatcgctcggggtggtctaaaagcctcttga

SEQ ID NO: 45 - protein, 676 aa, Bemisia tabaci
MTVLFVNIFAAAIVATCFVTVTCAVAGADNSKTEDCDDTALFEQSMATHPKQSKLGAGSERKADEKLEWWQTSVFYQIYPMSFKDANGDGKGDLQGIASKADHFVDIGVGAVWLSPIYSSPMADFGYDISNYTEINEIFGNLDDLVHLREKLHARGVKLILDFVPNHSSNEHPWFLNSVKKIDPYTNYYVWRDPVIDANGKKSPPNNWLSIFNSGSAWEWNEERGQYYLHQFAVQQPDLNYNCKELVEEMKEVLRFWLSHGVDGFRMDAVPYLFEDSLFRNEPYVDENIKNSKMYDSLIHIYSMDRPKTYEMIGQFRKVLDDFAKEHNTPPKVILSEAYTSLNYTMKYYGDAKNPGSHMPFNFLMINECHKDSSANDFNAAIHNWMDNMPDDHWANWVMGNHDQPRIATRYGPELVDAINMLVMLLPGTAITYNGEEIGMSNGYIRWDQTIDPAGINVGPKEYEKYSRDGCRTPFQWDDSISAGFSSNSRTWLPVNPNSYYLNLKNQKEESYSHYHIYKRLTTLRKTRTFQRGAWKTYVLSNWVLAIERMMEGEDSYVLVINLGSEYEPIKLSDQIKSLPDTMKVHTASVNSAIKNGESVNTASSGFLMRPKSALVLTTSKEDPPNPAPASSSVSLKTSLCVLVFGILFAILSVKAICYCRTSKSSTVITDLKSGQ

SEQ ID NO: 46 - DNA, 2031 bp, Bemisia tabaci
  source 1..2031: mol_type = genomic DNA
atgaccgttttatttgtgaatattttcgctgctgctattgttgctacctgtttcgtgacggtaacatgcgcagtagcaggagcagataattctaaaacggaggactgtgacgatacggccttatttgagcaatcaatggctactcacccaaaacaatcgaaattgggtgcaggatcggaacggaaggctgatgagaaacttgaatggtggcagacaagtgttttctatcaaatctaccccatgtctttcaaagacgcgaacggggatggaaaaggagatttacaagggatcgcatccaaggccgatcactttgtagacattggcgtgggagcagtatggctgtctcccatttattcctctccaatggccgattttgggtacgacatctcaaattacaccgagataaacgaaatttttggcaacctggatgacttggttcacctcagagaaaaactgcatgcccgtggagtcaagctgattttggattttgtgcccaatcattccagcaatgaacacccttggttcctcaattctgtaaagaaaattgacccttacactaactactacgtatggagggatccagtgattgacgctaacggcaagaaaagccctccaaacaactggttgagtattttcaacagtggatctgcatgggaatggaacgaagagagaggacaatactacctgcatcaatttgcagttcagcaacctgatttgaactacaattgtaaggagctcgtagaggaaatgaaggaagttctcagattttggcttagtcatggcgtggacggttttcgtatggatgcagtaccttacttgtttgaggattctttgttccgtaatgagccgtacgtcgacgagaatattaaaaactccaaaatgtacgactctcttattcacatttactccatggatcgaccgaagacttacgaaatgatcggccagttcagaaaagttctagacgatttcgccaaagaacataacacacctccaaaggttattttgagcgaggcttacacatctttgaactatacaatgaaatactacggagacgctaaaaatcccgggagccacatgcccttcaacttcttgatgatcaacgaatgtcataaagattcgagtgccaacgatttcaatgccgccattcacaactggatggacaatatgcctgatgatcattgggccaactgggtgatgggaaaccatgatcaacctcgaattgcgacacgatacggacctgaattagtagatgctattaatatgcttgtgatgctactaccaggaacggccatcacctataacggcgaagaaattggcatgtcaaatggttacattcgatgggatcaaactattgatcctgcggggataaatgtcggaccgaaagaatatgagaaatactcgcgtgatggatgcaggaccccctttcaatgggacgattcaattagtgcaggattttcttctaacagccggacttggttacctgtcaaccctaattcctactatttgaatttaaagaaccaaaaagaggaaagttacagccactaccacatttacaaaaggctcacaactcttcgtaagacgagaacattccaaagaggggcatggaagacttatgtcctttccaactgggtcttagctattgaaaggatgatggaaggagaggactcttacgttctggtcataaatcttggaagtgaatatgaaccaataaaactttccgatcaaattaagtccttaccagacacgatgaaagtccatacggccagtgtcaactccgcgattaaaaatggcgaaagcgtgaacacggcgagttccggctttttaatgcgccctaagtcagctctagttctgacgacatccaaggaggacccaccgaaccccgctccggccagcagttccgtttccttgaagacaagtctatgtgtcctcgtttttggaattctcttcgcaatattgagtgtcaaagccatttgttattgtcgaacatccaaatccagcactgtcataactgatttaaaatcgggacaatga

SEQ ID NO: 47 - protein, 559 aa, Bemisia tabaci
MQGFQMALWGGRFSEAADQRFKLFNDSLRFDYRLAEQDILGSIAWSKALVTVNVLTSQEQKELESALNELLAEVRSHPEQIPQSDAEDIHSWVESRLIEKVGALGKKLHTGRSRNDQVATDLKLWCKAQVKDLILATRGLQQALITTAEATQDILMPGYTHLQRAQPISFAHWCLAYVEMLARDETRLRDTLKRLDVSPLGCGALAGTAYPIDREQLAGWLGFASATRNSLDTVSDRDHVLEILSNAAIGMVHLSRFAEDLIFFNTGEASFVELSDKVTSGSSLMPQKKNPDALELIRGKCGRVQGALTGMLTTLKALPLAYNKDMQEDKEGLFDAVDTWLGCLQMAALVLDGIKVKKSRCREAAEQGYANSTELADYLVGKGLPFREAHHIVGRVVVEAIAQGVPLGAMSLQELKKFSDVIEGDVYPVLELQSCLEKRCAKGGVAPAQVALAIAEAKERLLGSEPKAFSLDEAPLKRFCAFLRTQESLRHLDRVKVIQWTPCFLSGRHAIYRINWFHFFRYSSFFSKFEIAILLSGDQRTHLPILTNKFNVIKAWFFL

SEQ ID NO: 48 - DNA, 1686 bp, Bemisia tabaci
  source 1..1686: mol_type = genomic DNA
atgcaagggtttcagatggcgctctggggcgggcgattctcggaggcggcggaccagcgcttcaagctcttcaacgactcgctgcgcttcgactaccgcctcgcggagcaggacatcctcggctccatcgcttggtccaaggctctcgtcaccgtcaacgtcttgaccagccaagagcagaaggaactcgagagcgccctcaacgagcttctggccgaggtccggagccaccccgagcagatacctcaaagcgacgccgaggacatacactcctgggtcgagtctcggctcatcgagaaagtcggggctttgggcaagaagctccacaccggccgcagccgcaatgatcaggtggcgaccgacctaaagctctggtgtaaggcacaagtcaaggatctgatcctggcgacgcgcgggctacagcaagcgctcatcaccaccgccgaagcgacccaagacatcctcatgcccggctacacccacctgcagcgcgctcagcctatcagcttcgcgcactggtgcctggcctacgtggagatgctggcgcgggatgagacccgtctaagggacaccctcaagcggctggacgtgagtcctctgggatgcggggccctcgccggcactgcctaccccatcgaccgcgagcagctcgcaggctggctcggcttcgcttcggccacgaggaatagtctcgacacggtctcggatcgggaccatgtcttggagattctgtccaacgccgccatcggcatggtccatctctcgcgcttcgccgaggatctcattttcttcaacacgggcgaggcgtccttcgtggaactgtcggacaaggtgacctcgggctcctcgctgatgccgcagaagaagaacccagacgccctggagttgatccgcgggaagtgcgggcgggttcagggggcgctgacggggatgctgacaacgctgaaggcgctcccgctggcctacaacaaggacatgcaggaagacaaggaagggctcttcgacgccgtcgacacgtggctagggtgcctccagatggcagcactcgtccttgacggcatcaaggtgaaaaaatctcgttgccgagaggcggccgagcaaggctacgccaactcgacggagctggccgactacctggtgggcaaagggctccccttccgcgaggcccaccacatcgtcggccgggttgtcgtcgaggccatcgcccaaggcgtgcccctcggggcgatgtccctgcaggagctcaagaaattctccgacgtcatcgagggagacgtctaccccgtcctcgagctccagtcctgcctcgagaagcggtgcgccaagggaggcgtcgcgcccgcccaagtggcgctggccatcgccgaggcaaaagaacgtctcctcggaagtgaaccgtgaaaagcgttctctctggactgagaggctcccttaaaaaggtaattctgcgcatttctgagaactcaggagtctcttcgacatttggaccgcgttaaggtgattcaatggacgccatgttttttgtcaggacggcatgcgatatatcgcatcaattggttccattttttccgctactcgtcatttttctccaaatttgagatcgcaattctgttgtcaggagaccaaagaactcacttaccaattttaacaaataaattcaacgtaataaaggcgtggtttttttta

SEQ ID NO: 49 - protein, 437 aa, Bemisia tabaci
MAGKAPLWNHFTYRNNELYCEDIPVANLAEKYGTPLYVYSQAAILKQLQALQEAFSELKPLVCYSVKANSNLSILRLLAEHGSGFDVVSGGELKRVSVAGGQARQTVFAGVGKTDEEIAAGLTAGVLMFNVESEEELDAIVRVAQSKKIQAPVALRINPDVDPKTHRYISTGHKESKFGMDIQRSLLLAEKFKGSESIKMIGIHMHIGSQITTTTPYSSAAAKAVDIITRLREIGHPIDWFNMGGGFGISYDGKTSPSIAEFAAAVVPSLRKTGCRLVMEPGRVIVGNAGILISRVIYTKVSGEKQFLIQDAAMNDLIRPSLYESFHGIWPVKVPAGFPSPPDDFETSRINGTKLCDVVGPVCESGDFLAQDRALPPLNRGELLAIFSVGAYGMTMASNYNSRPRPAELLVNGSGVVVARRRETFDDLIAEELNPVS

SEQ ID NO: 50 - DNA, 1314 bp, Bemisia tabaci
  source 1..1314: mol_type = genomic DNA
atggccggaaaagcgccactttggaatcacttcacgtatcgtaataatgaactctactgcgaagatattccagtcgcaaatcttgctgaaaaatatggcacaccattgtacgtatacagccaggcagcaattctcaagcagcttcaggcactccaggaggcattctctgagctgaagccactagtctgttattcagtaaaggcaaattccaatctatcaattcttcgcttgttggcggaacatggaagtggatttgatgtggtatctggtggtgagttgaagcgcgtcagtgtcgctggaggccaggctagacaaactgtctttgctggagttggcaaaaccgatgaggagattgctgctggcctgaccgctggggtcctaatgttcaacgtggagagtgaggaggaattggatgctattgtccgagtagctcaatccaaaaaaatccaggcacctgtggctctccgaatcaatcctgacgttgaccctaaaacgcatcggtatatttcgacaggccataaggagtcaaagttcggcatggatattcaacgatcattattgcttgcggaaaaattcaaaggctcagaatctataaagatgattggcattcatatgcatatagggtcgcagattacaacaacgaccccttacagcagtgcagcagctaaggctgttgatattattactcgtctgcgggaaataggccacccaatcgactggtttaacatgggtggcggctttggaatcagttacgacgggaagacgagtccatcaattgcagaatttgctgctgctgttgtacccagcttaaggaaaacggggtgccgtctggtgatggaacctggtcgggtgattgttggtaatgccggcattcttatcagtcgagtaatttacacaaaggtatcaggggaaaaacagtttctgatccaggacgccgcaatgaatgatctaatccgtccatctttatacgagtcgtttcatgggatttggccagtcaaagtccctgctggctttccttccccaccggatgattttgaaacatcgcgtattaatggaacaaaattatgcgatgtggttggcccagtatgtgaaagcggtgacttcctggcgcaagatcgagctttgccacccttaaatcggggtgagctcctagccattttttccgttggggcatacggaatgacaatggcctccaactacaattctcgccctcgccctgctgagctgttggttaatggatcaggcgttgttgtggctcgccggcgggagacctttgatgacttaattgccgaggagctaaatcctgttagttaa

SEQ ID NO: 51 - protein, 524 aa, Bemisia tabaci
MARNVPSSLLKLLKRQKLAVKQIVRRQVTAPATAPAYHNPHQSFQFSETSVRQCLPSQLKPKPPVSELGFGTQFTDHMLRVEYDVDKGGWQSPLIVPLEPLTLHPAAKGLHYALQLFEGMKAYRGVDGRIRVFRPDLNMARMNSSALRISLPQFDGEELLKCIKKLVSLDQEWVPISETASLYIRPTLVATDPVLGVNAPSSALLYVILSPVGPYFQTMKALTLLAEPNYVRAWPGGSGSTKVGANYGPTIYVQSVAKEVGAHQILWLFGENHAVTEAGAMNVFCVFINDKGRIELSTPPLNGLILPGVTRASILDLAREWNEFDVSERFLTMSDIVKLRSENRLLEFFGSGTAVLVNPVERLIYNNQVIDIPTMDQPDPIFLRLLRTLTDIQYGRLKHPWAEEIDDALKFHSTSMLLYSHRKQYLREFEINGLTRFLLKFQKEPLNHMCYLIYTPGQFLRETLEVRSNLFLWKNKFSVLGSASVQALFLPDLYIQMKFCTLNLYSLICNVFVTFNKSFLRKTL

SEQ ID NO: 52 - DNA, 1594 bp, Bemisia tabaci
  source 1..1594: mol_type = genomic DNA
atggcgcgcaacgtgccctcttccttactcaagctcttgaagcggcagaagctcgctgtcaagcagatagttcgtcgacaagtcactgccccggcaactgcgcctgcgtaccataatccacatcaaagttttcagttttcagagacctcagtgcgacaatgccttccgtcgcagctgaagccgaagccgccagtgtcggaactgggcttcggcacccaattcaccgaccacatgctcagagtcgagtatgacgtcgacaaaggcggctggcaatctcccctcatagttcctctggagcccctcacccttcatccggctgctaaagggctccactacgccctccagctgttcgaggggatgaaggcgtaccgcggcgtggacggtcggatccgggtgttccggccggacttgaacatggcgcggatgaacagctcggcgctgcggatcagcctgccccagttcgacggcgaggagctcctgaaatgcatcaagaaattggtcagtctcgaccaggagtgggtgcccatctcggagacggccagcctctacatccgacccacgctcgtcgccaccgatccggtgctgggtgtgaacgcgccctccagcgccctactctacgtgatcctcagcccagtgggcccgtatttccagacgatgaaggccctcacgctactcgccgagcccaactacgtccgcgcttggcccggcggcagtggctccaccaaagtcggcgccaactacggcccaaccatctacgtccagtcagtggcaaaagaagtcggtgctcatcaaattttatggctattcggagaaaatcatgctgtcactgaagccggagccatgaacgttttttgcgtcttcatcaacgacaaaggaagaatcgagttgagtaccccgcctttgaatggtctcatcctgccgggcgtaacccgtgcctcgattttggacctggcgcgggagtggaacgaattcgacgtcagcgagcggttcctcacaatgagcgacatcgtcaaactgaggtcggagaataggttactcgaattttttgggtctggaacagctgttctagtaaatccagtggagcgtctcatctacaacaaccaagtgatcgacattccgaccatggaccaaccagatcctatcttcttacggcttctcagaactctcacagacatccagtatggaaggcttaaacacccttgggcagaggagatcgattgagatgcattgaaatttcattctacatctatgttactataatactcacatcgcaaacaatatctgagggaattcgagataaatggtttgaccagattcctcctaaaatttcaaaaagaacctctcaatcatatgtaatgttatttaatttacactccaggtcagtttttgagggaaactttagaagtaaggtcaaacctatttttgtggaaaaacaagttttctgtgctgggttctgcgagcgttcaagcattgtttttaccagacctgtattagatacaaatgtgataaaaattttgtaccttaaacctgtaatattcgttgatttgcaatgtttttgttacttttaataaatcatttttacgtaaaacactga

SEQ ID NO: 53 - protein, 243 aa, Bemisia tabaci
MINIGVCGSTGKMGKMIIERIGSFDSCKISSTFSRQTNDSDEFCRNCQVIIDFSNGDLLEKLLKSAIKYKNKLVIGTTGFESKHSDLMHSASKEIAILYSANMSLGINLLSILTEKAAKILNKNYDIEILESFHRMKLDAPSGTALMFGKTLAEARNLDSNKVVIHNRHGKRLPGEIGIASIRGGQVCGEHEIMFLGDNEALSCKHQALNRESFADGAIKAAIWIASKETGFYSMRDVLGQLG

SEQ ID NO: 54 - DNA, 732 bp, Bemisia tabaci
  source 1..732: mol_type = genomic DNA
atgataaatattggagtctgtggttctaccggtaaaatgggaaaaatgataattgaaagaataggaagttttgattcttgtaaaatttcatcaacattttccaggcaaactaatgattcagatgaattttgtcgtaattgtcaggtaatcatagatttttcaaatggcgacttattggaaaaattgctcaaatcagcaataaaatacaaaaataaattggttattggtactaccggttttgagtcaaaacactcagatcttatgcattctgcttcaaaggaaatagctatattatattctgctaatatgagtctagggataaatttactttccatactaactgaaaaagcagcaaaaattctgaataaaaattatgatatagagatcttagagtcctttcacaggatgaagttagatgctccgtctggtactgctcttatgtttggcaaaacattagcggaagccagaaatttagattctaacaaggttgtaattcataaccgccatggtaaaagactaccaggcgaaattggtattgcttcaatcagaggaggacaggtgtgcggagagcatgagataatgtttctaggtgataatgaagctctcagctgtaaacaccaagctcttaatagagaatcttttgctgatggggcaattaaggctgcaatttggattgctagtaaagaaactggtttttattctatgcgtgatgtacttggtcaactcggttaa

SEQ ID NO: 55 - DNA, 20 bp, synthetic construct
  source 1..20: mol_type = other DNA
ttgtttcgcaagtttgccgt

SEQ ID NO: 56 - DNA, 20 bp, synthetic construct
  source 1..20: mol_type = other DNA
gactgattgacgccctggat

SEQ ID NO: 57 - DNA, 20 bp, synthetic construct
  source 1..20: mol_type = other DNA
aacactgcgaatagcgcatc

SEQ ID NO: 58 - DNA, 20 bp, synthetic construct
  source 1..20: mol_type = other DNA
cgccactctagatgttcgca

SEQ ID NO: 59 - DNA, 22 bp, synthetic construct
  source 1..22: mol_type = other DNA
cggtaaggtctgaaactgcgat

SEQ ID NO: 60 - DNA, 22 bp, synthetic construct
  source 1..22: mol_type = other DNA
gttttgctagatgtgcaaggca

SEQ ID NO: 61 - DNA, 20 bp, synthetic construct
  source 1..20: mol_type = other DNA
actacaattctcgccctcgc

SEQ ID NO: 62 - DNA, 20 bp, synthetic construct
  source 1..20: mol_type = other DNA
gtcatcaaaggtctcccgcc

SEQ ID NO: 63 - DNA, 20 bp, synthetic construct
  source 1..20: mol_type = other DNA
aagctctggtgtaaggcaca

SEQ ID NO: 64 - DNA, 20 bp, synthetic construct
  source 1..20: mol_type = other DNA
aggatgtcttgggtcgcttc

SEQ ID NO: 65 - DNA, 19 bp, synthetic construct
  source 1..19: mol_type = other DNA
cgtccagagtcagtggcaa

SEQ ID NO: 66 - DNA, 20 bp, synthetic construct
  source 1..20: mol_type = other DNA
gttcatggctccggcttcag

SEQ ID NO: 67 - DNA, 20 bp, synthetic construct
  source 1..20: mol_type = other DNA
ggtcctaccagttgtgcgaa

SEQ ID NO: 68 - DNA, 20 bp, synthetic construct
  source 1..20: mol_type = other DNA
agaatggagccgaacccaag

SEQ ID NO: 69 - DNA, 20 bp, synthetic construct
  source 1..20: mol_type = other DNA
cattccactacagagctcca

SEQ ID NO: 70 - DNA, 20 bp, synthetic construct
  source 1..20: mol_type = other DNA
tttcaggtttcggatggctt

SEQ ID NO: 71 - DNA, 23 bp, synthetic construct
  source 1..23: mol_type = other DNA
tgtcaggagtaacgacgtgtttg

SEQ ID NO: 72 - DNA, 20 bp, synthetic construct
  source 1..20: mol_type = other DNA
ttcgggaacggtaagtgctc

SEQ ID NO: 73 - DNA, 22 bp, synthetic construct
  source 1..22: mol_type = other DNA
tggtaaaagactaccaggcgaa

SEQ ID NO: 74 - DNA, 23 bp, synthetic construct
  source 1..23: mol_type = other DNA
agcttggtgtttacagctgagag

SEQ ID NO: 75 - protein, 7 aa, Bemisia tabaci
DAPGWLM

SEQ ID NO: 76 - protein, 7 aa, Bemisia tabaci
DAVEYLY

SEQ ID NO: 77 - protein, 7 aa, Bemisia tabaci
DAVNHLL

SEQ ID NO: 78 - protein, 7 aa, Bemisia tabaci
DAARHFF

SEQ ID NO: 79 - protein, 7 aa, Bemisia tabaci
DAPEFIF

SEQ ID NO: 80 - protein, 7 aa, Bemisia tabaci
DAVTYMY

SEQ ID NO: 81 - protein, 7 aa, Bemisia tabaci
DAVMTIM

SEQ ID NO: 82 - protein, 7 aa, Bemisia tabaci
DAVQILF

SEQ ID NO: 83 - protein, 7 aa, Bemisia tabaci
DAVTYMY

SEQ ID NO: 84 - protein, 7 aa, Bemisia tabaci
DAVPHLI

SEQ ID NO: 85 - protein, 7 aa, Bemisia tabaci
DAAKWLF

SEQ ID NO: 86 - protein, 7 aa, Bemisia tabaci
DAVPWLY

SEQ ID NO: 87 - protein, 7 aa, Bemisia tabaci
DAIKHLV

SEQ ID NO: 88 - protein, 7 aa, Bemisia tabaci
DAVVCLY

SEQ ID NO: 89 - protein, 7 aa, Bemisia tabaci
DAVPYLF

SEQ ID NO: 90 - protein, 7 aa, Bemisia tabaci
ETVSYLF

SEQ ID NO: 91 - protein, 7 aa, Bemisia tabaci
DAIKHVY

SEQ ID NO: 92 - protein, 7 aa, Bemisia tabaci
DAINFMF

SEQ ID NO: 93 - protein, 7 aa, Bemisia tabaci
DAVAYLF

SEQ ID NO: 94 - protein, 7 aa, Bemisia tabaci
DAVHTMF

SEQ ID NO: 95 - protein, 7 aa, Bemisia tabaci
DAIPILF

SEQ ID NO: 96 - protein, 7 aa, Bemisia tabaci
DAVNYLF

SEQ ID NO: 97 - DNA, 534 bp, Bemisia tabaci
  source 1..534: mol_type = genomic DNA
atgcaacgcgtactttattttgtcatttttgtcctttttctattcggccagctgggcagccgacaaactgcaaaggcagttgactggtgggaaaataccataatctaccagatatatccaaggtcattcaaggattcgaacggtgatggcctcggagacttgaaaggtatttacgagaaactggattatgtaaaagaaataggagtcgggaccatttggattcaatcattctataaatctccgatggttgatttggggtacgatatcaccgatttcagagctgtagatccaatatttggaaccatggatgacttcaagagagtagtgcaggggatccatgaaaggggtatgaggattatcctagacttcgtgccaaaccatacaagtaacacctgtgaatggtttaaactttctgaacgtcgcatcgaaccatatacaaatttttatgtttggcaagatccaaagcagttaaatgattcccactggacatatccgaataactgggtagctatacctaccgatgttatttattga

SEQ ID NO: 98 - protein, 177 aa, Bemisia tabaci
MQRVLYFVIFVLFLFGQLGSRQTAKAVDWWENTIIYQIYPRSFKDSNGDGLGDLKGIYEKLDYVKEIGVGTIWIQSFYKSPMVDLGYDITDFRAVDPIFGTMDDFKRVVQGIHERGMRIILDFVPNHTSNTCEWFKLSERRIEPYTNFYVWQDPKQLNDSHWTYPNNWVAIPTDVIY

SEQ ID NO: 99 - DNA, 1773 bp, Bemisia tabaci
  source 1..1773: mol_type = genomic DNA
atgtttaaggtaatcactgtgtgcttatggctattcgccttcaacagtcttaatgtgtcgtccgagtatgtttatgagggtttgaagagcgattcggtcgaaccagactggtggcagacggaaattatctatcaaatatacgtaagatcgtttaaagacagcgatggtgacggaatcggagacctgaacggtataacagagaaagtcccatattttaaaactatagatgttggcgccgtttggttgtcgccaatattcctctcaccacaaaatgatttcggatacgatatatcagactacaaagagatcgatcctatttatggctcaatggcagactttgaacggatgagggatgagtttcacaagcatggcataaaggtcttattggactttgtgccaaatcacacgagtgacgaacacgaatggttccaaaagtcaattaagaaaatagagcccttttcagactattacgtatggaaagatccgatccgtgatgtacatggaaataacacgcctccgagtaattggttaggtgtgttcaacagtgggtctgcgtgggaatggaatgaagaacgtcaacaatattatttacatcagttccaagtgaaacaacccgacttgaactacagaaatccgtcggttagagaagaaataaagaacacgctgttatattggttgggacgtggcgtcgacgggttcagattcgacgcggtgaactatctattcgaaagagaagatctagctgacgaacctaagtctaataaaattggctatttggacaccgattacgattctttaacgcatacgagcacacttgatcaacctgaaacttattccatcgttcgtcaatggaggcagatgctggacagttacaggactagggaaaagaaaaccaagtttatgatggtggaatgttactcaccatttgataaaactttgttgtactacggtagtaattcagagcctggcgctcattttccatttaactttttattcatcggaacgttcgatcaacagtctgatgctgctaaggtccataacatgatcaaatcatggattcgtggtatgcccaccggcatgtggcctaactgggtgttaggtaaccatgataacgcaagagtggcttcgaggagcaatccaatgttagttgatggactacacatgatccaacacctgttgccccgcacctctgtgacttattacggagacgaactaggtctgatcgacacaaccgttcgttgggaccaaacagttgatccagcggggcttaacgtgggcccctataggttcttgaaattcagcagagatcccgtgaggactccgttcccatgggacagttcgtataacgcaggtttttctaattcgtcttcgttgtggcttcctcttaacgccgattattggaaaaaaaatatggttgaagaatcaaggtttaaaagtaacctaaggtcatacaggcaattggctcggttaaggaggagtctcacatttgtcaaaggcgatttgcatttatacacactgtccaaatgggtgtttggattttcacggagtttttatgatcacccgacttacttcattgtagttaatttcgggagtgaaatagaaacagttaatttgatggaagctagaggtactttaccattaactatgaaagttaaggtatctagcattaactctggttttgttaccggaaatttagtacgtactgatagtgtattactgcgcccgaaagcagctcttgtacttacaacctcaagaatgaacgaagatgcataa

SEQ ID NO: 100 - protein, 590 aa, Bemisia tabaci
MFKVITVCLWLFAFNSLNVSSEYVYEGLKSDSVEPDWWQTEIIYQIYVRSFKDSDGDGIGDLNGITEKVPYFKTIDVGAVWLSPIFLSPQNDFGYDISDYKEIDPIYGSMADFERMRDEFHKHGIKVLLDFVPNHTSDEHEWFQKSIKKIEPFSDYYVWKDPIRDVHGNNTPPSNWLGVFNSGSAWEWNEERQQYYLHQFQVKQPDLNYRNPSVREEIKNTLLYWLGRGVDGFRFDAVNYLFEREDLADEPKSNKIGYLDTDYDSLTHTSTLDQPETYSIVRQWRQMLDSYRTREKKTKFMMVECYSPFDKTLLYYGSNSEPGAHFPFNFLFIGTFDQQSDAAKVHNMIKSWIRGMPTGMWPNWVLGNHDNARVASRSNPMLVDGLHMIQHLLPRTSVTYYGDELGLIDTTVRWDQTVDPAGLNVGPYRFLKFSRDPVRTPFPWDSSYNAGFSNSSSLWLPLNADYWKKNMVEESRFKSNLRSYRQLARLRRSLTFVKGDLHLYTLSKWVFGFSRSFYDHPTYFIVVNFGSEIETVNLMEARGTLPLTMKVKVSSINSGFVTGNLVRTDSVLLRPKAALVLTTSRMNEDA

SEQ ID NO: 101 - DNA, 1785 bp, Bemisia tabaci
  source 1..1785: mol_type = genomic DNA
atgaagctggtggcagtattttgtctcgttttggcttcggcctcctgcgaggtaatgaaagcataccctcaaagagacttagcgtggtgggaaaggggtgtcatttatcaaatttaccccaggtcgttcaaggactcaaacggagatggagtcggcgatctgaagggtatcgcagagaaaatcgattacctttcgaatttaggtgtcaaggcggtgtggctttctcctatttttcgttcaccgatggttgatttcggctacgatatctctgacttcagagctattgagcctatgttcggaaccatggaagattttgaagtactgaaagagttattacagaaaaatggactaaaaataattctggacttcgtgccgaaccatacaagtgatcaacacgactggtttatacgatccgtcgcaagagaggacccttacaccaattattacgtttgggttgatgccaagtacgtaaatgggaccagatcacccccaaacaactggctgtcagattttggaggctcagcttggacctggaatgaaaaacggaagcaatacttcttgcatcagttccaccctaaacaacccgatttaaactacagaaaccctctcgtggtgcaagaaatgaaggatgttttgacatactggatggacaaaggagtcgatggttttaggatggacgctgtgatgacgattatcgaagacgatcagctgagggacgagccgccgtccaacaaaccagacgttctgcccacagacgtagaattcctcacccacatccacactagggatcagccgggaacctatgatattattaaacagttccgaaagctcctggacgactattcggccaaaacaggcactgtcaagttcatggcgacagaagcgtactcaaatttaacaaacacgatgaaatactacggagacaaatattaccccggagctcacttcacgttcaatttcggaccaatcaccggactgactgctcaatcaagtgcaaagcaattcaaagaagtgatcgaaaactggtatagagagcttccagaaggcaaatggtctaactgggtgcttgggaaccacgaccaacaacgcgtggccactcgctacggtatcgacatgttggacggacttcacatgcttcaaatgtgccttcacggtacctccgtgtcgtattcgggtgatgagatcggaatgaccggtcccttcatccggtgggaccagactaaggacccgcccgccctcaacgttggacctgagaggtacctgagattcacaagggatcctgctaggactcccttccagtgggcttcatgcaccagtgcaggattttcgacgaaccctaagacttggctccctgtcaatcctaactactggtcgcacaacttggtcacagagaagaagaagaacagaagccacctgaaaaattatcagaagctcctggccttgaaagaatcaccagtcatacagtttggcaacttgaacgtttacacactatccgattgggtgcttgtcgtaacaagggctctggaaggacacccaacttatgtggtcgtactgaatataggttcagaaatagaatacacgacaaaactaagtgctgtcgcgaacctccccgaacaactcaaattacatatctgcagtcttaactgtgggtacacgcctggagttcaacttcatactgagaaaattcagctccgaccaaaagcgggtatggtcttatcaacccagaaaggtgttcaagctagcagagaagccgatgagtcaagttcagaacttcaaatggaataa

SEQ ID NO: 102 - protein, 594 aa, Bemisia tabaci
MKLVAVFCLVLASASCEVMKAYPQRDLAWWERGVIYQIYPRSFKDSNGDGVGDLKGIAEKIDYLSNLGVKAVWLSPIFRSPMVDFGYDISDFRAIEPMFGTMEDFEVLKELLQKNGLKIILDFVPNHTSDQHDWFIRSVAREDPYTNYYVWVDAKYVNGTRSPPNNWLSDFGGSAWTWNEKRKQYFLHQFHPKQPDLNYRNPLVVQEMKDVLTYWMDKGVDGFRMDAVMTIIEDDQLRDEPPSNKPDVLPTDVEFLTHIHTRDQPGTYDIIKQFRKLLDDYSAKTGTVKFMATEAYSNLTNTMKYYGDKYYPGAHFTFNFGPITGLTAQSSAKQFKEVIENWYRELPEGKWSNWVLGNHDQQRVATRYGIDMLDGLHMLQMCLHGTSVSYSGDEIGMTGPFIRWDQTKDPPALNVGPERYLRFTRDPARTPFQWASCTSAGFSTNPKTWLPVNPNYWSHNLVTEKKKNRSHLKNYQKLLALKESPVIQFGNLNVYTLSDWVLVVTRALEGHPTYVVVLNIGSEIEYTTKLSAVANLPEQLKLHICSLNCGYTPGVQLHTEKIQLRPKAGMVLSTQKGVQASREADESSSELQME

SEQ ID NO: 103 - DNA, 1506 bp, Bemisia tabaci
  source 1..1506: mol_type = genomic DNA
atgaccgttttatttgtgaatattttcgctgctgctattgttgctacctgtttcgtgacggtaacatgcgcagtagcaggagcagataattctaaaacggaggactgtgacgatacggccttatttgagcaatcaatggctactcacccaaaacaatcgaaattgggtgcaggatcggaacggaaggctgatgagaaacttgaatggtggcagacaagtgttttctatcaaatctaccccatgtctttcaaagacgcgaacggggatggaaaaggagatttacaagggatcgcatccaaggccgatcactttgtagacattggcgtgggagcagtatggctgtctcccatttattcctctccaatggccgattttgggtacgacatctcaaattacaccgagataaacgaaatttttggcaacctggatgacttggttcacctcagagaaaaactgcatgcccgtggagtcaagctgattttggattttgtgcccaatcattccagcaatgaacacccttggttcctcaattctgtaaagaaaattgacccttacactaactactacgtatggagggatccagtgattgacgctaacggcaagaaaagccctccaaacaactggttgagtattttcaacagtggatctgcatgggaatggaacgaagagagaggacaatactacctgcatcaatttgcagttcagcaacctgatttgaactacaattgtaaggagctcgtagaggaaatgaaggaagttctcagattttggcttagtcatggcgtggacggttttcgtatggatgcagtaccttacttgtttgaggattctttgttccgtaatgagccgtacgtcgacgagaatattaaaaactccaaaatgtacgactctcttattcacatttactccatggatcgaccgaagacttacgaaatgatcggccagttcagaaaagttctagacgatttcgccaaagaacataacacacctccaaaggttattttgagcgaggcttacacatctttgaactatacaatgaaatactacggagacgctaaaaatcccgggagccacatgcccttcaacttcttgatgatcaacgaatgtcataaagattcgagtgccaacgatttcaatgccgccattcacaactggatggacaatatgcctgatgatcattgggccaactgggtgatgggaaaccatgatcaacctcgaattgcgacacgatacggacctgaattagtagatgctattaatatgcttgtgatgctactaccaggaacggccatcacctataacggcgaagaaattggcatgtcaaatggttacattcgatgggatcaaactattgatcctgcggggataaatgtcggaccgaaagaatatgagaaatactcgcgtgatggatgcaggacccctttcaatgggacgattcaattagtgcaggattttcttctaacagccggacttggttacctgtcaaccctaattcctactatttga

SEQ ID NO: 104 - protein, 501 aa, Bemisia tabaci
MTVLFVNIFAAAIVATCFVTVTCAVAGADNSKTEDCDDTALFEQSMATHPKQSKLGAGSERKADEKLEWWQTSVFYQIYPMSFKDANGDGKGDLQGIASKADHFVDIGVGAVWLSPIYSSPMADFGYDISNYTEINEIFGNLDDLVHLREKLHARGVKLILDFVPNHSSNEHPWFLNSVKKIDPYTNYYVWRDPVIDANGKKSPPNNWLSIFNSGSAWEWNEERGQYYLHQFAVQQPDLNYNCKELVEEMKEVLRFWLSHGVDGFRMDAVPYLFEDSLFRNEPYVDENIKNSKMYDSLIHIYSMDRPKTYEMIGQFRKVLDDFAKEHNTPPKVILSEAYTSLNYTMKYYGDAKNPGSHMPFNFLMINECHKDSSANDFNAAIHNWMDNMPDDHWANWVMGNHDQPRIATRYGPELVDAINMLVMLLPGTAITYNGEEIGMSNGYIRWDQTIDPAGINVGPKEYEKYSRDGCRTPFNGTIQLVQDFLLTAGLGYLSTLIPTI

SEQ ID NO: 105 - DNA, 1860 bp, Bemisia tabaci
  source 1..1860: mol_type = genomic DNA
atgctgttagcgataatgtgcaaatttacggtagcattgatgattatcggtctagtgaccgaatttcacgcaaaatcggctgttttggagcaaaaacctcttgattggtggcaacgaggtgttttttaccaaatttaccctaggtccttcaaagacagtaacggcgatggtgttggagatctgcgaggtatagcggaaaaagtatggtatctaaaagatttaggcgtgtcagcgatatggttcaaccctatattcgattctcctggcttggatctgggatatgacatttccaactacacacgtatcgaccccctttttggaactttagaagatttggagtacttaagagacgctcttcataaagcaggtatacggcttctgctggattttgtcccaaatcatacaagtgatgagcatccatggtttcagtggtctatcaaaaaagtcgatccgtacacaaactattacgtttggaaggacgccaagatggtcaatggtcaaaggaaaccaccaaccaactggataagcgagtttggaggctccatgtggaaatggaacgacgaaagacagcaatacttcttacaccagtttcaccagaagcaaccagacttgaattatgaatgtaaagaactggtccaagagatgatggatgttgtcaggttttggctggaccgcggtgtagatgggctgcgtttagacgccatcaactttatgtttgaggatccgctatggagagatcaagagctcctagcgcctgatctggaccccaacttcttcttgagctacaatagatcaaggacgatggatcagccggccacatacgagatggtcacgagattcagagaaattttcgactcttacagtaaaaaagacggccgaaccagagtaatgatgactgaggcttatacaacgctggataagacactgggtttctatcagtacaacggcaagccgggcgctcacatgccatttaatttctttttcatcacgcatgttaatggacggtcacccgcaaaagattaccagagagctatccaaggctggatgcagggcatgccagaaggacactcggcgaactgggtgataggaaaccatgacaatcatcggatagcggcaagatacggcacggatctcgtagacggtatgaacatgatagcgctcttactcccagggactgggaacacgtactacggggatgaaatcggtatggacgacgcagctatcagattcgacgagggtgtcgacccacaggcctgccagttcgacaaggattggttcaatcgcgtcaccagggaccccgagagaacacctttccagtgggacacatccttgaacgcaggtttttcgacgagcatgaaaacttggctaccggtgaattccaacttttggcgtttaaacctgaaggaccagatgcaaagtaacgaaaaaagtcattacaaggtatacaagagactgatggatgttcgcaagacagacacgatgctgtatggggatgtggagacctacgtacttgccaaatgggtcttcgcctttactaggcgtttgactggcagtgacacttatgtggtggtcgtcaatcttgggagtgacactacatctgtcgatctctctgctttcatgagtgatctaccggagactttaacagtccacacgggcagtgtcaatacccagtatgatccaaatgacaaagtaccaactggtgtattttacatgcgaccaaaagcctccatccttttaactactgcccaagaagtgccgcctgctgcatattcggattccaaaactagtggagtcgcaacaaggacatccactgcaatcggagtaattgggttccttttcgctcataggttactcctatag

SEQ ID NO: 106 - protein, 619 aa, Bemisia tabaci
MLLAIMCKFTVALMIIGLVTEFHAKSAVLEQKPLDWWQRGVFYQIYPRSFKDSNGDGVGDLRGIAEKVWYLKDLGVSAIWFNPIFDSPGLDLGYDISNYTRIDPLFGTLEDLEYLRDALHKAGIRLLLDFVPNHTSDEHPWFQWSIKKVDPYTNYYVWKDAKMVNGQRKPPTNWISEFGGSMWKWNDERQQYFLHQFHQKQPDLNYECKELVQEMMDVVRFWLDRGVDGLRLDAINFMFEDPLWRDQELLAPDLDPNFFLSYNRSRTMDQPATYEMVTRFREIFDSYSKKDGRTRVMMTEAYTTLDKTLGFYQYNGKPGAHMPFNFFFITHVNGRSPAKDYQRAIQGWMQGMPEGHSANWVIGNHDNHRIAARYGTDLVDGMNMIALLLPGTGNTYYGDEIGMDDAAIRFDEGVDPQACQFDKDWFNRVTRDPERTPFQWDTSLNAGFSTSMKTWLPVNSNFWRLNLKDQMQSNEKSHYKVYKRLMDVRKTDTMLYGDVETYVLAKWVFAFTRRLTGSDTYVVVVNLGSDTTSVDLSAFMSDLPETLTVHTGSVNTQYDPNDKVPTGVFYMRPKASILLTTAQEVPPAAYSDSKTSGVATRTSTAIGVIGFLFAHRLLL

SEQ ID NO: 107 - DNA, 987 bp, Bemisia tabaci
  source 1..987: mol_type = genomic DNA
atggccctgagatggtcgctggaggaggctttgcgagtgttcaaattaccgatggcagacttgatgtatcaggctcagtctacccaccgggccaacttcaaccctaacgaaatgcaaatcagtacgcttctgagcatcaagacgggcgcttgcccagaaaactgctcgtactgtccccagtcagcctactacaaaacagagatcaagaaggagcctctgatggatttagcggaggttgttgccgccgcgaaggcagccaaagagagtggaagtacccgtttctgcatgggtgctgcttggcgtggacccacggacaggaatctcccgttggtctgtgatatggtgaaagaggtaaagaagttgggcttagagacttgcgtaacattagggcttctaaaggaccgccacgccgtacaactaaaagaagccggactggacttctacaaccacaacatcgacacgtcaccggagtactacaagaagatcatctcgacgaggactttcgaggatcgaatccggaccctggagcacgtgcgcaacgcagggatcaaggtctgctgcggcgggatcctaggaatgggtgagaacaccgaggaccggatcaagatgctcttggtattggcgaatctggaggagcctcctgaatcagtgccgatcaatcagctgattccgatccctgggactcctctcgccgacgcggaccctgtcgagggcaccgacttcgtgcggaccatcgctctcacccgggtcatgatgcccaaggcctacatccgactctccgctggccgggagaacatgtccgaggagatgcagacgctctgtttcttggcaggagtcaactcgattttctacggtgagaagctcctcacggccaagaacttccggccgagcaaggatgatcaattactcagtaagctcggcttcaagaagatggagattgatgaggcaccggccaacagtcagtgtaaagaggcggccattgggtga

SEQ ID NO: 108 - DNA, 1785 bp, Bemisia tabaci
  source 1..1785: mol_type = genomic DNA
cataaaaggagcttaaacttaaatgttttatgtattttccctttagaaagtagcgcatgaaagcaacttattttcctttttttttttttttttttaaattttaatttgtttgttctgtgtgtttcaggtatgtaatgacaactttaaattctctctccccatttactccagcccatttgctatcctgcgacacgagtttgacgatggaaacatttttttcaccctgtcagaagtgatgaggggagcgacgaggcaagagcggactcgctagtggatggattcggttgagcggaaagacaagtccaccgatgagaggagcggtggatgagcggatgagccctcggttcgactgcgatggatagaccttactcgtgggatcaatgcggatcagtgctgaaggttgaagcgggggtgggcggggtcggggtcgcgggggcgggggtaggaagcaaagccctggaggactcggacgacgggcccgggtgcttctgctgcgactcggacagcgacgacagcgccgagcaggtgcggatccgcaacacgaacaagaagtgcgcccgcgggtgcctcaacaacatcgtcgtctgctcggtcgtcatcttgtacaccttcttcggggccgtcctcttcctcctcatcgagggcggcggggtcttcaccgtcgacgacaagatcctcgacgagtccgaggagccctccttcctcaacgacgagaagatctcggccgtcaagcggaacctcggctccttcaacaacctcacgctccagtggatcgagaacatcaacgccgagaaccgtgagcgcactgtcgagaacatctgggacatcacggtgaacttgaacatcctctaccgggagaactggacccggttggccgccccggagatcaaccggttccagcagcagctcgtcgagcgcatcacgcgggaggtcaccgcctccggtccctccgagtccgagcttcaggtcccggtgcccgtgccggttcaggtcgaacgtcacctcgtcgactcttcctatcggatggatcagtatcctctggagtggacactcgccaaggctttcctcttctccctcaccatcatcacgacaataggtgagcattggacggccttcactcccgagcaatttttcaagccttggcataaaatcggagcgaaaatggggctcattgaatcagcattaatggccatggcggtatcactgtgcttattcgtcttcgcacactcgaaaagcgtggcggtatgccagccgcaaaccgcggaagtatgccacccgcaaacggcggaagcgaatgcggacgcttcgacaggagctatgatcatcctatccctggcctcgacgctgaacaatcggatggagtacatgaaagacgtggccgggaacaagttcaaagccaacaagccggtcgaggatctaccccgcgaggagaaggtcatgaactcggccgtcgccaacgcctcggccgaggggctggatccgaattcgatacggcccttcatccaggcgcagatggacgtggccaaggacatccagcgcgtctatattgccggctggaaggttaggccagagaagtgggatcccagggcgctcgaggaggtcagggagctcatctccgcggacgacgccaagatcctgaggctcagtcgacagcaacttacggttggagggttttgcgaggcctatagggagcagatctacaggagcctcacagcgccttacatcactgatgcgcatagaaaactcctcgtggatacgctgttttgcgttaggcttaagcagtag

SEQ ID NO: 109 - DNA, 801 bp, Bemisia tabaci
  source 1..801: mol_type = genomic DNA
catcttgaattttcacccatctataaaattacagacatgcacggcctgggcaatgacttcatggtcgtggacaccgtcacccagaacatctccttcacgacggaccagatccgtctcttggccgaccgacacctgggcgtcgggttcgaccagctcctggtcgtcgagccctcgtcggagcccgacgtggacttccactaccggatcttcaacgtggacggaagcgaggtggcccaatgcgggaacggcgcccggtgtttcgcccgcttcgtccggctccagcgcttgagccggaagcgcgacctccacgtcagcaccgcctccgggcgccgcatggtcctctccatcacgccggacgaccaggtccgcgtcaacatgggcgagcccactctttaccacatggagaacgtgacgttcggttgcgtgtcgctaggcaacccgcactgcgtggtgcaggtagagagtgttaagaccgcgcctgtggacacgctcggggccatgctggagacccacgagcgcttcccggagcgaaccaatgtgggctttatggaggtcgtcgatcccgggcatatccgccttcgcgtgttcgagcgtggtgtgggcgagacgagggcctgtggcagcaacgcctgtgctgctgttgtctgcggcgttcgccagggtgcgctgacgtcgccggtgcgcgtggatctccctggcggcacgctccgtgtcgcctgggcggggcttggacagccggtctacttgaccggtccggcggttcatgtttatgatggcttcgttgatttgggaaatacatga

SEQ ID NO: 110 - RNA, 370 nt, synthetic construct
  source 1..370: mol_type = other RNA; note = dsRNA
tgattttgttccaaaccacactagtgaccgtcacttgtggtttcgaaagtctgtcgccagggaagaaccttatacagatttctacgtttggagagatgccaaagtcgtcaatggtctacggttacccccaaataattggcggagtcagtttgccggatctgcgtggacttggaatgaagagagacagcaatatttctatcatcagttccatactaaacagccagatttgaattacaactgtaaagagctagtcagagaaatgatctcaacgttaaaattttggttggatcgaggtgtggatggcttccgtatggatgctgtgacgtacatgtacgaagacccgaactgggcggatgaggaattgctgaat

SEQ ID NO: 111 - RNA, 497 nt, synthetic construct
  source 1..497: mol_type = other RNA; note = dsRNA
tcgaaagacagacacgatgctgtacggagatgtggagacctacgtgctcgccaaatgggtcttcgcctttaccaggcgtttgactggaagtgacacttatgtggtggtcgttaatcttgggagcgacactacatctgtcgatctctctgctttcatgagtgatctaccggagactttaacagtccacacgggcagtgtcaatacccagtatgatccaaatgacaaagtaccaactggtgtattttacatgcgaccaaaagcctccatccttttaaccactgcccaagaagtgccgcctgctgcatattcggattcaaaaactagtggagtcgcaacgaggacatccacttcaatcggaataattgtatttcttttcactcataggttactcctataggctaatgtccattccttttatgtgtatttttattgtaatttatgataatgaaaattaaaaagatgaaacaaaagaagaaggaactacccgacggaaatca

SEQ ID NO: 112 - RNA, 501 nt, synthetic construct
  source 1..501: mol_type = other RNA; note = dsRNA
gagactactctactttgttgtgtgcgctaacatttggattggattttcggaaagccatcagattctaagcagcggcggaatgaaggagtggtgggagagcactctgatttatcagatttacccacgctcctttaaggattcaaatggggatggctacggtgatctaaatggtattcttgaaagactggattatgtcaaggagataggcgtggacacaatttggattcaaccgttctacaagtcgccgatggctgatatgggttacgacgttgctgattatcgagctgttgatccaatgttcggtaccatggatgattttaagaggttactaaaaaatagtcatgagaaagggatgaaagtgattgttgactttgtgccgaatcatagtagtgatcaaggtgagtggtttaagctctccgaacaacgtatcgaaccgttcacagatttctacgtttggaaggacggtaaacggattaacgaaactcacacaacacatccaaa

SEQ ID NO: 113 - RNA, 481 nt, synthetic construct
  source 1..481: mol_type = other RNA; note = dsRNA
cagcgaaaggagaaaggatgccaccgacgaactgggtcagtgaatttggaggctacacttggagctggaatgaggaaaggcaacaatattatcttcatattttccattacaagcaacctgacttaaactacaacagcaaagaactcgttagagagatgacggatgtgatcagattttggctagacagagggattgatggtctcagaatggatgctgttacctacatgtatgaagatccgcagtatagagatcaagaattattggccccagatttagatccatgggatttcaaaagctacaaccgctgccacacgatggaccaaccggcaacatacagattaataaccaagttcagggagatttttgacgcctactccaagagagatggcgagactaaggtcatgataacagaggcatacacatcattggataaaactatggaatactacaagttcggtgataaacctggtgctcatatgcc

SEQ ID NO: 114 - RNA, 481 nt, synthetic construct
  source 1..481: mol_type = other RNA; note = dsRNA
cggcgatctaaaaggaatcaaagagaaagtccactatctttgtgagctgggagtgaccgcaatttggataaacccgttctacgaatccccgctgaaagacaacggttatgacatctctaatttcaagaaaatccaccacaagttcggtaacatgtgtgactttaaggcactcatgaaaagacttgataataatccttgcggtgataacaaagagatcagagtgatcatggatttcgtacccaaccacacgagcgacgaacatgaatggtttcagaagtcagtcgatcgggtgcctccatacaatgattactacgtatggatggatcctaaaggatgggacaaaaatggaaatccaatccctcccaacaattgggcatcagtcttcagctacggagaggaagcgtctggatgggagtggaatgagaagagacaacaattttactttcacgctttcatgagggaacagccggaccttaacctt

SEQ ID NO: 115 - RNA, 897 nt, synthetic construct
  source 1..897: mol_type = other RNA; note = dsRNA
gagcaacaaacggacaaaggaaaagcgcctatggaatcgccaactgaattgaatcaatcagagaagtctgtgggtgaaagtgaggaaaaacataaaaagacaaaattcaaaataagagctggtggtggaaatgagaagagagatgatatagataaaattccaaccaacgctctagaattccggaagagcttcaaaccaccaaaagtgtcacaagcagcatatgtatggataccacgttcgcaaagagataatttgacacctgatgtcatacagaattttctagcgtacgtgcctccatcacatgctatagacaatcaattggcttctggagttgaagttgagaattgggccattgaggtttcaaaagcttatggagtcaccattcaagagttttatagaacgatcctaccagcttggattgttaattgtattgtgaatgggactagtgatgaaaggaaaaatgagaaatcgtggagagctgttgagctaaatacgcagggtgaggatattgatgattcagaataccctatggaaccaatgtacaaatttgctctcccgacaatgagaaaaataatgagaaatttttccagccaagcaatcctaatgtatcagaatagtgtgtctgctggaaaagcttttgtaataaaagcagcccgaaatgccgggtatacaagcattgaaaataagtggttgggtattgacttcctagccgaagcacaattgtcacaaagccaacttgatatcaaacatcaaatattggctgctaatgttggtagaagtaaaactaagttgtttgctttagctgctcctggtgacgataataatgtggataaagaaaggcacactacgcgggatgttagtgcaactaggcacagctacgcgggtgccgctatcgaa

SEQ ID NO: 116 - DNA, 20 bp, synthetic construct
  source 1..20: mol_type = other DNA
cattccactacagagctcca

SEQ ID NO: 117 - DNA, 20 bp, synthetic construct
  source 1..20: mol_type = other DNA
tttcaggtttcggatggctt

SEQ ID NO: 118 - DNA, 20 bp, synthetic construct
  source 1..20: mol_type = other DNA
ggcagtatcaacagtcgtca

SEQ ID NO: 119 - DNA, 20 bp, synthetic construct
  source 1..20: mol_type = other DNA
ggttcttgtagcctggagac

SEQ ID NO: 120 - DNA, 20 bp, synthetic construct
  source 1..20: mol_type = other DNA
atacggcttctgctggattt

SEQ ID NO: 121 - DNA, 20 bp, synthetic construct
  source 1..20: mol_type = other DNA
tggttggtggtttcctttga

SEQ ID NO: 122 - DNA, 19 bp, synthetic construct
  source 1..19: mol_type = other DNA
taacagccactacaaagta

SEQ ID NO: 123 - DNA, 18 bp, synthetic construct
  source 1..18: mol_type = other DNA
gacccatttagagagaac

SEQ ID NO: 124 - DNA, 20 bp, synthetic construct
  source 1..20: mol_type = other DNA
atctacctacacgctcactg

SEQ ID NO: 125 - DNA, 20 bp, synthetic construct
  source 1..20: mol_type = other DNA
ccaagtctggaatctcgtcc

SEQ ID NO: 126 - DNA, 20 bp, synthetic construct
  source 1..20: mol_type = other DNA
tgctggaggaaatggtgttc

SEQ ID NO: 127 - DNA, 20 bp, synthetic construct
  source 1..20: mol_type = other DNA
acagtgaggctgaatcttgg

SEQ ID NO: 128 - DNA, 19 bp, synthetic construct
  source 1..19: mol_type = other DNA
tttcttcaacacgggcgag

SEQ ID NO: 129 - DNA, 20 bp, synthetic construct
  source 1..20: mol_type = other DNA
gtctgggttcttcttctgcg

SEQ ID NO: 130 - DNA, 20 bp, synthetic construct
  source 1..20: mol_type = other DNA
aataggtgagcattggacgg

SEQ ID NO: 131 - DNA, 20 bp, synthetic construct
  source 1..20: mol_type = other DNA
gcgaagacgaataagcacag

SEQ ID NO: 132 - DNA, 20 bp, synthetic construct
  source 1..20: mol_type = other DNA
gccaacttcaaccctaacga

SEQ ID NO: 133 - DNA, 20 bp, synthetic construct
  source 1..20: mol_type = other DNA
aacaacctccgctaaatcca

SEQ ID NO: 134 - DNA, 100 bp, Bemisia tabaci
  source 1..100: mol_type = genomic DNA
gctgtgtcgggacatgttagcataataaaaggtatgctgtacattgtcgcgcaatgccttggagccatctgtggagcaatcattttgaatgaaatcacgc

SEQ ID NO: 135 - DNA, 135 bp, Bemisia tabaci
  source 1..135: mol_type = genomic DNA
ccacccgcaaacggcggaagcgaatgcggacgcttcgacaggagctatgatcatcctatccctggcctcgacgctgaacaatcggatggagtacatgaaagacgtggccgggaacaagttcaaagccaacaagcc

SEQ ID NO: 136 - DNA, 100 bp, synthetic construct
  source 1..100: mol_type = other DNA
agccgaaagccagactgaagacctgctacctaagtcaaaagaaataaaatacaaaaatccggcagatcgacattctcacagatcgtggctatgcttgtct

SEQ ID NO: 137 - DNA, 100 bp, synthetic construct
  source 1..100: mol_type = other DNA
gaaaacggagtgccgcaaccgcttcgagatgatagtgaaagttattggaggaaaaaattccgggaaattaccaacaaaaaactgtatttaccactgcgta

(19) United States
(12) Patent Application Publication
(10) Pub. No.: US 2025/0194603 A1
(43) Pub. Date: Jun. 19, 2025

(54) COMPOSITIONS AND METHODS FOR MANAGEMENT OF WHITEFLY

(71) Applicants: National Resources Institute, Chatham Maritime (GB); Yissum Research Development Company of the Hebrew University of Jerusalem Ltd., Jerusalem (IL); Donald Danforth Plant Science Center, St. Louis, MO (US); National Agricultural Research Organization, Entebbe (UG)

(72) Inventors: John Colvin, Newington (GB); Sharon L. van Brunschot, Highgate Hill (AU); Rekha Swamy, Gillingham (GB); Susan Seal, Maidstone (GB); Shai Morin, Jerusalem (IL); Osnat Malka, Givatayim (IL); Dor Wintraube, Tel-Aviv (IL); Nigel Taylor, St. Louis, MO (US); Narayanan Narayanan, St. Louis, MO (US); Titus Alicai, Kampala (UG); Tadeo Kaweesi, Kampala (UG)

(21) Appl. No.: 18/979,189

(22) Filed: Dec. 12, 2024

Related U.S. Application Data
(60) Provisional application No. 63/610,376, filed on Dec. 14, 2023.

(51) Int. Cl. A01N 57/16; A01P 7/04; C12N 15/113; C12N 15/52

(52) U.S. Cl. CPC A01N 57/16; A01P 7/04; C12N 15/1137; C12N 15/52; C12N 2310/14

(57) ABSTRACT

The present disclosure is directed to controlling pest infestation by inhibiting one or more biological functions in an invertebrate pest. The disclosure discloses methods and compositions for use in controlling pest infestation by feeding one or more different recombinant double stranded RNA molecules to the pest in order to achieve a reduction in pest infestation through suppression of gene expression. The disclosure also discloses methods and compositions for targeted genome editing in the pest in order to achieve a reduction in pest infestation through disruption of protein activity. The disclosure is also directed to methods for making transgenic plants that express the double stranded RNA molecules and targeted genome editing constructs for use in protecting plants from pest infestation.

### CROSS-REFERENCE TO RELATED APPLICATIONS

This application claims priority to U.S. Provisional Application Ser. No. 63/610,376 filed on Dec. 14, 2023, the entire disclosure of which is incorporated herein by reference.

### INCORPORATION OF SEQUENCE LISTING

A sequence listing containing the file named “AGOE014US_ST26.xml” which is 206 kilobytes (measured in MS-Windows®) and created on Dec. 12, 2024, and comprises 137 sequences, is incorporated herein by reference in its entirety.

### FIELD OF THE INVENTION

The present disclosure relates generally to genetic control of pest infestations in plants. More specifically, the present disclosure relates to the methods for modifying endogenous expression of coding sequences in the cell or tissue of a particular pest to achieve intended levels of pest control.

### BACKGROUND

Controlling pest infestation is an essential component of horticultural management. Many of the world's farmers face pressure from a wide variety of agricultural pests, which can result in low yield or plant death. Bemisia. tabaci (sensu latu), commonly known as whitefly, is a globally distributed pest affecting agricultural production, both by direct damage and as a vector of plant viruses. In recent years, whitefly populations have risen to extreme levels, particularly in Africa. This has resulted in significant annual losses of important food security crops like cassava, which is a staple food for approximately 800 million people in Sub-Saharan Africa. Therefore, the development of compositions and methods for controlling pest populations, including superabundant whitefly populations, is needed to reduce both the direct damage caused by pests and also the spread of devastating viral diseases transmitted by these pests.

### SUMMARY

In one aspect, the present disclosure provides a recombinant polynucleotide molecule comprising a first polynucleotide sequence having at least about 85% sequence identity to at least 18 contiguous nucleotides of a target nucleotide sequence, wherein said target nucleotide sequence: a) encodes an enzyme involved in the trehalose biosynthesis pathway or sugar transporters, optionally, a hexokinase (Hx), trehalose 6-phosphate synthase (TPS), or UTP-glucose-1-phosphate uridylyltransferase (UGP1), ST1, or ST2; and/or b) genes encoding proteins and enzymes involved in detoxification of plant toxins, optionally, UDP-glucosyltransferase (e.g., UDP-GT1, UDP-GT2, UDP-GT3) or ABC transporters (e.g., ABC1); wherein said recombinant polynucleotide molecule disrupts the activity of said one or more polypeptides when provided in the diet of an invertebrate pest. In some embodiments, said target nucleotide sequence: a) encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40; and/or b) has a sequence selected from the group consisting of SEQ ID NO:1, 3, 5, 7, 9, 11, 13, 15, 17, 19, 21, 23, 25, 27, 29, 31, 33, 35, 37, 39, 134, and 135; wherein said recombinant polynucleotide molecule disrupts the activity of said one or more polypeptides when provided in the diet of an invertebrate pest. In further embodiments, said recombinant polynucleotide molecule further comprises at least one polynucleotide sequence having at least about 85% sequence identity to at least 18 contiguous nucleotides of a second target nucleotide sequence, wherein said second target nucleotide sequence: a) encodes a polypeptide of an osmoregulation gene in an invertebrate pest optionally, a water-carrying aquaporin, optionally, AQP1; or an α-glucosidase gene, optionally, SUC1, SUC2, SUC3, SUC4, SUC5, SUC7, SUC12; and/or b) encodes a polypeptide of an essential symbiosis gene, optionally an aspartate aminotransferase (AAT); Arginosuccinate lyase (argH); a diaminopimclate decarboxylase (LysA); a branched-chain-amino-acid aminotransferase gene (BCAT); a 4-hydroxy-tetrahydrodipicolinate reductase (dapB); and/or Chorismate mutasc (CM); Diaminopimclate epimerase (DapF); Biotin synthase (e.g., BioA, BioB, bioD). In certain embodiments, said second target nucleotide sequence a) encodes a polypeptide having a sequence selected from the group consisting of SEQ ID NO:41, 43, 45, 47, 49, 51, 53, 98, 100, 102, 104, and 106; and/or b) has a sequence of any of SEQ ID NO:42, 44, 46, 48, 50, 52, 54, 97, 99, 101, 103, 105, 107-115, 136, and 137. In some embodiments, said recombinant polynucleotide molecule comprises at least two polynucleotides, at least three polynucleotides, at least four polynucleotides, or at least five polynucleotides, optionally singular, stacked, linear, repeat, multimer, or inverted repeat. In further embodiments, said at least one polynucleotide is a first polynucleotide sequence operably linked to a heterologous promoter; or encodes an interfering RNA molecule; or encodes a ssRNA molecule or a dsRNA molecule. In other embodiments, the first polynucleotide sequence has at least about 90%, at least about 95%, or 100% sequence identity to at least 18 contiguous nucleotides of said target sequence. In certain embodiments, the first polynucleotide sequence has at least about 85% sequence identity to at least 19 contiguous nucleotides, at least 20 contiguous nucleotides, or at least 21 contiguous nucleotides of said target sequence. In certain embodiments, said polynucleotide sequence disrupts trehalose biosynthesis activity, sugar transporter activity, or detoxification activity; or disrupts osmoregulation or biosynthesis of nutrients. In some embodiments, the invertebrate pest is a pest of the order Hemiptera, e.g. a Bemisia species pest such as Bemisia. tabaci. In another aspect, plants, plant parts, plant cells, seeds, or commodity products comprising the recombinant polynucleotide molecule provided herein are described. In yet another aspect, compositions comprising the recombinant polynucleotides provided herein are described. In some embodiments, the present disclosure provides a composition comprising a polynucleotide molecule comprising a first polynucleotide sequence having at least about 85% sequence identity to at least 18 contiguous nucleotides of a target nucleotide sequence, wherein said target nucleotide sequence a) encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40; or b) has a sequence selected from the group consisting of SEQ ID NO: 1, 3, 5, 7, 9, 11, 13, 15, 17, 19, 21, 23, 25, 27, 29, 31, 33, 35, 37, 39, 134, 135; and wherein said composition disrupts the activity of said polypeptide when provided in the diet of an invertebrate pest. In certain embodiments, the polynucleotide molecule is an interfering RNA molecule or encodes an interfering RNA molecule; or a ssRNA molecule or a dsRNA molecule or encodes a ssRNA molecule or a dsRNA molecule. Further provided are methods for controlling invertebrate pest infestation, the methods comprising providing a polynucleotide molecule comprising a first polynucleotide sequence having at least 85% sequence identity to at least 18 contiguous nucleotides of a target nucleotide sequence in the diet of an invertebrate pest, wherein said polynucleotide molecule disrupts the activity of a polypeptide encoded by said target nucleotide sequence, wherein said target nucleotide sequence: a) encodes a polypeptide of a sugar transport and storage gene in an invertebrate pest optionally, a hexokinase (Hx), trehalose 6-phosphate synthase (TPS), or UTP-glucose-1-phosphate uridylyltransferase (UGP1), ST1, or ST2; and/or b) encodes a polypeptide of phytotoxin detoxification gene in an invertebrate pest optionally, UDP-glucosyltransferase (e.g., UDP-GT1, UDP-GT2, UDP-GT3) or

ABC transporters (e.g., ABC1). In some embodiments, said polynucleotide molecule further comprises at least one polynucleotide sequence having at least about 85% sequence identity to at least 18 contiguous nucleotides of a second target nucleotide sequence, wherein said second target nucleotide sequence: a) encodes a polypeptide of an osmoregulation gene in an invertebrate pest optionally, a water-carrying aquaporin, optionally, AQP1; or an α-glucosidase gene, optionally, SUC1, SUC2, SUC3, SUC4, SUC5, SUC7, SUC12; and/or b) encodes a polypeptide of an essential symbiosis gene, optionally an aspartate aminotransferase (AAT); Arginosuccinate lyase (argH); a diaminopimelate decarboxylase (LysA); a branched-chain-amino-acid aminotransferase gene (BCAT); a 4-hydroxy-tetrahydrodipicolinate reductase (dapB); and/or Chorismate mutase (CM); Diaminopimelate epimerase (DapF); Biotin synthase (e.g., BioA, BioB, bioD). In some embodiments, said methods comprise providing a polynucleotide molecule comprising a first polynucleotide sequence having at least 85% sequence identity to at least 18 contiguous nucleotides of a target nucleotide sequence in the diet of an invertebrate pest, wherein said polynucleotide molecule disrupts the activity of a polypeptide encoded by said target nucleotide sequence, wherein said target nucleotide sequence: a) encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40; or b) has a sequence selected from the group consisting of SEQ ID NO: 1, 3, 5, 7, 9, 11, 13, 15, 17, 19, 21, 23, 25, 27, 29, 31, 33, 35, 37, 39, 134, and 135. In further embodiments, said methods comprise providing a second polynucleotide sequence having at least about 85% sequence identity to at least 18 contiguous nucleotides of a second target nucleotide sequence, wherein said second target nucleotide sequence: a) encodes a polypeptide having a sequence selected from the group consisting of SEQ ID NO:41, 43, 45, 47, 49, 51, 53, 98, 100, 102, 104, and 106; and/or b) has a sequence of any of SEQ ID NO:42, 44, 46, 48, 50, 52, 54, 97, 99, 101, 103, 105, 107-115, 136, and 137. In certain embodiments of the methods described, the polynucleotide molecule is an interfering RNA molecule or encodes an interfering RNA molecule; or a ssRNA molecule or a dsRNA molecule or encodes a ssRNA molecule or a dsRNA molecule. In other embodiments, providing the polynucleotide molecule comprises providing a plant, plant part, plant cell, seed, or composition comprising said polynucleotide molecule in the diet of the invertebrate pest. In further embodiments, the invertebrate pest is a pest of the order Hemiptera, e.g. a Bemisia species pest. Throughout this specification and the claims, unless the context requires otherwise, the word “comprise” and its variations, such as “comprises” and “comprising,” will be understood to imply the inclusion of a stated composition, step, and/or value, or group thereof, but not the exclusion of any other composition, step, and/or value, or group thereof.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows relative survival of insects that were fed for five days on artificial diets containing dsST1, dsST2, dsHx, dsUGP1, and dsTPS, normalized to the survival proportion of feeding on dsCBSV (n=15 replicates per treatment). Error bars represent SEM. * correspond to a P value of ≤0.05.

FIG. 2 shows plant performance experiments. Panel (A): Relative, mean survival proportion of seven pairs of B. tabaci adults after feeding for five days on artificial diets containing dsABC1, dsUDP-GT1, dsUDP-GT2, dsUDP-GT3, and dsCBSV, followed by 48 hours on tobacco plants (n=4 replicates per treatment). Error bars represent SEM. Significant differences between each treatment to the control are denoted with * or ***, corresponding to a P value of <0.05 and <0.0001, respectively Panel (B): The proportion of progeny of the silenced insects, that remained undeveloped (dark grey) or reached early (light grey) or advanced (medium grey) nymph developmental stages after ˜3 weeks. Significant differences between each treatment and the control are denoted with ***, corresponding to P values of <0.0001.

FIG. 3 shows percent adult mortality on transgenic and control cassava plants after 7 days normalized to the mortality on the NASE 13 WT plants. Many lines were found to cause significant higher mortality of adults when compared to the control plants (P<0.05). Analysis was conducted using one-way ANOVA model (lines), followed by paired comparisons. The control cassava varieties were NASE 13 WT and a line expressing dsRNA against the GFP gene (WF17-GFP004).

FIG. 4 shows the proportion of progeny that were in advanced development stage (red-eyed 4th nymphs) or completed development (emerged as adults leaving the remains of their exoskeleton (exuviac) behind) 25 days after egg laying normalized to the mortality on the NASE 13 WT plants. Fourteen lines were significantly different (causing lower development rate) from the control plants (P<0.05). Analysis was conducted using an ANOVA one-way model (lines), followed by paired comparisons. The control cassava varieties were NASE 13 WT and NASE 13 expressing dsRNA against the GFP gene (WF17-GFP004).

FIG. 5 shows the mean number of adults (Panel A) and nymphs (Panel B) over five sampling periods (one to five months after planting; including DWF 55 and DWF56), after normalizing to the counts of NASE 13 WT. Only one line, DWF56-N13004, had lower adult counts when compared to the control NASE 13 WT plants (P≤0.05). Analysis was conducted using an ANOVA one-way model (lines), followed by paired comparisons. The control cassava varieties were NASE 13 WT, NASE 12 (a whitefly susceptible line) and Mkumba (a whitefly resistant line).

FIG. 6 shows the mean number of adults (Panel A) and nymphs (Panel B) over six sampling periods (one to six months after planting; including DWF 56, DWF66, DWF67 and DWF68), after normalizing to the counts of NASE 13 WT. Both in adults and nymphs, no line had had lower counts when compared to the control NASE 13 WT plants (P<0.05). Analysis was conducted using an ANOVA one-way model (lines), followed by paired comparisons. The control cassava varieties were NASE 13 WT, NASE 12 (a whitefly susceptible line) and Mkumba (a whitefly resistant line).

FIG. 7 shows the proportion of progeny that were in advanced development stage two to seven months after planting, normalized to the development rate on the NASE 13 WT plants. One line was significantly different (causing lower development rate) from the control NASE 13 WT plants (P<0.05). Analysis was conducted using an ANOVA one-way model (lines), followed by paired comparisons. NASE 12 (a whitefly susceptible line) and Mkumba (a whitefly resistant line).

### BRIEF DESCRIPTION OF THE SEQUENCES

SEQ ID NO:1 is a nucleotide sequence encoding MEAMI Sugar Transporter 1 (ST1)—(Bta11863, XM_019057683.1).

SEQ ID NO:2 is the amino acid sequence of MEAMI Sugar Transporter 1 (ST1)—(XP_018913228.1_1).

SEQ ID NO:3 a nucleotide sequence encoding SSA1-SG1 Sugar Transporter 1 (ST1)—(ENSSSA1UGG009783).

SEQ ID NO:4 is an amino acid sequence of SSA1-SG1 Sugar Transporter 1 (ST1).

SEQ ID NO:5 is a nucleotide sequence encoding MEAMI Sugar Transporter 2 (ST2)−(Bta02283, XM_019047740.1).

SEQ ID NO:6 is an amino acid sequence of MEAMI Sugar Transporter 2 (ST2)−(XP_018903285.1_1).

SEQ ID NO:7 is a nucleotide sequence encoding SSA1-SG1 Sugar Transporter 2 (ST2)−(ENSSSA1UGG004531).

SEQ ID NO:8 is an amino acid sequence of SSA1-SG1 Sugar Transporter 2 (ST2).

SEQ ID NO:9 is a nucleotide sequence encoding MEAMI Trehalose 6-Phosphate Synthase (TPS)—(Bta15703, XM_019060419.1).

SEQ ID NO:10 is an amino acid sequence of MEAMI Trehalose 6-Phosphate Synthase (TPS)—(XP_018915964.1_1).

SEQ ID NO:11 is a nucleotide sequence encoding SSA1-SG1 Trehalose 6-Phosphate Synthase (TPS).

SEQ ID NO:12 is an amino acid sequence of SSA1-SG1 Trehalose 6-Phosphate Synthase (TPS).

SEQ ID NO:13 is a nucleotide sequence encoding MEAMI Hexokinase (Hx)—(Bta00587, XM_019054050.1).

SEQ ID NO:14 is an amino acid sequence of MEAMI Hexokinase (Hx)—(XP_018909595.1_1).

SEQ ID NO:15 is a nucleotide sequence encoding SSA1-SG1 Hexokinase (Hx) CDS.

SEQ ID NO:16 is an amino acid sequence of SSA1-SG1 Hexokinase (Hx).

SEQ ID NO:17 is a nucleotide sequence encoding MEAMI UTP-Glucose-1-phosphate Uridylyltransferase (UGP1)—(Bta10225, XM_019043910.1).

SEQ ID NO:18 is an amino acid sequence of MEAMI UTP-Glucose-1-phosphate Uridylyltransferase (UGP1)—(XP_018899455.1_1)

SEQ ID NO: 19 is a nucleotide sequence encoding SSA1-SG1 UTP-Glucose-1-phosphate Uridylyltransferase (UGP1).

SEQ ID NO:20 is an amino acid sequence of SSA1-SG1 UTP-Glucose-1-phosphate Uridylyltransferase (UGP1).

SEQ ID NO:21 is a nucleotide sequence encoding MEAMI ABC transporter 1 (ABC1)—(Bta05312, XM_019053037.1).

SEQ ID NO:22 is an amino acid sequence of MEAMI ABC transporter 1 (ABC1)—(XP_018908582.1).

SEQ ID NO:23 is a nucleotide sequence encoding SSA1-SG1 ABC transporter 1 (ABC1).

SEQ ID NO:24 is an amino acid sequence of SSA1-SG1 ABC transporter 1 (ABC1).

SEQ ID NO:25 is a nucleotide sequence encoding MEAMI ABC transporter (ABC)—(Bta08686, XM_019042727.1).

SEQ ID NO:26 is an amino acid sequence of MEAMI ABC transporter (ABC)—(XP_018898272.1).

SEQ ID NO:27 is a nucleotide sequence encoding SSA1-SG1 ABC transporter (ABC).

SEQ ID NO:28 is an amino acid sequence of SSA1-SG1 ABC transporter (ABC).

SEQ ID NO:29 is a nucleotide sequence encoding MEAMI UDP-glucosyltransferase 1 (UDP-GT1)—(Bta07606, XM_019041304.1).

SEQ ID NO:30 is an amino acid sequence of MEAMI UDP-glucosyltransferase 1 (UDP-GT1)—(XP_018896849.1).

SEQ ID NO:31 is a nucleotide sequence encoding SSA1-SG1 UDP-glucosyltransferase 1 (UDP-GT1).

SEQ ID NO:32 is an amino acid sequence of SSA1-SG1 UDP-glucosyltransferase 1 (UDP-GT1).

SEQ ID NO:33 is a nucleotide sequence encoding MEAMI UDP-glucosyltransferase 2 (UDP-GT2)—(Bta13755, XM_019047746.1).

SEQ ID NO:34 is an amino acid sequence of MEAMI UDP-glucosyltransferase 2 (UDP-GT2)—(XP_018903291.1).

SEQ ID NO:35 is a nucleotide sequence encoding SSA1-SG1 UDP-glucosyltransferase 2 (UDP-GT2).

SEQ ID NO:36 is an amino acid sequence of SSA1-SG1 UDP-glucosyltransferase 2 (UDP-GT2).

SEQ ID NO:37 is a nucleotide sequence encoding MEAMI UDP-glucosyltransferase 3 (UDP-GT3)—(Bta11108, XM_019056922.1).

SEQ ID NO:38 is an amino acid sequence of MEAMI UDP-glucosyltransferase 3 (UDP-GT3)—(XP_018912467.1).

SEQ ID NO:39 is a nucleotide sequence encoding SSA1-SG1 UDP-glucosyltransferase 3 (UDP-GT3).

SEQ ID NO:40 is an amino acid sequence of SSA1-SG1 UDP-glucosyltransferase 3 (UDP-GT3).

SEQ ID NO:41 is the amino acid sequence of the B. tabaci water-carrying aquaporin, known as AQP1.

SEQ ID NO:42 is a nucleotide sequence encoding SEQ ID NO:41. Nucleotides 67-480 were identified as a target region for suppression using dsRNA as described herein.

SEQ ID NO:43 is the amino acid sequence of the B. tabaci α-glucosidase gene, known as SUC1.

SEQ ID NO:44 is a nucleotide sequence encoding SEQ ID NO:43. Nucleotides 61-416 were identified as a target region for suppression using dsRNA as described herein.

SEQ ID NO:45 is the amino acid sequence of the B. tabaci α-glucosidase gene, known as SUC2.

SEQ ID NO:46 is a nucleotide sequence encoding SEQ ID NO:45.

SEQ ID NO:47 is the amino acid sequence of the arginosuccinate lyase gene identified in the bacteriocyte of B. tabaci SSA1-SG1, known as argH.

SEQ ID NO:48 is a nucleotide sequence encoding SEQ ID NO:47. Nucleotides 362-715 were identified as a target region for suppression using dsRNA as described herein.

SEQ ID NO:49 is the amino acid sequence of the diaminopimelate decarboxylase gene identified in the bacteriocyte of B. tabaci SSA1-SG1, known as LysA.

SEQ ID NO:50 is a nucleotide sequence encoding SEQ ID NO:49. Nucleotides 128-472 were identified as a target region for suppression using dsRNA as described herein.

SEQ ID NO:51 is the amino acid sequence of the branched-chain-amino-acid aminotransferase gene identified in the bacteriocyte of B. tabaci SSA1-SG1, known as BCAT.

SEQ ID NO:52 is a nucleotide sequence encoding SEQ ID NO:51.

SEQ ID NO:53 is the amino acid sequence of the 4-hydroxy-tetrahydrodipicolinate reductase gene identified in the bacteriocyte of B. tabaci SSA1-SG1, known as dapB.

SEQ ID NO:54 is a nucleotide sequence encoding SEQ ID NO:53.

SEQ ID NOs: 55-74 are primer sequences used in the validation of gene expression of selected gene.

SEQ ID NOs: 75-96 are characteristic catalytic site residue for α-glucosidase enzymes.

SEQ ID NO:97 is the nucleotide sequence encoding ENSSSA1UG%028740 (Ssa12486).

SEQ ID NO:98 is the protein sequence encoded by SEQ ID NO:97.

SEQ ID NO:99 is the nucleotide sequence encoding NP_001119607.1_Aphid.

SEQ ID NO: 100 is the protein sequence encoded by SEQ ID NO:99.

SEQ ID NO: 101 is the nucleotide sequence encoding ENSSSAIUGT002066 (Ssa05164).

SEQ ID NO:102 is the protein sequence encoded by SEQ ID NO:101.

SEQ ID NO: 103 is the nucleotide sequence encoding ENSSSAIUGT002057 (Ssa05154).

SEQ ID NO:104 is the protein sequence encoded by SEQ ID NO: 103.

SEQ ID NO: 105 is the nucleotide sequence encoding Ssa12230 (Bta15649).

SEQ ID NO: 106 is the protein sequence encoded by SEQ ID NO: 105.

SEQ ID NO: 107 is the nucleotide sequence of ENSSSAIUGT008510 (BioB). Nucleotides 460-894 were identified as a target region for suppression using dsRNA as described herein.

SEQ ID NO: 108 is the nucleotide sequence of ENSSSAIUGT027679 (CM). Nucleotides 1203-1663 were identified as a target region for suppression using dsRNA as described herein.

SEQ ID NO:109 is the nucleotide sequence of ENSSSAIUGT023765 (DapF).

Nucleotides 198-565 were identified as a target region for suppression using dsRNA as described herein.

SEQ ID NO:110 is a dsRNA construct sequence directed against SUC3.

SEQ ID NO:111 is a dsRNA construct sequence directed against SUC4.

SEQ ID NO:112 is a dsRNA construct sequence directed against SUC5.

SEQ ID NO:113 is a dsRNA construct sequence directed against SUC7.

SEQ ID NO:114 is a dsRNA construct sequence directed against SUC12.

SEQ ID NO:115 is a dsRNA construct sequence directed against the coat protein of the Cassava Brown Streak virus CBSV.

SEQ ID NO:116 is the qRT-PCR: Bta04282 (RPL13A) Forward primer.

SEQ ID NO:117 is the qRT-PCR: Bta04282 (RPL13A) Reverse primer.

SEQ ID NO: 118 is the qRT-PCR: Bta04298 (SUC3) Forward primer.

SEQ ID NO:119 is the qRT-PCR: Bta04298 (SUC3) Reverse primer.

SEQ ID NO: 120 is the qRT-PCR: Bta15649 (SUC4) Forward primer.

SEQ ID NO:121 is the qRT-PCR: Bta15649 (SUC4) Reverse primer.

SEQ ID NO: 122 is the qRT-PCR: Bta07453 (SUC7) Forward primer.

SEQ ID NO:123 is the qRT-PCR: Bta07453 (SUC7) Reverse primer.

SEQ ID NO:124 is the qRT-PCR: Bta12682 (SUC12) Forward primer.

SEQ ID NO:125 is the qRT-PCR: Bta12682 (SUC12) Reverse primer.

SEQ ID NO:126 is the DWF_57_F Forward primer.

SEQ ID NO: 127 is the DWF_57_R Reverse primer.

SEQ ID NO: 128 is the DWF_61_F Forward primer.

SEQ ID NO:129 is the DWF_61_R Reverse primer.

SEQ ID NO: 130 is the DWF62_9_F Forward primer.

SEQ ID NO:131 is the DWF62_9_R Reverse primer.

SEQ ID NO:132 is the 65_B_Forward Forward primer.

SEQ ID NO:133 is the 65_B_Reverse Reverse primer.

SEQ ID NO:134 is a nucleotide sequence of aquaporin 1 (AQP1).

SEQ ID NO:135 is a nucleotide sequence of chorismite mutase (CM).

SEQ ID NO:136 is a nucleotide sequence of a sugar transporter (ST).

SEQ ID NO:137 is a nucleotide sequence of in silico predicted effective siRNA producing regions of ST.

### DETAILED DESCRIPTION

The global resurgence of whiteflies has led to increasing demand for novel management options that can selectively inhibit or downregulate target gene expression in hemipterans such as whitefly with high specificity and fidelity for use in integrated pest management programs. To date, more than 39 morphologically indistinguishable species within the whitefly species of Bemisia tabaci (B. tabaci) complex have been reported. Collectively, members of the B. tabaci species complex can transmit more than 300 plant viruses, some of which cause crop diseases that have been listed among the top 10 most economically damaging plant viruses. In Sub-Saharan Africa, African cassava whitefly (a.k.a. Bemisia tabaci, Sub-Saharan Africa 1-subgroup 1 (SSA1-SG1)) serves as a vector for two devastating cassava plant virus types, cassava mosaic viruses and cassava brown streak virus (CMVs and CBSVs, respectively). Increasing populations of B. tabaci SSA1-SG1, in combination with CMV and CBSV, have resulted in estimated annual losses of cassava between USD 1.9-2.7 billion in areas affected by B. tabaci-transmitted cassava mosaic virus diseases. Management of cassava whiteflies will not only reduce the direct damage caused by high whitefly population but will also reduce the spread of devastating viral diseases transmitted by whiteflies.

Whiteflies (e.g., Bemisia tabaci), like other phloem feeders, must express a specific set of genes that can respond to the high osmolarity of ingested sap, metabolize carbohydrates to produce and store energy while reducing gut osmolarity, and synthesize haemolymph sugars to maintain the osmotic balance between the insect's body fluids and the ingested sap in the gut lumen. The instant disclosure therefore provides novel gene targets involved in transport and storage of sugars for the survival of Whitefly.

In order to utilize their host plant as a food resource, mating site, oviposition site, and habitat, insect herbivores must overcome plant defenses. Numerous plant-defense strategies exist based on an ability to synthesize, constitutively or in response to stress, more than 200,000 estimated specialized metabolites (i.e., chemical compounds that evolved in response to particular ecological challenges). Herbivorous insects have coevolved with their host plants and learned to overcome defensive plant compounds by employing various strategies, such as detoxification, sequestration, or secretion. Both specialized and general insect strategies have evolved, including both physiological adaptations that allow an insect to tolerate specific host plant defenses, and general adaptations that allow an insect to tolerate an array of plant defenses. The instant disclosure therefore provides novel gene targets utilized by B. tabaci to overcome plant defenses.

Genes involved in these systems and related pathways for sugar transport and detoxification are evaluated herein as potential targets for whitefly management. While putative RNAi targets have previously been proposed for the management of whiteflies, these prior studies failed to yield beneficial results for a variety of reasons.

The present disclosure overcomes the limitations of the prior art by identifying and validating critical gene targets utilized by B. tabaci for sugar transport and detoxification, which can be used in the management of cassava whitefly. Importantly, the evaluation of these key target genes was further coupled with phylogenetic analysis of other insects, including other Hemipterans, whitefly, and B. tabaci species for identification of osmoregulation genes and application of genome-scale metabolic reconstruction and constraint-based modelling to identify key sugar transport and detoxification genes.

As disclosed herein, the inventors identified candidate sugar transport gene targets, ST1 (Bta11863) for example SEQ ID NO: 1 and 3; and ST2 (Bta02283) for example SEQ ID NO:5 and 7. Three additional genes encoding enzymes involved in the trehalose biosynthesis pathway: a hexokinase (Hx; Bta00587) for example SEQ ID NO:13 and 15, trehalose 6-phosphate synthase (TPS; Bta15703) for example SEQ ID NO:9 and 11, and UTP-glucose-1-phosphate uridylyltransferase (UGP1; Bta10225) for example SEQ ID NO: 17 and 19.

Three overexpressed UDP-glucosyltransferase genes were also identified as targets and characterized, UDP-GT1 (Bta07606) for example SEQ ID NO:29 or 30; UDP-GT2 (Bta13755) for example SEQ ID NO:33 or 35; and UDP-GT3 (Bta11108) for example SEQ ID NO:37 or 39 and an overexpressed ABC transporter gene ABC1 (Bta05312) for example SEQ ID NO:21 or 23 were selected as potential targets and were further examined using a gene-silencing system. An ABC (ABC transporter) gene for example SEQ ID NO:25 and 27 was further investigated as a gene target. The present disclosure therefore represents a significant advance in the art.

In particular, the present disclosure provides novel recombinant polynucleotide molecules comprising a first polynucleotide sequence having at least about 85% sequence identity to at least 18 contiguous nucleotides of a target nucleotide sequence, wherein said target nucleotide sequence encodes a polypeptide having a sequence provided herein, and wherein said recombinant polynucleotide molecule disrupts the activity of said polypeptide when provided in the diet of an invertebrate pest, such as a Hemipteran. As described herein, ingestion by a target pest of compositions containing one or more dsRNA's, at least one segment of which corresponds to at least a substantially identical segment of RNA produced in the cells of the target pest, resulted in death, stunting, or other inhibition of the target pest. The instant disclosure demonstrates that that the disclosed nucleotide sequences, either DNA or RNA, derived from an invertebrate pest can be used to construct a recombinant pest host (i.e., transgenic plant) that is a target for infestation by the pest. The pest host can be transformed to contain one or more of the nucleotide sequences derived from the invertebrate pest. The nucleotide sequence transformed into the pest host or symbiont encodes one or more RNA's that form into a dsRNA, or serve as a gRNA sequence for a site-specific nuclease, in the cells or biological fluids within the transformed host or symbiont, thus making the polynucleotide molecule available in the diet of the pest if/when the pest feeds upon the transgenic host, resulting in the suppression of expression of one or more genes in the cells of the pest and ultimately the death, stunting, or other inhibition of the pest.

The present disclosure therefore relates generally to genetic control of invertebrate pest infestations in host organisms. More particularly, the present disclosure includes the methods for delivery of pest control agents to an invertebrate pest. Such pest control agents cause, directly or indirectly, an impairment in the ability of the pest to maintain itself, grow or otherwise infest a target host or symbiont. The present disclosure provides methods for employing stabilized polynucleotide molecules, e.g., dsRNA and gRNA molecules, in the diet of the pest as a means for suppression of targeted genes in the pest, thus achieving desired control of pest infestations in, or about the host or symbiont targeted by the pest. Transgenic plants can be produced using the methods of the present disclosure that express recombinant stabilized dsRNA and siRNA molecules, or gRNA in combination with a site-specific nuclease.

In accomplishing the foregoing, the present disclosure provides a method of inhibiting expression of a target gene in an invertebrate pest, and in particular, in African cassava whitefly or other Bemisia insect species, resulting in the cessation of feeding, growth, development, reproduction, infectivity, and eventually may result in the death of the pest. The method comprises introducing partial or fully, stabilized guide RNA molecules (gRNA) in combination with a site-specific nuclease, double-stranded RNA (dsRNA) nucleotide molecules, or their modified forms such as small interfering RNA (siRNA) molecules into a nutritional composition that the pest relies on as a food source and making the nutritional composition available to the pest for feeding. Ingestion of the nutritional composition containing the polynucleotide molecules results in the uptake of the molecules by the cells of the pest, resulting in the inhibition of expression of at least one target gene in the cells of the pest, or the modification of at least one target gene in the cells of the pest. Inhibition or modification of the target gene exerts a deleterious effect upon the pest. The polynucleotide molecules of the present disclosure may comprise a first polynucleotide sequence having at least about 85% sequence identity to at least 18 contiguous nucleotides of a target nucleotide sequence, wherein said target nucleotide sequence encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40, and wherein said recombinant polynucleotide molecule disrupts the activity of said polypeptide when provided in the diet of an invertebrate pest. The polynucleotide molecules of the present disclosure may comprise a first polynucleotide sequence having at least about 85% sequence identity to at least 18 contiguous nucleotides of a target nucleotide sequence, wherein said target nucleotide sequence has a sequence of any of SEQ ID NO: 1, 3, 5, 7, 9, 11, 13, 15, 17, 19, 21, 23, 25, 27, 29, 31, 33, 35, 37, 39, 134, and 135, and wherein said recombinant polynucleotide molecule disrupts the activity of said polypeptide when provided in the diet of an invertebrate pest. In some embodiments, such disruption may include the reduction or removal of a protein or nucleotide sequence agent that is essential for the pests' growth and development or other biological function. The method is effective in disrupting and/or inhibiting the expression of at least one target gene and can be used to disrupt and/or inhibit many different types of target genes in the pest.

In further embodiments, the polynucleotide molecules of the present disclosure may further comprise a second polynucleotide sequence having at least about 85% sequence identity to at least 18 contiguous nucleotides of a target nucleotide sequence, wherein said target nucleotide sequence encodes a polypeptide having a sequence selected from the group consisting of SEQ ID NO:41, 43, 45, 47, 49, 51, 53, 98, 100, 102, 104, and 106, and wherein said recombinant polynucleotide molecule disrupts the activity of said polypeptide when provided in the diet of an invertebrate pest. Polynucleotide sequences capable of disrupting the activity of target polypeptides when provided in the diet of an invertebrate pest are described in U.S. patent application Ser. No. 18/773,683, filed Jul. 16, 2024, the entirety of which is incorporated herein by reference. Examples include, but are not limited to, polynucleotide molecules that disrupt the activity of proteins encoded by SUC1, SUC2, AQP1, argH, lysA, BCAT, and dapB, BioB, CM, DapF, SUC3, SUC4, SUC5, SUC7, or SUC12 or variants, fragments, or homologues thereof. The polynucleotide molecules of the present disclosure may comprise a second polynucleotide sequence having at least about 85% sequence identity to at least 18 contiguous nucleotides of a target nucleotide sequence, wherein said target nucleotide sequence has a sequence of any of SEQ ID NO:42, 44, 46, 48, 50, 52, 54, 97, 99, 101, 103, 105, 107-115, 136, and 137, and wherein said recombinant polynucleotide molecule disrupts the activity of said polypeptide when provided in the diet of an invertebrate pest. In some embodiments, such disruption may include the reduction or removal of a protein or nucleotide sequence agent that is essential for the pests' growth and development or other biological function. The method is effective in disrupting and/or inhibiting the expression of at least one target gene and can be used to disrupt and/or inhibit many different types of target genes in the pest.

In yet further embodiments, the polynucleotide molecules, cells, plants, plant parts, or seeds of the present disclosure may comprise a first polynucleotide sequence having at least about 85% sequence identity to at least 18 contiguous nucleotides of a target nucleotide sequence, wherein said target nucleotide sequence encodes a polypeptide having a sequence selected from the group consisting of SEQ ID NO: 2, 4, 6, 8, 10, 12, 14, 16, 18, 20, 22, 24, 26, 28, 30, 32, 34, 36, 38, and 40, and wherein said recombinant polynucleotide molecule disrupts the activity of said polypeptide when provided in the diet of an invertebrate pest; and a second polynucleotide sequence having at least about 85% sequence identity to at least 18 contiguous nucleotides of a target nucleotide sequence, wherein said target nucleotide sequence encodes a polypeptide having a sequence selected from the group consisting of SEQ ID NO:41, 43, 45, 47, 49, 51, 53, 98, 100, 102, 104, and 106, and wherein said recombinant polynucleotide molecule disrupts the activity of said polypeptide when provided in the diet of an invertebrate pest.

The polynucleotide molecules, cells, plants, plant parts, or seeds of the present disclosure may comprise a first polynucleotide sequence having at least about 85% sequence identity to at least 18 contiguous nucleotides of a target nucleotide sequence, wherein said target nucleotide sequence has a sequence of any of SEQ ID NO: 1, 3, 5, 7, 9, 11, 13, 15, 17, 19, 21, 23, 25, 27, 29, 31, 33, 35, 37, and 39 and wherein said recombinant polynucleotide molecule disrupts the activity of said polypeptide when provided in the diet of an invertebrate pest; and a second polynucleotide sequence having at least about 85% sequence identity to at least 18 contiguous nucleotides of a target nucleotide sequence, wherein said target nucleotide sequence has a sequence of any of SEQ ID NO: 42, 44, 46, 48, 50, 52, 54, 97, 99, 101, 103, 105, and 107-115, and wherein said recombinant polynucleotide molecule disrupts the activity of said polypeptide when provided in the diet of an invertebrate pest. In some embodiments, such disruption may include the reduction or removal of a protein or nucleotide sequence agent that is essential for the pests' growth and development or other biological function. The method is effective in disrupting and/or inhibiting the expression of at least one target gene and can be used to disrupt and/or inhibit many different types of target genes in the pest.

The present disclosure also provides different forms of the pest control agents to achieve the desired reduction in pest infestation. In one form, the pest control agents comprise dsRNA molecules. In another form, the pest control agents comprise siRNA molecules. In still another form, the pest control agents comprise gRNA molecules alone or in combination with a site-specific nuclease (e.g., a Cas nuclease, a Cpf1 nuclease, or a variant of either). In still another form, the pest control agents comprise recombinant DNA constructs that can be used to stably transform microorganisms or plants, enabling the transformed microbes or plants to encode the dsRNA, siRNA, gRNA molecules. In another form, the pest control agents contain the recombinant DNA constructs encoding the dsRNA, siRNA, gRNA molecules.

The present disclosure provides recombinant DNA constructs for use in achieving stable transformation of particular host pest targets. Transformed host pest targets express pesticidally effective levels of preferred dsRNA, siRNA, gRNA molecules (alone or in combination with a site-specific nuclease) from the recombinant DNA constructs and provide the molecules in the diet of the pest.

The present disclosure also provides, as an example of a transformed host pest target organism, transformed plant cells and transformed plants and their progeny. The transformed plant cells and transformed plants express one or more of the dsRNA or siRNA sequences, or a fragment of any of the disclosed sequences, of the present disclosure from one or more of the DNA sequences as set forth in, SEQ ID NO:1, 3, 5, 7, 9, 11, 13, 15, 17, 19, 21, 23, 25, 27, 29, 31, 33, 35, 37, and 39 as set forth in the sequence listing, or the complement thereof.

In particular embodiments, the present disclosure provides methods of using the DNA molecule provided herein to control pests, including B. tabaci, a wide range of crops, including vegetables, fruits, and ornamental plants.

In certain examples DNA molecules and methods for controlling pests in cassava crops are provided, which reduce yield loss in cassava crops, as well as reduce transmission of viruses which can reduce quality and yield.

In certain examples DNA molecules and methods for controlling pests in tomato crops are provided, which reduce yield loss in tomato crops, as well as reduce transmission of viruses which can reduce fruit quality and yield.

In other examples DNA molecules and methods for controlling pests in cotton crops are provided, which reduce yield loss and increase quality in cotton crops. B. tabaci also secrete honeydew, which can promote the growth of sooty mold that further damages cotton and other crop plants.

B. tabaci can also cause significant damage to citrus crops, reducing yields and causing fruit drop. They can also transmit viruses that can affect the quality of the fruit. The DNA molecules and methods of the present disclosure can reduce or eliminate this loss in fruit quality or yield.

In some examples DNA molecules and methods for controlling pests in cucurbit crops are provided, which reduce yield loss and increase quality in cucurbit crops. B. tabaci can cause significant yield losses in cucurbit crops such as cucumbers, melons, and squash, as well as transmit viruses that can reduce fruit quality.

In other examples, DNA molecules and methods for controlling pests in vegetable crops (e.g., tomatoes, peppers, eggplants, cucumbers, squash, beans, sweet potatoes, cassava) are provided, which reduce yield loss and increase quality in vegetable crops. Whiteflies can cause significant damage to vegetable crops, reducing yields and quality. They can also transmit viruses that can affect the quality of the vegetables.

In other examples DNA molecules and methods for controlling pests in potato crops are provided, which reduce yield loss and increase quality in potato crops. Whitefly can cause significant damage to potato crops, reducing yields and quality. They can also transmit viruses that can affect the quality of the tubers.

In further examples DNA molecules and methods for controlling pests in ornamental plants are provided, which prevent loss of aesthetic value and marketability due to pests. B. tabaci can cause significant damage to ornamental plants such as poinsettias, hibiscus, and petunias.

Overall, whiteflies can cause significant economic losses in a wide range of crops, and effective management strategies are essential to reduce their impact on agricultural production.

### I. Gene Suppression

As used herein the words “gene suppression”, when taken together, are intended to refer to any of the well-known methods for reducing the levels of protein produced as a result of gene transcription to mRNA and subsequent translation of the mRNA. Gene suppression is also intended to mean the reduction of protein expression from a gene or a coding sequence including posttranscriptional gene suppression and transcriptional suppression. Posttranscriptional gene suppression is mediated by the homology between of all or a part of a mRNA transcribed from a gene or coding sequence targeted for suppression and the corresponding double stranded RNA used for suppression and refers to the substantial and measurable reduction of the amount of available mRNA available in the cell for binding by ribosomes. The transcribed RNA can be in the sense orientation to effect what is called co-suppression, in the anti-sense orientation to effect what is called anti-sense suppression, or in both orientations producing a dsRNA to effect what is called RNA interference (RNAi). RNAi is a natural cellular process where double-stranded RNA (dsRNA) molecules can silence specific gene expression. When applied to pest control, RNAi can target essential genes in pests (like insects, nematodes, or fungal pathogens), effectively inhibiting their growth or reproductive capacity without harming non-target organisms. This precise approach makes RNAi appealing for environmentally friendly pest control.

Transcriptional suppression is mediated by the presence in the cell of a dsRNA, a gene suppression agent, exhibiting substantial sequence identity to a promoter DNA sequence or the complement thereof to effect what is referred to as promoter trans suppression. Gene suppression may be effective against a native plant gene associated with a trait, e.g., to provide plants with reduced levels of a protein encoded by the native gene or with enhanced or reduced levels of an affected metabolite. Gene suppression can also be effective against target genes in plant pests that may ingest or contact plant material containing gene suppression agents, specifically designed to inhibit or suppress the expression of one or more homologous or complementary sequences in the cells of the pest.

Post-transcriptional gene suppression by anti-sense or sense-oriented RNA to regulate gene expression in plant cells is disclosed in U.S. Pat. Nos. 5,107,065, 5,759,829, 5,283,184, and 5,231,020. The use of dsRNA to suppress genes in plants is disclosed in WO 99/53050, WO 99/49029, U.S. Patent Application Publication No. 2003/0175965, and 2003/0061626, U.S. patent application Ser. No. 10/465,800, and U.S. Pat. Nos. 6,506,559, and 6,326,193.

A preferred method of post transcriptional gene suppression in plants employs both sense-oriented and anti-sense-oriented, transcribed RNA which is stabilized, e.g., as a hairpin and stem and loop structure. A preferred DNA construct for effecting post transcriptional gene suppression one in which a first segment encodes an RNA exhibiting an anti-sense orientation exhibiting substantial identity to a segment of a gene targeted for suppression, which is linked to a second segment encoding an RNA exhibiting substantial complementarity to the first segment. Such a construct would be expected to form a stem and loop structure by hybridization of the first segment with the second segment and a loop structure from the nucleotide sequences linking the two segments (see WO94/01550, WO98/05770, US 2002/0048814, and US 2003/0018993).

As used herein, the term “nucleic acid” refers to a single or double-stranded polymer of deoxyribonucleotide or ribonucleotide bases read from the 5′ to the 3′ end. The “nucleic acid” may also optionally contain non-naturally occurring or altered nucleotide bases that permit correct read through by a polymerase and do not reduce expression of a polypeptide encoded by that nucleic acid. The term “nucleotide sequence” or “nucleic acid sequence” refers to both the sense and antisense strands of a nucleic acid as either individual single strands or in the duplex. The term “ribonucleic acid” (RNA) is inclusive of RNAi (inhibitory RNA), dsRNA (double stranded RNA), siRNA (small interfering RNA), mRNA (messenger RNA), miRNA (micro-RNA), IRNA (transfer RNA, guide RNA (gRNA), whether charged or discharged with a corresponding acylated amino acid), and cRNA (complementary RNA) and the term “deoxyribonucleic acid” (DNA) is inclusive of cDNA and genomic DNA and DNA-RNA hybrids. The words “nucleic acid segment”, “nucleotide sequence segment”, or more generally “segment” will be understood by those in the art as a functional term that includes both genomic sequences, ribosomal RNA sequences, transfer RNA sequences, messenger RNA sequences, operon sequences and smaller engineered nucleotide sequences that express or may be adapted to express, proteins, polypeptides, or peptides.

As used herein, the term “pest” refers to insects, arachnids, crustaceans, fungi, bacteria, viruses, nematodes, flatworms, roundworms, pinworms, hookworms, tapeworms, trypanosomes, schistosomes, botflies, fleas, ticks, mites, and lice and the like that are pervasive in the human environment and that may ingest or contact one or more cells, tissues, or fluids produced by a pest host transformed to express or coated with a double stranded gene suppression agent or that may ingest plant material containing the gene suppression agent. As used herein, a “pest resistance” trait is a characteristic of a transgenic plant, transgenic host that causes the plant host to be resistant to attack from a pest that typically is capable of inflicting damage or loss to the plant host. Such pest resistance can arise from a natural mutation or more typically from incorporation of recombinant DNA that confers pest resistance. To impart insect resistance to a transgenic plant a recombinant DNA can be transcribed into an RNA molecule that forms a dsRNA molecule within the tissues or fluids of the recombinant plant. The dsRNA molecule is comprised in part of a segment of RNA that is identical to a corresponding RNA segment encoded from a DNA sequence within an insect pest that prefers to feed on the recombinant plant. Expression of the gene within the target insect pest, e.g., within the gut or bacteriocyte of said pest, is suppressed by the dsRNA, and the suppression of expression of the gene in the target insect pest results in the plant being insect resistant. Fire, et al. (U.S. Pat. No. 6,506,599) generically described inhibition of pest infestation, providing specifics only about several nucleotide sequences that were effective for inhibition of gene function in the nematode species Caenorhabditis elegans. Similarly, Plactinck, et al. (US 2003/0061626) describe the use of dsRNA for inhibiting gene function in a variety of nematode pests. Mesa, et al. (US 2003/0150017) describe using dsDNA sequences to transform host cells to express corresponding dsRNA sequences that are substantially identical to target sequences in specific pathogens, and particularly describe constructing recombinant plants expressing such dsRNA sequences for ingestion by various plant pests, facilitating down-regulation of a gene in the genome of the pest and improving the resistance of the plant to the pest infestation.

The present disclosure provides for inhibiting gene expression of one or multiple target genes in a target insect pest using stabilized dsRNA methods. The disclosure is particularly useful in the modulation of gene expression in the gut and bacteriocytes of insect pests such as cassava whitefly. The modulatory effect is applicable to a variety of genes expressed in the pests including, for example, endogenous genes responsible for cellular metabolism or cellular transformation, including housekeeping genes, transcription factors and other genes which encode polypeptides involved in cellular metabolism. The modulatory effect is also applicable to cassava whitefly's intracellular symbiont, Portiera, which is restricted to the cytoplasm of insect cells known as bacteriocytes; and provides several essential amino-acids or metabolites for intermediate reactions within different essential amino-acid biosynthesis pathways in the insect pest.

RNAis, as described herein, can be expressed in a multimeric structure such as a concatemer. Multimeric RNAis are well-known in the art (see e.g., Chandra et al. SCI, 75 (2) (2019) 506-514 and Kim et al. Journal of Controlled Release 345 (2022) 770-785). For example, the multimeric RNAi can target one, two, three, four, or more genes in an invertebrate pest. For example, any RNAis described herein, including single, stacked, or multimeric RNAis can target or interfere with one or more sugar transport or storage genes (e.g., TPS, UGP1, and STs, causing reduced growth and molting), genes for detoxification of phytotoxins (e.g., UDP and ABC, causing intoxication), osmoregulation genes (e.g., GH-13 and AQP, causing dehydration), and/or genes associated with the biosynthesis of nutrients (e.g., SYM, reduced growth and mortality). As another example, RNAis can be singular, an inverted repeat, repeat, and/or stacked (e.g., ST-ST-ST-CM-CM-CM-AQP-AQP-AQP) or in a multimeric structure (e.g., ST-ST-ST, CM-CM-CM, AQP-AQP-AQP).

As used herein, the term “expression” refers to the transcription and stable accumulation of sense or antisense RNA derived from the nucleic acids disclosed in the present disclosure.

Expression may also refer to translation of mRNA into a polypeptide or protein. As used herein, the term “sense” RNA refers to an RNA transcript corresponding to a sequence or segment that, when produced by the target pest, is in the form of a mRNA that is capable of being translated into protein by the target pest cell. As used herein, the term “antisense RNA” refers to an RNA transcript that is complementary to all or a part of a mRNA that is normally produced in a cell of a target pest. The complementarity of an antisense RNA may be with any part of the specific gene transcript, i.e., at the 5′ non-coding sequence, 3′ non-translated sequence, introns, or the coding sequence. As used herein, the term “RNA transcript” refers to the product resulting from RNA polymerase-catalyzed transcription of a DNA sequence. When the RNA transcript is a perfect complementary copy of the DNA sequence, it is referred to as the primary transcript or it may be an RNA sequence derived from post-transcriptional processing of the primary transcript and is referred to as the mature RNA.

As used herein, the phrase “inhibition of gene expression” or “inhibiting expression of a target gene in the cell of an insect” refers to the absence (or observable decrease) in the level of protein and/or mRNA product from the target gene. Specificity refers to the ability to inhibit the target gene without manifest effects on other genes of the cell and without any effects on any gene within the cell that is producing the dsRNA molecule. The inhibition of gene expression of the target gene in the insect pest may result in novel phenotypic traits in the insect pest.

Without limiting the scope of the present invention, there is provided, in one aspect, a method for controlling infestation of a target insect using the stabilized dsRNA strategies. The method involves generating stabilized dsRNA molecules as one type of the insect control agents to induce gene silencing in an insect pest. The insect control agents of the present disclosure induce directly or indirectly post-transcriptional gene silencing events of target genes in the insect. Down-regulation of expression of the target gene prevents or at least retards the insect's growth, development, reproduction, and infectivity to hosts. As used herein, the phrase “generating stabilized dsRNA molecule” refers to the methods of employing recombinant DNA technologies readily available in the art (e.g., by Sambrook, et al., In: Molecular Cloning, A Laboratory Manual, 2nd Edition, Cold Spring Harbor Press, Cold Spring Harbor, New York, 1989) to construct a DNA nucleotide sequence that transcript the stabilized dsRNA. The detailed construction methods of the present disclosure are disclosed below in this disclosure. As used herein, the term “silencing” refers the effective “down-regulation” of expression of the targeted nucleotide sequence and, hence, the elimination of the ability of the sequence to cause an effect within the insect's cell.

The present disclosure provides in part a delivery system for the delivery of the insect control agents to insects through their exposure to a diet containing the insect control agents of the present disclosure. In accordance with one of the embodiments, the stabilized dsRNA or siRNA molecules may be incorporated in the insect diet or may be overlaid on the top of the diet for consumption by an insect.

The present disclosure also provides systems for the delivery of the insect control agents to insects through their exposure to a microorganism or a host such as a plant containing the insect control agents of the present disclosure by ingestion of the microorganism or the host cells or the contents of the cells. In accordance with other embodiments, the present disclosure involves generating a transgenic plant cell or a plant that contains a recombinant DNA construct transcribing the stabilized dsRNA molecules of the present disclosure. As used herein, the phrase “generating a transgenic plant cell or a plant” refers to the methods of employing the recombinant DNA technologies readily available in the art (e.g., by Sambrook, et al.) to construct a plant transformation vector transcribing the stabilized dsRNA molecules of the present disclosure, to transform the plant cell or the plant and to generate the transgenic plant cell or the transgenic plant that contain the transcribed, stabilized dsRNA molecules. In particular, the method of the present disclosure may comprise the recombinant construct in a cell of a plant that results in dsRNA transcripts that are substantially homologous to an RNA sequence encoded by a nucleotide sequence within the genome of an insect. Where the nucleotide sequence within the genome of an insect encodes a gene essential to the viability and infectivity of the insect, its down-regulation results in a reduced capability of the insect to survive and infect host cells. Hence, such down-regulation results in a “deleterious effect” on the maintenance viability and infectivity of the insect, in that it prevents or reduces the insect's ability to feed off and survive on nutrients derived from the host cells. By virtue of this reduction in the insect's viability and infectivity, resistance and/or enhanced tolerance to infection by an insect is facilitated in the cells of a plant. Genes in the insect may be targeted at the mature (adult), immature (larval), or egg stages.

In still another embodiment, non-pathogenic, attenuated strains of microorganisms may be used as a carrier for the insect control agents and, in this perspective, the microorganisms carrying such agents are also referred to as insect control agents. The microorganisms may be engineered to express a nucleotide sequence of a target gene to produce RNA molecules comprising RNA sequences homologous or complementary to RNA sequences typically found within the cells of an insect. Exposure of the insects to the microorganisms result in ingestion of the microorganisms and down-regulation of expression of target genes mediated directly or indirectly by the RNA molecules or fragments or derivatives thereof.

The present disclosure alternatively provides exposure of an insect to the insect control agents of the present disclosure incorporated in a spray mixer and applied to the surface of a host, such as a host plant. In an exemplary embodiment, ingestion of the insect control agents by an insect delivers the insect control agents to the gut of the insect and subsequently to the cells within the body of the insect. In another embodiment, infection of the insect by the insect control agents through other means such as by injection or other physical methods also permits delivery of the insect control agents. In yet another embodiment, the RNA molecules themselves are encapsulated in a synthetic matrix such as a polymer and applied to the surface of a host such as a plant. Ingestion of the host cells by an insect permits delivery of the insect control agents to the insect and results in down-regulation of a target gene in the host.

In further embodiments, insect control agents such a RNAi agents as described herein can be used as a topical application or in biosoil amendments aimed at controlling pests and enhancing plant health. In a biosoil application, RNAi molecules can be delivered through soil amendments that contain dsRNA tailored to target pest genes. As pests feed on plants or interact with soil containing these amendments, they absorb the dsRNA, triggering gene silencing in critical pathways. This method shows promise for controlling various soil-borne pests and pathogens while reducing the need for chemical pesticides.

Using RNAi applied to a crop such as in biosoil, can offer several benefits. For example, RNAi can be used as a targeted pest control: Unlike broad-spectrum pesticides, RNAi targets specific pests, reducing unintended effects on beneficial soil organisms. As another example, RNAis are environmentally safe. RNAi degrades relatively quickly in the soil, reducing long-term environmental residues. As yet another example, RNAis have reduced resistance risks. Because RNAi targets unique genetic sequences, pests can be less likely to develop resistance as quickly as they do with traditional chemical pesticides. As yet another example, RNAi can have soil and Plant health benefits. By controlling pests in the soil, RNAi-based biosoil amendments can help plants grow more robustly, increase yield, and reduce the spread of soil-borne diseases.

It is envisioned that the compositions of the present disclosure can be incorporated within the seeds of a plant species either as a product of expression from a recombinant gene incorporated into a genome of the plant cells, or incorporated into a coating or seed treatment that is applied to the seed before planting. The plant cell containing a recombinant gene is considered herein to be a transgenic event.

It is believed that a pesticidal seed treatment can provide significant advantages when combined with a transgenic event that provides protection from invertebrate pest infestation that is within the preferred effectiveness range against a target pest. In addition, it is believed that there are situations that are well known to those having skill in the art, where it is advantageous to have such transgenic events within the preferred range of effectiveness.

The present disclosure also includes seeds and plants having more than one transgenic event. Such combinations are referred to as “stacked” transgenic events. These stacked transgenic events can be events that are directed at the same target pest, or they can be directed to different target pests.

It is believed that the combination of a transgenic seed exhibiting bioactivity against a target pest as a result of the production of an insecticidal amount of an insecticidal dsRNA within the cells of the transgenic seed or plant grown from the seed coupled with treatment of the seed with certain chemical or protein pesticides, including insecticides, provides synergistic advantages to seeds having such treatment, including unexpectedly superior efficacy for protection against damage to the resulting transgenic plant by the target pest. The seeds of the present disclosure are also believed to have the property of decreasing the cost of pesticide use, because less of the pesticide can be used to obtain a required amount of protection than if the innovative composition and method is not used. Moreover, because less pesticide is used and because it is applied prior to planting and without a separate field application, it is believed that the subject method is therefore safer to the operator and to the environment and is potentially less expensive than conventional methods.

When it is said that some effects are “synergistic”, it is meant to include the synergistic effects of the combination on the pesticidal activity (or efficacy) of the combination of the transgenic event and the pesticide. However, it is not intended that such synergistic effects be limited to the pesticidal activity, but that they should also include such unexpected advantages as increased scope of activity, advantageous activity profile as related to type and amount of damage reduction, decreased cost of pesticide and application, decreased pesticide distribution in the environment, decreased pesticide exposure of personnel who produce, handle and plant corn seeds, and other advantages known to those skilled in the art.

Pesticides and insecticides that are useful in compositions in combination with the methods and compositions of the present disclosure, including as seed treatments and coatings as well as methods for using such compositions can be found, for example, in U.S. Pat. No. 6,551,962, the entirety of which is incorporated herein by reference.

The subject pesticides can be applied to a seed as a component of a seed coating. Seed coating methods and compositions that are known in the art are useful when they are modified by the addition of one of the embodiments of the combination of pesticides of the present disclosure. Such coating methods and apparatus for their application are disclosed in, for example, U.S. Pat. Nos. 5,918,413, 5,891,246, 5,554,445, 5,389,399, 5,107,787, 5,080,925, 4,759,945 and 4,465,017. Seed coating compositions are disclosed, for example, in U.S. Pat. Nos. 5,939,356, 5,882,713, 5,876,739, 5,849,320, 5,834,447, 5,791,084, 5,661,103, 5,622,003, 5,580,544, 5,328,942, 5,300,127, 4,735,015, 4,634,587, 4,383,391, 4,372,080, 4,339,456, 4,272,417 and 4,245,432, among others.

As used herein, the term “insect control agent”, or “gene suppression agent” refers to a particular RNA molecule consisting of a first RNA segment and a second RNA segment linked by a third RNA segment. The first and the second RNA segments lie within the length of the RNA molecule and are substantially inverted repeats of each other and are linked together by the third RNA segment. The complementarity between the first and the second RNA segments results in the ability of the two segments to hybridize in vivo and in vitro to form a double stranded molecule, i.e., a stem, linked together at one end of each of the first and second segments by the third segment which forms a loop, so that the entire structure forms into a stem and loop structure, or even more tightly hybridizing structures may form into a stem-loop knotted structure. The first and the second segments correspond invariably and not respectively to a sense and an antisense sequence with respect to the target RNA transcribed from the target gene in the target insect pest that is suppressed by the ingestion of the dsRNA molecule. The insect control agent can also be a substantially purified (or isolated) nucleic acid molecule and more specifically nucleic acid molecules or nucleic acid fragment molecules thereof from a genomic DNA (gDNA) or cDNA library. Alternatively, the fragments may comprise smaller oligonucleotides having from about 15 to about 250 nucleotide residues, and more preferably, about 15 to about 30 nucleotide residues. The “insect control agent” may also refer to a DNA construct that comprises the isolated and purified nucleic acid molecules or nucleic acid fragment molecules thereof from a gDNA or cDNA library. The “insect control agent” may further refer to a microorganism comprising such a DNA construct that comprises the isolated and purified nucleic acid molecules or nucleic acid fragment molecules thereof from a gDNA or cDNA library. As used herein, the phrase “generating an insect control agent” refers to the methods of employing the recombinant DNA technologies readily available in the art (e.g., by Sambrook, et al.) to prepare a recombinant DNA construct transcribing the stabilized dsRNA or siRNA molecules, to construct a vector transcribing the stabilized dsRNA or siRNA molecules, and/or to transform and generate the cells or the microorganisms that contain the transcribed, stabilized dsRNA or siRNA molecules. The methods of the present disclosure provide for the production of a dsRNA transcript, the nucleotide sequence of which is substantially homologous to a targeted RNA sequence encoded by a target nucleotide sequence within the genome of a target insect pest.

As used herein, the term “genome” as it applies to cells of an insect or a host encompasses not only chromosomal DNA found within the nucleus, but organelle DNA found within subcellular components of the cell. The DNA of the present disclosure introduced into plant cells can therefore be either chromosomally integrated or organelle localized. The term “genome” as it applies to bacteria encompasses both the chromosome and plasmids within a bacterial host cell. The DNAs of the present disclosure introduced into bacterial host cells can therefore be either chromosomally integrated or plasmid localized.

Inhibition of target gene expression may be quantified by measuring either the endogenous target RNA or the protein produced by translation of the target RNA and the consequences of inhibition can be confirmed by examination of the outward properties of the cell or organism. Techniques for quantifying RNA and proteins are well known to one of ordinary skill in the art. Multiple selectable markers are available that confer resistance to ampicillin, bleomycin, chloramphenicol, gentamycin, hygromycin, kanamycin, lincomycin, methotrexate, phosphinothricin, puromycin, spectinomycin, rifampicin, and tetracyclin, and the like.

In certain preferred embodiments gene expression is inhibited by at least 10%, preferably by at least 33%, more preferably by at least 50%, and yet more preferably by at least 80%. In particularly preferred embodiments of the disclosure gene expression is inhibited by at least 80%, more preferably by at least 90%, more preferably by at least 95%, or by at least 99% within cells in the insect so a significant inhibition takes place. Significant inhibition is intended to refer to sufficient inhibition that results in a detectable phenotype (e.g., cessation of larval growth, paralysis, or mortality, etc.) or a detectable decrease in RNA and/or protein corresponding to the target gene being inhibited. Although in certain embodiments of the disclosure inhibition occurs in substantially all cells of the insect, in other preferred embodiments inhibition occurs in only a subset of cells expressing the gene. For example, if the gene to be inhibited plays an essential role in cells in the insect alimentary tract, inhibition of the gene within these cells is sufficient to exert a deleterious effect on the insect.

The advantages of the present disclosure may include, but are not limited to, the following: the case of introducing dsRNA into the insect cells, the low concentration of dsRNA or siRNA which can be used, the stability of dsRNA or siRNA, and the effectiveness of the inhibition. The ability to use a low concentration of a stabilized dsRNA avoids several disadvantages of anti-sense interference. The present disclosure is not limited to in vitro use or to specific sequence compositions, to a particular set of target genes, a particular portion of the target gene's nucleotide sequence, or a particular transgene or to a particular delivery method, as opposed to the some of the available techniques known in the art, such as antisense and co-suppression. Furthermore, genetic manipulation becomes possible in organisms that are not classical genetic models.

In practicing the present disclosure, it is important that the presence of the nucleotide sequences that are transcribed from the recombinant construct are neither harmful to cells of the plant in which they are expressed in accordance with the disclosure, nor harmful to an animal food chain and in particular humans. Because the produce of the plant may be made available for human ingestion, the downregulation of expression of the target nucleotide sequence occurs only in the insect.

Therefore, in order to achieve inhibition of a target gene selectively within an insect species that it is desired to control, the target gene should preferably exhibit a low degree of sequence identity with corresponding genes in a plant or a vertebrate animal. Preferably the degree of the sequence identity is less than approximately 80%. More preferably the degree of the sequence identity is less than approximately 70%. Most preferably the degree of the sequence identity is less than approximately 60%.

According to one embodiment of the present disclosure, there is provided a nucleotide sequence, for which in vitro expression results in transcription of a stabilized RNA sequence that is substantially homologous to an RNA molecule of a targeted gene in an insect that comprises an RNA sequence encoded by a nucleotide sequence within the genome of the insect. Thus, after the insect ingests the stabilized RNA sequence incorporated in a diet or sprayed on a plant surface, a down-regulation of the nucleotide sequence corresponding to the target gene in the cells of a target insect is affected. The down-regulated nucleotide sequence in the insect results in a deleterious effect on the maintenance, viability, proliferation, reproduction, and infectivity of the insect. Therefore, the nucleotide sequence of the present disclosure may be useful in modulating or controlling infestation by a range of insects.

According to another embodiment of the present disclosure, there is provided a nucleotide sequence, the expression of which in a microbial cell results in a transcription of an RNA sequence which is substantially homologous to an RNA molecule of a targeted gene in an insect that comprises an RNA sequence encoded by a nucleotide sequence within the genome of the insect. Thus, after the insect ingests the stabilized RNA sequence contained in the cell of the microorganism, it will affect down-regulation of the nucleotide sequence of the target gene in the cells of the insect. The down-regulated nucleotide sequence in the insect results in a deleterious effect on the maintenance, viability, proliferation, reproduction, and infestation of the insect. Therefore, the nucleotide sequence of the present disclosure may be useful in modulating or controlling infestation by a range of insects.

According to yet another embodiment of the present disclosure, there is provided a nucleotide sequence, the expression of which in a plant cell results in a transcription of an RNA sequence which is substantially homologous to an RNA molecule of a targeted gene in an insect that comprises an RNA sequence encoded by a nucleotide sequence within the genome of the insect. Thus, after the insect ingests the stabilized RNA sequence contained in the cell of the plant, it will affect down-regulation of the nucleotide sequence of the target gene in the cells of the insect. The down-regulated nucleotide sequence in the insect results in a deleterious effect on the maintenance, viability, proliferation, reproduction, and infestation of the insect. Therefore, the nucleotide sequence of the present disclosure may be useful in modulating or controlling infestation by a range of insects in plants.

As used herein, the term “substantially homologous” or “substantial homology”, with reference to a nucleic acid sequence, refers to a nucleotide sequence that hybridizes under stringent conditions to a nucleotide sequence encoding a polypeptide having a sequence selected from the group consisting of SEQ ID NO:2, 4, 6, 8, 10, 12, 14, 16, 18, 20, 22, 24, 26, 28, 30, 32, 34, 36, 38, and 40, or the group consisting of SEQ ID NO:41, 43, 45, 47, 49, 51, 53, 98, 100, 102, 104, and 106, and wherein said recombinant polynucleotide molecule disrupts the activity of said polypeptide when provided in the diet of an invertebrate pest. Sequences that hybridize under stringent conditions to a nucleotide sequence encoding a polypeptide having a sequence as set forth in the sequence listing, or the complements thereof, are those that allow an antiparallel alignment to take place between the two sequences, and the two sequences are then able, under stringent conditions, to form hydrogen bonds with corresponding bases on the opposite strand to form a duplex molecule that is sufficiently stable under the stringent conditions to be detectable using methods well known in the art. Such substantially homologous sequences have preferably from about 65% to about 70% sequence identity, or more preferably from about 80% to about 85% sequence identity, or most preferable from about 90% to about 95% sequence identity, to about 99% sequence identity, to the referent nucleotide sequences, or the complements thereof.

As used herein, the term “sequence identity”, “sequence similarity” or “homology” is used to describe sequence relationships between two or more nucleotide sequences. The percentage of “sequence identity” between two sequences is determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. A sequence that is identical at every position in comparison to a reference sequence is said to be identical to the reference sequence and vice-versa. A first nucleotide sequence when observed in the 5′ to 3′ direction is said to be a “complement” of, or complementary to, a second or reference nucleotide sequence observed in the 3′ to 5′ direction if the first nucleotide sequence exhibits complete complementarity with the second or reference sequence. As used herein, nucleic acid sequence molecules are said to exhibit “complete complementarity” when every nucleotide of one of the sequences read 5′ to 3′ is complementary to every nucleotide of the other sequence when read 3′ to 5′. A nucleotide sequence that is complementary to a reference nucleotide sequence will exhibit a sequence identical to the reverse complement sequence of the reference nucleotide sequence. These terms and descriptions are well defined in the art and are easily understood by those of ordinary skill in the art.

As used herein, a “comparison window” refers to a conceptual segment of at least 6 contiguous positions, usually about 50 to about 100, more usually about 100 to about 150, in which a sequence is compared to a reference sequence of the same number of contiguous positions after the two sequences are optimally aligned. The comparison window may comprise additions or deletions (i.e. gaps) of about 20% or less as compared to the reference sequence (which does not comprise additions or deletions) for optimal alignment of the two sequences Those skilled in the art should refer to the detailed methods used for sequence alignment in the Wisconsin Genetics Software Package Release 7.0, Genetics Computer Group, 575 Science Drive Madison, Wis., USA) or refer to Ausubel, et al. (1998) for a detailed discussion of sequence analysis.

The target gene of the present disclosure is derived from an insect cell, e.g., a bacteriocyte, or alternatively, a foreign gene such as a foreign genetic sequence from an endosymbiont, a virus, a fungus, an insect, or a nematode, among others. By “derived” it is intended that a sequence is all or a part of the naturally occurring nucleotide sequence of the target gene from the genome of an insect cell, particularly all or a part of the naturally occurring nucleotide sequence of the capped, spliced, and polyadenylated mRNA expressed from the naturally occurring DNA sequence as found in the cell if the gene is a structural gene, or the sequence of all or a part of an RNA that is other than a structural gene including but not limited to a tRNA, a catalytic RNA, a ribosomal RNA, a micro-RNA, and the like. A sequence is derived from one of these naturally occurring RNA sequences if the derived sequence is produced based on the nucleotide sequence of the native RNA, exhibits from about 80% to about 100% sequence identity to the native sequence, and hybridizes to the native sequence under stringent hybridization conditions. In one embodiment, the target gene comprises a nucleotide sequence as set forth in any of SEQ ID NO:1, 3, 5, 7, 9, 11, 13, 15, 17, 19, 21, 23, 25, 27, 29, 31, 33, 35, 37, and 39 as set forth in the sequence listing, or fragments or complements thereof. Depending on the particular target gene and the dose of dsRNA molecules delivered, this process may provide partial or complete loss of function for the target gene, or any desired level of suppression in between.

The present disclosure also provides an artificial DNA sequence capable of being expressed in a cell or microorganism and which is capable of inhibiting target gene expression in a cell, tissue or organ of an insect, wherein the artificial DNA sequence at least comprises a dsDNA molecule coding for one or more different nucleotide sequences, wherein each of the different nucleotide sequences comprises a sense nucleotide sequence and an antisense nucleotide sequence connected by a spacer sequence coding for a dsRNA molecule of the present disclosure. The spacer sequence constitutes part of the sense nucleotide sequence or the antisense nucleotide sequence and will form within the dsRNA molecule between the sense and antisense sequences. The sense nucleotide sequence or the antisense nucleotide sequence is substantially identical to the nucleotide sequence of the target gene or a derivative thereof or a complementary sequence thereto. The dsDNA molecule is placed operably under the control of a promoter sequence that functions in the cell, tissue or organ of the host expressing the dsDNA to produce dsRNA molecules.

The disclosure also provides an artificial DNA sequence for expression in a cell of a plant, and that, upon expression of the DNA to RNA and ingestion by a target pest achieves suppression of a target gene in a cell, tissue, or organ of an insect pest. The dsRNA at least comprises one or multiple structural gene sequences, wherein each of the structural gene sequences comprises a sense nucleotide sequence and an antisense nucleotide sequence connected by a spacer sequence that forms a loop within the complementary and antisense sequences. The sense nucleotide sequence or the antisense nucleotide sequence is substantially identical to the nucleotide sequence of the target gene, derivative thereof, or sequence complementary thereto. The one or more structural gene sequences is placed operably under the control of one or more promoter sequences, at least one of which is operable in the cell, tissue, or organ of a prokaryotic or eukaryotic organism, particularly an insect.

As used herein, the term “non-naturally occurring gene”, “non-naturally occurring coding sequences”, “artificial sequence”, or “synthetic coding sequences” for transcribing the dsRNA or siRNA of the present disclosure or fragments thereof refers to those prepared in a manner involving any sort of genetic isolation or manipulation that results in the preparation of a coding sequence that transcribes a dsRNA or a siRNA of the present disclosure or fragments thereof. This includes isolation of the coding sequence from its naturally occurring state, manipulation of the coding sequence as by (1) nucleotide insertion, deletion, or substitution, (2) segment insertion, deletion, or substitution, (3) chemical synthesis such as phosphoramidite chemistry and the like, site-specific mutagenesis, truncation of the coding sequence or any other manipulative or isolative method.

The non-naturally occurring gene sequence or fragment thereof according to this aspect of the disclosure for cassava whitefly control may be cloned between two tissue specific promoters, such as two phloem specific promoters which are operable in a transgenic plant cell and therein expressed to produce mRNA in the transgenic plant cell that form dsRNA molecules thereto. The dsRNA molecules contained in plant tissues are ingested by an insect so that the intended suppression of the target gene expression is achieved.

The present disclosure also provides a method for obtaining a nucleic acid comprising a nucleotide sequence for producing a dsRNA or siRNA of the present disclosure. In a preferred embodiment, the method of the present disclosure for obtaining the nucleic acid comprising: (a) probing a cDNA or gDNA library with a hybridization probe comprising all or a portion of a nucleotide sequence or a homolog thereof from a targeted insect; (b) identifying a DNA clone that hybridizes with the hybridization probe; (c) isolating the DNA clone identified in step (b); and (d) sequencing the cDNA or gDNA fragment that comprises the clone isolated in step (c) wherein the sequenced nucleic acid molecule transcribes all or a substantial portion of the RNA nucleotide acid sequence or a homolog thereof.

In another preferred embodiment, the method of the present disclosure for obtaining a nucleic acid fragment comprising a nucleotide sequence for producing a substantial portion of a dsRNA or siRNA of the present disclosure comprising: (a) synthesizing a first and a second oligonucleotide primers corresponding to a portion of one of the nucleotide sequences from a targeted insect; and (b) amplifying a cDNA or gDNA insert present in a cloning vector using the first and second oligonucleotide primers of step (a) wherein the amplified nucleic acid molecule transcribes a substantial portion of the a substantial portion of a dsRNA or siRNA of the present disclosure.

In practicing the present disclosure, a target gene may be derived from a whitefly, such as an African cassava whitefly or B. tabaci SSA1-SG1, or any insect species that cause damages to the crop plants and subsequent yield losses or serves as a vector for plant virus transmission. The present inventors contemplate that several criteria may be employed in the selection of preferred target genes. The gene is one whose protein product has a rapid turnover rate, so that dsRNA inhibition will result in a rapid decrease in protein levels. In certain embodiments it is advantageous to select a gene for which a small drop in expression level results in deleterious effects for the insect. If it is desired to target a broad range of insect species a gene is selected that is highly conserved across these species. Conversely, for the purpose of conferring specificity, in certain embodiments of the disclosure, a gene is selected that contains regions that are poorly conserved between individual insect species, or between insects and other organisms. In certain embodiments it may be desirable to select a gene that has no known homologs in other organisms.

As used herein, the term “derived from” refers to a specified nucleotide sequence that may be obtained from a particular specified source or species, albeit not necessarily directly from that specified source or species.

In one embodiment, a gene is selected that is expressed in the insect gut. Targeting genes expressed in the gut avoids the requirement for the dsRNA to spread within the insect. Target genes for use in the present disclosure may include, for example, those that share substantial homologies to the nucleotide sequences of gut-expressed genes that encode protein components that mediate phloem sap sugar transformations or maintain optimum osmotic pressure within the gut. Among the osmoregulation genes, target genes include, but are not limited to, genes ST1, ST2, TPS, Hx, UGP1, ABC1, ABC, UDP-GT1, UDP-GT2, and UDP-GT3.

The present disclosure is not limited to the specific genes described herein but encompasses any gene, the inhibition of which exerts a deleterious effect on an insect pest. In order to obtain a DNA segment from the corresponding gene in an insect species, PCR primers may be designed based on the sequence as found in cassava whitefly or other insects from which the gene has been cloned. The primers are designed to amplify a DNA segment of sufficient length for use in the present disclosure. DNA (either genomic DNA or cDNA) is prepared from the insect species, and the PCR primers are used to amplify the DNA segment. Amplification conditions are selected so that amplification will occur even if the primers do not exactly match the target sequence. Alternately, the gene (or a portion thereof) may be cloned from a gDNA or cDNA library prepared from the insect pest species, using the cassava whitefly gene or another known insect gene as a probe. Techniques for performing PCR and cloning from libraries are known. Further details of the process by which DNA segments from target insect pest species may be isolated based on the sequence of genes previously cloned from cassava whitefly or other insect species are provided in the Examples.

The present disclosure provides for a recombinant polynucleotide molecule that disrupts the activity of said polypeptide when provided in the diet of an invertebrate pest. The invertebrate pest can be a pest in the order of Hemiptera. For example, the Hemipteran can belong to the family Alcyrodidae, comprising over 1,200 known species of whitefly, many of which are pests to various plants and crops worldwide. For example, the whitefly can be of species Aleurocanthus spiniferus (Orange spiny whitefly), Alcuroclava lefroyi (Coconut whitefly), Aleuroclava manii, Aleurodicus dispersus (Spiralling whitefly), Aleurodicus rugioperculatus (Rugose spiralling whitefly), Alcurothrixus floccosus (Woolly whitefly), Alcurotrachelus atratus (Palm infesting whitefly), Alcyrodes proletella (Cabbage whitefly), Bemisia argentifolii (Silverleaf whitefly), Bemisia tabaci (Sweet potato whitefly/Cassava whitefly, SSA1, SSA2, MEAMI, also known as B biotype; MED, also known as Q biotype), Paraleyrodes bondari (Bondar's nesting whitefly), or Trialeurodes vaporariorum (Greenhouse whitefly). These species are known for their impact on agriculture and horticulture, causing significant damage to various crops through direct feeding and transmission of plant viruses. For example, cassava whitefly/sweet potato whitefly (Bemisia tabaci) is a significant pest for many agricultural crops, spreading several plant viruses; greenhouse whitefly (Trialeurodes vaporariorum) commonly affects a wide range of herbaceous plants in greenhouses and gardens; silverleaf whitefly (Bemisia argentifolii) is known for infesting numerous plants including tomatoes, beans, and various ornamentals, and causing specific damage symptoms such as silvering of leaves and irregular ripening of fruits; citrus blackfly (Alcurocanthus woglumi), despite its name, is a whitefly that targets citrus plants; cabbage whitefly (Alcyrodes proletella) is a pest of various Brassica crops. In addition to being vectors for plant viruses, which make them particularly dangerous to crops, whiteflies excrete a sticky substance called honeydew, which can lead to sooty mold growth, further harming the plants.

Current control methods can include regular inspection, biological control using natural predators, mechanical control with sticky traps, and chemical treatments, although resistance to traditional insecticides can be a problem. Integrated pest management strategies are often recommended for effective control of whitefly populations.

It has been found that the present disclosure is particularly effective when the insect pest is a B. tabaci., and especially when the pest is B. tabaci SSA1-SG1. The present disclosure is also particularly effective for controlling species of insects that pierce and/or suck the fluids from the cells and tissues of plants, including but not limited to phloem-sap feeders. Modifications of the methods disclosed herein are also surprisingly particularly useful in controlling crop pests within the order Hemiptera.

Hemiptera can include various pest species that affect crops worldwide. These pests often feed on plant sap, weakening plants, and spreading plant diseases. Common Hemipteran pests for crops can include Aphids (Aphididae), Whiteflies (Aleyrodidae), Leafhoppers (Cicadellidae), Stink Bugs (Pentatomidae), Mealybugs (Pseudococcidae), Psyllids (Psyllidae), or Planthoppers (Delphacidae).

For example, the compositions and methods disclosed herein are effective at controlling Aphids (Aphididae). Aphids can affect almost all crop types, including vegetables, grains, fruits, and ornamentals. Aphids can damage plants and crops by sucking plant sap, leading to stunted growth, curled leaves, and reduced yield. They can also excrete honeydew, promoting sooty mold growth and attract ants. Aphids can transmit diseases as they can be a vector for many plant viruses.

As another example, the compositions and methods disclosed herein are effective at controlling Whiteflies (Aleyrodidae). Whiteflies can affect crops including Vegetables (tomato, eggplant), fruits, and ornamentals. Whiteflies, similar to aphids, can damage plants and crops by sucking sap, causing plant stress, yellowing, and reduced growth. They can also produce honeydew, leading to sooty mold. Whiteflies can transmit diseases as they can be a vector for many plant viruses like Tomato yellow leaf curl virus and other viral pathogens.

Yet another example, the compositions and methods disclosed herein are effective at controlling Leafhoppers (Cicadellidae). Leafhoppers can affect crops including grains, alfalfa, grapes, citrus, and potatoes. Leafhoppers can damage plants and crops by feeding on plant juices, which can cause yellowing, stunting, and leaf curling. Their feeding can also inject toxins that further damage plants. Leafhoppers can transmit diseases as they can be a vector for many plant viruses and spread several plant pathogens, including those causing curly top virus and Pierce's disease in grapes.

As yet another example, the compositions and methods disclosed herein are effective at controlling Stink Bugs (Pentatomidae). Stink Bugs can affect crops including corn, soybean, fruits (apple, peach), and various vegetables. Stink Bugs can damage plants and crops by piercing fruits and seeds, leading to scarring and deformation and causes direct crop damage. In grains, they can cause yield loss and reduced quality.

As yet another example, the compositions and methods disclosed herein are effective at controlling Mealybugs (Pseudococcidae). Mealybugs can affect crops including citrus, grapes, coffee, and ornamentals. Mealybugs can damage plants and crops by feeding on plant sap, weakening plants, and causing wilting, yellowing, and stunted growth. They can also excrete honeydew, promoting mold. Mealybugs can transmit some plant diseases, although less commonly than aphids or whiteflies.

As yet another example, the compositions and methods disclosed herein are effective at controlling Psyllids (Psyllidae). Psyllids can affect crops including citrus, tomato, and potato. Psyllids can damage plants by feeding on sap, causing curling, yellowing, and stunted growth. Psyllids can be vectors of significant diseases, such as citrus greening disease and zebra chip disease in potatoes.

As yet another example, the compositions and methods disclosed herein are effective at controlling Planthoppers (Delphacidae). Planthoppers can affect crops including rice, sugarcane, and other grasses. Planthoppers can damage crops and plants by feeding on the phloem of plants, leading to wilting and hopper burn, where leaves turn brown. Planthoppers can transmit plant viruses, such as Rice tungro virus.

Methods of targeting various whitefly species and Hemiptera or symbionts thereof can be performed using the methods as described herein. For example, the genome of various whitefly species and other Hemipteran pests are known in the art (see e.g., www.whiteflygenomics.org, accession numbers: GCA_902825415.1; GCA_902825425.1; GCA_903994125.1; GCA_903994115.1; GCA_903994105.1; GCA_903994095.1; GCA_001854935.1; GCA_003994315.1 (Campbell, et al. BMC Genomics 24, 408 (2023); accession number VMOF00000000, SRP223456 (Xic, et al. Molecular Ecology Resources, 20 (4) 2020)). Targets as described herein can be extrapolated to various Hemipteran and whitefly species as described in Tables 2-5.

The present disclosure provides stabilized dsRNA or siRNA molecules for control of insect infestations. The dsRNA or siRNA nucleotide sequences comprise double strands of polymerized ribonucleotide and may include modifications to either the phosphate-sugar backbone or the nucleoside. Modifications in RNA structure may be tailored to allow specific genetic inhibition.

In one embodiment, the dsRNA molecules may be modified through an enzymatic process so the siRNA molecules may be generated. The siRNA can efficiently mediate the down-regulation effect for some target genes in some insects. This enzymatic process may be accomplished by utilizing an RNAse III enzyme or a DICER enzyme, present in the cells of an insect, a vertebrate animal, a fungus, or a plant in the eukaryotic RNAi pathway (Elbashir et al., 2002, Methods, 26 (2): 199-213; Hamilton and Baulcombe, 1999, Science 286:950-952). This process may also utilize a recombinant DICER or RNAse III introduced into the cells of a target insect through recombinant DNA techniques that are readily known to the skilled in the art. Both the DICER enzyme and RNAse III, being naturally occurring in an insect or being made through recombinant DNA techniques, cleave larger dsRNA strands into smaller oligonucleotides. The DICER enzymes specifically cut the dsRNA molecules into siRNA pieces each of which is about 19-25 nucleotides in length while the RNAse III enzymes normally cleave the dsRNA molecules into 12-15 base-pair siRNA. The siRNA molecules produced by the either of the enzymes have 2 to 3 nucleotide 3′ overhangs, and 5′ phosphate and 3′ hydroxyl termini. The siRNA molecules generated by RNAse III enzyme are the same as those produced by Dicer enzymes in the eukaryotic RNAi pathway and are hence then targeted and degraded by an inherent cellular RNA-degrading mechanism after they are subsequently unwound, separated into single-stranded RNA, and hybridize with the RNA sequences transcribed by the target gene. This process results in the effective degradation or removal of the RNA sequence encoded by the nucleotide sequence of the target gene in the insect. The outcome is the silencing of a particularly targeted nucleotide sequence within the insect. Detailed descriptions of enzymatic processes can be found in Hannon (2002, Nature, 418:244-251).

The compositions and methods disclosed herein are effective at controlling Whitefly species, including, but not limited to, the species listed in Table 1.

TABLE 1
Whitefly species and target plants.

| Whitefly Species | Target Plants/Crops |
|---|---|
| Bemisia tabaci | Cotton, Tomato, Tobacco, |
| (Silverleaf/Tobacco Whitefly) | Soybean, Cucurbits, Beans |
| Bemisia afer (Castor | Castor, Cassava, Ornamentals |
| Whitefly) | |
| Trialeurodes vaporariorum | Tomato, Cucumber, Eggplant, |
| (Greenhouse Whitefly) | Peppers, Ornamentals |
| Trialeurodes ricini (Castor | Castor, Bean, Cassava |
| Bean Whitefly) | |
| Trialeurodes abutiloneus | Cotton, Beans, Okra |
| (Bandwinged Whitefly) | |
| Aleurodicus dugesii (Giant | Hibiscus, Citrus, Avocado, |
| Whitefly) | Ornamentals |
| Aleurodicus dispersus | Coconut, Banana, Papaya, |
| (Spiraling Whitefly) | Ornamentals |
| Dialeurodes citri (Citrus | Citrus Species |
| Whitefly) | |
| Dialeurodes kirkaldyi | Mango, Citrus |
| (Ceylon Whitefly) | |
| Aleurocanthus woglumi | Citrus, Guava |
| (Citrus Blackfly) | |
| Aleurocanthus spiniferus | Citrus, Tea, Mango, Guava |
| (Orange Spiny Whitefly) | |
| Parabemisia myricae (Citrus | Citrus |
| Whitefly) | |
| Singhiella simplex (Ficus | Ficus Species |
| Whitefly) | |
| Singhiella citrifolii (Citrus | Citrus |
| Whitefly) | |
| Pealius quercus (Oak | Oak |
| Whitefly) | |

Inhibition of a target gene using the stabilized dsRNA technology of the present disclosure is sequence-specific in that nucleotide sequences corresponding to the duplex region of the RNA are targeted for genetic inhibition. RNA containing a nucleotide sequence identical to a portion of the target gene is preferred for inhibition. RNA sequences with insertions, deletions, and single point mutations relative to the target sequence have also been found to be effective for inhibition. In performance of the present disclosure, it is preferred that the inhibitory dsRNA and the portion of the target gene share at least from about 80% sequence identity, or from about 90% sequence identity, or from about 95% sequence identity, or from about 99% sequence identity, or even about 100% sequence identity. Alternatively, the duplex region of the RNA may be defined functionally as a nucleotide sequence that is capable of hybridizing with a portion of the target gene transcript. A less than full length sequence exhibiting a greater homology compensates for a longer less homologous sequence. The length of the identical nucleotide sequences may be at least about 25, 50, 100, 200, 300, 400, 500 or at least about 1000 bases. Normally, a sequence of greater than 20-100 nucleotides should be used, though a sequence of greater than about 200-300 nucleotides would be preferred, and a sequence of greater than about 500-1000 nucleotides would be especially preferred depending on the size of the target gene. The disclosure has the advantage of being able to tolerate sequence variations that might be expected due to genetic mutation, strain polymorphism, or evolutionary divergence. The introduced nucleic acid molecule may not need to be absolute homology, may not need to be full length, relative to either the primary transcription product or fully processed mRNA of the target gene. Therefore, those skilled in the art need to realize that, as disclosed herein, 100% sequence identity between the RNA and the target gene is not required to practice the present disclosure.

The dsRNA molecules may be synthesized either in vivo or in vitro. The dsRNA may be formed by a single self-complementary RNA strand or from two complementary RNA strands. Endogenous RNA polymerase of the cell may mediate transcription in vivo, or cloned RNA polymerase can be used for transcription in vivo or in vitro. Inhibition may be targeted by specific transcription in an organ, tissue, or cell type; stimulation of an environmental condition (e. g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

The RNA, dsRNA, siRNA, or miRNA of the present disclosure may be produced chemically or enzymatically by one skilled in the art through manual or automated reactions or in vivo in another organism. RNA may also be produced by partial or total organic synthesis; any modified ribonucleotide can be introduced by in vitro enzymatic or organic synthesis. The RNA may be synthesized by a cellular RNA polymerase or a bacteriophage RNA polymerase (e.g., T3, T7, SP6). The use and production of an expression construct are known in the art (see, for example, WO 97/32016; U.S. Pat. No's. 5,593,874, 5,698,425, 5,712,135, 5,789,214, and 5,804,693). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

For transcription from a transgene in vivo or an expression construct, a regulatory region (e.g., promoter, enhancer, silencer, and polyadenylation) may be used to transcribe the RNA strand (or strands). Therefore, in one embodiment, the nucleotide sequences for use in producing RNA molecules may be operably linked to one or more promoter sequences functional in a microorganism, a fungus, or a plant host cell. Ideally, the nucleotide sequences are placed under the control of an endogenous promoter, normally resident in the host genome. The nucleotide sequence of the present disclosure, under the control of an operably linked promoter sequence, may further be flanked by additional sequences that advantageously affect its transcription and/or the stability of a resulting transcript. Such sequences are generally located upstream of the operably linked promoter and/or downstream of the 3′ end of the expression construct and may occur both upstream of the promoter and downstream of the 3′ end of the expression construct, although such an upstream sequence only is also contemplated.

In another embodiment, the nucleotide sequence of the present disclosure may comprise an inverted repeat separated by a “spacer sequence”. The spacer sequence may be a region comprising any sequence of nucleotides that facilitates secondary structure formation between each repeat, where this is required. In one embodiment of the present disclosure, the spacer sequence is part of the sense or antisense coding sequence for mRNA. The spacer sequence may alternatively comprise any combination of nucleotides or homologues thereof that are capable of being linked covalently to a nucleic acid molecule. The spacer sequence may comprise a sequence of nucleotides of at least about 10-100 nucleotides in length, or alternatively at least about 100-200 nucleotides in length, at least about 200-400 nucleotides in length, or at least about 400-500 nucleotides in length.

For the purpose of the present disclosure, the dsRNA or siRNA molecules may be obtained from the cassava whitefly by polymerase chain (PCR) amplification of a target gene sequences derived from a cassava whitefly gDNA or cDNA library or portions thereof. The whitefly pupa may be prepared using methods known to the ordinary skilled in the art and DNA/RNA may be extracted. Pupa with various sizes ranging from 1^{st} instars to fully-grown whitefly may be used for the purpose of the present disclosure for DNA/RNA extraction. Genomic DNA or cDNA libraries generated from whitefly may be used for PCR amplification for production of the dsRNA or siRNA.

The target genes may then be PCR amplified and sequenced using the methods readily available in the art. One skilled in the art may be able to modify the PCR conditions to ensure optimal PCR product formation. The confirmed PCR product may be used as a template for in vitro transcription to generate sense and antisense RNA with the included minimal promoters.

The nucleic acids from whitefly or other insects that may be used in the present disclosure may also comprise isolated and substantially purified Unigenes and EST nucleic acid molecules or nucleic acid fragment molecules thereof. EST nucleic acid molecules may encode significant portions of, or indeed most of, the polypeptides. Alternatively, the fragments may comprise smaller oligonucleotides having from about 15 to about 250 nucleotide residues, and more preferably, about 15 to about 30 nucleotide residues. Alternatively, the nucleic acid molecules for use in the present disclosure may be from cDNA libraries from whitefly, or from any other invertebrate pest species.

As used herein, the phrase “a substantially purified nucleic acid”, “an artificial sequence”, “an isolated and substantially purified nucleic acid”, or “an isolated and substantially purified nucleotide sequence” refers to a nucleic acid that is no longer accompanied by some of the materials with which it is associated in its natural state or to a nucleic acid the structure of which is not identical to that of any of naturally occurring nucleic acid. Examples of a substantially purified nucleic acid include: (1) DNAs which have the sequence of part of a naturally occurring genomic DNA molecules but are not flanked by two coding sequences that flank that part of the molecule in the genome of the organism in which it naturally occurs; (2) a nucleic acid incorporated into a vector or into the genomic DNA of a prokaryote or eukaryote in a manner such that the resulting molecule is not identical to any naturally occurring vector or genomic DNA; (3) a separate molecule such as a cDNA, a genomic fragment, a fragment produced by polymerase chain reaction (PCR), or a restriction fragment; (4) recombinant DNAs; and (5) synthetic DNAs. A substantially purified nucleic acid may also be comprised of one or more segments of cDNA, genomic DNA, or synthetic DNA.

Nucleic acid molecules and fragments thereof from cassava whitefly or other invertebrate pest species may be employed to obtain other nucleic acid molecules from other species for use in the present disclosure to produce desired dsRNA and siRNA molecules. Such nucleic acid molecules include the nucleic acid molecules that encode the complete coding sequence of a protein and promoters and flanking sequences of such molecules. In addition, such nucleic acid molecules include nucleic acid molecules that encode for gene family members. Such molecules can be readily obtained by using the above-described nucleic acid molecules or fragments thereof to screen cDNA or gDNA libraries obtained from whitefly, such as the B. tabaci species complex. Methods for forming such libraries are well known in the art.

Nucleic acid molecules and fragments thereof from the whitefly, such as the B. tabaci species complex may also be employed to obtain other nucleic acid molecules such as nucleic acid homologues for use in the present disclosure to produce desired dsRNA and siRNA molecules. Such homologues include the nucleic acid molecules that encode, in whole or in part, protein homologues of other species, plants or other organisms. Such molecules can be readily obtained by using the above-described nucleic acid molecules or fragments thereof to screen EST, cDNA or gDNA libraries. Methods for forming such libraries are well known in the art. Such homologue molecules may differ in their nucleotide sequences disclosed herein, because complete complementarity is not needed for stable hybridization. These nucleic acid molecules also include molecules that, although capable of specifically hybridizing with the nucleic acid molecules may lack complete complementarity. In a particular embodiment, methods for 3′ or 5′ RACE may be used to obtain such sequences (Frohman, M. A. et al., Proc. Natl. Acad. Sci. (U.S.A.) 85:8998-9002 (1988); Ohara, O. et al., Proc. Natl. Acad. Sci. (U.S.A.) 86:5673-5677 (1989)). In general, any of the above-described nucleic acid molecules or fragments may be used to generate dsRNAs or siRNAs that are suitable for use in a diet, in a spray-on mixer or in a recombinant DNA construct of the present disclosure.

As used herein, the phrase “coding sequence”, “structural nucleotide sequence” or “structural nucleic acid molecule” refers to a nucleotide sequence that is translated into a polypeptide, usually via mRNA, when placed under the control of appropriate regulatory sequences. The boundaries of the coding sequence are determined by a translation start codon at the 5′-terminus and a translation stop codon at the 3′-terminus. A coding sequence can include, but is not limited to, genomic DNA, cDNA, EST, and recombinant nucleotide sequences.

The nucleic acid molecules or fragment of the nucleic acid molecules or other nucleic acid molecules from an invertebrate pest, such as a cassava whitefly are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the complement of another nucleic acid molecule if they exhibit complete complementarity. Two molecules are said to be “minimally complementary” if they can hybridize to one another with sufficient stability to permit them to remain annealed to one another under at least conventional “low-stringency” conditions. Similarly, the molecules are said to be complementary if they can hybridize to one another with sufficient stability to permit them to remain annealed to one another under conventional “high-stringency” conditions. Conventional stringency conditions are described by Sambrook, et al., and by Haymes, et al. In: Nucleic Acid Hybridization, A Practical Approach, IRL Press, Washington, DC (1985).

Departures from complete complementarity are therefore permissible, as long as such departures do not completely preclude the capacity of the molecules to form a double-stranded structure. Thus, in order for a nucleic acid molecule or a fragment of the nucleic acid molecule to serve as a primer or probe it needs only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

A nucleic acid for use in the present disclosure may specifically hybridize to one or more of nucleic acid molecules from cassava whitefly or complements thereof under moderately stringent conditions, for example at about 2.0×SSC and about 65° C. A nucleic acid for use in the present disclosure will include those nucleic acid molecules that specifically hybridize to one or more of the nucleic acid molecules disclosed in any of SEQ ID NO: 1, 3, 5, 7, 9, 11, 13, 15, 17, 19, 21, 23, 25, 27, 29, 31, 33, 35, 37, and 39 as set forth in the sequence listing, or fragments or complements thereof under high stringency conditions. Preferably, a nucleic acid for use in the present disclosure will exhibit at least from about 80%, or at least from about 90%, or at least from about 95%, or at least from about 98% or even about 100% sequence identity with one or more nucleic acid molecules as set forth in SEQ ID NO:1, 3, 5, 7, 9, 11, 13, 15, 17, 19, 21, 23, 25, 27, 29, 31, 33, 35, 37, and 39, or as disclosed herein; or a nucleic acid for use in the present disclosure will exhibit at from about 80%, or at least from about 90%, or at least from about 95%, or at least from about 98% or even about 100% sequence identity with one or more nucleic acid molecules as set forth in SEQ ID NO: 1, 3, 5, 7, 9, 11, 13, 15, 17, 19, 21, 23, 25, 27, 29, 31, 33, 35, 37, and 39 in the sequence listing isolated from the genomic DNA of an insect pest.

Nucleic acids of the present disclosure may also be synthesized, either completely or in part, especially where it is desirable to provide plant-preferred sequences, by methods known in the art. Thus, all or a portion of the nucleic acids of the present disclosure may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The present disclosure provides in part a delivery system for the delivery of insect control agents to insects. The stabilized dsRNA or siRNA molecules of the present disclosure may be directly introduced into the cells of an insect, or introduced into an extracellular cavity, interstitial space, lymph system, digestive system, into the circulation of the insect through oral ingestion or other means that one skilled in the art may employ. Methods for oral introduction may include direct mixing of RNA with food of the insect, as well as engineered approaches in which a species that is used as food is engineered to express the dsRNA or siRNA, then fed to the insect to be affected. In one embodiment, for example, the dsRNA or siRNA molecules may be incorporated into, or overlaid on the top of, the insect's diet. In another embodiment, the RNA may be sprayed onto a plant surface. In still another embodiment, the dsRNA or siRNA may be expressed by microorganisms and the microorganisms may be applied onto a plant surface or introduced into a root, stem by a physical means such as an injection. In still another embodiment, a plant may be genetically engineered to express the dsRNA or siRNA in an amount sufficient to kill the insects known to infect the plant.

Specifically, in practicing the present disclosure in cassava whitefly, the stabilized dsRNA or siRNA may be introduced in the midgut or bacteriocyte inside the insect and achieve the desired inhibition of the targeted genes. The dsRNA or siRNA molecules may be incorporated into a diet or be overlaid on the diet as discussed above and may be ingested by the insects. In any event, the dsRNA's of the present disclosure are provided in the diet of the target pest. The digestive tract of a target pest is defined herein as the location within the pest where food that is ingested by the target pest is exposed to an environment that is favorable for the uptake of the dsRNA molecules of the present disclosure without suffering a pH so extreme that the hydrogen bonding between the double-strands of the dsRNA are caused to dissociate and form single stranded molecules.

Further, for the purpose of controlling insect infestations in plants, delivery of insect control dsRNAs to the surfaces of a plant via a spray-on application affords another means of protecting the plants. In this instance, a bacterium engineered to produce and accumulate dsRNAs may be fermented and the products of the fermentation formulated as a spray-on product compatible with common agricultural practices. The formulations may include the appropriate stickers and wetters required for efficient foliar coverage as well as UV protectants to protect dsRNAs from UV damage. Such additives are commonly used in the bioinsecticide industry and are well known to those skilled in the art. Likewise, formulations for soil application may include granular formulations that serve as a bait for insect pests such as the cassava whitefly.

It is also anticipated that dsRNA's produced by chemical or enzymatic synthesis may be formulated in a manner consistent with common agricultural practices and used as spray-on products for controlling insect infestations. The formulations may include the appropriate stickers and wetters required for efficient foliar coverage as well as UV protectants to protect dsRNAs from UV damage. Such additives are commonly used in the bioinsecticide industry and are well known to those skilled in the art. Such applications could be combined with other spray-on insecticide applications, biologically based or not, to enhance plant protection from insect feeding damage.

The present inventors contemplate that bacterial strains producing insecticidal proteins may be used to produce dsRNAs for insect control purposes. These strains may exhibit improved insect control properties. A variety of different bacterial hosts may be used to produce insect control dsRNAs. Exemplary bacteria may include E. coli, B. thuringiensis, Pseudomonas sp., Photorhabdus sp., Xenorhabdus sp., Serratia entomophila and related Serratia sp., B. sphaericus, B. cereus, B. laterosporus, B. popilliae, Clostridium bifermentans and other Clostridium species, or other spore-forming gram-positive bacteria.

The present disclosure also relates to recombinant DNA constructs for expression in a microorganism. Exogenous nucleic acids from which an RNA of interest is transcribed can be introduced into a microbial host cell, such as a bacterial cell or a fungal cell, using methods known in the art.

The nucleotide sequences of the present disclosure may be introduced into a wide variety of prokaryotic and eukaryotic microorganism hosts to produce the stabilized dsRNA or siRNA molecules. The term “microorganism” includes prokaryotic and eukaryotic microbial species such as bacteria and fungi. Fungi include yeasts and filamentous fungi, among others. Illustrative prokaryotes, both Gram-negative and Gram-positive, include Enterobacteriaceae, such as Escherichia, Erwinia, Shigella, Salmonella, and Proteus; Bacillaceae; Rhizobiceae, such as Rhizobium; Spirillaceae, such as photobacterium, Zymomonas, Serratia, Aeromonas, Vibrio, Desulfovibrio, Spirillum; Lactobacilluseae; Pseudomonadaceae, such as Pseudomonas and Acetobacter; Azotobacteraceae, Actinomycetales, and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like.

For the purpose of plant protection against insects, a large number of microorganisms known to inhabit the phylloplane (the surface of the plant leaves) and/or the rhizosphere (the soil surrounding plant roots) of a wide variety of important crops may also be desirable host cells for manipulation, propagation, storage, delivery and/or mutagenesis of the disclosed recombinant constructs. These microorganisms include bacteria, algae, and fungi. Of particular interest are microorganisms, such as bacteria, e.g., genera Bacillus (including the species and subspecies B. thuringiensis kurstaki HD-1, B. thuringiensis kurstaki HD-73, B. thuringiensis sotto, B. thuringiensis berliner, B. thuringiensis, B. thuringiensis tolworthi, B. thuringiensis dendrolimus, B. thuringiensis alesti, B. thuringiensis galleriae, B. thuringiensis aizawai, B. thuringiensis subtoxicus, B. thuringiensis entomocidus, B. thuringiensis tenebrionis and B. thuringiensis san diego); Pseudomonas, Erwinia, Serratia, Klebsiella, Zanthomonas, Streptomyces, Rhizobium, Rhodopseudomonas, Methylophilius, Agrobacterium, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobacterium tumefaciens, Rhodobacter sphaeroides, Xanthomonas campestris, Rhizobium melioti, Alcaligenes eutrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans.

The term “operably linked”, as used in reference to a regulatory sequence and a structural nucleotide sequence, means that the regulatory sequence causes regulated expression of the linked structural nucleotide sequence. “Regulatory sequences” or “control elements” refer to nucleotide sequences located upstream (5′ noncoding sequences), within, or downstream (3′ non-translated sequences) of a structural nucleotide sequence, and which influence the timing and level or amount of transcription, RNA processing or stability, or translation of the associated structural nucleotide sequence. Regulatory sequences may include promoters, translation leader sequences, introns, enhancers, stem-loop structures, repressor binding sequences, and polyadenylation recognition sequences and the like.

The present disclosure also contemplates transformation of a nucleotide sequence of the present disclosure into a plant to achieve pest inhibitory levels of expression of one or more dsRNA molecules. A transformation vector can be readily prepared using methods available in the art. The transformation vector comprises one or more nucleotide sequences that is/are capable of being transcribed to an RNA molecule and that is/are substantially homologous and/or complementary to one or more nucleotide sequences encoded by the genome of the insect, such that upon uptake of the RNA transcribed from the one or more nucleotide sequences molecules by the insect, there is down-regulation of expression of at least one of the respective nucleotide sequences of the genome of the insect.

### II. Genome Editing

The present disclosure provides, in certain embodiments, insect, insect parts, insect cells, and bacteriocytes produced through genome modification using site-specific integration or genome editing. Ingestion by a target pest of compositions containing one or more gRNAs in combination with a site-specific nuclease, resulting in targeted editing at least one gene of interest in the cells of the target pest, results in death, stunting, or other inhibition of the target pest. Genome editing can be used to make one or more edit(s) or mutation(s) at a desired target site in the genome of an insect, including symbiosis genes (amino acid synthesis, transport and horizontally transferred genes), to change expression and/or activity of one or more genes, or to integrate an insertion sequence or transgene at a desired location in a genome. Any site or locus within the genome of an insect may potentially be chosen for making a genomic edit (or gene edit) or site-directed integration of a transgene, construct, or transcribable DNA sequence. As used herein, a “target site” for genome editing or site-directed integration refers to the location of a polynucleotide sequence within an insect genome that is bound and cleaved by a site-specific nuclease to introduce a double-stranded break (DSB) or single-stranded nick into the nucleic acid backbone of the polynucleotide sequence and/or its complementary DNA strand within the genome. A target site may comprise, for example, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. A “target site” for an RNA-guided nuclease may comprise the sequence of either complementary strand of a double-stranded nucleic acid (DNA) molecule or chromosome at the target site. A site-specific nuclease may bind to a target site, such as via a non-coding guide RNA (e.g., without being limiting, a CRISPR RNA (crRNA) or a single-guide RNA (sgRNA) as described further herein). A non-coding guide RNA provided herein may be complementary to a target site (e.g., complementary to either strand of a double-stranded nucleic acid molecule or chromosome at the target site). It will be appreciated that perfect identity or complementarity may not be required for a non-coding guide RNA to bind or hybridize to a target site. For example, at least 1, at least 2, at least 3, at least 4, at least 5, at least 6, at least 7, or at least 8 mismatches (or more) between a target site and a non-coding RNA may be tolerated. A “target site” also refers to the location of a polynucleotide sequence within a genome that is bound and cleaved by any other site-specific nuclease that may not be guided by a non-coding RNA molecule, such as a zinc finger nuclease (ZFN), a transcription activator-like effector nuclease (TALEN), a meganuclease, etc., to introduce a DSB or single-stranded nick into the polynucleotide sequence and/or its complementary DNA strand. As used herein, a “target region” or a “targeted region” refers to a polynucleotide sequence or region that is flanked by two or more target sites. Without being limiting, in some embodiments a target region may be subjected to a mutation, deletion, insertion, substitution, inversion, or duplication. As used herein, “flanked” when used to describe a target region of a polynucleotide sequence or molecule, refers to two or more target sites of the polynucleotide sequence or molecule surrounding the target region, with one target site on each side of the target region.

As used herein, a “targeted genome editing technique” refers to any method, protocol, or technique that allows the precise and/or targeted editing of a specific location in a genome of an insect (i.e., the editing is largely or completely non-random) using a site-specific nuclease, such as a meganuclease, a zinc-finger nuclease (ZFN), an RNA-guided endonuclease (e.g., the CRISPR/Cas9 or Cas12a system), a TALE (transcription activator-like effector)-endonuclease (TALEN), a recombinase, or a transposase. In particular embodiments, a “targeted genome editing technique” refers to an RNA-guided Cas12a system. As used herein, “editing” or “genome editing” refers to generating a targeted mutation, deletion, insertion, substitution, inversion or duplication of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides of an endogenous insect genome nucleic acid sequence. As used herein, “editing” or “genome editing” may also encompass the targeted insertion or site-directed integration of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 750, at least 1000, at least 1500, at least 2000, at least 2500, at least 3000, at least 4000, at least 5000, at least 10,000, or at least 25,000 nucleotides into the endogenous genome of an insect. An “edit” or “genomic edit” in the singular refers to one such targeted mutation, deletion, insertion, substitution, inversion, or duplication, whereas “edits” or “genomic edits” refers to two or more targeted mutation(s), deletion(s), insertion(s), substitution(s), inversion(s), and/or duplication(s), with each “edit” being introduced via a targeted genome editing technique.

According to some embodiments, a site-specific nuclease may be co-delivered with a donor template molecule to serve as a template for making a desired edit, mutation, or insertion into the genome at the desired target site through repair of the double strand break (DSB) or nick created by the site-specific nuclease. According to some embodiments, a site-specific nuclease may be co-delivered with a DNA molecule comprising a selectable or screenable marker gene.

A site-specific nuclease may be an RNA-guided nuclease. According to some embodiments, an RNA-guided endonuclease may be selected from the group consisting of Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Csc2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, and homologs or modified versions of any thereof, as well as Argonaute proteins (non-limiting examples of Argonaute proteins include Thermus thermophilus Argonaute (TtAgo), Pyrococcus furiosus Argonaute (PfAgo), Natronobacterium gregoryi Argonaute (NgAgo), and homologs or modified versions of any thereof). According to some embodiments, an RNA-guided endonuclease is a Cas9 or Cpf1 (also referred to herein as Cas12a) enzyme. The RNA-guided nuclease may be delivered as a protein or a recombinant DNA construct comprising a polynucleotide sequence encoding said nuclease, with or without a guide RNA; or the guide RNA may be complexed with the RNA-guided nuclease enzyme and delivered as a ribonucleoprotein (RNP).

For RNA-guided endonucleases, a guide RNA molecule may be further provided to direct the endonuclease to a target site in the genome of the insect via base-pairing or hybridization to cause a DSB or nick at or near the target site. As described herein, the guide RNA may be transformed or introduced into an insect cell or tissue as a gRNA molecule, or as a recombinant DNA molecule, construct or vector comprising a transcribable DNA sequence encoding one or more guide RNAs operably linked to a single promoter or individual promoters. As understood in the art, a guide RNA may comprise, for example, a CRISPR RNA (crRNA), a single-chain guide RNA (sgRNA), or any other RNA molecule that may guide or direct an endonuclease to a specific target site in the genome. A prototypical CRISPR associated protein, Cas9 from S. pyogenes, naturally binds two RNAs, a CRISPR RNA (crRNA) guide and a trans-acting CRISPR RNA (tracrRNA), to assemble a CRISPR ribonucleoprotein (crRNP). In comparison, the CRISPR-Cas 12a system does not require a trans-activating crispr RNA (tracrRNA) for biogenesis of mature crRNA. Instead, the RuvC endonuclease domain of Cas12a processes its mature crRNA directly. A “single-chain guide RNA” (or “sgRNA”) is an RNA molecule comprising a crRNA covalently linked a tracrRNA by a linker sequence, which may be expressed as a single RNA transcript or molecule. The guide RNA comprises a guide or targeting sequence (also referred to herein as a “spacer sequence”) that is identical or complementary to a target site within the insect genome, such as at or near a gene. The guide RNA is typically a non-coding RNA molecule that does not encode a protein. The guide sequence of the guide RNA may be at least 10 nucleotides in length, such as 12-40 nucleotides, 12-30 nucleotides, 12-20 nucleotides, 12-35 nucleotides, 12-30 nucleotides, 15-30 nucleotides, 17-30 nucleotides, or 17-25 nucleotides in length, or about 12, 13, 14, 15, 16, 17, 18, 19, 20, 21, 22, 23, 24, 25 or more nucleotides in length. The guide sequence may be at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of a DNA sequence at the genomic target site.

As mentioned above, a target gene for genome editing may be any insect gene of interest. For knockdown mutations of the gene of interest through genome editing, an RNA-guided endonuclease may be targeted to an upstream or downstream sequence, such as a promoter and/or enhancer sequence, or an intron, 5′UTR, and/or 3′UTR sequence of the gene to mutate one or more promoter and/or regulatory sequences of the gene to affect or reduce its level of expression. Similarly, mutations of the gene of interest through genome editing, an RNA-guided endonuclease may be targeted to a transcribable DNA sequence (i.e., a transcribable region) of said gene, such as a region of the gene comprising a coding sequence, a specific DNA sequence encoding a protein domain, an exon region, an intron region, or a combination thereof. For example, in certain embodiments a transcribable DNA sequence targeted for genome editing may comprise an exon/intron boundary or may be in close proximity to an exon/intron boundary. If the resulting modification spans an exon/intron boundary, the modification may be referred to as a modification in an exon region and an intron region. For genetic modification of the gene of interest, a guide RNA may be used, which comprises a guide sequence that is at least 90%, at least 95%, at least 96%, at least 97%, at least 99% or 100% identical or complementary to at least 10, at least 11, at least 12, at least 13, at least 14, at least 15, at least 16, at least 17, at least 18, at least 19, at least 20, at least 21, at least 22, at least 23, at least 24, at least 25, or more consecutive nucleotides of any one of SEQ ID NO: 1, 3, 5, 7, 9, 11, 13, 15, 17, 19, 21, 23, 25, 27, 29, 31, 33, 35, 37, and 39 as set forth in the sequence listing, or a sequence complementary thereto, although alternative splicing and different exon/intron boundaries may occur. As used herein, the term “consecutive” in reference to a polynucleotide or protein sequence means without deletions or gaps in the sequence.

As used herein, with respective to a given sequence, a “complement”, a “complementary sequence” and a “reverse complement” are used interchangeably. All three terms refer to the inversely complementary sequence of a nucleotide sequence, i.e., to a sequence complementary to a given sequence in reverse order of the nucleotides.

Antisense RNA molecules are single-stranded nucleic acids which can combine with a sense RNA strand or sequence or mRNA to form duplexes due to complementarity of the sequences. The term “antisense strand” refers to a nucleic acid strand that is complementary to the “sense” strand. The “sense strand” of a gene or locus is the strand of DNA or RNA that has the same sequence as an RNA molecule transcribed from the gene or locus (with the exception of uracil in RNA and thymine in DNA).

A protospacer-adjacent motif (PAM) may be present in the genome immediately adjacent and upstream to the 5′ end of the genomic target site sequence complementary to the targeting sequence of the guide RNA—i.e., immediately downstream (3′) to the sense (+) strand of the genomic target site (relative to the targeting sequence of the guide RNA) as known in the art. See, e.g., Wu et al. (Quant Biol. 2 (2): 59-70, 2014). The genomic PAM sequence on the sense (+) strand adjacent to the target site (relative to the targeting sequence of the guide RNA) may comprise 5′-NGG-3′ for Cas9; or 5′-TTTN-3′ for Cas12a. However, the corresponding sequence of the guide RNA (i.e., immediately downstream (3′) to the targeting sequence of the guide RNA) may generally not be complementary to the genomic PAM sequence.

As used herein, a “donor molecule”, “donor template”, or “donor template molecule” (collectively a “donor template”), which may be a recombinant polynucleotide, DNA or RNA donor template or sequence, is defined as a nucleic acid molecule having a homologous nucleic acid template or sequence (e.g., homology sequence) and/or an insertion sequence for site-directed, targeted insertion or recombination into the genome of an insect cell via repair of a nick or DSB in the genome of an insect cell. A donor template may be a separate DNA molecule comprising one or more homologous sequence(s) and/or an insertion sequence for targeted integration, or a donor template may be a sequence portion (i.e., a donor template region) of a DNA molecule further comprising one or more other expression cassettes, genes/transgenes, and/or transcribable DNA sequences. For example, a “donor template” may be used for site-directed integration of a transgene or construct, or as a template to introduce a mutation, such as an insertion, deletion, substitution, etc., into a target site within the genome of an insect. A targeted genome editing technique provided herein may comprise the use of one or more, two or more, three or more, four or more, or five or more donor molecules or templates. A donor template provided herein may comprise at least one, at least two, at least three, at least four, at least five, at least six, at least seven, at least eight, at least nine, or at least ten gene(s) or transgene(s) and/or transcribable DNA sequence(s). Alternatively, a donor template may comprise no genes, transgenes, or transcribable DNA sequences.

Any method known in the art for site-directed integration may be used with the present disclosure. In the presence of a donor template molecule with an insertion sequence, the DSB or nick can be repaired by homologous recombination between homology arm(s) of the donor template and the genome, or by non-homologous end joining (NHEJ), resulting in site-directed integration of the insertion sequence into the genome to create the targeted insertion event at the site of the DSB or nick. Thus, site-specific insertion or integration of a transgene, transcribable DNA sequence, construct, or sequence may be achieved if the transgene, transcribable DNA sequence, construct or sequence is located in the insertion sequence of the donor template.

The introduction of a DSB or nick may also be used to introduce targeted mutations in the genome of an insect. According to this approach, mutations, such as deletions, insertions, substitutions, inversions, and/or duplications may be introduced at a target site via imperfect repair of the DSB or nick to produce a genetic modification within a gene. Such mutations may be generated by imperfect repair of the targeted locus even without the use of a donor template molecule. A modification of a gene may be achieved by inducing a DSB or nick at or near the endogenous locus of the gene that results in expression of a non-functional protein, interfering protein, or a protein having reduced, disrupted, or altered activity as compared to a protein expressed from the gene lacking said modification.

Similarly, such targeted mutations of a gene may be generated with a donor template molecule to direct a particular or desired mutation at or near the target site via repair of the DSB or nick. The donor template molecule may comprise a homologous sequence with or without an insertion sequence and comprising one or more mutations, such as one or more deletions, insertions, substitutions, inversions, and/or duplications, relative to the targeted genomic sequence at or near the site of the DSB or nick. For example, targeted mutations of a gene may be achieved by deleting, inserting, substituting, inverting, or duplicating at least a portion of the gene, such as by introducing a frame shift or premature stop codon into the coding sequence of the gene or introducing a modification into a transcribable DNA sequence. A deletion of a portion of a gene may also be introduced by generating DSBs or nicks at two target sites and causing a deletion of the intervening target region flanked by the target sites. A modification of a targeted gene may result in expression of a non-functional protein, interfering protein, or a protein having reduced, disrupted, or altered activity as compared to a protein expressed from the gene lacking said modification.

### III. Constructs for Genome Editing

Recombinant DNA constructs and vectors are provided comprising a polynucleotide sequence encoding a site-specific nuclease, such as an RNA-guided endonuclease, wherein the coding sequence is operably linked to a plant expressible promoter. For RNA-guided endonucleases, recombinant DNA constructs and vectors are further provided comprising a polynucleotide sequence encoding one or more guide RNA(s), wherein the guide RNA(s) comprise a guide sequence of sufficient length having a percent identity or complementarity to a target site within the genome of an insect, such as at or near a targeted gene of interest. A polynucleotide sequence of a recombinant DNA construct and vector that encodes a site-specific nuclease or a guide RNA(s) may be operably linked to a plant expressible promoter, such as an inducible promoter, a constitutive promoter, a tissue-specific promoter, etc.

As used herein, a “gene” refers to a nucleic acid sequence forming a genetic and functional unit and coding for one or more sequence-related RNA and/or polypeptide molecules. A gene generally contains a coding region operably linked to appropriate regulatory sequences that regulate the expression of a gene product (e.g., a polypeptide or a functional RNA). A gene can have various sequence elements, including, but not limited to, a promoter, an untranslated region (UTR), exons, introns, and other upstream or downstream regulatory sequences.

As used herein, an “allele” refers to an alternative nucleic acid sequence of a gene or at a particular locus (e.g., a nucleic acid sequence of a gene or locus that is different than other alleles for the same gene or locus). Such an allele can be considered (i) wild-type or (ii) mutant if one or more mutations or edits are present in the nucleic acid sequence of the mutant allele relative to the wild-type allele. A mutant or edited allele for a gene may have reduced, disrupted, altered, or eliminated activity, or a reduced or eliminated expression level for the gene relative to the wild-type allele. For example, a mutant or edited allele for a gene of interest may have a deletion in the transcribable region of the endogenous gene that reduces, disrupts, or alters the activity of the protein encoded by the mutant allele as compared to the activity of the protein encoded by the wild-type allele in an otherwise identical insect. For diploid organisms, e.g., female whiteflies, a first allele can occur on one chromosome, and a second allele can occur at the same locus on a second homologous chromosome. If one allele at a locus on one chromosome of an insect is a mutant or edited allele and the other corresponding allele on the homologous chromosome of the insect is wild-type, then the insect is described as being heterozygous for the mutant or edited allele. However, if both alleles at a locus are mutant or edited alleles, then the insect is described as being homozygous for the mutant or edited alleles. An insect homozygous for mutant or edited alleles at a locus may comprise the same mutant or edited allele or different mutant or edited alleles if heteroallelic or biallelic.

As used herein, a “wild-type gene” or “wild-type allele” refers to a gene or allele having a sequence or genotype that is most common in a particular whitefly species, or another sequence or genotype having only natural variations, polymorphisms, or other silent mutations relative to the most common sequence or genotype that do not significantly impact the expression and activity of the gene or allele. Indeed, a “wild-type” gene or allele contains no variation, polymorphism, or any other type of mutation that substantially affects the normal function, activity, expression, or phenotypic consequence of the gene or allele relative to the most common sequence or genotype. In general, the term “variant” refers to molecules with some differences, generated synthetically or naturally, in their nucleotide or amino acid sequences as compared to a reference (native) polynucleotides or polypeptides, respectively. These differences include substitutions, insertions, deletions, inversions, duplications, or any desired combinations of such changes in a native polynucleotide or amino acid sequence.

The term “recombinant” in reference to a polynucleotide (DNA or RNA) molecule, protein, construct, vector, etc., refers to a polynucleotide or protein molecule or sequence that is man-made and not normally found in nature, and/or is present in a context in which it is not normally found in nature, including a polynucleotide (DNA or RNA) molecule, protein, construct, etc., comprising a combination of two or more polynucleotide or protein sequences that would not naturally occur together in the same manner without human intervention, such as a polynucleotide molecule, protein, construct, etc., comprising at least two polynucleotide or protein sequences that are operably linked but heterologous with respect to each other. For example, the term “recombinant” can refer to any combination of two or more DNA or protein sequences in the same molecule (e.g., a plasmid, construct, vector, chromosome, protein, etc.) where such a combination is man-made and not normally found in nature. As used in this definition, the phrase “not normally found in nature” means not found in nature without human introduction. A recombinant polynucleotide or protein molecule, construct, etc., can comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., can also refer to a polynucleotide or protein molecule or sequence that has been genetically engineered and/or constructed outside of a cell. For example, a recombinant DNA molecule can comprise any engineered or man-made plasmid, vector, etc., and can include a linear or circular DNA molecule. Such plasmids, vectors, etc., can contain various maintenance elements including a prokaryotic origin of replication and selectable marker, as well as one or more transgenes or expression cassettes perhaps in addition to a plant selectable marker gene, etc.

Reference in this application to an “isolated DNA molecule” or an “isolated polynucleotide”, or an equivalent term or phrase, is intended to mean that the DNA molecule or polynucleotide is one that is present alone or in combination with other compositions, but not within its natural environment. For example, nucleic acid elements such as a coding sequence, intron sequence, untranslated leader sequence, promoter sequence, transcriptional termination sequence, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element is not within the genome of the organism and at the location within the genome in which it is naturally found. Similarly, a nucleotide sequence encoding a protein or any naturally occurring variant of that protein would be an isolated nucleotide sequence so long as the nucleotide sequence was not within the DNA of the organism in which the sequence encoding the protein is naturally found. A synthetic nucleotide sequence encoding the amino acid sequence of the naturally occurring protein would be considered to be isolated for the purposes of this disclosure. For the purposes of this disclosure, any transgenic nucleotide sequence, i.e., the nucleotide sequence of the DNA inserted into the genome of the cells of a plant or bacterium, or present in an extrachromosomal vector, would be considered to be an isolated nucleotide sequence whether it is present within the plasmid or similar structure used to transform the cells, within the genome of the plant or bacterium, or present in detectable amounts in tissues, progeny, biological samples or commodity products derived from the plant or bacterium.

As commonly understood in the art, the term “promoter” can generally refer to a DNA sequence that contains an RNA polymerase binding site, transcription start site, and/or TATA box and assists or promotes the transcription and expression of an associated transcribable polynucleotide sequence and/or gene (or transgene). A promoter can be synthetically produced, varied, or derived from a known or naturally occurring promoter sequence or other promoter sequence. A promoter can also include a chimeric promoter comprising a combination of two or more heterologous sequences. A promoter of the present disclosure can thus include variants or fragments of promoter sequences that are similar in composition, but not identical to, other promoter sequence(s) known or provided herein. A promoter provided herein, or variant or fragment thereof, may comprise a “minimal promoter” which provides a basal level of transcription and is comprised of a TATA box or equivalent DNA sequence for recognition and binding of the RNA polymerase II complex for initiation of transcription. A promoter can be classified according to a variety of criteria relating to the pattern of expression of an associated coding or transcribable sequence or gene (including a transgene) operably linked to the promoter, such as constitutive, developmental, tissue-specific, inducible, etc. Promoters that drive expression in all or most tissues of the plant are referred to as “constitutive” promoters. Promoters that drive expression during certain periods or stages of development are referred to as “developmental” promoters. Promoters that drive enhanced expression in certain tissues of the plant relative to other plant tissues are referred to as “tissue-enhanced” or “tissue-preferred” promoters. Thus, a “tissue-preferred” promoter causes relatively higher or preferential expression in a specific tissue(s) of the plant, but with lower levels of expression in other tissue(s) of the plant. Promoters that express within a specific tissue(s) of the plant, with little or no expression in other plant tissues, are referred to as “tissue-specific” promoters. An “inducible” promoter is a promoter that initiates transcription in response to an environmental stimulus such as cold, drought or light, or other stimuli, such as wounding or chemical application. A promoter can also be classified in terms of its origin, such as being heterologous, homologous, chimeric, synthetic, etc.

As used herein, a “plant-expressible promoter” refers to a promoter that can initiate, assist, affect, cause, and/or promote the transcription and expression of its associated transcribable DNA sequence, coding sequence or gene in a plant cell or tissue.

The term “heterologous” in reference to a promoter or other regulatory sequence in relation to an associated polynucleotide sequence (e.g., a transcribable DNA sequence or coding sequence or gene) is a promoter or regulatory sequence that is not operably linked to such associated polynucleotide sequence in nature without human introduction—e.g., the promoter or regulatory sequence has a different origin relative to the associated polynucleotide sequence and/or the promoter or regulatory sequence is not naturally occurring in a plant species to be transformed with the promoter or regulatory sequence. Similarly, “heterologous” in reference to a coding sequence may refer to the use of a recombinant DNA molecule codon-optimized for a different organism as compared to the organism said DNA molecule is being expressed in—e.g., the recombinant DNA sequence encoding a Cas12a is codon-optimized for expression in humans but is expressed in a plant cell.

As used herein, an “untranslated region (UTR)” of a gene refers to a segment of an RNA molecule or sequence (e.g., a mRNA molecule) expressed from a gene (or transgene) but excluding the exon and intron sequences of the RNA molecule. An “untranslated region (UTR)” also refers to a DNA segment or sequence encoding such a UTR segment of an RNA molecule. An untranslated region can be a 5′-UTR or a 3′-UTR depending on whether it is located at the 5′ or 3′ end of a DNA or RNA molecule or sequence relative to a coding region of the DNA or RNA molecule or sequence (i.e., upstream (5′) or downstream (3′) of the exon and intron sequences, respectively).

As used herein, a “transcribable region” or “transcribable DNA sequence” refers to a nucleic acid sequence expressed from a gene (or transgene).

As used herein, a “transcription termination sequence” refers to a nucleic acid sequence containing a signal that triggers the release of a newly synthesized transcript RNA molecule from an RNA polymerase complex and marks the end of transcription of a gene or locus.

The terms “percent identity,” “% identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.” Sequences having a percent identity to a base sequence may exhibit the activity of the base sequence.

Homologs are inferred from sequence similarity, by comparison of protein sequences, for example, manually or by use of a computer-based tool. For optimal alignment of sequences to calculate their percent identity, various pair-wise or multiple sequence alignment algorithms and programs are known in the art, such as ClustalW or Basic Local Alignment Search Tool® (BLAST), etc., that can be used to compare the sequence identity or similarity between two or more nucleotide or protein sequences. BLAST, can also be used, for example to search query protein sequences of a base organism against a database of protein sequences of various organisms, to find similar sequences. The generated summary Expectation value (E-value) can be used to measure the level of sequence similarity. Because a protein hit with the lowest E-value for a particular organism may not necessarily be an ortholog or be the only ortholog, a reciprocal query is used to filter hit sequences with significant E-values for ortholog identification. The reciprocal query entails search of the significant hits against a database of protein sequences of the base organism. A hit can be identified as an ortholog, when the reciprocal query's best hit is the query protein itself or a paralog of the query protein. With the reciprocal query process orthologs are further differentiated from paralogs among all the homologs, which allows for the inference of functional equivalence of genes.

The terms “percent complementarity” or “percent complementary”, as used herein in reference to two nucleotide sequences, is similar to the concept of percent identity but refers to the percentage of nucleotides of a query sequence that optimally base-pair or hybridize to nucleotides of a subject sequence when the query and subject sequences are linearly arranged and optimally base paired without secondary folding structures, such as loops, stems or hairpins. Such a percent complementarity may be between two DNA strands, two RNA strands, or a DNA strand and an RNA strand. The “percent complementarity” is calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences may be determined based on the known pairings of nucleotide bases, such as G-C, A-T, and A-U, through hydrogen bonding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present disclosure, when two sequences (query and subject) are optimally base-paired (with allowance for mismatches or non-base-paired nucleotides but without folding or secondary structures), the “percent complementarity” for the query sequence is equal to the number of base-paired positions between the two sequences divided by the total number of positions in the query sequence over its length (or by the number of positions in the query sequence over a comparison window), which is then multiplied by 100%.

As used herein, a “fragment” of a polynucleotide refers to a sequence comprising at least about 50, at least about 75, at least about 95, at least about 100, at least about 125, at least about 150, at least about 175, at least about 200, at least about 225, at least about 250, at least about 275, at least about 300, at least about 500, at least about 600, at least about 700, at least about 750, at least about 800, at least about 900, or at least about 1000 contiguous nucleotides, or longer, of a DNA molecule or protein as disclosed herein. Methods for producing such fragments from a starting promoter molecule are well known in the art. Fragments of a DNA molecule or protein may exhibit the activity of the DNA molecule or protein from which they are derived.

A plant selectable marker transgene in a transformation vector or construct of the present disclosure may be used to assist in the selection of transformed cells or tissue due to the presence of a selection agent, such as an antibiotic or herbicide, wherein the plant selectable marker transgene provides tolerance or resistance to the selection agent. Thus, the selection agent may bias or favor the survival, development, growth, proliferation, etc., of transformed cells expressing the plant selectable marker gene, such as to increase the proportion of transformed cells or tissues in the R₀ plant. Commonly used plant selectable marker genes include, for example, those conferring tolerance or resistance to antibiotics, such as kanamycin and paromomycin (nptll), hygromycin B (aph IV), streptomycin or spectinomycin (aadA) and gentamycin (aac3 and aacC4), or those conferring tolerance or resistance to herbicides such as glufosinate (bar or pat), dicamba (DMO) and glyphosate (proA or EPSPS). Plant screenable marker genes may also be used, which provide an ability to visually screen for transformants, such as luciferase or green fluorescent protein (GFP), or a gene expressing a beta glucuronidase or uidA gene (GUS) for which various chromogenic substrates are known. Plant transformation may also be carried out in the absence of selection during one or more steps or stages of culturing, developing, or regenerating transformed explants, tissues, plants and/or plant parts.

### IV. Transformation Methods

Methods and compositions are provided for transforming a plant cell, tissue or explant with a recombinant DNA molecule or construct encoding one or more molecules required for gene suppression, or targeted genome editing (e.g., guide RNA(s) and/or site-directed nuclease(s)) as described herein. Suitable methods for transformation of host plant cells include virtually any method by which DNA or RNA can be introduced into a cell (for example, where a recombinant DNA construct is stably integrated into a plant chromosome or where a recombinant DNA construct or an RNA is transiently provided to a plant cell) and are well known in the art. Two effective methods for cell transformation are bacterially-mediated transformation, such as Agrobacterium-mediated or Rhizobium-mediated transformation, and microprojectile or particle bombardment-mediated transformation. Microprojectile bombardment methods are illustrated, for example, in U.S. Pat. Nos. 5,550,318; 5,538,880; 6,160,208; and 6,399,861. Agrobacterium-mediated transformation methods are described, for example in U.S. Pat. No. 5,591,616, Hinchliffe and Harwood (2019), and Sparrow and Irwin (2015). Other methods for plant transformation, such as microinjection, electroporation, vacuum infiltration, pressure, sonication, silicon carbide fiber agitation, PEG-mediated transformation, etc., are also known in the art.

Any of the polynucleotide molecules of the present disclosure may be introduced into a plant cell in a permanent or transient manner in combination with other genetic elements such as promoters, introns, enhancers, and untranslated leader sequences, etc. Any of the nucleic acid molecules encoding an invertebrate pest, such as B. tabaci RNA or an RNA from a piercing and sucking insect species, or preferably a B. tabaci SSA1-SG1 RNA, may be fabricated and introduced into a plant cell in a manner that allows for production of the dsRNA molecules within the plant cell, providing an insecticidal amount of one or more particular dsRNA's in the diet of a target insect pest.

In one embodiment the plant transformation vector is an isolated and purified DNA molecule comprising a promoter operatively linked to one or more nucleotide sequences of the present disclosure. The nucleotide sequence may be selected from the group consisting of SEQ ID NO: 1, 3, 5, 7, 9, 11, 13, 15, 17, 19, 21, 23, 25, 27, 29, 31, 33, 35, 37, and 39 as set forth in the sequence listing, or fragments or complements thereof. The nucleotide sequence includes a segment coding all or part of an RNA present within a targeted pest RNA transcript and may comprise inverted repeats of all or a part of a targeted pest RNA. The DNA molecule comprising the expression vector may also contain a functional intron sequence positioned either upstream of the coding sequence or even within the coding sequence and may also contain a five prime (5′) untranslated leader sequence (i.e., a UTR or 5′-UTR) positioned between the promoter and the point of translation initiation.

A plant transformation vector may contain sequences from more than one gene, thus allowing production of more than one dsRNA for inhibiting expression of two or more genes in cells of a target pest. One skilled in the art will readily appreciate that segments of DNA whose sequence corresponds to that present in different genes can be combined into a single composite DNA segment for expression in a transgenic plant. Alternatively, a plasmid of the present disclosure already containing at least one DNA segment can be modified by the sequential insertion of additional DNA segments between the enhancer and promoter and terminator sequences. In the insect control agent of the present disclosure designed for the inhibition of multiple genes, the genes to be inhibited can be obtained from the same insect species in order to enhance the effectiveness of the insect control agent. In certain embodiments, the genes can be derived from different insects in order to broaden the range of insects against which the agent is effective. When multiple genes are targeted for suppression or a combination of expression and suppression, a polycistronic DNA element can be fabricated as illustrated and disclosed in Fillatti, Application Publication No. US 2004-0029283.

Where a nucleotide sequence of the present disclosure is to be used to transform a plant, a promoter exhibiting the ability to drive expression of the coding sequence in that particular species of plant is selected. Promoters that function in different plant species are also well known in the art. Promoters useful for expression of polypeptides in plants are those that are inducible, viral, synthetic, or constitutive as described in Odell et al. (1985, Nature 313:810-812), and/or promoters that are temporally regulated, spatially regulated, and spatio-temporally regulated. Preferred promoters include the enhanced CaMV35S promoters, and the FMV35S promoter. For the purpose of the present disclosure, e.g., for optimum control of species that feed on plant leaves, it is preferable to achieve the highest levels of expression of these genes within the leaves of plants. A number of phloem-specific promoters have been identified and are known in the art.

Transformation of plant material is practiced in tissue culture on nutrient media, for example a mixture of nutrients that allow cells to grow in vitro. Recipient cell targets include, but are not limited to, meristem cells, shoot tips, hypocotyls, calli, immature or mature embryos, and gametic cells such as microspores and pollen. Callus can be initiated from tissue sources including, but not limited to, immature or mature embryos, hypocotyls, seedling apical meristems, microspores and the like. Cells containing a transgenic nucleus are grown into transgenic plants. Any suitable method or technique for transformation of a plant cell known in the art may be used according to present methods. In transformation, DNA is typically introduced into only a small percentage of target plant cells in any one transformation experiment. Marker genes are used to provide an efficient system for identification of those cells that are stably transformed by receiving and integrating a recombinant DNA molecule into their genomes.

As used herein, the terms “regeneration” and “regenerating” refer to a process of growing or developing a plant from one or more plant cells through one or more culturing steps. Transformed or edited cells, tissues or explants containing a DNA sequence insertion or edit may be grown, developed, or regenerated into transgenic plants in culture, plugs, or soil according to methods known in the art. Certain embodiments of the disclosure therefore relate to methods and constructs for regenerating a plant from a cell with modified genomic DNA resulting from genome editing. The regenerated plant can then be used to propagate additional plants, e.g., by vegetative propagation.

According to an aspect of the present disclosure, regenerated plants or a progeny plant, plant part or seed thereof can be screened or selected based on a marker, trait, or phenotype produced by the edit or mutation, or by the site-directed integration of an insertion sequence, transgene, etc., in the developed or regenerated plant, or a progeny plant, plant part or seed thereof. If a given mutation, edit, trait or phenotype is recessive, one or more generations or crosses (e.g., selfing) from the initial R₀ plant may be necessary to produce a plant homozygous for the edit or mutation so the trait or phenotype can be observed. Progeny plants, such as plants grown from R₁ seed or in subsequent generations, can be tested for zygosity using any known zygosity assay, such as by using a single nucleotide polymorphism (SNP) assay, DNA sequencing, thermal amplification, or polymerase chain reaction (PCR), and/or Southern blotting that allows for the distinction between heterozygote, homozygote, and wild-type plants.

Methods and techniques are provided for screening for, and/or identifying, cells or plants, etc., for the presence of targeted edits or transgenes, and selecting cells or plants comprising targeted edits or transgenes, which may be based on one or more phenotypes or traits, or on the presence or absence of a molecular marker or polynucleotide or protein sequence in the cells or plants. As used herein, a “molecular technique” refers to any method known in the fields of molecular biology, biochemistry, genetics, plant biology, or biophysics that involves the use, manipulation, or analysis of a nucleic acid, a protein, or a lipid. Without being limiting, molecular techniques useful for detecting the presence of a modified sequence in a genome include phenotypic screening; molecular marker technologies such as SNP analysis by TaqMan® or Illumina/Infinium technology; Southern blot; PCR; enzyme-linked immunosorbent assay (ELISA); and sequencing (e.g., Sanger, Illumina®, 454, Pac-Bio, Ion Torrent™). In one aspect, a method of detection provided herein comprises phenotypic screening. In another aspect, a method of detection provided herein comprises SNP analysis. In a further aspect, a method of detection provided herein comprises a Southern blot. In a further aspect, a method of detection provided herein comprises PCR. In an aspect, a method of detection provided herein comprises ELISA. In a further aspect, a method of detection provided herein comprises determining the sequence of a nucleic acid or a protein. Without being limiting, nucleic acids can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Nucleic acids can be isolated using techniques routine in the art. For example, nucleic acids can be isolated using any method including, without limitation, recombinant nucleic acid technology, and/or PCR. General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Detection (e.g., of an amplification product, of a hybridization complex, of a polypeptide) can be accomplished using detectable labels that may be attached or associated with a hybridization probe or antibody. The term “label” is intended to encompass the use of direct labels as well as indirect labels. Detectable labels include enzymes, prosthetic groups, fluorescent materials, luminescent materials, bioluminescent materials, and radioactive materials. The screening and selection of modified (e.g., edited) plants or plant cells can be through any methodologies known to those skilled in the art of molecular biology. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina®, PacBio®, Ion Torrent™, etc.) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known in the art.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Polypeptides can be detected using antibodies. Techniques for detecting polypeptides using antibodies include enzyme linked immunosorbent assays (ELISAs), Western blots, immunoprecipitations, and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The present disclosure can be, in practice, combined with other insect control traits in a plant to achieve desired traits for enhanced control of insect infestation. Combining insect control traits that employ distinct modes-of-action can provide insect-protected transgenic plants with superior durability over plants harboring a single insect control trait because of the reduced probability that resistance will develop in the field.

A plant that may be transformed with a recombinant DNA molecule or transformation vector comprising interfering RNA sequence(s) (e.g., ssRNA, dsRNA, siRNA, etc), guide RNA(s), or combination thereof, may include a variety of flowering plants or angiosperms, which may be further defined as including various dicotyledonous (dicot) plant species or monocotyledonous (monocot) plant species. A dicot plant could be members of the Fabaceae family (such as legumes), sunflower (Helianthus annuus), safflower (Carthamus tinctorius), sesame (Sesamum spp.), tobacco (Nicotiana tabacum), potato (Solanum tuberosum), cotton (Gossypium barbadense, Gossypium hirsutum), sweet potato (Ipomoea batatas), cassava (Manihot esculenta), coffee (Coffea spp.), tea (Camellia spp.), fruit trees, such as apple (Malus spp.), Prunus spp., such as plum, apricot, peach, cherry, etc., pear (Pyrus spp.), fig (Ficus carica), etc., citrus trees (Citrus spp.), cocoa (Theobroma cacao), avocado (Persea americana), olive (Olea europaea), almond (Prunus amygdalus), walnut (Juglans spp.), strawberry (Fragaria spp.), watermelon (Citrullus lanatus), pepper (Capsicum spp.), eggplant, beet (Beta vulgaris), grape (Vitis, Muscadinia), tomato (Lycopersicon esculentum, Solanum lycopersicum), cucumber (Cucumis sativus), and members of the Brassicaceae family, such as thale cress (Arabidopsis thaliana) and Brassica sp. (e.g., B. napus, B. rapa, B. juncea), particularly those Brassica species useful as sources of seed oil. Legumes and leguminous plants include peas (Pisum sativum) alfalfa (Medicago sativa), barrel clover (Medicago truncatula), pigeon pea (Cajanus cajan) guar (Cyamopsis tetragonoloba), carob (Ceratonia siliqua), fenugreek (Trigonella foenum-graecum), soybean (Glycine max), common bean (Phaseolus vulgaris), cowpea (Vigna unguiculata), mung bean (Vigna radiata), lima bean (Phaseolus lunatus), fava bean (Vicia faba), lentil (Lens culinaris or Lens esculenta), peanut (Arachis hypogaea), licorice (Glycyrrhiza glabra), and chickpea (Cicer arietinum). A monocot plant could be oil palm (Elaeis spp.), coconut (Cocos spp.), banana (Musa spp.), and cereals such as corn (Zea mays), barley (Hordeum vulgare), sorghum (Sorghum bicolor), rice (Oryza sativa), and wheat (Triticum aestivum). Given that the present disclosure may apply to a broad range of plant species, the present disclosure further applies to other botanical structures analogous to pods of leguminous plants, such as bolls, siliques, fruits, nuts, tubers, etc.

### V. Plants Comprising DNA Molecules

The term “transgenic plant cell” or “transgenic plant” as used herein includes a plant cell or a plant that contains an exogenous nucleic acid, which can be derived from an invertebrate pest, such as the Hemipteran, B. tabaci, or from a different insect species. The transgenic plants are also meant to comprise progeny (decedent, offspring, etc.) of any generation of such a transgenic plant or a seed of any generation of all such transgenic plants wherein said progeny or seed comprises a DNA sequence encoding the RNA, SRNA, dsRNA, siRNA, gRNA, or fragment thereof of the present disclosure is also an important aspect of the disclosure.

A transgenic plant formed using Agrobacterium transformation methods typically contains a single simple recombinant DNA sequence inserted into one chromosome and is referred to as a transgenic event. Such transgenic plants can be referred to as being heterozygous for the inserted exogenous sequence. A transgenic plant homozygous with respect to a transgene can be obtained by sexually mating (selfing) an independent segregant transgenic plant that contains a single exogenous gene sequence to itself, for example an F0 plant, to produce F1 seed. One fourth of the F1 seed produced will be heterozygous with respect to the transgene. Germinating F1 seed results in plants that can be tested for heterozygosity, typically using a SNP assay or a thermal amplification assay that allows for the distinction between heterozygotes and homozygotes (i.e., a zygosity assay). Crossing a heterozygous plant with itself or another heterozygous plant results in only heterozygous progeny.

As used herein, the term “transformed” refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is inherited by subsequent progeny. A “transgenic” or “transformed” cell or organism may also include progeny of the cell or organism and progeny produced from a breeding program employing such a transgenic organism as a parent in a cross and exhibiting an altered phenotype resulting from the presence of a foreign DNA molecule. The introduced DNA molecule may also be transiently introduced into the recipient cell such that the introduced DNA molecule is not inherited by subsequent progeny. The term “transgenic” refers to a bacterium, fungus, or plant containing one or more heterologous DNA molecules.

A transgenic plant subsequently may be regenerated from a transgenic plant cell of the present disclosure. Using conventional breeding techniques or self-pollination, seed may be produced from this transgenic plant. Such seed, and the resulting progeny plant grown from such seed, will contain the recombinant DNA molecule of the present disclosure, and therefore will be transgenic.

Transgenic plants of the disclosure can be self-pollinated to provide seed for homozygous transgenic plants of the disclosure (homozygous for the recombinant DNA molecule) or crossed with non-transgenic plants or different transgenic plants to provide seed for heterozygous transgenic plants of the present disclosure (heterozygous for the recombinant DNA molecule). Both such homozygous and heterozygous transgenic plants are referred to herein as “progeny plants.” Progeny plants are transgenic plants descended from the original transgenic plant and containing the recombinant DNA molecule of the present disclosure. Seeds produced using a transgenic plant of the present disclosure can be harvested and used to grow generations of transgenic plants, i.e., progeny plants of the present disclosure, comprising the construct of this disclosure and expressing a gene of agronomic interest. Descriptions of breeding methods that are commonly used for different crops can be found in one of several reference books, see, e.g., Allard, Principles of Plant Breeding, John Wiley & Sons, NY, U. of CA, Davis, CA, 50-98 (1960); Simmonds, Principles of Crop Improvement, Longman, Inc., NY, 369-399 (1979); Sneep and Hendriksen, Plant breeding Perspectives, Wageningen (ed), Center for Agricultural Publishing and Documentation (1979); Fehr, Soybeans: Improvement, Production and Uses, 2nd Edition, Monograph, 16:249 (1987); Fehr, Principles of Variety Development, Theory and Technique, (Vol. 1) and Crop Species Soybean (Vol. 2), Iowa State Univ., Macmillan Pub. Co., NY, 360-376 (1987).

The transformed plants may be analyzed for the presence of the gene or genes of interest and the expression level and/or profile conferred by the regulatory elements of the present disclosure. Those of skill in the art are aware of the numerous methods available for the analysis of transformed plants. For example, methods for plant analysis include, but are not limited to, Southern blots or northern blots, PCR-based approaches, biochemical analyses, phenotypic screening methods, field evaluations, and immunodiagnostic assays. The expression of a transcribable DNA molecule can be measured using TaqMan® (Applied Biosystems, Foster City, CA) reagents and methods as described by the manufacturer and PCR cycle times determined using the TaqMan® Testing Matrix. Alternatively, other methods and reagents for measuring expression of a transcribable DNA molecule are well known in the art. For example, the Invader® (Third Wave Technologies, Madison, WI) or SYBR Green (Thermo Fisher, A46012) reagents and methods as described by the manufacturer can be used to evaluate transgene expression.

The seeds of the plants of this disclosure can be harvested from fertile transgenic plants and be used to grow progeny generations of transformed plants of this disclosure including hybrid plant lines comprising the construct of this disclosure and expressing a gene of agronomic interest.

The present disclosure also provides for parts of the plants of the present disclosure. Plant parts, without limitation, include leaves, stems, roots, tubers, seeds, endosperm, ovule, and pollen. The disclosure also includes and provides transformed plant cells which comprise a nucleic acid molecule of the present disclosure.

The transgenic plant may pass along the transgenic polynucleotide molecule to its progeny. Progeny includes any regenerable plant part or seed comprising the transgene derived from an ancestor plant. The transgenic plant is preferably homozygous for the transformed polynucleotide molecule and transmits that sequence to all offspring as a result of sexual reproduction. Progeny may be grown from seeds produced by the transgenic plant. These additional plants may then be self-pollinated to generate a true breeding line of plants. Progeny from these plants are evaluated, among other things, for gene expression. The gene expression may be detected by several common methods such as western blotting, northern blotting, immuno-precipitation, and ELISA.

As an alternative to traditional transformation methods, a DNA molecule, such as a transgene, expression cassette(s), etc., may be inserted or integrated into a specific site or locus within the genome of a plant or plant cell via site-directed integration. Recombinant DNA construct(s) and molecule(s) of this disclosure may thus include a donor template sequence comprising at least one transgene, expression cassette, or other DNA sequence for insertion into the genome of the plant or plant cell. Such donor template for site-directed integration may further include one or two homology arms flanking an insertion sequence (i.e., the sequence, transgene, cassette, etc., to be inserted into the plant genome). The recombinant DNA construct(s) of this disclosure may further comprise an expression cassette(s) encoding a site-specific nuclease and/or any associated protein(s) to carry out site-directed integration. These nuclease-expressing cassette(s) may be present in the same molecule or vector as the donor template (in cis) or on a separate molecule or vector (in trans). Several methods for site-directed integration are known in the art involving different proteins (or complexes of proteins and/or guide RNA) that cut the genomic DNA to produce a double strand break (DSB) or nick at a desired genomic site or locus. Briefly as understood in the art, during the process of repairing the DSB or nick introduced by the nuclease enzyme, the donor template DNA may become integrated into the genome at the site of the DSB or nick. The presence of the homology arm(s) in the donor template may promote the adoption and targeting of the insertion sequence into the plant genome during the repair process through homologous recombination, although an insertion event may occur through non-homologous end joining (NHEJ). Examples of site-specific nucleases that may be used include zinc-finger nucleases, engineered or native meganucleases, TALE-endonucleases, and RNA-guided endonucleases (e.g., Cas9 or Cpf1). For methods using RNA-guided site-specific nucleases (e.g., Cas9 or Cpf1), the recombinant DNA construct(s) will also comprise a sequence encoding one or more guide RNAs to direct the nuclease to the desired site within the plant genome.

### VI. Commodity Products

The present disclosure provides a commodity product comprising DNA molecules according to the disclosure. As used herein, a “commodity product” refers to any composition or product which is comprised of material derived from a plant, seed, plant cell or plant part comprising a DNA molecule of the disclosure. Commodity products may be sold to consumers and may be viable or nonviable. Nonviable commodity products include but are not limited to nonviable seeds and grains; processed seeds, seed parts, and plant parts; dehydrated plant tissue, frozen plant tissue, and processed plant tissue; seeds and plant parts processed for animal feed for terrestrial and/or aquatic animal consumption, oil, meal, flour, flakes, bran, fiber, milk, cheese, paper, cream, wine, and any other food for human consumption; and biomasses and fuel products. Viable commodity products include but are not limited to seeds and plant cells. Plants comprising a DNA molecule according to the disclosure can thus be used to manufacture any commodity product typically acquired from plants or parts thereof.

### VI. Definitions

The following definitions are provided to define and clarify the meaning of these terms in reference to the relevant embodiments of the present disclosure as used herein and to guide those of ordinary skill in the art in understanding the present disclosure. Unless otherwise noted, terms are to be understood according to their conventional meaning and usage in the relevant art, particularly in the field of molecular biology and plant transformation.

When introducing elements of the present disclosure or the embodiment(s) thereof, the articles “a”, “an”, “the”, and “said” are intended to mean that there are one or more of the elements. The term “and/or”, when used in a list of two or more items, means any one of the items, any combination of the items, or all of the items with which this term is associated.

The terms “comprising”, “including”, and “having” are intended to be inclusive and mean that there may be additional elements other than the listed elements. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

As used herein, a “plant” includes a whole plant, explant, plant part, seedling, or plantlet at any stage of regeneration or development.

As used herein, a “plant part” can refer to any organ or intact tissue of a plant, such as a meristem, shoot organ/structure (e.g., leaf, stem or node), root, flower or floral organ/structure (e.g., bract, sepal, petal, stamen, carpel, anther and ovule), seed, embryo, endosperm, seed coat, fruit, the mature ovary, propagule, or other plant tissues (e.g., vascular tissue, dermal tissue, ground tissue, and the like), or any portion thereof. Plant parts of the present disclosure can be viable, nonviable, regenerable, and/or non-regenerable. A “propagule” can include any plant part that can grow into an entire plant.

As used herein, “genomic DNA” or “gDNA” refers to chromosomal DNA of an organism.

As used herein, a “genomic modification” (also referred to as “modification”) or “genomic edit” (also referred to as “edit”) refers to any modification to a genomic nucleotide sequence as compared to a wild-type or control plant. A genomic modification or genomic edit comprises a deletion, an insertion, a substitution, an inversion, a duplication, or any combination thereof.

As used herein, “T-DNA” or “transfer DNA” refers to the transferred DNA of the tumor-inducing (Ti) plasmid of some species of bacteria such as Agrobacterium tumefaciens.

As used herein, “a target structural motif,” or “target motif,” refers to any rationally selected sequence or combination of sequences in which the sequences or sequence(s) are chosen based on a three-dimensional configuration that is formed upon the folding of the target motif. There are a variety of target motifs known in the art. Protein target motifs include, but are not limited to, enzymatic active sites and signal sequences. Nucleic acid target motifs include, but are not limited to, promoter sequences, cis elements, hairpin structures and inducible expression elements (protein binding sequences). Specifically, a “target motif” may refer to a catalytic domain and/or a signal peptide required for an enzyme to function as an extracellular enzyme in the gut lumen of whitefly.

All methods described herein can be performed in any suitable order unless otherwise indicated herein or clearly contradicted by context. The use of any and all examples, or exemplary language (e.g., “such as”) provided with respect to certain embodiments herein is intended merely to illuminate the present disclosure and does not pose a limitation on the scope of the present disclosure otherwise claimed.

### EXAMPLES

The following examples are included to illustrate embodiments of the invention. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 1: Identification of Sugar Transporter and Detoxification Genes as Targets for Controlling Bemisia Tabaci

Studies were conducted in order to identify and characterize sugar transporter and detoxification genes as targets for controlling Bemisia tabaci. The bioinformatic analysis, gene-expression analysis, and survival studies reported in the instant disclosure were conducted essentially as follows.

Host plant and insect population: Brussels sprout plants, Brassica oleracea L var. gemmifera L. ‘Franklin’ cultivar (Eden Seeds, Israel), were used throughout this study as host plants for B. tabaci, both for colony maintenance and performance assays. The Brussels sprout seeds were germinated in square plastic pots (6×6×6.5 cm3) containing peat, coconut, tuff, NPK fertilizer, and micronutrients (Bental 11, Tuff Merom Golan, Israel). Two to three weeks later, the seedlings were transplanted into larger pots (12 cm diameter). The plants were used for rearing and experimenting when they developed four “true” leaves (seven weeks from germination). Greenhouse conditions were 30±4° C. with 30-40% relative humidity and a 14:10 (L:D) photoperiod. The colony of the B. tabaci, MEAMI species, was established using adults collected in Menahamya (North of the Jordan Valley, Israel). The colony was reared on Brussels sprout plants in fine mesh (160 μm) insect-rcaring tents (47.5×47.5×93 cm3) under greenhouse conditions (28±4° C., 40-60% relative humidity, and a 14:10 (L:D) photoperiod).

Phylogenetic analysis in B. tabaci: Target protein sequences of B. tabaci were downloaded from NCBI (https://www.ncbi.nlm.nih.gov/) and the whitefly genomics open databases (http://www.whiteflygenomics.org/). First, duplications and variant sequences with 98% sequence identity or higher were concatenated and de-replicated using CDHIT v4.6 with default parameters. The remaining 70 sequences were aligned using MAFFT v7.215 (default parameters) and blocks of phylogenetically informative positions were chosen using Gblocks v0.91b (most permissive parameters per alignment). A maximum-likelihood phylogenetic tree was built using the best-fit model of amino acid replacement selection and 5000 bootstraps in IQ-TREE v1.6.5, with the following command: iqtree-s all_CDHIT098.faa.aln-gb-st AA-nt 14-m TEST-bb 5000-alrt 1000. Trec representation was performed using iTOL version 6 (https://itol.embl.de/).

dsRNA design and synthesis: The dsRNA molecules were designed using E-RNAi version 3.2 (http://www.e-rnai.org/). Each designed dsRNA molecule was BLASTed using NCBI BLASTn (https://blast.ncbi.nlm.nih.gov/Blast.cgi/) and manually curated to make sure that no ≥14 nucleotide long fragments are identical between the designed dsRNA sequence and transcripts of Brussels sprout, beneficial insects, farm and household animals, and humans. The sequences were then sent to RNA Greentech LLC (Texas) for dsRNA synthesis. As a control, a dsRNA molecule targeting the gene coding for the coat protein of the Cassava Brown Streak virus (dsCBSV) was used, which is not present in the genome of B. tabaci.

Artificial diet bioassay: 100 newly emerged B. tabaci adults were placed in 30×200 mm glass vials and fed with 150 μl treatment solution containing autoclaved tap water, dsRNA at a final concentration of 0.5 μg/μl, and sucrose at a final concentration of 0.5M. The treatment solution was placed between two layers of 4×4 cm Parafilm pieces stretched over a self-designed (Fusion 360 modeling software, Autodesk Inc.), 3D-printed lid (35 mm diameter). Before each experiment was conducted, all vials, lids. and Parafilm pieces were sterilized for 15 min under UV-C light. All vials were wrapped with aluminum foil, and the lids were covered with green cellophane paper to mimic feeding on the abaxial side of the leaf. The artificial diet experiments were conducted in a rearing chamber under controlled conditions at 28±2° C., 50-55% relative humidity, and a 14:10 (L:D) photoperiod.

Analysis of survival assays: After five days of feeding, the number of surviving and dead insects in each vial was counted separately, and the survival proportion was calculated. The artificial diet experiments were conducted in three blocks, each with five replicates for each dsRNA treatment and five replicates for the control treatment. To enable the analysis of the combined data, the survival proportion of each replicate in each block was normalized to the mean survival proportion of the block's control treatment. Finally, the significance of the differences between the survival means was determined by a one-way ANOVA model followed by pairwise comparisons. Prior to the analysis, the proportional data were arcsin-square root transformed. As multiple tests were conducted, a false discovery rate (FDR) correction was applied. Statistical significance was assumed at P≤0.05. All described statistical analyses were conducted using JMP Pro 17.0 (SAS Institute, Cary, NC).

Development assays: Artificial diets were conducted as detailed above and on the fifth day, all vials of each treatment (silencing one specific gene) were collected and placed (after removing the lids) for 24 h in an insect-rearing tent containing an 8-week-old Brussels sprout plant. During this period, the surviving adults moved to the Brussels sprout plant, and the females laid “mature” eggs (i.e., eggs that were already ready to be laid before the beginning of the dsRNA feeding treatment or completed their development during the feeding period). After 24 h, samples of seven pairs (male and female) were collected into 2.5×5 cm glass clip-cages, and each clip-cage was placed on a new 8-weeks-old brussels sprout plant in a rearing tent. The females were allowed to lay eggs for 48 h, after which the clip-cages and all adults were gently removed. The development assays were conducted in greenhouse conditions (30±4° C. with 30-40% relative humidity and a 14:10 (L:D) photoperiod), and the plants were watered as needed every two to three days. Once newly emerged adults were detected in one of the treatments, all the infested leaves of all the treatments were removed from the plants, and a “snapshot” was conducted, meaning that we determined for each leaf the proportion of unhatched eggs, undeveloped nymphs (dried nymphs and nymphs with abnormal morphology), 2^{nd} instars, 3^{rd} instars, early 4^{th} instars, late 4^{th} instars (also known as “pupae”), and exuviac (indicating the successful completion of the development and emergence of an adult). Differences in the proportion of undeveloped progeny (undeveloped eggs and nymphs) and progeny at advanced developmental stages (early 4^{th} instars, “pupae”, and exuviac) between each treatment and the control were tested for significance using the log-likelihood ratio test. As multiple tests were conducted, a false discovery rate (FDR) correction was applied. Statistical significance was assumed at P≤0.05.

### Example 2: Sugar Transporter and Trehalose Biosynthesis Genes for Controlling Bemisia tabaci

Bemisia tabaci, like other phloem feeders, must express a specific set of genes that can respond the high osmolarity of ingested sap, metabolize carbohydrates to produce and store energy while reducing gut osmolarity, and synthesize haemolymph sugars to maintain the osmotic balance between the insect's body fluids and the ingested sap in the gut lumen. In this example, findings are reported on the importance of genes involved in transport and storage of sugars for the survival of B. tabaci.

The first set of genes described herein encode sugar transporters. Sugar transporters mediate the movement of sugar into and out of cells, either actively or by facilitating passive diffusion. This function is vital for all organisms, from bacteria to mammals. Insect sugar transporters belong to the major facilitator superfamily (MFS), which contains at least 24 distinct families of 249,360 sequenced members, all sharing a conserved 12 transmembrane domains. Sugar transporters are important for phloem-feeding insects due to the abundant presence of sugars in their diet and are needed both for utilization of fructose as a nutritional resource and for the maintenance of osmotic balance and energy storage by the conversion of the transported glucose into trehalose. Downregulation of sugar transporters results in increased osmotic pressure in the haemolymph and increased mortality in the potato psyllid Bactericera cockerelli, and reduced growth and fecundity in the brown planthopper Nilaparvata lugens. In addition, similar to the GH13 family, sugar transporters are highly expanded in the B. tabaci genome, with 137 putative members, compared to their number in other phloem feeders such as B. cockerelli, N. lugens, the pea aphid Acyrthosiphon pisum, and the Asian citrus psyllid Diaphorina citri, with 39, 18, 71, and 94 putative genes, respectively.

In addition to sugar transporters, genes encoding enzymes involved in the trehalose biosynthesis pathway were investigated. Trehalose is a nonreducing disaccharide containing two glucose molecules, widely distributed in microorganisms, plants, and invertebrates. In addition to being an energy and carbon source, trehalose also helps protect cellular membranes from environmental stresses such as dehydration, oxidation, and freezing. In insects, it is exclusively synthesized by the fat body and is the major sugar in the haemolymph. Furthermore, trehalose concentrations in phloem-feeding insects are higher than in other insect families, suggesting that the accumulation might be used to balance the osmotic potential between the haemolymph and the ingested sap in the gut lumen. It has been previously reported that the silencing of the trehalose-6-phosphate synthase (TPS) gene, a key gene in insects' trehalose biosynthesis pathway, in the phloem-feeding psyllid D. citri, resulted in a significantly increased mortality and abnormal phenotype. In addition, it has been reported that targeting homologous TPS genes in B. tabaci caused 90% adult mortality, decreased fecundity, and compromised growth and development.

Bioinformatic analyses were conducted as described in Example 1 in order to select the most promising target genes involved in the processes outlined above. Two genes that encode sugar transporters ST1 (Bta11863) for example SEQ ID NO:1 and 3; and ST2 (Bta02283) for example SEQ ID NO:5 and 7 were found to be highly expressed in the midgut of B. tabaci and these genes were selected for further investigation of how silencing of these genes affects the performance of B. tabaci.

Three additional genes encoding enzymes involved in the trehalose biosynthesis pathway: a hexokinase (Hx; Bta00587) for example SEQ ID NO:13 and 15, trehalose 6-phosphate synthase (TPS; Bta15703) for example SEQ ID NO:9 and 11, and UTP-glucose-1-phosphate uridylyltransferase ((GP1; Bta10225) for example SEQ ID NO:17 and 19, were found to be differentially expressed when different species of B. tabaci were compared. These genes were also selected for further investigation.

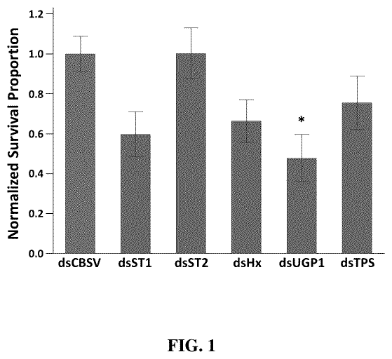

The survival proportions of B. tabaci adults after feeding on artificial diets of dsRNA targeting each of the five genes (ST1, ST2, Hx, TPS, and UGP1) were calculated and compared to survival proportions of B. tabaci fed a control diet of dsCBSV. Five days of feeding on artificial diets containing dsST1, dsST2, dsUGP1, dsHx, and dsTPS resulted in mortality rates of 70.1±6.7%, 54.52±6.11%, 78.06±5.39%, 68.34±5.53%, and 64.88±5.95, respectively, which were significantly higher in the dsUGP1 diet (P=0.0295) than the mortality rates in the control assay (55.13±4.42%) (FIG. 1).

### Example 3: Detoxification Genes for Controlling Bemisia Tabaci

In order to utilize their host plant as a food resource, mating site, oviposition site, and habitat, insect herbivores must overcome plant defenses. Numerous plant-defense strategies exist based on an ability to synthesize, constitutively or in response to stress, more than 200,000 estimated specialized metabolites (i.e., chemical compounds that evolved in response to particular ecological challenges). Herbivorous insects have coevolved with their host plants and learned to overcome defensive plant compounds by employing various strategies, such as detoxification, sequestration, or secretion. Both specialized and general insect strategies have evolved, including both physiological adaptations that allow an insect to tolerate specific host plant defenses, and general adaptations that allow an insect to tolerate an array of plant defenses.

Phloem-sap is relatively free of toxins and deterrents. However, due to the highly polyphagous nature of the phloem-feeding whitefly Bemisia tabaci, these insects are exposed to a large variety of plant toxins. Therefore, genes encoding proteins and enzymes involved in the detoxification processes were selected as candidates for an RNAi-based pest management technology. In order to select promising target genes with a putative role in detoxifying plant-defensive compounds found in cassava plants, transcriptomic data of cassava-adapted B. tabaci species were examined.

Three overexpressed UDP-glucosyltransferase genes (Bta07606, UDP-GT1 for example SEQ ID NO:29 or 30; Bta13755, UDP-GT2 for example SEQ ID NO:33 or 35; and Bta11108, UDP-GT3 for example SEQ ID NO:37 or 39) and an overexpressed ABC transporter gene (Bta05312, ABC1 for example SEQ ID NO:21 or 23) were selected as potential targets and were further examined using a gene-silencing system.

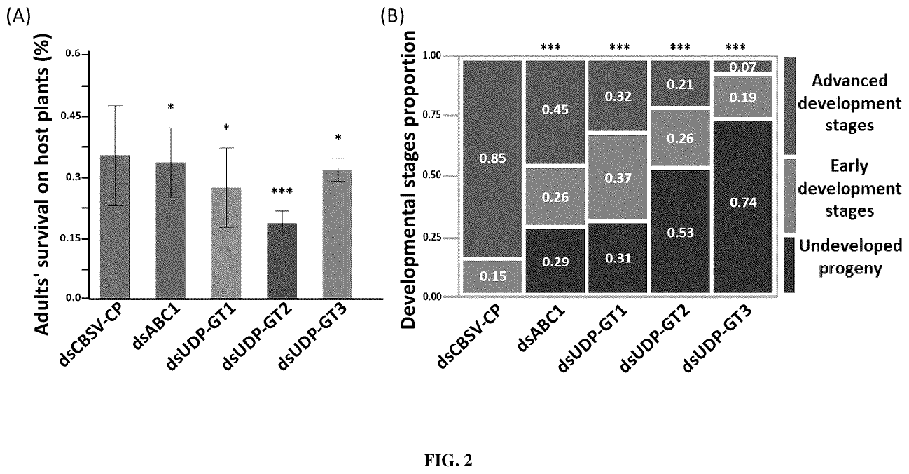

The artificial diet method described in previous examples is less suitable for detecting the silencing effects of these detoxification genes directly because treated insects could not be exposed directly during the diet period to the wide variety of phytotoxins present in their ‘natural’ diet. Therefore, after five days of artificial diets of dsRNA against each of the target detoxification genes, plant experiments were continued by opening all vials of the same dsRNA treatment in an insect-rearing tent containing tobacco (Nicotiana tabacum) plant, allowing the surviving whiteflies to move to the plant and deposit “old” eggs. 24 hours later, samples of seven pairs (a male and a female) were collected into a clip-cage, and each clip-cage was placed on a leaf of a tobacco plant, allowing the adult whiteflies to feed and lay eggs for 48 hours. Next, all clip-cages and adult whiteflies were gently removed from the leaves, and the survival proportion of the silenced adult whiteflies was calculated and normalized to the mean survival proportion of the control. The differences between the means were determined by a one-way ANOVA model followed by pairwise comparisons, and it was found that silencing UDP-GT1, UDP-GT2, UDP-GT3, and ABC1 caused significantly higher mortality of adult whiteflies after 48 hours on tobacco leaves, compared to the control (FIG. 2A).

In addition, progeny development was continuously monitored, and once newly emerged adult whiteflies were detected in any of the treatments, all leaves of all treatments were removed from the plants, and the number of nymphs at each nymphal stage was counted. The progeny of the silenced whiteflies was divided into three categories: undeveloped progeny (unhatched eggs and dried nymphs), early development stages (2^{nd} and 3^{rd} instars nymphs), and advanced development stages (4^{th} instar nymphs and exuviae). The differences between each treatment to the control were compared using the likelihood ratio chi-square test, and it was found that silencing each of the detoxification genes caused a significantly delayed development (FIG. 2B).

Finally, to examine the effect of the gene silencing on nymphs' survival, the proportion of the undeveloped progeny at each treatment was calculated and the differences between treatments analyzed. A significant increase in the undeveloped progeny proportion of whiteflies treated with dsRNA targeting UDP-GT1, UDP-GT2, and UDP-GT3 and ABC1 was found compared to the control (FIG. 2B, dark gray bars).

### Example 4: In Vitro Analysis of Transgenic Cassava Lines

Elite cassava lines (NASE 13) modified to express RNAi technology were them under laboratory conditions using a SS1-SG1 whitefly colony. The transgenic lines included plants with RNAi dsRNA targeting the sugar transporters ST1 (ST1—SEQ ID NO:3) gene in DWF55, ABC transporter gene involved in detoxification (ABC1—SEQ ID NO:23) in DWF56, trehalose 6-phosphate synthase gene involved in the trehalose biosynthesis pathway (TPS—SEQ ID NO:11) in DWF60, UTP-glucose-1-phosphate uridylyltransferase gene involved in the trehalose biosynthesis pathway (UGP1—SEQ ID NO:19) in DWF66, and two UDP-glucosyltransferase genes involved in detoxification (UDP-GT2—SEQ ID NO:35 and UDP-GT3—SEQ ID NO:39) in DWF68 and DWF67, respectively. Two bioassays were used: adult survival after 7 days of exposure to the RNAi plants, and nymph development rate (the development stage reached 25 days after the egg was laid).

In the adult survival assays, whiteflies were released onto six-week-old cassava plants in Lock-Lock cages within an insectary (25±2° C.; 14:10 h light:dark cycle at 40% relative humidity). Five plants (replications) per transgenic event/line were tested with fifteen pairs (male and female) of 1-2-day-old, whitefly adults. Survival was recorded on the 7th day after the release of the whiteflies.

In the nymph development assays, fifteen pairs (male and female) of 1-2-day-old whitefly adults were released onto six-week-old cassava plants in Lock-Lock cages within an insectary (25±2° C.; 14:10 h light:dark cycle at 40% relative humidity) for a 24 h egg laying period. Six plants (replications) per transgenic event/line were tested. After 25 days, the development status of the progeny was determined in each replicate (number of 2nd and 3^{rd} nymphs, number of red-eyed 4th nymphs, number of progeny that completed development and emerged as adults leaving the remains of their exoskeleton (exuviac) behind).

### Laboratory Adult Survival Assays

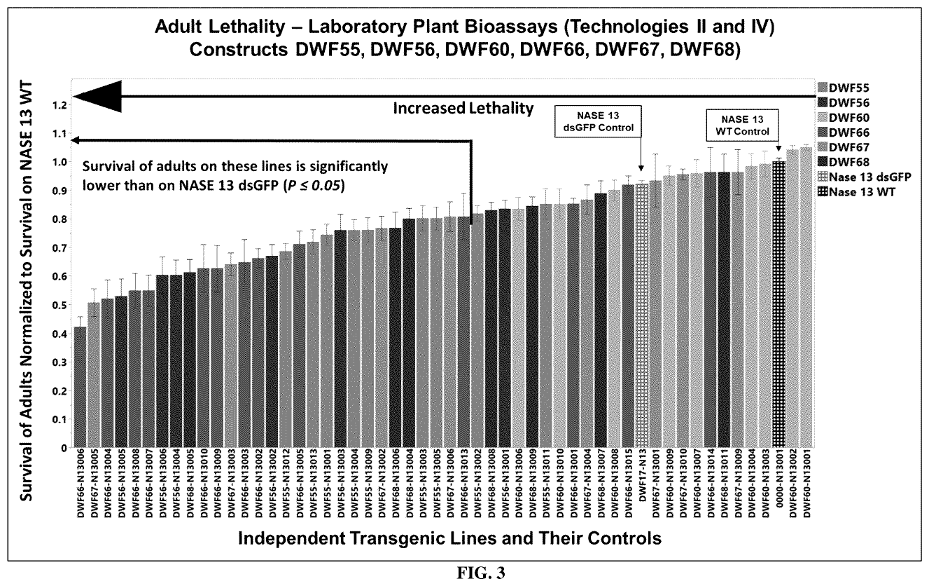

The most effective transgenic lines caused ˜58% increased adult mortality after seven days when compared to the control line NASE 13 WT. There were 29 transgenic lines that showed significant effect (increased adult mortality) when compared to a line expressing dsRNA against the GFP gene (WF17-GFP004) (FIG. 3).

### Laboratory Nymph Development Assays

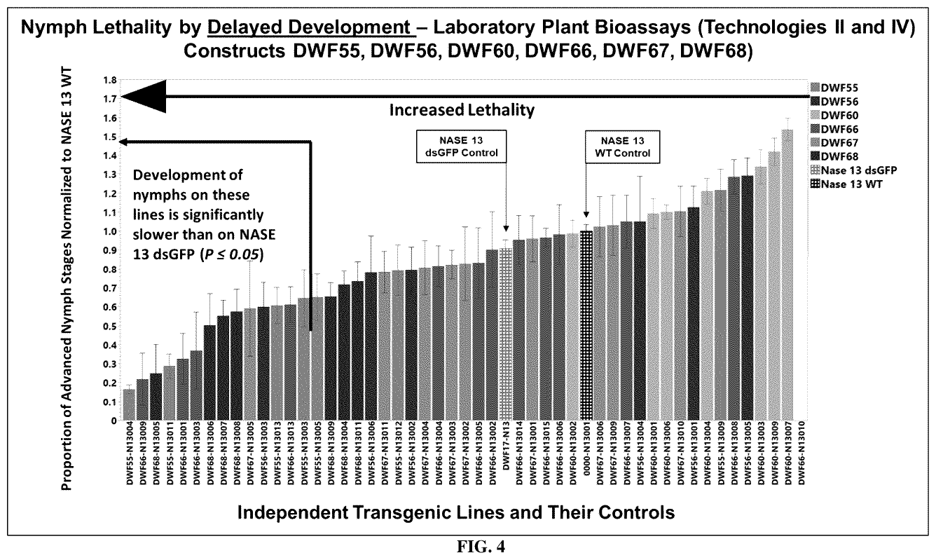

In the nymph development assays, the proportion of progeny that were in advanced development stage (red-eyed 4th nymphs) or completed their development (newly emerged adults) significantly differed between 14 of the transgenic lines and the controls (expressing dsRNA against the green fluorescent protein [GFP] or not expressing dsRNA at all [WT plants]. The most effective transgenic lines caused ˜84% delay in development when the normalized proportion of progeny that were in advanced stages or completed development was compared to control lines (FIG. 4). It Is likely that the vast majority of progeny are not only delayed but will actually never complete their development achieving significant whitefly population suppression.

### Example 5: Field Trials of Transgenic Cassava Lines

Two confined field trials (CFTs) were implemented and completed to evaluate the resistance of the relevant transgenic cassava events to whiteflies. Both field trials tested lines that target sugar transporter, trehalose biosynthesis, and detoxification genes (Examples 2 and 3).

### Whitefly Resistance Trait Selection Trial I

The first field trial was established using disease-free tissue culture-derived plants. It evaluated 11 transgenic events including 5 related to the sugar transporter and detoxification technologies. The transgenic lines included plants with RNAi dsRNA targeting the sugar transporters ST1 (ST1—SEQ ID NO:3) gene in DWF55, and an ABC transporter gene involved in detoxification (ABC1—SEQ ID NO:23) in DWF56. The lines included: DWF55-N13004, DWF55-N13011, DWF55-N13013, DWF56-N13004 and DWF56-N13005. These were received as tissue-culture plantlets, micro-propagated, and planted alongside disease-free plants of 0000-N13001 (WT-NASE 13), and NASE 12 and Mkumba as whitefly susceptible and tolerant checks, respectively. Each plot consisted of ten plants with four replications planted in a RCBD.

To determine the number of adult B. tabaci on each entry, in-field observations were made on the underside of the top five fully-expanded leaves of the tallest shoot from all the 10 plants in the plot. For the total B. tabaci nymph count, the 14^{th} leaf from the top, known to host the highest number of 3^{rd} and 4^{th} instar nymphs were also manually counted in the field (also called raw counts).

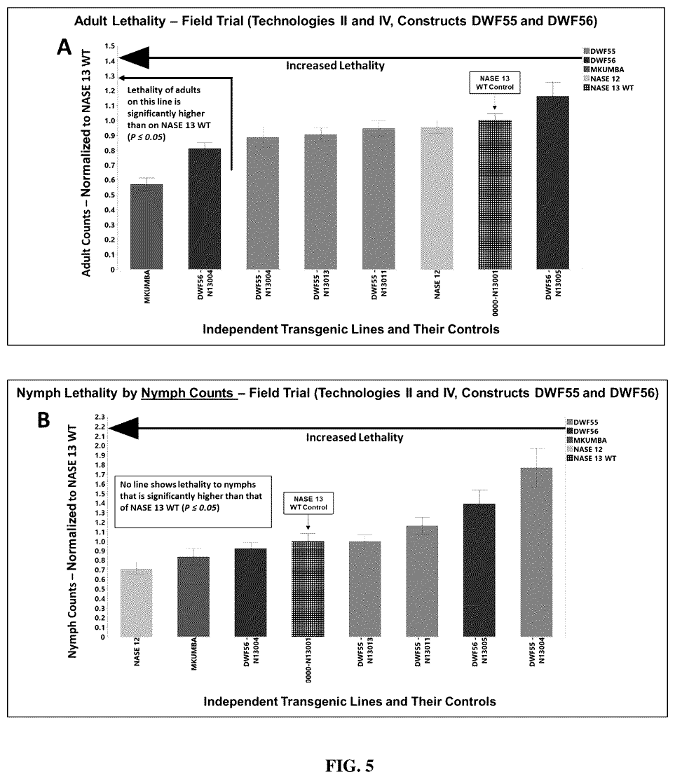

Data was collected over a five-month period after which the plants were too tall to allow accurate visual assessment. Data showed that only one transgenic event, DWF56-N13004, had significantly lower adult whitefly numbers and no transgenic event had lower nymph numbers when compared to the non-transgenic control variety, NASE 13 WT (FIG. 5, Panels A and B).

### Whitefly Resistance Trait Selection Trial II

The second field trail was established using disease-free tissue culture-derived plants. It evaluated 19 transgenic events including 9 related to the trehalose biosynthesis and detoxification technologies. The transgenic lines included plants with RNAi dsRNA targeting the ABC transporter gene involved in detoxification (ABC1—SEQ ID NO:23) in DWF56, the UTP-glucose-1-phosphate uridylyltransferase gene involved in the trehalose biosynthesis pathway (UGP1—SEQ ID NO: 19) in DWF66, and two UDP-glucosyltransferase genes involved in detoxification (UDP-GT2—SEQ ID NO:35 and UDP-GT3—SEQ ID NO:39) in DWF68 and DWF67, respectively. The lines included: DWF56-N13006, DWF66-N13001, DWF66-N13006, DWF66-N13009, DWF67-N13004, DWF67-N13005, DWF68-N13005, DWF68-N13006 and DWF68-N13008. These were received as tissue-culture plantlets, micro-propagated, and planted alongside disease-free plants of 0000-N13001 (WT-NASE 13), and NASE 12 and Mkumba as whitefly susceptible and tolerant checks, respectively. Each plot consists of ten plants with four replications planted in a RCBD.

To determine the number of adult B. tabaci on each entry, in-field observations were made on the underside of the top five fully-expanded leaves of the tallest shoot from all the 10 plants in the plot. For the total B. tabaci nymph count, the 22^{th} leaf from the top, newly discovered to host the highest number of 3^{rd} and 4^{th} instar nymphs was also manually counted in the field (also called raw counts).

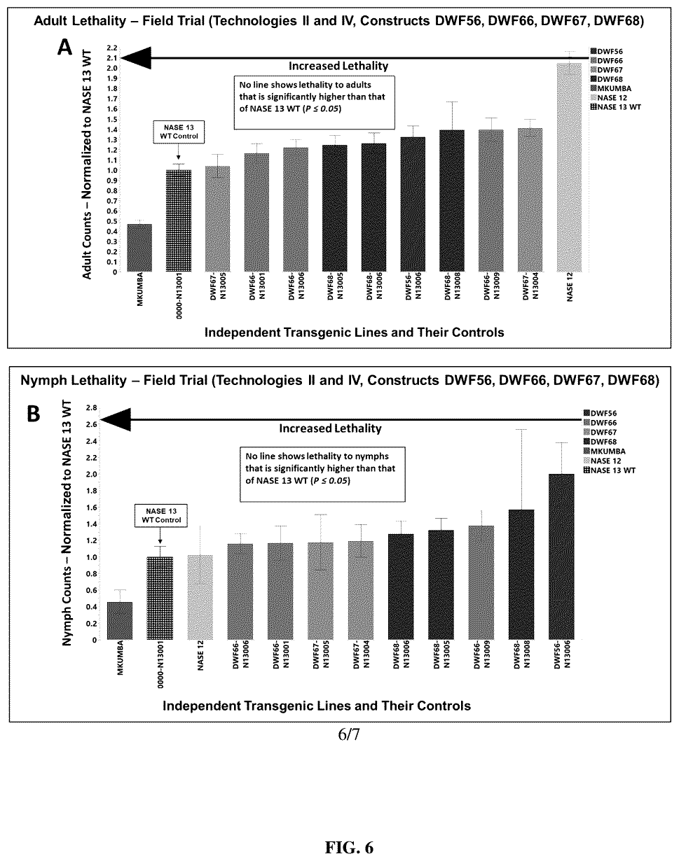

Data was collected over a six-month period after which the plants were too tall to allow accurate visual assessment. Data showed that both in adults and nymphs, no line had lower counts when compared to the control NASE 13 WT plants (P≤0.05). (FIG. 6, Panels A and B).

Nymph phenotyping in Trait Selection Trial II

As nymph counting was less effective in identifying transgenic events that have significant negative effects on nymphs in the second field trial, a possibility was raised that accurate assessment of the proportion of advanced nymphs present on leaves of plants within test trials relative to the total nymph population should be used to understand better the effect of the transgenes on whitefly development, and efficacy of the insecticidal RNAi technologies being tested.

An improved method has been developed, that captures images of nymphs using a newly acquired high-resolution camera (a Sony α7R Mark III 35 mm full-frame camera equipped with a Sony FE 90 mm f/2.8 Macro G OSS lens). These images are then used to count nymph developmental stages on a computer screen. Images were captured and processed using the accompanying ‘Imaging Edge Desktop’ software from Sony® to enable accurate counting of the different nymph developmental stages on a computer screen. This enhanced system allowed us to work from images captured directly in the field.

To capture images, leaves from position 22 are selected from three plants within a plot of ten. Selected leaves are carefully transported to a shaded area, where the camera is mounted on a tripod. The undersurface of the leaves is photographed against a black background, maintaining a fixed distance of 120 cm and consistent camera positioning. The images obtained are subsequently transferred to a computer for analysis, allowing for the identification of nymphs at various developmental stages (nymph phenotyping), including exuvia and late 4th instars with red eyes.

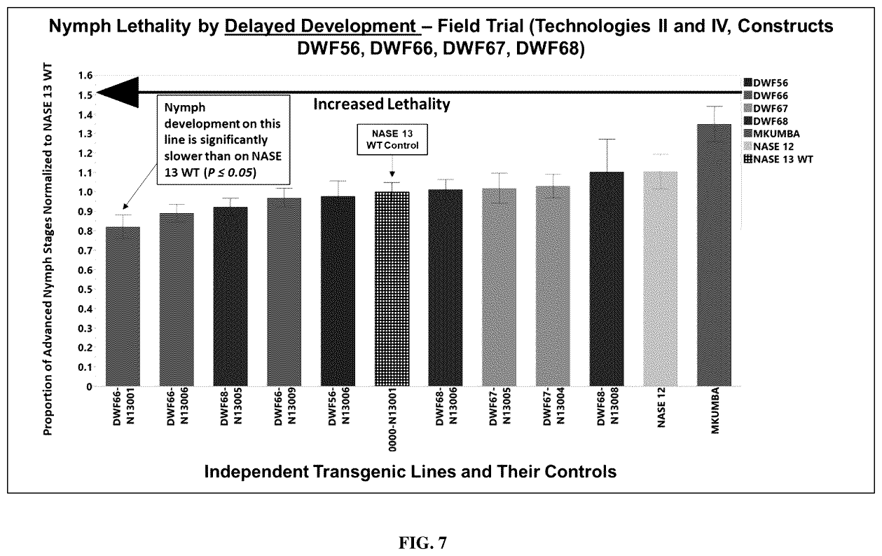

Considering the data from the nymph phenotyping one line, DWF66-N13001, was identified that showed a significant negative effect on nymph development when compared to the non-transgenic control variety, NASE 13 WT (FIG. 7).

TABLE 2
Target sequences in hemipteran species and accession numbers.

| | | Query Identifier - | | |
|---|---|---|---|---|
| | | US Application No. | | |
| | Align | 18/773,683 | | |
| Organism | % Id | Sequence Identifier | Identifier | Gene name |
| Acyrthosiphon pisum (pea aphid) | 100 | SEQ_NO_60 | ABB55878 | S1 |
| Myzus persicae (green peach aphid) | 92.54 | SEQ_NO_60 | XP_022171816 | LOC111034766 |
| Rhopalosiphum maidis (corn leaf | 90.66 | SEQ_NO_60 | XP_026808310 | LOC113550578 |
| aphid) | | | | |
| Aphis gossypii (cotton aphid) | 90.32 | SEQ_NO_60 | CAH1738054 | empty |
| Myzus persicae (green peach aphid) | 93.58 | SEQ_NO_60 | AKM95031 | SUC1 |
| Melanaphis sacchari | 87.46 | SEQ_NO_60 | XP_025200270 | LOC112598128 |
| Cinara cedri | 84.07 | SEQ_NO_60 | A0A5E4MJU0_9HEMI | CINCED_3A002419 |
| Aphis glycines | 83.19 | SEQ_NO_60 | A0A6G0U1C2_APHGL | AGLY_002806 |
| Sipha flava (yellow sugarcane aphid) | 83.16 | SEQ_NO_60 | XP_025411876 | LOC112684529 |
| Myzus persicae (green peach aphid) | 79.25 | SEQ_NO_60 | XP_022164993 | LOC111030011 |
| Adelges cooleyi (spruce gall adelgid) | 70.02 | SEQ_NO_60 | XP_050420932 | LOC126833558 |
| Rhopalosiphum maidis (corn leaf | 69.61 | SEQ_NO_60 | XP_026814773 | LOC113554883 |
| aphid) | | | | |
| Myzus persicae (green peach aphid) | 68.64 | SEQ_NO_60 | XP_022171483 | LOC111034518 |
| Acyrthosiphon pisum (pea aphid) | 68.53 | SEQ_NO_60 | XP_001952163 | LOC100160111 |
| Sipha flava (yellow sugarcane aphid) | 68.59 | SEQ_NO_60 | XP_025411887 | LOC112684539 |
| Aphis gossypii (cotton aphid) | 67.91 | SEQ_NO_60 | CAH1738053 | empty |
| Cinara cedri | 67.53 | SEQ_NO_60 | A0A5E4MF21_9HEMI | CINCED_3A009888 |
| Adelges cooleyi (spruce gall adelgid) | 63.87 | SEQ_NO_60 | XP_050420831 | LOC126833496 |
| Sipha flava | 68.12 | SEQ_NO_60 | A0A2S2QC39_9HEMI | Mal-B2_0; g.176981 |
| Adelges cooleyi (spruce gall adelgid) | 61.71 | SEQ_NO_60 | XP_050423627 | LOC126835230 |
| Daktulosphaira vitifoliae (grape | 59.61 | SEQ_NO_60 | XP_050543464 | LOC126906738 |
| phylloxera) | | | | |
| Cacopsylla melanoneura | 54.69 | SEQ_NO_60 | A0A8D8X9P8_9HEMI | empty |
| Diuraphis noxia (Russian wheat aphid) | 69.23 | SEQ_NO_60 | XP_015374661 | LOC107169446 |
| Rhopalosiphum maidis (corn leaf | 47.6 | SEQ_NO_60 | XP_026811817 | LOC113552953 |
| aphid) | | | | |
| Bactericera cockerelli (Paratrioza | 50.18 | SEQ_NO_60 | WEY18728 | empty |
| cockerelli) | | | | |
| Nilaparvata lugens (brown | 47.19 | SEQ_NO_60 | XP_039295760 | LOC111057129 |
| planthopper) | | | | |
| Aphis glycines | 48.76 | SEQ_NO_60 | A0A6G0U2S1_APHGL | AGLY_002830 |
| Rhopalosiphum maidis (corn leaf | 49.02 | SEQ_NO_60 | XP_026811826 | LOC113552953 |
| aphid) | | | | |
| Nilaparvata lugens (brown | 46.85 | SEQ_NO_60 | QOI16746 | empty |
| planthopper) | | | | |
| Nilaparvata lugens (brown | 47.14 | SEQ_NO_60 | XP_022191427 | LOC111049616 |
| planthopper) | | | | |
| Adelges cooleyi (spruce gall adelgid) | 47.13 | SEQ_NO_60 | XP_050421275 | LOC126833781 |
| Laodelphax striatellus | 45.33 | SEQ_NO_60 | A0A482WZA6_LAOST | LSTR_LSTR014704 |
| Daktulosphaira vitifoliae (grape | 47.37 | SEQ_NO_60 | XP_050545176 | LOC126907718 |
| phylloxera) | | | | |
| Aphis craccivora | 48.5 | SEQ_NO_60 | A0A6G0Z7W3_APHCR | FWK35_00006246 |
| Lygus hesperus | 43.42 | SEQ_NO_60 | A0A0A9Y982_LYGHE | Mal-A3_4; Mal-A3_1; |
| | | | | CM83_41776; g.29502 |
| Pristhesancus plagipennis | 42.93 | SEQ_NO_60 | A0A2K8JSD1_PRIPG | empty |
| Aphis glycines | 40.4 | SEQ_NO_60 | A0A6G0U2F7_APHGL | AGLY_003209 |
| Aphis craccivora | 43.6 | SEQ_NO_60 | A0A6G0YA82_APHCR | FWK35_00029484 |
| Nezara viridula | 43.45 | SEQ_NO_60 | A0A9P0HIJ6_NEZVI | NEZAVI_LOCUS11510 |
| Homalodisca vitripennis (glassy- | 43.09 | SEQ_NO_60 | XP_046668932 | LOC124359868 |
| winged sharpshooter) | | | | |
| Clastoptera arizonana | 42.78 | SEQ_NO_60 | A0A1B6CRX6_9HEMI | g.19812 |
| Pantoea ananatis | 82.89 | SEQ_NO_7 | USL58420 | argH |
| Serratia marcescens | 82.24 | SEQ_NO_7 | WPJ23873 | argH |
| Symbiopectobacterium purcellii | 81.58 | SEQ_NO_7 | QZN96734 | argH |
| Homalodisca vitripennis (glassy- | 58.33 | SEQ_NO_1 | XP_046669585 | LOC124360215 |
| winged sharpshooter) | | | | |
| Cicadella viridis | 56.69 | SEQ_NO_1 | AQP_CICVR | AQP; CIC |
| Nilaparvata lugens (brown | 53.79 | SEQ_NO_1 | XP_039289071 | LOC111055546 |
| planthopper) | | | | |
| Cacopsylla melanoneura | 55.33 | SEQ_NO_1 | A0A8D9A4Q8_9HEMI | empty |
| Triatoma infestans | 53.09 | SEQ_NO_1 | A0A023F9Y4_TRIIF | empty |
| Diaphorina citri (Asian citrus psyllid) | 56.39 | SEQ_NO_1 | XP_008484232 | LOC103520911 |
| Panstrongylus lignarius | 52.26 | SEQ_NO_1 | A0A224XW70_9HEMI | empty |
| Panstrongylus megistus | 51.85 | SEQ_NO_1 | A0A069DRB7_9HEMI | empty |
| Rhodnius prolixus | 49.79 | SEQ_NO_1 | AEV57513 | empty |
| Cacopsylla melanoneura | 48.98 | SEQ_NO_1 | A0A8D8M266_9HEMI | empty |
| Bactericera cockerelli | 47.6 | SEQ_NO_1 | V5V0L3_9HEMI | empty |
| Daktulosphaira vitifoliae (grape | 47.58 | SEQ_NO_1 | XP_050523174 | LOC126895395 |
| phylloxera) | | | | |
| Adelges cooleyi (spruce gall adelgid) | 47.39 | SEQ_NO_1 | XP_050425395 | LOC126836221 |
| Laodelphax striatellus | 46.25 | SEQ_NO_1 | A0A482X2G4_LAOST | LSTR_LSTR002442 |
| Diaphorina citri (Asian citrus psyllid) | 49.58 | SEQ_NO_1 | XP_026677945 | LOC103507223 |
| Myzus persicae (green peach aphid) | 46.4 | SEQ_NO_1 | XP_022173314 | LOC111035833 |
| Melanaphis sacchari | 43.77 | SEQ_NO_1 | XP_025200968 | LOC112598656 |
| Triatoma infestans | 52.97 | SEQ_NO_1 | A0A171AZN8_TRIIF | empty |
| Adelges cooleyi (spruce gall adelgid) | 46.77 | SEQ_NO_1 | XP_050425394 | LOC126836221 |
| Myzus persicae (green peach aphid) | 43.94 | SEQ_NO_1 | XP_022173311 | LOC111035833 |
| Aphis gossypii (cotton aphid) | 43.89 | SEQ_NO_1 | XP_050065182 | LOC114122385 |
| Cimex lectularius (bed bug) | 46.75 | SEQ_NO_1 | XP_024081986 | LOC106666404 |
| Nesidiocoris tenuis | 47.52 | SEQ_NO_1 | A0A6H5G9F9_9HEMI | NTEN_LOCUS5724, |
| | | | | NTEN_LOCUS5726 |
| Adelges cooleyi (spruce gall adelgid) | 46.18 | SEQ_NO_1 | XP_050425393 | LOC126836221 |
| Rhopalosiphum maidis (corn leaf | 43.4 | SEQ_NO_1 | XP_026808959 | LOC113551120 |
| aphid) | | | | |
| Clastoptera arizonana | 45.97 | SEQ_NO_1 | A0A1B6DRL8_9HEMI | g.4352, g.4354 |
| Aphis gossypii (cotton aphid) | 43.35 | SEQ_NO_1 | XP_027840834 | LOC114122385 |
| Acyrthosiphon pisum (pea aphid) | 46.53 | SEQ_NO_1 | XP_029346917 | LOC100573582 |
| Nesidiocoris tenuis | 47.11 | SEQ_NO_1 | BET00971 | empty |
| Halyomorpha halys (brown | 49.78 | SEQ_NO_1 | XP_024214953 | LOC106692167 |
| marmorated stink bug) | | | | |
| Halyomorpha halys (brown | 49.78 | SEQ_NO_1 | XP_024214948 | LOC106692167 |
| marmorated stink bug) | | | | |
| Aphis gossypii | 45.78 | SEQ_NO_1 | A0A9POIM55_APHGO | APHIGO_LOCUS1225 |
| Rhopalosiphum padi | 45.38 | SEQ_NO_1 | A0A0C5B1A0_RHOPD | Aqp1 |
| Graphocephala atropunctata | 46.47 | SEQ_NO_1 | A0A1B6MCE0_9HEMI | g.22651 |
| Cuerna arida | 46.47 | SEQ_NO_1 | A0A1B6FT45_9HEMI | g.34842 |
| Melanaphis sacchari | 44.98 | SEQ_NO_1 | XP_025200970 | LOC112598656 |
| Nilaparvata lugens (brown | 48.07 | SEQ_NO_1 | XP_039289070 | LOC120352466 |
| planthopper) | | | | |
| Sipha flava (yellow sugarcane aphid) | 44.35 | SEQ_NO_1 | XP_025405493 | LOC112679791 |
| Cimex lectularius (bed bug) | 45.45 | SEQ_NO_1 | AMZ04826 | AQP1 |
| Cimex lectularius (bed bug) | 45.45 | SEQ_NO_1 | XP_014250942 | LOC106667500 |
| Sipha flava (yellow sugarcane aphid) | 44.86 | SEQ_NO_1 | XP_025405494 | LOC112679791 |
| Nezara viridula | 42.29 | SEQ_NO_1 | A0A9P0HI98_NEZVI | NEZAVI_LOCUS10952 |
| Apolygus lucorum | 44.96 | SEQ_NO_1 | A0A8S9XY55_APOLU | GE061_013060 |
| Rhodnius neglectus | 42.62 | SEQ_NO_1 | A0A0P4VVX4_9HEMI | empty |
| Lygus hesperus (lygus bug) | 43.22 | SEQ_NO_1 | AHI85751 | AQP4B |
| Cacopsylla melanoneura | 55.15 | SEQ_NO_62 | A0A8D8UVT7_9HEMI | empty |
| Daktulosphaira vitifoliae (grape | 51.37 | SEQ_NO_62 | XP_050543700 | LOC126906844 |
| phylloxera) | | | | |
| Aphis gossypii (cotton aphid) | 50.17 | SEQ_NO_62 | CAH1738051 | empty |
| Sipha flava (yellow sugarcane aphid) | 50.34 | SEQ_NO_62 | XP_025411886 | LOC112684537 |
| Myzus persicae (green peach aphid) | 50.96 | SEQ_NO_62 | XP_022171472 | LOC111034509 |
| Nilaparvata lugens (brown | 50.09 | SEQ_NO_62 | ANJ04656 | empty |
| planthopper) | | | | |
| Aphis glycines | 48.28 | SEQ_NO_62 | A0A6G0U2C9_APHGL | AGLY_003182 |
| Nilaparvata lugens (brown | 48.53 | SEQ_NO_62 | AQW43009 | empty |
| planthopper) | | | | |
| Laodelphax striatellus | 47.66 | SEQ_NO_62 | A0A482WPG2_LAOST | LSTR_LSTR013009 |
| Daktulosphaira vitifoliae (grape | 52.85 | SEQ_NO_3 | XP_050543463 | LOC126906738 |
| phylloxera) | | | | |
| Melanaphis sacchari | 50 | SEQ_NO_3 | A0A2H8TQA6_9HEMI | Mal-B2_15 |
| Aphis craccivora | 51.74 | SEQ_NO_3 | A0A6G0ZAM4_APHCR | FWK35_00020053 |
| Sipha flava | 48.48 | SEQ_NO_3 | A0A2S2QF41_9HEMI | Mal-B2_5; g.170344 |
| Cimex lectularius (bed bug) | 51.5 | SEQ_NO_58 | XP_014259045 | LOC106672269 |
| Melanaphis sacchari | 47.46 | SEQ_NO_58 | XP_025200431 | LOC112598244 |
| Nilaparvata lugens (brown | 56.46 | SEQ_NO_58 | XP_039296117 | LOC111049617 |
| planthopper) | | | | |
| Melanaphis sacchari | 46.89 | SEQ_NO_58 | XP_025200461 | LOC112598276 |
| Diuraphis noxia (Russian wheat aphid) | 51.9 | SEQ_NO_58 | XP_015374282 | LOC107169145 |
| Clastoptera arizonana | 49.4 | SEQ_NO_58 | A0A1B6DD47_9HEMI | g.13732 |
| Cuerna arida | 47.95 | SEQ_NO_58 | A0A1B6H4U3_9HEMI | g.37381 |
| Homalodisca liturata | 48.52 | SEQ_NO_58 | A0A1B6IPK6_9HEMI | g.36964 |
| Melanaphis sacchari | 45.3 | SEQ_NO_58 | A0A2H8TI49_9HEMI | Mal-B1_0 |
| Cacopsylla melanoneura | 49.7 | SEQ_NO_58 | A0A8D9AN60_9HEMI | empty |
| Aphis gossypii (cotton aphid) | 47.65 | SEQ_NO_58 | XP_027847702 | LOC114127611 |
| Rhopalosiphum maidis (corn leaf | 48.21 | SEQ_NO_58 | XP_026811838 | LOC113552967 |
| aphid) | | | | |
| Homalodisca vitripennis (glassy- | 48.21 | SEQ_NO_58 | XP_046668928 | LOC124359866 |
| winged sharpshooter) | | | | |
| Aphis glycines | 56.25 | SEQ_NO_58 | A0A6G0U1Q4_APHGL | AGLY_002808 |
| Sipha flava (yellow sugarcane aphid) | 55.86 | SEQ_NO_58 | XP_025411875 | LOC112684528 |
| Cuerna arida | 47.37 | SEQ_NO_58 | A0A1B6EQY7_9HEMI | g.37384 |
| Sipha flava | 54.42 | SEQ_NO_58 | A0A2S2Q6B3_9HEMI | Mal-B2_4; g.117432 |
| Diuraphis noxia (Russian wheat aphid) | 47.62 | SEQ_NO_58 | XP_015375686 | LOC107170156 |
| Acyrthosiphon pisum (pea aphid) | 47.62 | SEQ_NO_58 | XP_001943582 | LOC100161439 |
| Homalodisca vitripennis (glassy- | 46.78 | SEQ_NO_58 | XP_046668930 | LOC124359866 |
| winged sharpshooter) | | | | |
| Schizaphis graminum | 54.86 | SEQ_NO_58 | A0A2S2NC11_SCHGA | Mal-B2_0; g.52810 |
| Cacopsylla melanoneura | 49.07 | SEQ_NO_58 | A0A8D8WHD0_9HEMI | empty |
| Daktulosphaira vitifoliae (grape | 44.63 | SEQ_NO_58 | XP_050541275 | LOC126905531 |
| phylloxera) | | | | |
| Homalodisca liturata | 53.42 | SEQ_NO_58 | A0A1B6HBI7_9HEMI | g.36966 |
| Homalodisca vitripennis (glassy- | 53.42 | SEQ_NO_58 | XP_046668931 | LOC124359867 |
| winged sharpshooter) | | | | |
| Aphis gossypii (cotton aphid) | 60.34 | SEQ_NO_64 | XP_050059434 | LOC114127615 |
| Sipha flava | 59.85 | SEQ_NO_64 | A0A2S2QGL3_9HEMI | Mal-B2_11; g.117446 |
| Sipha flava (yellow sugarcane aphid) | 59.85 | SEQ_NO_64 | XP_025411860 | LOC112684521 |
| Adelges cooleyi (spruce gall adelgid) | 57.75 | SEQ_NO_64 | XP_050421277 | LOC126833782 |
| Acyrthosiphon pisum (pea aphid) | 55.2 | SEQ_NO_64 | XP_029346983 | LOC100159394 |
| Diuraphis noxia (Russian wheat aphid) | 57 | SEQ_NO_64 | XP_015375687 | LOC107170157 |
| Melanaphis sacchari | 56.55 | SEQ_NO_64 | XP_025201971 | LOC112599327 |
| Sipha flava (yellow sugarcane aphid) | 57.86 | SEQ_NO_5 | XP_025411859 | LOC112684521 |
| Schizaphis graminum | 53.19 | SEQ_NO_5 | A0A2S2PLN2_SCHGA | Mal-B2_9; g.163548 |
| Cinara cedri | 50.76 | SEQ_NO_5 | A0A5E4MKZ9_9HEMI | CINCED_3A023501 |
| Rhopalosiphum maidis (corn leaf | 50.09 | SEQ_NO_5 | XP_026808062 | LOC113550436 |
| aphid) | | | | |
| Clastoptera arizonana | 46.67 | SEQ_NO_5 | A0A1B6C3E2_9HEMI | g.7495 |
| Cuerna arida | 46.87 | SEQ_NO_5 | A0A1B6EIG7_9HEMI | g.27198 |
| Lygus hesperus | 45 | SEQ_NO_5 | A0A0A9WYH6_LYGHE | Mal-A3_1; CM83_47508 |
| Melanaphis sacchari | 49.06 | SEQ_NO_5 | A0A2H8TXR9_9HEMI | Mal-B2_5 |
| Nesidiocoris tenuis | 44.1 | SEQ_NO_5 | BES95725 | empty |
| Graphocephala atropunctata | 46.72 | SEQ_NO_5 | A0A1B6LZ29_9HEMI | g.33221 |
| Myzus persicae (green peach aphid) | 49.04 | SEQ_NO_66 | XP_022171970 | LOC111034870 |
| Sipha flava (yellow sugarcane aphid) | 46.2 | SEQ_NO_66 | XP_025411858 | LOC112684520 |
| Sipha flava (yellow sugarcane aphid) | 48.1 | SEQ_NO_66 | XP_025411872 | LOC112684526 |
| Rhopalosiphum maidis (corn leaf | 47.73 | SEQ_NO_66 | XP_026808060 | LOC113550436 |
| aphid) | | | | |
| Cacopsylla melanoneura | 46.22 | SEQ_NO_66 | A0A8D8WG33_9HEMI | empty |
| Candidatus Regiella (Candidatus | 38.78 | SEQ_NO_13 | WP_176487875 | dapB |
| Adiacens aphidicola) | | | | |
| Candidatus Regiella (Candidatus | 38.02 | SEQ_NO_13 | WP_006707111 | dapB |
| Adiacens aphidicola) | | | | |
| Candidatus Regiella insecticola LSR1 | 38.02 | SEQ_NO_13 | E0WSC1_9ENTR | dapB; REG_0859 |
| Wolbachia endosymbiont | 37.79 | SEQ_NO_13 | A0A178GVS8_9RICK | dapB; TV42_00810 |

TABLE 3
Target sequences in whitefly species and accession numbers.

| | | Query Identifier - | | |
|---|---|---|---|---|
| | | US Application No. | | |
| | | 18/773,683 Sequence | | |
| Organism | Align % Id | Identifier | Identifier | Gene name |
| Bemisia tabaci | 100 | SEQ_NO_1 | QXU69570 | empty |
| Bemisia tabaci | 100 | SEQ_NO_1 | ABW96354 | empty |
| Bemisia tabaci | 100 | SEQ_NO_1 | XP_018906592 | LOC109036687 |
| Bemisia tabaci | 100 | SEQ_NO_1 | B5L019_BEMTA | empty |
| Bemisia tabaci | 99.62 | SEQ_NO_1 | CAH0390211 | empty |
| Bemisia tabaci | 99.62 | SEQ_NO_1 | A0A9P0F5T7_BEMTA | BEMITA_LOCUS8954 |
| Bemisia tabaci | 100 | SEQ_NO_1 | AQU43104 | APQ1 |
| Bemisia tabaci | 100 | SEQ_NO_1 | A0A219YQI6_BEMTA | APQ1 |
| Bemisia tabaci | 69.85 | SEQ_NO_1 | CAH0389865 | empty |
| Bemisia tabaci | 69.85 | SEQ_NO_1 | A0A9P0F5I9_BEMTA | BEMITA_LOCUS8647 |
| Bemisia tabaci | 69.85 | SEQ_NO_1 | XP_018903312 | LOC109034550 |
| Bemisia tabaci | 69.85 | SEQ_NO_1 | CAH0389864 | empty |
| Bemisia tabaci | 69.85 | SEQ_NO_1 | XP_018903310 | LOC109034550 |
| Bemisia tabaci | 69.85 | SEQ_NO_1 | A0A9P0F4Y1_BEMTA | BEMITA_LOCUS8647 |
| Bemisia tabaci | 73.68 | SEQ_NO_1 | APA28756 | empty |
| Bemisia tabaci | 73.68 | SEQ_NO_1 | A0A1I9WA76_BEMTA | empty |
| Bemisia tabaci | 73.68 | SEQ_NO_1 | APA28755 | empty |
| Bemisia tabaci | 73.68 | SEQ_NO_1 | A0A1I9WA75_BEMTA | empty |
| Bemisia tabaci | 73.28 | SEQ_NO_1 | XP_018903313 | LOC109034550 |
| Bemisia tabaci | 73.28 | SEQ_NO_1 | XP_018903311 | LOC109034550 |
| Bemisia tabaci | 74.14 | SEQ_NO_1 | CAH0389867 | empty |
| Bemisia tabaci | 74.14 | SEQ_NO_1 | A0A9P0AF38_BEMTA | BEMITA_LOCUS8647 |
| Bemisia tabaci | 74.59 | SEQ_NO_1 | APA28757 | empty |
| Bemisia tabaci | 74.59 | SEQ_NO_1 | A0A1I9WA77_BEMTA | empty |
| Bemisia tabaci | 45.16 | SEQ_NO_1 | APA28758 | empty |
| Bemisia tabaci | 45.16 | SEQ_NO_1 | XP_018913501 | LOC109041566 |
| Bemisia tabaci | 45.16 | SEQ_NO_1 | XP_018913508 | LOC109041566 |
| Bemisia tabaci | 45.16 | SEQ_NO_1 | A0A1I9WA78_BEMTA | BEMITA_LOCUS11769 |
| Bemisia tabaci | 37.5 | SEQ_NO_1 | APA28759 | empty |
| Bemisia tabaci | 37.5 | SEQ_NO_1 | A0A9P0C8J2_BEMTA | BEMITA_LOCUS1246 |
| Bemisia tabaci | 37.5 | SEQ_NO_1 | A0A1I9WA79_BEMTA | empty |
| Bemisia tabaci | 37.5 | SEQ_NO_1 | XP_018910495 | LOC109039467 |
| Bemisia tabaci | 100 | SEQ_NO_1 | AID65140 | aqp1 |
| Bemisia tabaci | 100 | SEQ_NO_1 | A0A068FH39_BEMTA | aqp1 |
| Bemisia tabaci | 31.44 | SEQ_NO_1 | XP_018908128 | LOC109037738 |
| Bemisia tabaci | 31.44 | SEQ_NO_1 | A0A9P0C8T8_BEMTA | BEMITA_LOCUS4444 |
| Bemisia tabaci | 32.87 | SEQ_NO_1 | APA28760 | empty |
| Bemisia tabaci | 32.87 | SEQ_NO_1 | XP_018910506 | LOC109039478 |
| Bemisia tabaci | 32.87 | SEQ_NO_1 | A0A1I9WA80_BEMTA | empty |
| Bemisia tabaci | 32.7 | SEQ_NO_1 | A0A9P0EY04_BEMTA | BEMITA_LOCUS1247 |
| Bemisia tabaci | 31.8 | SEQ_NO_1 | APA28761 | empty |
| Bemisia tabaci | 31.8 | SEQ_NO_1 | A0A1I9WA81_BEMTA | empty |
| Arsenophonus endosymbiont | 29.63 | SEQ_NO_1 | WP_119711725 | empty |
| Bemisia tabaci | 25 | SEQ_NO_1 | A0A9P0EZ83_BEMTA | BEMITA_LOCUS1245 |
| Bemisia tabaci | 72.31 | SEQ_NO_1 | CAH0389866 | empty |
| Bemisia tabaci | 72.31 | SEQ_NO_1 | A0A9P0AEH6_BEMTA | BEMITA_LOCUS8648 |
| Bemisia tabaci | 98.55 | SEQ_NO_66 | XP_018916040 | LOC109043316 |
| Bemisia tabaci | 73.94 | SEQ_NO_66 | A0A9P0AJ90_BEMTA | BEMITA_LOCUS11413 |
| Bemisia tabaci | 71.71 | SEQ_NO_66 | A0A9P0AB57_BEMTA | BEMITA_LOCUS6189 |
| Bemisia tabaci | 71.54 | SEQ_NO_66 | XP_018910471 | LOC109039447 |
| Bemisia tabaci | 72.57 | SEQ_NO_66 | A0A9P0A9M8_BEMTA | BEMITA_LOCUS6188 |
| Bemisia tabaci | 72.4 | SEQ_NO_66 | XP_018910472 | LOC109039448 |
| Bemisia tabaci | 66.5 | SEQ_NO_66 | CAH0390269 | empty |
| Bemisia tabaci | 66.5 | SEQ_NO_66 | XP_018907630 | LOC109037423 |
| Bemisia tabaci | 66.5 | SEQ_NO_66 | A0A9P0AFT6_BEMTA | BEMITA_LOCUS9011 |
| Bemisia tabaci | 50.43 | SEQ_NO_66 | XP_018895757 | LOC109029656 |
| Bemisia tabaci | 50.43 | SEQ_NO_66 | A0A9P0F7L0_BEMTA | BEMITA_LOCUS11485 |
| Bemisia tabaci | 53.33 | SEQ_NO_66 | CAH0390271 | empty |
| Bemisia tabaci | 53.33 | SEQ_NO_66 | XP_018907799 | LOC109037530 |
| Bemisia tabaci | 53.33 | SEQ_NO_66 | XP_018907800 | LOC109037530 |
| Bemisia tabaci | 53.33 | SEQ_NO_66 | A0A9P0AGV8_BEMTA | BEMITA_LOCUS9013 |
| Bemisia tabaci | 50.09 | SEQ_NO_66 | XP_018896838 | LOC109030361 |
| Bemisia tabaci | 50.09 | SEQ_NO_66 | XP_018896845 | LOC109030361 |
| Bemisia tabaci | 50.09 | SEQ_NO_66 | XP_018896853 | LOC109030361 |
| Bemisia tabaci | 48.79 | SEQ_NO_66 | XP_018907650 | LOC109037436 |
| Bemisia tabaci | 48.79 | SEQ_NO_66 | CAH0390263 | empty |
| Bemisia tabaci | 48.79 | SEQ_NO_66 | XP_018907649 | LOC109037436 |
| Bemisia tabaci | 48.79 | SEQ_NO_66 | A0A9P0F3D7_BEMTA | BEMITA_LOCUS9005 |
| Bemisia tabaci | 46.38 | SEQ_NO_66 | XP_018917587 | LOC109044344 |
| Bemisia tabaci | 46.38 | SEQ_NO_66 | XP_018917588 | LOC109044344 |
| Bemisia tabaci | 46.38 | SEQ_NO_66 | XP_018917589 | LOC109044344 |
| Bemisia tabaci | 47.15 | SEQ_NO_66 | XP_018917500 | LOC109044306 |
| Bemisia tabaci | 47.15 | SEQ_NO_66 | XP_018917501 | LOC109044306 |
| Bemisia tabaci | 46.05 | SEQ_NO_66 | A0A9P0C961_BEMTA | BEMITA_LOCUS5479 |
| Bemisia tabaci | 49.03 | SEQ_NO_66 | XP_018895643 | LOC109029590 |
| Bemisia tabaci | 49.03 | SEQ_NO_66 | XP_018895644 | LOC109029590 |
| Bemisia tabaci | 49.03 | SEQ_NO_66 | XP_018895645 | LOC109029590 |
| Bemisia tabaci | 49.03 | SEQ_NO_66 | XP_018895646 | LOC109029590 |
| Bemisia tabaci | 49.03 | SEQ_NO_66 | A0A9P0AHL_1_BEMTA | BEMITA_LOCUS11490 |
| Bemisia tabaci | 49.82 | SEQ_NO_66 | XP_018908637 | LOC109038134 |
| Bemisia tabaci | 49.82 | SEQ_NO_66 | A0A9P0AHV7_BEMTA | BEMITA_LOCUS11643 |
| Bemisia tabaci | 46.88 | SEQ_NO_66 | XP_018917350 | LOC109044219 |
| Bemisia tabaci | 47.68 | SEQ_NO_66 | A0A9P0F059_BEMTA | BEMITA_LOCUS5480 |
| Bemisia tabaci | 47.93 | SEQ_NO_66 | A0A9P0CDN9_BEMTA | BEMITA_LOCUS5480 |
| Bemisia tabaci | 47.67 | SEQ_NO_66 | XP_018917351 | LOC109044220 |
| Bemisia tabaci | 47.67 | SEQ_NO_66 | XP_018917352 | LOC109044220 |
| Bemisia tabaci | 48.18 | SEQ_NO_66 | XP_018917258 | LOC109044153 |
| Bemisia tabaci | 45.58 | SEQ_NO_66 | CAH0770039 | empty |
| Bemisia tabaci | 45.58 | SEQ_NO_66 | A0A9P0G520_BEMTA | BEMITA_LOCUS6953 |
| Bemisia tabaci | 45.88 | SEQ_NO_66 | XP_018907832 | LOC109037554 |
| Bemisia tabaci | 39.27 | SEQ_NO_66 | XP_018916662 | LOC109043802 |
| Bemisia tabaci | 47.88 | SEQ_NO_66 | CAH0777257 | empty |
| Bemisia tabaci | 47.88 | SEQ_NO_66 | A0A9P0CFF2_BEMTA | BEMITA_LOCUS13241 |
| Bemisia tabaci | 43.97 | SEQ_NO_66 | XP_018909322 | LOC109038649 |
| Bemisia tabaci | 43.97 | SEQ_NO_66 | A0A9P0ACJ1_BEMTA | BEMITA_LOCUS10092 |
| Bemisia tabaci | 41.24 | SEQ_NO_66 | XP_018895821 | LOC109029693 |
| Bemisia tabaci | 41.24 | SEQ_NO_66 | A0A9P0CEN2_BEMTA | BEMITA_LOCUS11489 |
| Bemisia tabaci | 43.26 | SEQ_NO_66 | A0A9P0F2R5_BEMTA | BEMITA_LOCUS6283 |
| Bemisia tabaci | 41.91 | SEQ_NO_66 | XP_018912709 | LOC109041002 |
| Bemisia tabaci | 42.93 | SEQ_NO_66 | XP_018897321 | LOC109030688 |
| Bemisia tabaci | 50.31 | SEQ_NO_66 | AQU43105 | empty |
| Bemisia tabaci | 50.31 | SEQ_NO_66 | A0A219YQK0_BEMTA | empty |
| Bemisia tabaci | 41.6 | SEQ_NO_66 | A0A9P0AJF2_BEMTA | BEMITA_LOCUS11099 |
| Bemisia tabaci | 40.3 | SEQ_NO_66 | XP_018916687 | LOC109043823 |
| Bemisia tabaci | 39.97 | SEQ_NO_66 | A0A9P0AB29_BEMTA | BEMITA_LOCUS5917 |
| Bemisia tabaci | 42.28 | SEQ_NO_66 | XP_018902424 | LOC109033992 |
| Bemisia tabaci | 42.33 | SEQ_NO_66 | XP_018914092 | LOC109042010 |
| Bemisia tabaci | 42.23 | SEQ_NO_66 | CAH0388042 | empty |
| Bemisia tabaci | 42.23 | SEQ_NO_66 | XP_018911739 | LOC109040303 |
| Bemisia tabaci | 42.23 | SEQ_NO_66 | A0A9P0AD48_BEMTA | BEMITA_LOCUS6992 |
| Bemisia tabaci | 40.89 | SEQ_NO_66 | CAH0394460 | empty |
| Bemisia tabaci | 40.89 | SEQ_NO_66 | A0A9P0AH54_BEMTA | BEMITA_LOCUS12756 |
| Bemisia tabaci | 41.29 | SEQ_NO_66 | CAH0388202 | empty |
| Bemisia tabaci | 41.29 | SEQ_NO_66 | A0A9P0F1P9_BEMTA | BEMITA_LOCUS7131 |
| Bemisia tabaci | 40.51 | SEQ_NO_66 | XP_018913708 | LOC109041747 |
| Bemisia tabaci | 41.51 | SEQ_NO_66 | CAH0388443 | empty |
| Bemisia tabaci | 41.51 | SEQ_NO_66 | XP_018907169 | LOC109037120 |
| Bemisia tabaci | 41.51 | SEQ_NO_66 | A0A9P0F1X7_BEMTA | BEMITA_LOCUS7355 |
| Bemisia tabaci | 40.76 | SEQ_NO_66 | XP_018913709 | LOC109041748 |
| Bemisia tabaci | 40.45 | SEQ_NO_66 | XP_018897812 | LOC109031004 |
| Bemisia tabaci | 40.38 | SEQ_NO_66 | XP_018917998 | LOC109044628 |
| Bemisia tabaci | 40.94 | SEQ_NO_66 | XP_018902898 | LOC109034300 |
| Bemisia tabaci | 37.86 | SEQ_NO_66 | XP_018898882 | LOC109031679 |
| Bemisia tabaci | 39.07 | SEQ_NO_66 | XP_018900521 | LOC109032711 |
| Bemisia tabaci | 39.07 | SEQ_NO_66 | A0A9P0F599_BEMTA | BEMITA_LOCUS11230 |
| Bemisia tabaci | 39.86 | SEQ_NO_66 | XP_018897744 | LOC109030960 |
| Bemisia tabaci | 41.55 | SEQ_NO_66 | CAH0394021 | empty |
| Bemisia tabaci | 41.55 | SEQ_NO_66 | A0A9P0AGF7_BEMTA | BEMITA_LOCUS12367 |
| Bemisia tabaci | 40.24 | SEQ_NO_66 | CAH0388077 | empty |
| Bemisia tabaci | 40.24 | SEQ_NO_66 | A0A9P0A7B8_BEMTA | BEMITA_LOCUS7020 |
| Bemisia tabaci | 41.4 | SEQ_NO_66 | A0A9P0A5L1_BEMTA | BEMITA_LOCUS5298 |
| Bemisia tabaci | 41.25 | SEQ_NO_66 | XP_018895826 | LOC109029699 |
| Bemisia tabaci | 41.25 | SEQ_NO_66 | XP_018895827 | LOC109029699 |
| Bemisia tabaci | 40.78 | SEQ_NO_66 | XP_018903919 | LOC109034943 |
| Bemisia tabaci | 41.14 | SEQ_NO_66 | A0A9P0F588_BEMTA | BEMITA_LOCUS11060 |
| Bemisia tabaci | 41.37 | SEQ_NO_66 | CAH0389831 | empty |
| Bemisia tabaci | 41.37 | SEQ_NO_66 | A0A9P0AEF7_BEMTA | BEMITA_LOCUS8615 |
| Bemisia tabaci | 41.04 | SEQ_NO_66 | XP_018897806 | LOC109030998 |
| Bemisia tabaci | 41.04 | SEQ_NO_66 | A0A9P0A808_BEMTA | BEMITA_LOCUS5298 |
| Bemisia tabaci | 41.04 | SEQ_NO_66 | A0A9P0A824_BEMTA | BEMITA_LOCUS5298 |
| Bemisia tabaci | 41.04 | SEQ_NO_66 | A0A9P0F002_BEMTA | BEMITA_LOCUS5298 |
| Bemisia tabaci | 41.04 | SEQ_NO_66 | CAH0383170 | empty |
| Bemisia tabaci | 41.04 | SEQ_NO_66 | A0A9P0A3B4_BEMTA | BEMITA_LOCUS2640 |
| Bemisia tabaci | 41.04 | SEQ_NO_66 | A0A9P0A9I1_BEMTA | BEMITA_LOCUS5298 |
| Bemisia tabaci | 39.18 | SEQ_NO_66 | CAH0383050 | empty |
| Bemisia tabaci | 39.18 | SEQ_NO_66 | A0A9P0EX99_BEMTA | BEMITA_LOCUS2530 |
| Bemisia tabaci | 39.93 | SEQ_NO_66 | A0A7S5LJY7_BEMTA | empty |
| Bemisia tabaci | 40.35 | SEQ_NO_66 | A0A9P0F8N7_BEMTA | BEMITA_LOCUS11095 |
| Bemisia tabaci | 40.61 | SEQ_NO_66 | A0A9P0F7K0_BEMTA | BEMITA_LOCUS11448 |
| Bemisia tabaci | 40.68 | SEQ_NO_66 | XP_018897805 | LOC109030997 |
| Bemisia tabaci | 40.18 | SEQ_NO_66 | XP_018914104 | LOC109042018 |
| Bemisia tabaci | 37.77 | SEQ_NO_66 | XP_018901734 | LOC109033525 |
| Bemisia tabaci | 37.77 | SEQ_NO_66 | XP_018901733 | LOC109033525 |
| Bemisia tabaci | 40.36 | SEQ_NO_66 | CAH0383533 | empty |
| Bemisia tabaci | 40.36 | SEQ_NO_66 | A0A9P0A3R3_BEMTA | BEMITA_LOCUS2973 |
| Bemisia tabaci | 40.57 | SEQ_NO_66 | A0A9P0AMG8_BEMTA | BEMITA_LOCUS12036 |
| Bemisia tabaci | 39.27 | SEQ_NO_66 | XP_018897820 | LOC109031009 |
| Bemisia tabaci | 38.66 | SEQ_NO_66 | A0A9P0F7J4_BEMTA | BEMITA_LOCUS10935 |
| Bemisia tabaci | 36.64 | SEQ_NO_66 | XP_018912131 | LOC109040601 |
| Bemisia tabaci | 37.44 | SEQ_NO_66 | CAH0770169 | empty |
| Bemisia tabaci | 37.44 | SEQ_NO_66 | A0A9P0CFC7_BEMTA | BEMITA_LOCUS7062 |
| Bemisia tabaci | 40.65 | SEQ_NO_66 | CAH0394020 | empty |
| Bemisia tabaci | 40.65 | SEQ_NO_66 | A0A9P0AN19_BEMTA | BEMITA_LOCUS12366 |
| Bemisia tabaci | 39.93 | SEQ_NO_66 | XP_018901727 | LOC109033519 |
| Bemisia tabaci | 40.29 | SEQ_NO_66 | A0A9P0A7A7_BEMTA | BEMITA_LOCUS5296 |
| Bemisia tabaci | 40.04 | SEQ_NO_66 | XP_018900862 | LOC109032958 |
| Bemisia tabaci | 40.04 | SEQ_NO_66 | XP_018901743 | LOC109033531 |
| Bemisia tabaci | 39.22 | SEQ_NO_66 | XP_018900822 | LOC109032928 |
| Bemisia tabaci | 39.22 | SEQ_NO_66 | XP_018900821 | LOC109032928 |
| Bemisia tabaci | 39.68 | SEQ_NO_66 | CAH0388130 | empty |
| Bemisia tabaci | 39.68 | SEQ_NO_66 | A0A9P0A7E6_BEMTA | BEMITA_LOCUS7069 |
| Bemisia tabaci | 38.7 | SEQ_NO_66 | QHB15643 | empty |
| Bemisia tabaci | 38.7 | SEQ_NO_66 | A0A7S5LK57_BEMTA | empty |
| Bemisia tabaci | 39.43 | SEQ_NO_66 | XP_018898883 | LOC109031680 |
| Bemisia tabaci | 38.53 | SEQ_NO_66 | A0A9P0G3P6_BEMTA | BEMITA_LOCUS6084 |
| Bemisia tabaci | 38.06 | SEQ_NO_66 | XP_018900831 | LOC109032934 |
| Bemisia tabaci | 38.06 | SEQ_NO_66 | XP_018900830 | LOC109032934 |
| Bemisia tabaci | 38.06 | SEQ_NO_66 | XP_018900829 | LOC109032934 |
| Bemisia tabaci | 38.62 | SEQ_NO_66 | XP_018900819 | LOC109032925 |
| Bemisia tabaci | 37.56 | SEQ_NO_66 | XP_018901718 | LOC109033513 |
| Bemisia tabaci | 41.44 | SEQ_NO_66 | XP_018901720 | LOC109033514 |
| Bemisia tabaci | 41.44 | SEQ_NO_66 | CAH0388135 | empty |
| Bemisia tabaci | 41.44 | SEQ_NO_66 | XP_018901719 | LOC109033514 |
| Bemisia tabaci | 41.44 | SEQ_NO_66 | A0A9P0ACY7_BEMTA | BEMITA_LOCUS7074 |
| Bemisia tabaci | 38.27 | SEQ_NO_66 | A0A9P0F2K0_BEMTA | BEMITA_LOCUS6082 |
| Bemisia tabaci | 37.22 | SEQ_NO_66 | CAH0388137 | empty |
| Bemisia tabaci | 37.22 | SEQ_NO_66 | A0A9P0F1P2_BEMTA | BEMITA_LOCUS7075 |
| Bemisia tabaci | 40.53 | SEQ_NO_66 | A0A9P0F811_BEMTA | BEMITA_LOCUS11449 |
| Bemisia tabaci | 34.86 | SEQ_NO_66 | XP_018900818 | LOC109032924 |
| Bemisia tabaci | 34.86 | SEQ_NO_66 | A0A9P0G0C0_BEMTA | BEMITA_LOCUS6081 |
| Bemisia tabaci | 34.86 | SEQ_NO_66 | A0A9P0F3U6_BEMTA | BEMITA_LOCUS6081 |
| Bemisia tabaci | 36.33 | SEQ_NO_66 | CAH0383534 | empty |
| Bemisia tabaci | 36.33 | SEQ_NO_66 | A0A9P0A122_BEMTA | BEMITA_LOCUS2974 |
| Bemisia tabaci | 38.25 | SEQ_NO_66 | XP_018908028 | LOC109037694 |
| Bemisia tabaci | 36.59 | SEQ_NO_66 | XP_018902427 | LOC109033993 |
| Bemisia tabaci | 36.59 | SEQ_NO_66 | XP_018902425 | LOC109033993 |
| Bemisia tabaci | 37.08 | SEQ_NO_66 | XP_018903811 | LOC109034875 |
| Bemisia tabaci | 36.9 | SEQ_NO_66 | XP_018903820 | LOC109034875 |
| Bemisia tabaci | 36.9 | SEQ_NO_66 | XP_018903803 | LOC109034875 |
| Bemisia tabaci | 38.49 | SEQ_NO_66 | XP_018916689 | LOC109043823 |
| Bemisia tabaci | 38.7 | SEQ_NO_66 | CAH0383266 | empty |
| Bemisia tabaci | 38.7 | SEQ_NO_66 | A0A9P0F0K5_BEMTA | BEMITA_LOCUS2727 |
| Bemisia tabaci | 39.14 | SEQ_NO_66 | CAH0388124 | empty |
| Bemisia tabaci | 39.14 | SEQ_NO_66 | A0A9P0F439_BEMTA | BEMITA_LOCUS7063 |
| Bemisia tabaci | 33.79 | SEQ_NO_66 | XP_018898876 | LOC109031671 |
| Bemisia tabaci | 43.64 | SEQ_NO_66 | XP_018901566 | LOC109033419 |
| Bemisia tabaci | 39.83 | SEQ_NO_66 | A0A9P0AIQ8_BEMTA | BEMITA_LOCUS10936 |
| Bemisia tabaci | 37.75 | SEQ_NO_66 | XP_018916688 | LOC109043823 |
| Bemisia tabaci | 33.27 | SEQ_NO_66 | XP_018909439 | LOC109038724 |
| Bemisia tabaci | 33.27 | SEQ_NO_66 | A0A9P0AIK9_BEMTA | BEMITA_LOCUS9896 |
| Bemisia tabaci | 38.48 | SEQ_NO_66 | A0A9P0F000_BEMTA | BEMITA_LOCUS5298 |
| Bemisia tabaci | 38.51 | SEQ_NO_66 | A0A9P0A7Z5_BEMTA | BEMITA_LOCUS5298 |
| Bemisia tabaci | 38.38 | SEQ_NO_66 | XP_018897807 | LOC109030998 |
| Bemisia tabaci | 41.53 | SEQ_NO_66 | XP_018895606 | LOC109029563 |
| Bemisia tabaci | 41.96 | SEQ_NO_66 | XP_018917354 | LOC109044222 |
| Bemisia tabaci | 37.35 | SEQ_NO_66 | A0A9P0G4X0_BEMTA | BEMITA_LOCUS6083 |
| Bemisia tabaci | 34.17 | SEQ_NO_66 | XP_018901575 | LOC109033428 |
| Bemisia tabaci | 38.07 | SEQ_NO_66 | XP_018902423 | LOC109033991 |
| Bemisia tabaci | 37.87 | SEQ_NO_66 | CAH0383675 | empty |
| Bemisia tabaci | 37.87 | SEQ_NO_66 | A0A9P0A5A4_BEMTA | BEMITA_LOCUS3106 |
| Bemisia tabaci | 48.62 | SEQ_NO_66 | XP_018909206 | LOC109038568 |
| Bemisia tabaci | 34.06 | SEQ_NO_66 | A0A9P0G5K3_BEMTA | BEMITA_LOCUS10000 |
| Bemisia tabaci | 37.17 | SEQ_NO_66 | A0A9P0A8M5_BEMTA | BEMITA_LOCUS5336 |
| Bemisia tabaci | 33.25 | SEQ_NO_66 | A0A9P0G4F8_BEMTA | BEMITA_LOCUS10000 |
| Bemisia tabaci | 51.14 | SEQ_NO_66 | A0A9P0A437_BEMTA | BEMITA_LOCUS5119 |
| Bemisia tabaci | 45.7 | SEQ_NO_66 | A0A9P0A4D2_BEMTA | BEMITA_LOCUS5297 |
| Bemisia tabaci | 58.37 | SEQ_NO_66 | XP_018909256 | LOC109038607 |
| Bemisia tabaci | 44.32 | SEQ_NO_66 | A0A9P0AGA5_BEMTA | BEMITA_LOCUS12291 |
| Bemisia tabaci | 44.32 | SEQ_NO_66 | A0A9P0F775_BEMTA | BEMITA_LOCUS11095 |
| Bemisia tabaci | 35.38 | SEQ_NO_66 | A0A9P0A9X4_BEMTA | BEMITA_LOCUS5296 |
| Bemisia tabaci | 36.45 | SEQ_NO_66 | CAH0383535 | empty |
| Bemisia tabaci | 36.45 | SEQ_NO_66 | A0A9P0A3L5_BEMTA | BEMITA_LOCUS2975 |
| Bemisia tabaci | 35.57 | SEQ_NO_66 | XP_018910318 | LOC109039338 |
| Arsenophonus endosymbiont | 46.19 | SEQ_NO_66 | WP_238146309 | empty |
| Bemisia tabaci | 44.35 | SEQ_NO_66 | A0A9P0ALQ3_BEMTA | BEMITA_LOCUS11821 |
| Bemisia tabaci | 25.83 | SEQ_NO_66 | XP_018899628 | LOC109032118 |
| Bemisia tabaci | 25.83 | SEQ_NO_66 | A0A9P0AK56_BEMTA | BEMITA_LOCUS11907 |
| Bemisia tabaci | 47.8 | SEQ_NO_66 | XP_018908149 | LOC109037773 |
| Bemisia tabaci | 34.94 | SEQ_NO_66 | XP_018912120 | LOC109040592 |
| Bemisia tabaci | 36.44 | SEQ_NO_66 | XP_018896584 | LOC109030199 |
| Bemisia tabaci | 75.73 | SEQ_NO_66 | AID65143 | empty |
| Bemisia tabaci | 75.73 | SEQ_NO_66 | A0A068FIK3_BEMTA | empty |
| Bemisia tabaci | 47.24 | SEQ_NO_66 | XP_018896539 | LOC109030162 |
| Bemisia tabaci | 51.03 | SEQ_NO_66 | XP_018910376 | LOC109039371 |
| Bemisia tabaci | 51.03 | SEQ_NO_66 | XP_018910377 | LOC109039371 |
| Bemisia tabaci | 51.03 | SEQ_NO_66 | XP_018910378 | LOC109039371 |
| Bemisia tabaci | 51.03 | SEQ_NO_66 | XP_018910379 | LOC109039371 |
| Bemisia tabaci | 30.8 | SEQ_NO_66 | XP_018899702 | LOC109032172 |
| Bemisia tabaci | 30.8 | SEQ_NO_66 | A0A9N9ZZ05_BEMTA | BEMITA_LOCUS315 |
| Bemisia tabaci | 45.1 | SEQ_NO_66 | XP_018913620 | LOC109041666 |
| Bemisia tabaci | 62 | SEQ_NO_66 | A0A9P0F189_BEMTA | BEMITA_LOCUS6630 |
| Bemisia tabaci | 40.82 | SEQ_NO_66 | A0A9P0F717_BEMTA | BEMITA_LOCUS10934 |
| Bemisia tabaci | 37.58 | SEQ_NO_66 | XP_018911278 | LOC109039987 |
| Bemisia tabaci | 48.21 | SEQ_NO_66 | XP_018911897 | LOC109040416 |
| Bemisia tabaci | 35.76 | SEQ_NO_66 | XP_018895605 | LOC109029562 |
| Bemisia tabaci | 42.4 | SEQ_NO_66 | XP_018907434 | LOC109037291 |
| Bemisia tabaci | 56.52 | SEQ_NO_66 | A0A9P0AJQ7_BEMTA | BEMITA_LOCUS11450 |
| Bemisia tabaci | 49.02 | SEQ_NO_66 | A0A9P0F2H8_BEMTA | BEMITA_LOCUS5293 |
| Bemisia tabaci | 42.98 | SEQ_NO_66 | A0A9P0F5Z0_BEMTA | BEMITA_LOCUS11822 |
| Bemisia tabaci | 40.18 | SEQ_NO_66 | CAH0389256 | empty |
| Bemisia tabaci | 40.18 | SEQ_NO_66 | A0A9P0AE01_BEMTA | BEMITA_LOCUS8103 |
| Bemisia tabaci | 82.93 | SEQ_NO_62 | XP_018912043 | LOC109040523 |
| Bemisia tabaci | 23.98 | SEQ_NO_5 | A0A9P0A559_BEMTA | BEMITA_LOCUS3504 |
| Bemisia tabaci | 23.98 | SEQ_NO_5 | XP_018901974 | LOC109033706 |
| Bemisia tabaci | 23.98 | SEQ_NO_5 | XP_018901973 | LOC109033706 |
| Bemisia tabaci | 30.92 | SEQ_NO_5 | XP_018895627 | LOC109029581 |
| Bemisia tabaci | 97.48 | SEQ_NO_9 | UHM21684 | empty |
| Bemisia tabaci | 97.48 | SEQ_NO_9 | UHM21685 | empty |
| Bemisia tabaci | 97.25 | SEQ_NO_9 | UHM21687 | empty |
| Bemisia tabaci | 97.25 | SEQ_NO_9 | XP_018917081 | LOC109044051 |
| Bemisia tabaci | 97.25 | SEQ_NO_9 | A0A9N9ZZA0_BEMTA | BEMITA_LOCUS2008 |
| Bemisia tabaci | 96.8 | SEQ_NO_9 | UHM21686 | empty |
| Bemisia tabaci | 96.57 | SEQ_NO_9 | UHM21682 | empty |
| Bemisia tabaci | 96.57 | SEQ_NO_9 | UHM21683 | empty |
| Bemisia tabaci | 98.01 | SEQ_NO_9 | UJH94051 | LysA |
| Bemisia tabaci | 94.98 | SEQ_NO_9 | XP_018916964 | LOC109043991 |
| Sodalis endosymbiont | 29.08 | SEQ_NO_9 | SNC58291 | lysA |
| Bemisia tabaci | 27.56 | SEQ_NO_9 | XP_018916434 | LOC109043619 |
| Bemisia tabaci | 27.56 | SEQ_NO_9 | A0A9P0A5P4_BEMTA | BEMITA_LOCUS3774 |
| Bemisia tabaci | 25.65 | SEQ_NO_9 | XP_018908759 | LOC109038228 |
| Bemisia tabaci | 25.36 | SEQ_NO_9 | A0A9P0AL47_BEMTA | BEMITA_LOCUS13374 |
| Bemisia tabaci | 24.78 | SEQ_NO_9 | XP_018908757 | LOC109038226 |
| Bemisia tabaci | 26.78 | SEQ_NO_9 | A0A9P0C8K4_BEMTA | BEMITA_LOCUS3774 |
| Candidatus Hamiltonella defensa | 21.5 | SEQ_NO_9 | ASX26863 | empty |
| (Bemisia tabaci) (Bemisia tabaci) | | | | |
| Candidatus Hamiltonella | 21.5 | SEQ_NO_9 | A0A249DZ94_9ENTR | speA; BA171_07640 |
| Arsenophonus endosymbiont | 23.64 | SEQ_NO_9 | WP_129108786 | speA |
| Candidatus Hamiltonella defensa | 26.55 | SEQ_NO_9 | ASX26586 | empty |
| (Bemisia tabaci) (Bemisia tabaci) | | | | |
| Candidatus Hamiltonella | 26.55 | SEQ_NO_9 | A0A249DYG1_9ENTR | BA171_05965 |
| Bemisia tabaci | 91.36 | SEQ_NO_13 | A0A9P0C5D3_BEMTA | BEMITA_LOCUS748 |
| Bemisia tabaci | 90.53 | SEQ_NO_13 | UHM21690 | empty |
| Bemisia tabaci | 90.12 | SEQ_NO_13 | UHM21688 | empty |
| Bemisia tabaci | 90.12 | SEQ_NO_13 | UHM21689 | empty |
| Bemisia tabaci | 90.12 | SEQ_NO_13 | UHM21691 | empty |
| Bemisia tabaci | 90.12 | SEQ_NO_13 | UHM21692 | empty |
| Bemisia tabaci | 89.71 | SEQ_NO_13 | UHM21693 | empty |
| Rickettsia sp (Bemisia tabaci) | 53.75 | SEQ_NO_13 | ASX28567 | empty |
| Rickettsia sp | 53.75 | SEQ_NO_13 | A0A249E610_9RICK | dapB; BA173_04970 |
| Bemisia tabaci | 87.23 | SEQ_NO_13 | XP_018912770 | LOC109041064 |
| Wolbachia endosymbiont | 36.5 | SEQ_NO_13 | AZU37066 | empty |
| Wolbachia endosymbiont | 36.5 | SEQ_NO_13 | WP_127463563 | dapB |
| Wolbachia endosymbiont | 36.5 | SEQ_NO_13 | A0A3T0GHK6_9RICK | dapB; BBB02_00115 |
| Wolbachia endosymbiont | 36.12 | SEQ_NO_13 | WP_108784544 | dapB |
| Sodalis endosymbiont | 33.08 | SEQ_NO_13 | SNC58563 | dapB |
| Arsenophonus endosymbiont | 32.7 | SEQ_NO_13 | WP_119712333 | dapB |
| Arsenophonus endosymbiont | 32.7 | SEQ_NO_13 | WP_129109115 | dapB |
| Candidatus Hamiltonella defensa | 32.58 | SEQ_NO_13 | ASX26814 | empty |
| (Bemisia tabaci) (Bemisia tabaci) | | | | |
| Candidatus Hamiltonella | 32.58 | SEQ_NO_13 | A0A249DZ50_9ENTR | dapB; BA171_07360 |
| Arsenophonus endosymbiont | 32.32 | SEQ_NO_13 | WP_181994678 | dapB |
| Candidatus Portiera | 42.62 | SEQ_NO_13 | AUI73062 | empty |
| Candidatus Portiera | 40.43 | SEQ_NO_13 | AFT80821 | empty |
| aleyrodidarum BT-B-HRs | | | | |
| Candidatus Portiera | 40.43 | SEQ_NO_13 | AFT80541 | empty |
| aleyrodidarum BT-QVLC | | | | |
| Bemisia tabaci | 99.51 | SEQ_NO_11 | XP_018899812 | LOC109032243 |
| Bemisia tabaci | 99.51 | SEQ_NO_11 | A0A9P0F5S9_BEMTA | BEMITA_LOCUS11621 |
| Bemisia tabaci | 99.17 | SEQ_NO_11 | XP_018899814 | LOC109032243 |
| Bemisia tabaci | 99.17 | SEQ_NO_11 | A0A9P0F5K8_BEMTA | BEMITA_LOCUS11621 |
| Bemisia tabaci | 28.77 | SEQ_NO_60 | XP_018900450 | LOC109032660 |
| Wolbachia endosymbiont | 24.61 | SEQ_NO_7 | WP_108784306 | purB |
| Arsenophonus endosymbiont | 24.67 | SEQ_NO_7 | WP_119711900 | aspA |
| Wolbachia endosymbiont | 23.66 | SEQ_NO_7 | AZU37331 | empty |
| Wolbachia endosymbiont | 23.66 | SEQ_NO_7 | WP_127463792 | purB |
| Wolbachia endosymbiont | 23.66 | SEQ_NO_7 | A0A3T0GIN6_9RICK | BBB02_01815 |
| Candidatus Hamiltonella defensa | 25.44 | SEQ_NO_7 | ASX26704 | empty |
| (Bemisia tabaci) (Bemisia tabaci) | | | | |
| Candidatus Hamiltonella | 25.44 | SEQ_NO_7 | A0A249DYT9_9ENTR | BA171_06655 |
| Arsenophonus endosymbiont | 23.75 | SEQ_NO_7 | WP_119711075 | fumC |
| Bemisia tabaci | 26.56 | SEQ_NO_7 | XP_018917343 | LOC109044214 |
| Bemisia tabaci | 26.56 | SEQ_NO_7 | A0A7S5HFZ5_BEMTA | BEMITA_LOCUS5487 |
| Wolbachia endosymbiont | 23.85 | SEQ_NO_7 | AZU37931 | empty |
| Wolbachia endosymbiont | 23.85 | SEQ_NO_7 | WP_127464267 | fumC |
| Wolbachia endosymbiont | 23.85 | SEQ_NO_7 | A0A3T0GKF6_9RICK | fumC; BBB02_05745 |
| Candidatus Hamiltonella defensa | 26.64 | SEQ_NO_7 | ASX26054 | empty |
| (Bemisia tabaci) (Bemisia tabaci) | | | | |
| Candidatus Hamiltonella | 26.64 | SEQ_NO_7 | A0A249DWZ0_9ENTR | fumC; BA171_02720 |
| Wolbachia endosymbiont | 23.43 | SEQ_NO_7 | WP_108784602 | fumC |
| Rickettsia sp (Bemisia tabaci) | 25.24 | SEQ_NO_7 | ASX28229 | empty |
| Rickettsia sp | 25.24 | SEQ_NO_7 | A0A249E3Q0_9RICK | fumC; BA173_05380 |
| Arsenophonus endosymbiont | 25.91 | SEQ_NO_7 | WP_181994326 | fumC |
| Arsenophonus endosymbiont | 26.6 | SEQ_NO_7 | WP_129108948 | fumC |
| Arsenophonus endosymbiont | 29.81 | SEQ_NO_7 | WP_181994813 | pnp |

TABLE 4
Target sequences in hemipteran species and accession numbers.

| | | Query Identifier- | | |
|---|---|---|---|---|
| | Align | Instant Application | | |
| Organism | % Id | Sequence Identifier | Identifier | Gene name |
| Bemisia tabaci | 91.57 | SEQ_NO_28 | CAH0387825 | empty |
| Bemisia tabaci | 91.57 | SEQ_NO_28 | A0A9P0A6Y6_BEMTA | BEMITA_LOCUS6793 |
| Bemisia tabaci | 91.51 | SEQ_NO_28 | XP_018898272 | LOC109031284 |
| Acyrthosiphon pisum (pea aphid) | 38.46 | SEQ_NO_28 | XP_029346266 | LOC100168860 |
| Homalodisca vitripennis (glassy- | 38.71 | SEQ_NO_28 | XP_046662415 | LOC124355349 |
| winged sharpshooter) | | | | |
| Cacopsylla melanoneura | 36.3 | SEQ_NO_28 | A0A8D9EL40_9HEMI | empty |
| Diaphorina citri (Asian citrus psyllid) | 36.36 | SEQ_NO_28 | QAA95898 | ABCA2 |
| Rhopalosiphum maidis (corn leaf | 37.98 | SEQ_NO_28 | XP_026811930 | LOC113553012 |
| aphid) | | | | |
| Rhopalosiphum maidis (corn leaf | 37.98 | SEQ_NO_28 | XP_026811914 | LOC113553012 |
| aphid) | | | | |
| Acyrthosiphon pisum (pea aphid) | 36.67 | SEQ_NO_28 | XP_016657976 | LOC100168860 |
| Melanaphis sacchari | 36.64 | SEQ_NO_28 | XP_025190795 | LOC112591254 |
| Diuraphis noxia (Russian wheat | 36.41 | SEQ_NO_28 | XP_015365481 | LOC107162874 |
| aphid) | | | | |
| Diuraphis noxia (Russian wheat | 36.33 | SEQ_NO_28 | XP_015365482 | LOC107162874 |
| aphid) | | | | |
| Melanaphis sacchari | 38.22 | SEQ_NO_28 | XP_025190796 | LOC112591254 |
| Schizaphis graminum | 37.01 | SEQ_NO_28 | A0A2S2P9P8_SCHGA | ABCA1_2; g.134090 |
| Aphis gossypii (cotton aphid) | 35.93 | SEQ_NO_28 | XP_027850520 | LOC114129866 |
| Diuraphis noxia (Russian wheat | 36.12 | SEQ_NO_28 | XP_015365483 | LOC107162874 |
| aphid) | | | | |
| Daktulosphaira vitifoliae (grape | 36.42 | SEQ_NO_28 | XP_050530373 | LOC126899455 |
| phylloxera) | | | | |
| Myzus persicae (green peach aphid) | 38.91 | SEQ_NO_28 | XP_022171731 | LOC111034705 |
| Adelges cooleyi (spruce gall adelgid) | 40.48 | SEQ_NO_28 | XP_050431317 | LOC126839910 |
| Sipha flava (yellow sugarcane aphid) | 38.47 | SEQ_NO_28 | XP_025419273 | LOC112689665 |
| Aphis glycines | 35.56 | SEQ_NO_28 | A0A6G0U0E1_APHGL | AGLY_003530 |
| Aphis gossypii (cotton aphid) | 32.92 | SEQ_NO_28 | CAH1731091 | empty |
| Graphocephala atropunctata | 81.74 | SEQ_NO_10 | A0A1B6M5T0_9HEMI | g.27510 |
| Cacopsylla melanoneura | 80.96 | SEQ_NO_10 | A0A8D8ZSV2_9HEMI | empty |
| Nilaparvata lugens (brown | 80.55 | SEQ_NO_10 | XP_039285067 | LOC111059194 |
| planthopper) | | | | |
| Homalodisca vitripennis (glassy- | 80.12 | SEQ_NO_10 | XP_046664126 | LOC124356891 |
| winged sharpshooter) | | | | |
| Sogatella furcifera (white-backed | 80.43 | SEQ_NO_10 | AFK64819 | empty |
| planthopper) | | | | |
| Clastoptera arizonana | 80.89 | SEQ_NO_10 | A0A1B6DMS5_9HEMI | g.22215 |
| Diuraphis noxia (Russian wheat | 80.28 | SEQ_NO_10 | XP_015365486 | LOC107162876 |
| aphid) | | | | |
| Myzus persicae (green peach aphid) | 80.28 | SEQ_NO_10 | XP_022171658 | LOC111034660 |
| Rhopalosiphum maidis (corn leaf | 80.84 | SEQ_NO_10 | XP_026807687 | LOC113550184 |
| aphid) | | | | |
| Aphis gossypii (cotton aphid) | 80.71 | SEQ_NO_10 | CAH1736844 | empty |
| Aphis glycines (soybean aphid) | 80.71 | SEQ_NO_10 | QII15890 | empty |
| Acyrthosiphon pisum (pea aphid) | 80.71 | SEQ_NO_10 | XP_001944221 | LOC100158991 |
| Sipha flava (yellow sugarcane aphid) | 79.62 | SEQ_NO_10 | XP_025425873 | LOC112694573 |
| Adelges cooleyi (spruce gall adelgid) | 80.2 | SEQ_NO_10 | XP_050440661 | LOC126845800 |
| Cinara cedri | 80.08 | SEQ_NO_10 | A0A5E4MMD6_9HEMI | CINCED_3A025750 |
| Daktulosphaira vitifoliae (grape | 80.08 | SEQ_NO_10 | XP_050535102 | LOC126902117 |
| phylloxera) | | | | |
| Diaphorina citri (Asian citrus psyllid) | 79.13 | SEQ_NO_10 | UUA81697 | TPS1 |
| Aphis gossypii (cotton aphid) | 77.32 | SEQ_NO_10 | CAH1732262 | empty |
| Diuraphis noxia (Russian wheat | 79.25 | SEQ_NO_10 | XP_015372030 | LOC107167490 |
| aphid) | | | | |
| Rhopalosiphum maidis (corn leaf | 77.03 | SEQ_NO_10 | XP_026815837 | LOC113555590 |
| aphid) | | | | |
| Melanaphis sacchari | 77.62 | SEQ_NO_10 | A0A2H8TZA4_9HEMI | tpsA_8 |
| Graphocephala atropunctata | 82.08 | SEQ_NO_10 | A0A1B6KCL4_9HEMI | g.27515 |
| Cacopsylla melanoneura | 82.5 | SEQ_NO_10 | A0A8D8XVI2_9HEMI | empty |
| Lygus hesperus | 66.28 | SEQ_NO_10 | A0A0K8TFI0_LYGHE | empty |
| Nesidiocoris tenuis | 65.79 | SEQ_NO_10 | BES87211 | empty |
| Sogatella furcifera (white-backed | 82.39 | SEQ_NO_10 | AFE85961 | empty |
| planthopper) | | | | |
| Nesidiocoris tenuis | 61.44 | SEQ_NO_10 | A0A6H5GXT8_9HEMI | NTEN_LOCUS11506 |
| Halyomorpha halys (brown | 60.59 | SEQ_NO_10 | XP_014279588 | LOC106682946 |
| marmorated stink bug) | | | | |
| Nezara viridula | 60.79 | SEQ_NO_10 | A0A9P0HP55_NEZVI | NEZAVI_LOCUS14331 |
| Homalodisca vitripennis (glassy- | 60.34 | SEQ_NO_10 | XP_046672371 | LOC124362167 |
| winged sharpshooter) | | | | |
| Nilaparvata lugens (brown | 61.28 | SEQ_NO_10 | AQT25641 | empty |
| planthopper) | | | | |
| Cimex lectularius (bed bug) | 64.34 | SEQ_NO_10 | XP_014255923 | LOC106670259 |
| Graphocephala atropunctata | 61.13 | SEQ_NO_10 | A0A1B6KBB1_9HEMI | g.9688 |
| Schizaphis graminum | 82.94 | SEQ_NO_10 | A0A2S2PET2_SCHGA | tpsA_2; g.132358 |
| Cacopsylla melanoneura | 62.39 | SEQ_NO_10 | A0A8D8S8X9_9HEMI | empty |
| Panstrongylus lignarius | 62.06 | SEQ_NO_10 | A0A224X7J0_9HEMI | empty |
| Lygus hesperus | 61.26 | SEQ_NO_10 | A0A0K8TDS6_LYGHE | empty |
| Sipha flava (yellow sugarcane aphid) | 60.51 | SEQ_NO_10 | XP_025425631 | LOC112694394 |
| Sipha flava | 60.51 | SEQ_NO_10 | A0A8B8GSL5_9HEMI | LOC112694394 |
| Sipha flava (yellow sugarcane aphid) | 60.51 | SEQ_NO_10 | XP_025425630 | LOC112694394 |
| Daktulosphaira vitifoliae (grape | 61.91 | SEQ_NO_10 | XP_050537028 | LOC126903096 |
| phylloxera) | | | | |
| Myzus persicae (green peach aphid) | 60.8 | SEQ_NO_10 | XP_022165508 | LOC111030364 |
| Lygus hesperus | 68.5 | SEQ_NO_10 | A0A146M502_LYGHE | tpsB; g.68162 |
| Rhodnius prolixus | 70.7 | SEQ_NO_10 | A0A4P6D7T2_RHOPR | empty |
| Nesidiocoris tenuis | 55.9 | SEQ_NO_10 | A0A6H5GNX7_9HEMI | NTEN_LOCUS11251 |
| Melanaphis sacchari | 80.2 | SEQ_NO_12 | XP_025203747 | LOC112600665 |
| Adelges cooleyi (spruce gall adelgid) | 77.9 | SEQ_NO_12 | XP_050440659 | LOC126845800 |
| Myzus persicae (green peach aphid) | 79.12 | SEQ_NO_12 | XP_022177708 | LOC111038784 |
| Acyrthosiphon pisum (pea aphid) | 80.54 | SEQ_NO_12 | XP_001945523 | LOC100166104 |
| Rhopalosiphum maidis (corn leaf | 78.55 | SEQ_NO_12 | XP_026815836 | LOC113555590 |
| aphid) | | | | |
| Lygus hesperus | 66.15 | SEQ_NO_12 | A0A146MF01_LYGHE | TPS1_2; TPS1_1; |
| | | | | g.68158; g.68160 |
| Graphocephala atropunctata | 83.28 | SEQ_NO_12 | A0A1B6MV38_9HEMI | g.27513 |
| Cinara cedri | 61.79 | SEQ_NO_12 | A0A5E4NAN4_9HEMI | CINCED_3A005873 |
| Cimex lectularius (bed bug) | 61.5 | SEQ_NO_12 | XP_014261998 | LOC106674065 |
| Diuraphis noxia (Russian wheat | 60.54 | SEQ_NO_12 | XP_015365045 | LOC107162595 |
| aphid) | | | | |
| Aphis glycines (soybean aphid) | 60.65 | SEQ_NO_12 | QII15889 | empty |
| Melanaphis sacchari | 60.39 | SEQ_NO_12 | XP_025193063 | LOC112593044 |
| Cuerna arida | 81.03 | SEQ_NO_12 | A0A1B6FEU9_9HEMI | g.36271 |
| Sogatella furcifera | 60.58 | SEQ_NO_12 | A0A6G6CSW6_SOGFU | empty |
| Clastoptera arizonana | 73.92 | SEQ_NO_18 | A0A1B6CPT6_9HEMI | g.20147 |
| Clastoptera arizonana | 75 | SEQ_NO_18 | A0A1B6DXY4_9HEMI | g.20141 |
| Rhodnius prolixus | 70.22 | SEQ_NO_18 | R4FNV2_RHOPR | empty |
| Cuerna arida | 73.77 | SEQ_NO_18 | A0A1B6EKX9_9HEMI | g.18386 |
| Laodelphax striatellus | 73.57 | SEQ_NO_18 | A0A482WWD3_LAOST | LSTR_LSTR005429 |
| Cuerna arida | 74.3 | SEQ_NO_18 | A0A1B6GT97_9HEMI | g.18390 |
| Cacopsylla melanoneura | 74.4 | SEQ_NO_18 | A0A8D8TNG5_9HEMI | empty |
| Graphocephala atropunctata | 72.89 | SEQ_NO_18 | A0A1B6MUL7_9HEMI | g.14637, g.14638 |
| Panstrongylus megistus | 73.21 | SEQ_NO_18 | A0A069DZ74_9HEMI | empty |
| Homalodisca vitripennis (glassy- | 73.51 | SEQ_NO_18 | XP_046670040 | LOC124360446 |
| winged sharpshooter) | | | | |
| Daktulosphaira vitifoliae (grape | 69.57 | SEQ_NO_18 | XP_050544717 | LOC126907457 |
| phylloxera) | | | | |
| Aphis gossypii (cotton aphid) | 72.67 | SEQ_NO_18 | CAH1726762 | empty |
| Sipha flava (yellow sugarcane aphid) | 72.76 | SEQ_NO_18 | XP_025408719 | LOC112682362 |
| Sipha flava (yellow sugarcane aphid) | 72.85 | SEQ_NO_18 | XP_025408720 | LOC112682362 |
| Diuraphis noxia (Russian wheat | 72.46 | SEQ_NO_18 | XP_015371655 | LOC107167192 |
| aphid) | | | | |
| Schizaphis graminum | 72.17 | SEQ_NO_18 | A0A2S2N7R9_SCHGA | UGP2_1; g.42221 |
| Rhopalosiphum maidis (corn leaf | 71.97 | SEQ_NO_18 | XP_026806423 | LOC113549361 |
| aphid) | | | | |
| Aphis craccivora | 72.48 | SEQ_NO_20 | A0A6G0ZEI4_APHCR | FWK35_00000855 |
| Cinara cedri | 72.62 | SEQ_NO_20 | A0A5E4M1P7_9HEMI | CINCED_3A005302 |
| Aphis gossypii (cotton aphid) | 72.76 | SEQ_NO_20 | XP_027836611 | LOC114119298 |
| Diuraphis noxia (Russian wheat | 72.37 | SEQ_NO_20 | XP_015371727 | LOC107167192 |
| aphid) | | | | |
| Essigella californica | 72.22 | SEQ_NO_20 | QBH73770 | empty |
| Adelges cooleyi (spruce gall adelgid) | 72.51 | SEQ_NO_20 | XP_050421516 | LOC126833951 |
| Melanaphis sacchari | 72.17 | SEQ_NO_20 | XP_025200047 | LOC112597979 |
| Myzus persicae (green peach aphid) | 71.97 | SEQ_NO_20 | XP_022181825 | LOC111041733 |
| Acyrthosiphon pisum (pea aphid) | 71.97 | SEQ_NO_20 | NP_001153815 | LOC100160720 |
| Adelges cooleyi (spruce gall adelgid) | 72.4 | SEQ_NO_20 | XP_050431133 | LOC126839768 |
| Melanaphis sacchari | 72.26 | SEQ_NO_20 | XP_025200045 | LOC112597979 |
| Acyrthosiphon pisum (pea aphid) | 72.06 | SEQ_NO_20 | NP_001153814 | LOC100160720 |
| Myzus persicae (green peach aphid) | 72.06 | SEQ_NO_20 | XP_022181818 | LOC111041733 |
| Rhopalosiphum maidis (corn leaf | 72.06 | SEQ_NO_20 | XP_026806422 | LOC113549361 |
| aphid) | | | | |
| Lygus hesperus | 70.75 | SEQ_NO_20 | A0A0K8SDZ6_LYGHE | empty |
| Cimex lectularius (bed bug) | 70.36 | SEQ_NO_20 | XP_014251467 | LOC106667804 |
| Triatoma infestans | 76.09 | SEQ_NO_20 | A0A170XLE0_TRIIF | empty |
| Riptortus pedestris | 70.56 | SEQ_NO_20 | BAN21402 | empty |
| Nilaparvata lugens (brown | 68.39 | SEQ_NO_20 | XP_039288389 | LOC111054366 |
| planthopper) | | | | |
| Halyomorpha halys (brown | 67.94 | SEQ_NO_20 | XP_014282356 | LOC106684659 |
| marmorated stink bug) | | | | |
| Diaphorina citri (Asian citrus psyllid) | 73.26 | SEQ_NO_20 | XP_017302584 | LOC103516542 |
| Cacopsylla chinensis (pear psyllid) | 62.28 | SEQ_NO_14 | WPA94603 | empty |
| Cacopsylla melanoneura | 62.42 | SEQ_NO_14 | A0A8D8LFK4_9HEMI | empty |
| Homalodisca liturata | 58.51 | SEQ_NO_14 | A0A1B6HI11_9HEMI | g.50825 |
| Homalodisca vitripennis (glassy- | 58.3 | SEQ_NO_14 | XP_046668033 | LOC124359384 |
| winged sharpshooter) | | | | |
| Nezara viridula (southern green stink | 60.18 | SEQ_NO_14 | CAH1407806 | empty |
| bug) | | | | |
| Graphocephala atropunctata | 57.45 | SEQ_NO_14 | A0A1B6MP98_9HEMI | g.18937 |
| Halyomorpha halys (brown | 59.51 | SEQ_NO_14 | XP_014282722 | LOC106684904 |
| marmorated stink bug) | | | | |
| Nilaparvata lugens (brown | 56.89 | SEQ_NO_14 | XP_022184109 | LOC111043460 |
| planthopper) | | | | |
| Panstrongylus lignarius | 57.37 | SEQ_NO_14 | A0A224XCI1_9HEMI | empty |
| Rhodnius neglectus | 55.38 | SEQ_NO_14 | A0A0P4VIK7_9HEMI | empty |
| Rhodnius prolixus | 56.92 | SEQ_NO_14 | T1HIT8_RHOPR | empty |
| Triatoma infestans | 58.49 | SEQ_NO_14 | A0A161N3T0_TRIIF | empty |
| Lygus hesperus | 52.75 | SEQ_NO_14 | A0A0A9Z2D5_LYGHE | Hex-t2_6; Hex-t2_7; |
| | | | | CM83_45220; g.43572 |
| Nesidiocoris tenuis | 51.61 | SEQ_NO_14 | BES87692 | empty |
| Apolygus lucorum | 52.31 | SEQ_NO_14 | A0A8S9XHX3_APOLU | GE061_017061 |
| Homalodisca liturata | 58.72 | SEQ_NO_16 | A0A1B6I221_9HEMI | g.50823, g.50828 |
| Clastoptera arizonana | 59.56 | SEQ_NO_16 | A0A1B6CBM9_9HEMI | g.3554 |
| Laodelphax striatellus | 56.83 | SEQ_NO_16 | A0A482X1R5_LAOST | LSTR_LSTR001047 |
| Triatoma infestans | 56.7 | SEQ_NO_16 | A0A023EZW9_TRIIF | empty |
| Cacopsylla melanoneura | 56.14 | SEQ_NO_24 | A0A8D8QSP7_9HEMI | empty |
| Diaphorina citri (Asian citrus psyllid) | 56.16 | SEQ_NO_24 | QAA95906 | ABCC1 |
| Nilaparvata lugens (brown | 55.98 | SEQ_NO_24 | XP_039283753 | LOC111055319 |
| planthopper) | | | | |
| Sipha flava (yellow sugarcane aphid) | 56.24 | SEQ_NO_24 | XP_025410477 | LOC112683596 |
| Melanaphis sacchari | 55.75 | SEQ_NO_24 | XP_025201051 | LOC112598699 |
| Sipha flava (yellow sugarcane aphid) | 55.65 | SEQ_NO_24 | XP_025410476 | LOC112683596 |
| Cinara cedri | 55.45 | SEQ_NO_24 | A0A5E4NJ82_9HEMI | CINCED_3A019450 |
| Nilaparvata lugens (brown | 55.13 | SEQ_NO_24 | XP_039283751 | LOC111055319 |
| planthopper) | | | | |
| Rhopalosiphum padi (bird cherry-oat | 55.39 | SEQ_NO_24 | AMD40296 | ABCC1 |
| aphid) | | | | |
| Rhopalosiphum maidis (corn leaf | 55.26 | SEQ_NO_24 | XP_026813138 | LOC113553801 |
| aphid) | | | | |
| Melanaphis sacchari | 55.16 | SEQ_NO_24 | XP_025201032 | LOC112598699 |
| Halyomorpha halys (brown | 55.23 | SEQ_NO_24 | XP_014276422 | LOC106680913 |
| marmorated stink bug) | | | | |
| Nezara viridula | 55.2 | SEQ_NO_24 | A0A9P0MNW7_NEZVI | NEZAVI_LOCUS10814 |
| Aphis gossypii (cotton aphid) | 55.03 | SEQ_NO_24 | XP_027847517 | LOC114127472 |
| Acyrthosiphon pisum (pea aphid) | 55.07 | SEQ_NO_24 | XP_008180937 | LOC100167620 |
| Adelges cooleyi (spruce gall adelgid) | 54.86 | SEQ_NO_24 | XP_050432450 | LOC126840625 |
| Laodelphax striatellus (small brown | 54.65 | SEQ_NO_24 | AIN44102 | ABCC1 |
| planthopper) | | | | |
| Laodelphax striatellus | 54.65 | SEQ_NO_24 | A0A161ANL7_LAOST | ABCC1 |
| Daktulosphaira vitifoliae (grape | 54.9 | SEQ_NO_24 | XP_050541856 | LOC126905820 |
| phylloxera) | | | | |
| Rhopalosiphum maidis (corn leaf | 54.66 | SEQ_NO_24 | XP_026813137 | LOC113553801 |
| aphid) | | | | |
| Myzus persicae (green peach aphid) | 53.69 | SEQ_NO_24 | XP_022169430 | LOC111033129 |
| Triatoma infestans | 52.88 | SEQ_NO_24 | A0A023F4D8_TRIIF | empty |
| Panstrongylus megistus | 52.75 | SEQ_NO_24 | A0A069DZT4_9HEMI | empty |
| Homalodisca vitripennis (glassy- | 52.69 | SEQ_NO_24 | XP_046664340 | LOC124357012 |
| winged sharpshooter) | | | | |
| Diuraphis noxia (Russian wheat | 53.26 | SEQ_NO_24 | XP_015366208 | LOC107163343 |
| aphid) | | | | |
| Nezara viridula (southern green stink | 50.92 | SEQ_NO_24 | CAH1406917 | empty |
| bug) | | | | |
| Cuerna arida | 48.09 | SEQ_NO_24 | A0A1B6G2H4_9HEMI | g.43462, g.43465, g.43468 |
| Aphis gossypii | 52.89 | SEQ_NO_24 | A0A9P0ISC1_APHGO | APHIGO LOCUS2380 |
| Diaphorina citri (Asian citrus psyllid) | 48.76 | SEQ_NO_24 | QER78494 | ABCC4 |
| Clastoptera arizonana | 56.06 | SEQ_NO_22 | A0A1B6E1F3_9HEMI | g.30401, g.30408, g.30420 |
| Cacopsylla melanoneura | 55.19 | SEQ_NO_22 | A0A8D8XP03_9HEMI | empty |
| Cinara cedri | 54.79 | SEQ_NO_22 | A0A5E4NFP1_9HEMI | CINCED_3A019450 |
| Aphis gossypii | 54.9 | SEQ_NO_22 | A0A9P0IRES_APHGO | APHIGO_LOCUS2380 |
| Cimex lectularius (bed bug) | 54.82 | SEQ_NO_22 | XP_014250127 | LOC106667036 |
| Aphis gossypii (cotton aphid) | 54.5 | SEQ_NO_22 | XP_027847515 | LOC114127472 |
| Myzus persicae (green peach aphid) | 53.69 | SEQ_NO_22 | XP_022169423 | LOC111033129 |
| Homalodisca vitripennis (glassy- | 51.62 | SEQ_NO_22 | XP_046660547 | LOC124354263 |
| winged sharpshooter) | | | | |
| Panstrongylus lignarius | 53.42 | SEQ_NO_22 | A0A224X9P4_9HEMI | empty |
| Rhodnius prolixus | 59.06 | SEQ_NO_22 | T1HX40_RHOPR | empty |
| Trialeurodes vaporariorum | 58.25 | SEQ_NO_34 | QPA18374 | UGT352P4 |
| (greenhouse whitefly) | | | | |
| Trialeurodes vaporariorum | 57.25 | SEQ_NO_34 | QPA18388 | UGT352P3 |
| (greenhouse whitefly) | | | | |
| Trialeurodes vaporariorum | 56.76 | SEQ_NO_34 | QPA18408 | UGT352P2 |
| (greenhouse whitefly) | | | | |
| Trialeurodes vaporariorum | 55.05 | SEQ_NO_34 | QPA18376 | UGT352P8 |
| (greenhouse whitefly) | | | | |
| Trialeurodes vaporariorum | 52.96 | SEQ_NO_34 | QPA18373 | UGT352P6 |
| (greenhouse whitefly) | | | | |
| Trialeurodes vaporariorum | 53.57 | SEQ_NO_34 | QPA18401 | UGT352S1 |
| (greenhouse whitefly) | | | | |
| Trialeurodes vaporariorum | 52.64 | SEQ_NO_34 | QPA18379 | UGT352T1 |
| (greenhouse whitefly) | | | | |
| Trialeurodes vaporariorum | 52.16 | SEQ_NO_34 | QPA18380 | UGT352P7 |
| (greenhouse whitefly) | | | | |
| Trialeurodes vaporariorum | 52.65 | SEQ_NO_34 | QPA18381 | UGT352P5 |
| (greenhouse whitefly) | | | | |
| Trialeurodes vaporariorum | 51.13 | SEQ_NO_34 | QPA18378 | UGT352R1 |
| (greenhouse whitefly) | | | | |
| Trialeurodes vaporariorum | 50.57 | SEQ_NO_34 | QPA18383 | UGT352U1 |
| (greenhouse whitefly) | | | | |
| Trialeurodes vaporariorum | 56.58 | SEQ_NO_34 | QPA18393 | UGT352P1 |
| (greenhouse whitefly) | | | | |
| Trialeurodes vaporariorum | 50.64 | SEQ_NO_34 | QPA18391 | UGT352V1 |
| (greenhouse whitefly) | | | | |
| Trialeurodes vaporariorum | 59.46 | SEQ_NO_34 | QPA18375 | UGT352Q1 |
| (greenhouse whitefly) | | | | |
| Trialeurodes vaporariorum | 41.92 | SEQ_NO_34 | A0A873P516_TRIVP | UGT358B1 |
| Laodelphax striatellus | 36.7 | SEQ_NO_34 | A0A482XBF2_LAOST | LSTR_LSTR001426 |
| Cacopsylla melanoneura | 35.99 | SEQ_NO_34 | A0A8D9A7V0_9HEMI | empty |
| Nilaparvata lugens (brown | 36 | SEQ_NO_34 | XP_039292305 | LOC111045820 |
| planthopper) | | | | |
| Trialeurodes vaporariorum | 35.59 | SEQ_NO_34 | QPA18409 | UGT387A1 |
| (greenhouse whitefly) | | | | |
| Trialeurodes vaporariorum | 35.59 | SEQ_NO_34 | A0A873P549_TRIVP | UGT387A1 |
| Nilaparvata lugens (brown | 35.81 | SEQ_NO_34 | XP_039292306 | LOC111045820 |
| planthopper) | | | | |
| Diaphorina citri (Asian citrus psyllid) | 34.44 | SEQ_NO_34 | QBQ34577 | UGT381B1 |
| Diaphorina citri (Asian citrus psyllid) | 34.87 | SEQ_NO_34 | QBQ34576 | UGT381A2 |
| Homalodisca vitripennis (glassy- | 32.6 | SEQ_NO_34 | XP_046661735 | LOC124354950 |
| winged sharpshooter) | | | | |
| Clastoptera arizonana | 32.9 | SEQ_NO_34 | A0A1B6DQ16_9HEMI | g.7472, g.7473 |
| Melanaphis sacchari | 31.17 | SEQ_NO_34 | XP_025195300 | LOC112594625 |
| Halyomorpha halys (brown | 31.39 | SEQ_NO_34 | XP_014292889 | LOC106691584 |
| marmorated stink bug) | | | | |
| Nezara viridula (southern green stink | 32.12 | SEQ_NO_34 | CAH1398448 | empty |
| bug) | | | | |
| Dactylopius coccus | 31.03 | SEQ_NO_34 | ATL15304 | empty |
| Trialeurodes vaporariorum | 30.9 | SEQ_NO_34 | QPA18405 | UGT355E1 |
| (greenhouse whitefly) | | | | |
| Trialeurodes vaporariorum | 52.05 | SEQ_NO_36 | A0A873P514_TRIVP | UGT352P7 |
| Cacopsylla melanoneura | 37.43 | SEQ_NO_36 | A0A8D8YUM3_9HEMI | empty |
| Laodelphax striatellus | 35.15 | SEQ_NO_36 | A0A482WMJ0_LAOST | LSTR_LSTR011762 |
| Laodelphax striatellus | 31.75 | SEQ_NO_36 | A0A482X3M6_LAOST | LSTR_LSTR000394 |
| Homalodisca liturata | 30.87 | SEQ_NO_36 | A0A1B6IH48_9HEMI | g.51432 |
| Graphocephala atropunctata | 30.67 | SEQ_NO_36 | A0A1B6MM02_9HEMI | g.28230 |
| Cuerna arida | 30.56 | SEQ_NO_36 | A0A1B6H4G8_9HEMI | g.25595 |
| Nilaparvata lugens (brown | 36.05 | SEQ_NO_32 | XP_039292341 | LOC111044292 |
| planthopper) | | | | |
| Nilaparvata lugens (brown | 36.24 | SEQ_NO_32 | QVG59857 | UGT386B2 |
| planthopper) | | | | |
| Cacopsylla melanoneura | 34.18 | SEQ_NO_30 | A0A8D8V7F4_9HEMI | empty |
| Laodelphax striatellus | 35.28 | SEQ_NO_30 | A0A482WTF3_LAOST | LSTR_LSTR010663 |
| Nilaparvata lugens (brown | 35.2 | SEQ_NO_30 | XP_039293137 | LOC111053419 |
| planthopper) | | | | |
| Diaphorina citri (Asian citrus psyllid) | 32.9 | SEQ_NO_30 | XP_026676214 | LOC103505006 |
| Trialeurodes vaporariorum | 30.53 | SEQ_NO_30 | QPA18398 | UGT359B1 |
| (greenhouse whitefly) | | | | |
| Homalodisca vitripennis (glassy- | 31.85 | SEQ_NO_30 | XP_046659562 | LOC124353660 |
| winged sharpshooter) | | | | |
| Cuerna arida | 32.03 | SEQ_NO_30 | A0A1B6H0K8_9HEMI | g.25593 |
| Halyomorpha halys (brown | 29.37 | SEQ_NO_30 | XP_014286418 | LOC106687194 |
| marmorated stink bug) | | | | |
| Daktulosphaira vitifoliae (grape | 30.8 | SEQ_NO_30 | XP_050522156 | LOC126894889 |
| phylloxera) | | | | |
| Halyomorpha halys (brown | 30.56 | SEQ_NO_30 | XP_014286412 | LOC106687193 |
| marmorated stink bug) | | | | |
| Nilaparvata lugens (brown | 30.5 | SEQ_NO_30 | XP_039292857 | LOC111053971 |
| planthopper) | | | | |
| Rhopalosiphum padi (bird cherry-oat | 29.7 | SEQ_NO_30 | WMV02799 | empty |
| aphid) | | | | |
| Halyomorpha halys (brown | 30.37 | SEQ_NO_30 | XP_014286417 | LOC106687193 |
| marmorated stink bug) | | | | |
| Homalodisca liturata | 34.43 | SEQ_NO_40 | A0A1B6IW17_9HEMI | g.51434 |
| Cacopsylla melanoneura | 36.03 | SEQ_NO_40 | A0A8D8U3N7_9HEMI | empty |
| Graphocephala atropunctata | 34.01 | SEQ_NO_40 | A0A1B6MBE9_9HEMI | g.41505 |
| Nilaparvata lugens (brown | 34.34 | SEQ_NO_40 | QVG59856 | UGT386A2 |
| planthopper) | | | | |
| Cuerna arida | 33.52 | SEQ_NO_40 | A0A1B6FAC1_9HEMI | g.31647 |
| Cacopsylla melanoneura | 34.58 | SEQ_NO_38 | A0A8D9A7K3_9HEMI | empty |
| Cacopsylla melanoneura | 35.27 | SEQ_NO_38 | A0A8D9AUF6_9HEMI | empty |
| Nilaparvata lugens (brown | 33.21 | SEQ_NO_38 | QVG59859 | UGT386D2 |
| planthopper) | | | | |
| Nilaparvata lugens (brown | 34.42 | SEQ_NO_38 | XP_039287572 | LOC111059173 |
| planthopper) | | | | |
| Diaphorina citri (Asian citrus psyllid) | 32.48 | SEQ_NO_38 | QBQ34567 | UGT374A2 |
| Homalodisca liturata | 33.71 | SEQ_NO_38 | A0A1B6ITG3_9HEMI | g.51438 |
| Homalodisca vitripennis (glassy- | 32.24 | SEQ_NO_38 | XP_046659554 | LOC124353657 |
| winged sharpshooter) | | | | |
| Cacopsylla melanoneura | 34.37 | SEQ_NO_38 | A0A8D8M379_9HEMI | empty |
| Graphocephala atropunctata | 32.95 | SEQ_NO_38 | A0A1B6MJE9_9HEMI | g.28232 |
| Trialeurodes vaporariorum | 33.33 | SEQ_NO_38 | QPA18377 | UGT360A5 |
| (greenhouse whitefly) | | | | |
| Homalodisca vitripennis (glassy- | 38.4 | SEQ_NO_26 | XP_046662414 | LOC124355349 |
| winged sharpshooter) | | | | |
| Cinara cedri | 36.9 | SEQ_NO_26 | A0A5E4MWX8_9HEMI | CINCED_3A007897 |
| Diuraphis noxia (Russian wheat | 36.34 | SEQ_NO_26 | XP_015365480 | LOC107162874 |
| aphid) | | | | |
| Adelges cooleyi (spruce gall adelgid) | 36.07 | SEQ_NO_26 | XP_050431314 | LOC126839910 |
| Cacopsylla melanoneura | 31.85 | SEQ_NO_26 | A0A8D8SAH2_9HEMI | empty |
| Diaphorina citri (Asian citrus psyllid) | 56.39 | SEQ_NO_26 | QER78485 | ABCA3 |
| Rhopalosiphum maidis (corn leaf | 41.33 | SEQ_NO_2 | XP_026817841 | LOC113556851 |
| aphid) | | | | |
| Myzus persicae (green peach aphid) | 40.08 | SEQ_NO_2 | XP_022175298 | LOC111037207 |
| Acyrthosiphon pisum (pea aphid) | 42.32 | SEQ_NO_2 | XP_001943832 | LOC100159435 |
| Melanaphis sacchari | 39.88 | SEQ_NO_2 | XP_025205275 | LOC112601732 |
| Aphis gossypii (cotton aphid) | 39.44 | SEQ_NO_2 | XP_050055621 | LOC114127088 |
| Aphis gossypii (cotton aphid) | 39.44 | SEQ_NO_2 | CAH1723921 | empty |
| Diaphorina citri (Asian citrus psyllid) | 37.67 | SEQ_NO_2 | XP_008473869 | LOC103510942 |
| Sipha flava (yellow sugarcane aphid) | 38.51 | SEQ_NO_2 | XP_025415984 | LOC112687477 |
| Cimex lectularius (bed bug) | 38.28 | SEQ_NO_2 | XP_014254493 | LOC112126112 |
| Adelges cooleyi (spruce gall adelgid) | 37.84 | SEQ_NO_2 | XP_050427895 | LOC126837930 |
| Diuraphis noxia (Russian wheat | 40.92 | SEQ_NO_2 | XP_015364700 | LOC107162356 |
| aphid) | | | | |
| Laodelphax striatellus | 37.07 | SEQ_NO_2 | A0A482X6Z7_LAOST | LSTR_LSTR011630 |
| Homalodisca vitripennis (glassy- | 39.71 | SEQ_NO_2 | XP_046672093 | LOC124362004 |
| winged sharpshooter) | | | | |
| Aphis craccivora | 40.04 | SEQ_NO_2 | A0A6GOZQA0_APHCR | FWK35_00001432 |
| Cuerna arida | 39.15 | SEQ_NO_2 | A0A1B6GQD0_9HEMI | g.11553 |
| Nilaparvata lugens (brown | 38.25 | SEQ_NO_2 | BAI83421 | Nlst7 |
| planthopper) | | | | |
| Cinara cedri | 38.73 | SEQ_NO_2 | A0A5E4N692_9HEMI | CINCED_3A010887 |
| Halyomorpha halys (brown | 37.03 | SEQ_NO_2 | XP_014285743 | LOC106686762 |
| marmorated stink bug) | | | | |
| Melanaphis sacchari | 34.32 | SEQ_NO_2 | XP_025190759 | LOC112591219 |
| Homalodisca liturata | 37.88 | SEQ_NO_2 | A0A1B6J4W6_9HEMI | g.23486 |
| Cacopsylla melanoneura | 35.66 | SEQ_NO_2 | A0A8D8W7D5_9HEMI | empty |
| Aphis gossypii (cotton aphid) | 37.58 | SEQ_NO_2 | CAH1724170 | empty |
| Diaphorina citri (Asian citrus psyllid) | 34.53 | SEQ_NO_2 | XP_008475503 | LOC103512517 |
| Clastoptera arizonana | 39.14 | SEQ_NO_2 | A0A1B6CRC6_9HEMI | g.19952, g.19953 |
| Rhopalosiphum maidis (corn leaf | 33.96 | SEQ_NO_2 | XP_026812036 | LOC113553090 |
| aphid) | | | | |
| Acyrthosiphon pisum (pea aphid) | 34.35 | SEQ_NO_2 | XP_008180278 | LOC100160681 |
| Homalodisca vitripennis (glassy- | 34.94 | SEQ_NO_2 | XP_046669547 | LOC124360194 |
| winged sharpshooter) | | | | |
| Clastoptera arizonana | 36.5 | SEQ_NO_4 | A0A1B6BX46_9HEMI | g.8053 |
| Graphocephala atropunctata | 38.42 | SEQ_NO_4 | A0A1B6M0V4_9HEMI | g.16986 |
| Cacopsylla melanoneura | 38.14 | SEQ_NO_4 | A0A8D8UXV6_9HEMI | empty |
| Clastoptera arizonana | 38.62 | SEQ_NO_4 | A0A1B6E8P1_9HEMI | g.36302 |
| Sipha flava (yellow sugarcane aphid) | 36.65 | SEQ_NO_4 | XP_025416371 | LOC112687711 |
| Nilaparvata lugens (brown | 36.22 | SEQ_NO_4 | XP_022195529 | LOC111053007 |
| planthopper) | | | | |
| Halyomorpha halys (brown | 37.3 | SEQ_NO_4 | XP_014273037 | LOC106678795 |
| marmorated stink bug) | | | | |
| Schizaphis graminum | 40 | SEQ_NO_4 | A0A2S2NHE6_SCHGA | Tret1_1; g.74357 |
| Adelges cooleyi (spruce gall adelgid) | 36.12 | SEQ_NO_4 | XP_050427896 | LOC126837930 |
| Nilaparvata lugens (brown | 37.37 | SEQ_NO_4 | XP_039290544 | LOC111053325 |
| planthopper) | | | | |
| Cacopsylla melanoneura | 33.58 | SEQ_NO_4 | A0A8D8RC31_9HEMI | empty |
| Cimex lectularius (bed bug) | 37.89 | SEQ_NO_4 | XP_024085216 | LOC106669487 |
| Cimex lectularius (bed bug) | 37.89 | SEQ_NO_4 | XP_024085215 | LOC106669487 |
| Daktulosphaira vitifoliae (grape | 37.01 | SEQ_NO_4 | XP_050533259 | LOC126901082 |
| phylloxera) | | | | |
| Aphis glycines | 33.96 | SEQ_NO_4 | A0A6G0T978_APHGL | AGLY_012868 |
| Aphis gossypii (cotton aphid) | 33.96 | SEQ_NO_4 | XP_027844595 | LOC114125234 |
| Cuerna arida | 36.36 | SEQ_NO_4 | A0A1B6EU41_9HEMI | g.34403 |
| Cinara cedri | 33.58 | SEQ_NO_4 | A0A5E4MGY0_9HEMI | CINCED_3A019556 |
| Cuerna arida | 35.74 | SEQ_NO_4 | A0A1B6FHS8_9HEMI | g.14775 |
| Myzus persicae (green peach aphid) | 34.77 | SEQ_NO_4 | XP_022182954 | LOC111042603 |
| Nesidiocoris tenuis | 37.55 | SEQ_NO_6 | BET03142 | empty |
| Nesidiocoris tenuis | 36.99 | SEQ_NO_6 | BES90083 | empty |
| Apolygus lucorum | 37.36 | SEQ_NO_6 | A0A6A4KBM7_APOLU | GE061_000355 |
| Laodelphax striatellus | 37.22 | SEQ_NO_6 | A0A482X0H7_LAOST | LSTR_LSTR012604 |
| Cimex lectularius (bed bug) | 37.22 | SEQ_NO_6 | XP_014259104 | LOC106672297 |
| Homalodisca vitripennis (glassy- | 36.66 | SEQ_NO_6 | XP_046689255 | LOC124375191 |
| winged sharpshooter) | | | | |
| Clastoptera arizonana | 36.52 | SEQ_NO_6 | A0A1B6C0L1_9HEMI | g.21225 |
| Laodelphax striatellus | 37 | SEQ_NO_6 | A0A482X940_LAOST | LSTR_LSTR011129 |
| Nilaparvata lugens | 37 | SEQ_NO_6 | A0AOA8J8J2_NILLU | NIST |
| Nilaparvata lugens (brown | 37 | SEQ_NO_6 | XP_039278095 | LOC111062486 |
| planthopper) | | | | |
| Halyomorpha halys (brown | 36.84 | SEQ_NO_6 | XP_014285372 | LOC106686528 |
| marmorated stink bug) | | | | |
| Lygus hesperus | 36.92 | SEQ_NO_6 | A0A0A9X1R8_LYGHE | Tret1_98; Tret1_125; |
| | | | | Tret1_30; Tret1_5; |
| | | | | CM83_38142; |
| | | | | CM83_38145; g.47738; |
| | | | | g.47741 |
| Apolygus lucorum | 37.05 | SEQ_NO_6 | A0A8S9Y7F1_APOLU | GE061_001561 |
| Nezara viridula (southern green stink | 36.18 | SEQ_NO_6 | CAH1392675 | empty |
| bug) | | | | |
| Homalodisca liturata | 36.59 | SEQ_NO_6 | A0A1B6HT34_9HEMI | g.15697 |
| Rhodnius prolixus | 36.36 | SEQ_NO_6 | R4G3D5_RHOPR | empty |
| Cuerna arida | 34.75 | SEQ_NO_6 | A0A1B6FN52_9HEMI | g.29404 |
| Halyomorpha halys (brown | 36.38 | SEQ_NO_6 | XP_014282541 | LOC106684783 |
| marmorated stink bug) | | | | |
| Graphocephala atropunctata | 36.38 | SEQ_NO_6 | A0A1B6M474_9HEMI | g.11079 |
| Nilaparvata lugens (brown | 35.36 | SEQ_NO_6 | XP_022202398 | LOC111059089 |
| planthopper) | | | | |
| Nilaparvata lugens | 35.14 | SEQ_NO_6 | A0A0A8J7G8_NILLU | NIST |
| Panstrongylus lignarius | 35.94 | SEQ_NO_6 | A0A224XP17_9HEMI | empty |
| Nesidiocoris tenuis | 35.71 | SEQ_NO_6 | BES89866 | empty |
| Cimex lectularius (bed bug) | 34.97 | SEQ_NO_6 | XP_014242280 | LOC106662596 |
| Nezara viridula | 34.82 | SEQ_NO_6 | A0A9P0HMH5_NEZVI | NEZAVI_LOCUS12843 |
| Graphocephala atropunctata | 34.51 | SEQ_NO_6 | A0A1B6MPN8_9HEMI | g.22346 |
| Homalodisca vitripennis (glassy- | 33.41 | SEQ_NO_6 | XP_046669427 | LOC124360126 |
| winged sharpshooter) | | | | |
| Homalodisca liturata | 33.41 | SEQ_NO_6 | A0A1B6HXW2_9HEMI | g.11274 |
| Clastoptera arizonana | 34.22 | SEQ_NO_6 | A0A1B6C7Q8_9HEMI | g.17924 |
| Rhopalosiphum maidis (corn leaf | 33.48 | SEQ_NO_6 | XP_026808275 | LOC113550554 |
| aphid) | | | | |
| Sipha flava (yellow sugarcane aphid) | 33.48 | SEQ_NO_6 | XP_025417241 | LOC112688314 |
| Sipha flava (yellow sugarcane aphid) | 31.87 | SEQ_NO_6 | XP_025405227 | LOC112679574 |
| Laodelphax striatellus | 33.33 | SEQ_NO_6 | A0A482XLD1_LAOST | LSTR_LSTR008033 |
| Rhodnius prolixus | 33.93 | SEQ_NO_6 | T1H8Y8_RHOPR | empty |
| Graphocephala atropunctata | 33.04 | SEQ_NO_6 | A0A1B6MB27_9HEMI | g.22349 |
| Apolygus lucorum | 33.86 | SEQ_NO_6 | A0A8S9XNA2_APOLU | GE061_015840 |
| Cuerna arida | 31.59 | SEQ_NO_6 | A0A1B6FV13_9HEMI | g.16852 |
| Diaphorina citri (Asian citrus psyllid) | 31.59 | SEQ_NO_6 | XP_026687037 | LOC103519929 |
| Nilaparvata lugens (brown | 33.71 | SEQ_NO_6 | BAQ02354 | NIST |
| planthopper) | | | | |
| Laodelphax striatellus | 34.09 | SEQ_NO_6 | A0A482X9L3_LAOST | LSTR_LSTR011132 |
| Adelges cooleyi (spruce gall adelgid) | 32.33 | SEQ_NO_6 | XP_050436601 | LOC126843250 |
| Apolygus lucorum | 32.89 | SEQ_NO_6 | A0A6A4IVB0_APOLU | GE061_003853 |
| Nilaparvata lugens (brown | 32.26 | SEQ_NO_6 | XP_022200158 | LOC111057042 |
| planthopper) | | | | |
| Acyrthosiphon pisum (pea aphid) | 32.96 | SEQ_NO_6 | XP_029347518 | LOC100163094 |
| Lygus hesperus | 35.36 | SEQ_NO_8 | A0A0A9WMT7_LYGHE | Tret1_15; Tret1_121; |
| | | | | CM83_93671; |
| | | | | CM83_93685 |
| Clastoptera arizonana | 35.98 | SEQ_NO_8 | A0A1B6DV63_9HEMI | g.5037 |
| Apolygus lucorum | 34.35 | SEQ_NO_8 | A0A6A4JSM2_APOLU | GE061_003173 |
| Cacopsylla melanoneura | 35.27 | SEQ_NO_8 | A0A8D8Z012_9HEMI | empty |
| Lygus hesperus | 36.66 | SEQ_NO_8 | A0A146LAC8 LYGHE | Tret1_81; g.73575 |
| Rhodnius prolixus | 35.76 | SEQ_NO_8 | T1HSD1_RHOPR | empty |
| Lygus hesperus | 35.28 | SEQ_NO_8 | A0A0A9WV06_LYGHE | Tret1_28; CM83_37324 |
| Rhodnius prolixus | 36.53 | SEQ_NO_8 | T1HM60 RHOPR | empty |
| Graphocephala atropunctata | 34.44 | SEQ_NO_8 | A0A1B6M4V4_9HEMI | g.28505 |
| Cuerna arida | 34.6 | SEQ_NO_8 | A0A1B6EJD5_9HEMI | g.17526 |
| Rhopalosiphum maidis (corn leaf | 32.43 | SEQ_NO_8 | XP_026819645 | LOC113558390 |
| aphid) | | | | |
| Rhodnius prolixus | 34.38 | SEQ_NO_8 | T1I1F0_RHOPR | empty |
| Cinara cedri | 32.84 | SEQ_NO_8 | A0A5E4NHI2_9HEMI | CINCED_3A011398 |
| Myzus persicae (green peach aphid) | 32.01 | SEQ_NO_8 | XP_022174142 | LOC111036436 |
| Aphis gossypii (cotton aphid) | 31.94 | SEQ_NO_8 | XP_050056585 | LOC114131561 |
| Nilaparvata lugens (brown | 33.41 | SEQ_NO_8 | BAQ02376 | NIST |
| planthopper) | | | | |
| Laodelphax striatellus | 32.97 | SEQ_NO_8 | A0A482XKB3_LAOST | LSTR_LSTR010894 |
| Nilaparvata lugens (brown | 33.11 | SEQ_NO_8 | XP_039290142 | LOC111044534 |
| planthopper) | | | | |

TABLE 5
Target sequences in whitefly species and accession numbers.

| | | Query Identifier- | | |
|---|---|---|---|---|
| | Align | Instant Application | | |
| Organism | % Id | Sequence Identifier | Identifier | Gene name |
| Bemisia tabaci | 100 | SEQ_NO_10 | XP_018915964 | LOC109043277 |
| Bemisia tabaci | 94.06 | SEQ_NO_10 | A0A9P0A732_BEMTA | BEMITA_LOCUS5153 |
| Bemisia tabaci | 62.84 | SEQ_NO_10 | CAH0387807 | empty |
| Bemisia tabaci | 62.84 | SEQ_NO_10 | A0A9P0ACQ5_BEMTA | BEMITA_LOCUS6778 |
| Bemisia tabaci | 63.23 | SEQ_NO_10 | CAH0769874 | empty |
| Bemisia tabaci | 63.23 | SEQ_NO_10 | XP_018898257 | LOC109031271 |
| Bemisia tabaci | 63.23 | SEQ_NO_10 | A0A9P0CA09_BEMTA | BEMITA_LOCUS6809 |
| Bemisia tabaci | 63.23 | SEQ_NO_10 | CAH0387844 | empty |
| Bemisia tabaci | 63.23 | SEQ_NO_10 | XP_018898256 | LOC109031271 |
| Bemisia tabaci | 63.23 | SEQ_NO_10 | A0A9P0A8G2_BEMTA | BEMITA_LOCUS6809 |
| Bemisia tabaci | 46.65 | SEQ_NO_10 | XP_018897969 | LOC109031094 |
| Bemisia tabaci | 46.65 | SEQ_NO_10 | XP_018897970 | LOC109031094 |
| Bemisia tabaci | 46.21 | SEQ_NO_10 | A0A9P0AA69_BEMTA | BEMITA_LOCUS5660 |
| Bemisia tabaci | 100 | SEQ_NO_10 | A0A9P0A9N5_BEMTA | BEMITA_LOCUS5154 |
| Bemisia tabaci | 100 | SEQ_NO_2 | XP_018913228 | LOC109041346 |
| Bemisia tabaci | 99.25 | SEQ_NO_2 | XP_018913227 | LOC109041346 |
| Bemisia tabaci | 98.11 | SEQ_NO_2 | A0A9P0EVM9_BEMTA | BEMITA_LOCUS714 |
| Bemisia tabaci | 59.7 | SEQ_NO_2 | XP_018913236 | LOC109041349 |
| Bemisia tabaci | 59.7 | SEQ_NO_2 | A0A9N9ZZT8_BEMTA | BEMITA_LOCUS718 |
| Bemisia tabaci | 61.19 | SEQ_NO_2 | XP_018909685 | LOC109038887 |
| Bemisia tabaci | 61.19 | SEQ_NO_2 | XP_018909686 | LOC109038887 |
| Bemisia tabaci | 61.19 | SEQ_NO_2 | XP_018909687 | LOC109038887 |
| Bemisia tabaci | 61.19 | SEQ_NO_2 | A0A9P0A1K7_BEMTA | BEMITA_LOCUS1212 |
| Bemisia tabaci | 56.8 | SEQ_NO_2 | A0A9P0EXJ0_BEMTA | BEMITA_LOCUS716 |
| Bemisia tabaci | 57.09 | SEQ_NO_2 | A0A9P0A602_BEMTA | BEMITA_LOCUS3284 |
| Bemisia tabaci | 56.41 | SEQ_NO_2 | XP_018912743 | LOC109041039 |
| Bemisia tabaci | 56.49 | SEQ_NO_2 | XP_018906726 | LOC109036807 |
| Bemisia tabaci | 54.47 | SEQ_NO_2 | XP_018897050 | LOC109030509 |
| Bemisia tabaci | 51.98 | SEQ_NO_2 | A0A9P0F409_BEMTA | BEMITA_LOCUS6269 |
| Bemisia tabaci | 56.45 | SEQ_NO_2 | XP_018906727 | LOC109036807 |
| Bemisia tabaci | 46.68 | SEQ_NO_2 | XP_018912744 | LOC109041040 |
| Bemisia tabaci | 46.68 | SEQ_NO_2 | XP_018912745 | LOC109041040 |
| Bemisia tabaci | 46.68 | SEQ_NO_2 | XP_018912746 | LOC109041040 |
| Bemisia tabaci | 46.48 | SEQ_NO_2 | A0A9P0EYE1_BEMTA | BEMITA_LOCUS717 |
| Bemisia tabaci | 46.71 | SEQ_NO_2 | XP_018910589 | LOC109039541 |
| Bemisia tabaci | 46.71 | SEQ_NO_2 | A0A9N9ZZJ4_BEMTA | BEMITA_LOCUS561 |
| Bemisia tabaci | 46.98 | SEQ_NO_2 | XP_018913237 | LOC109041350 |
| Bemisia tabaci | 46.98 | SEQ_NO_2 | XP_018913238 | LOC109041350 |
| Bemisia tabaci | 44.11 | SEQ_NO_2 | XP_018912742 | LOC109041038 |
| Bemisia tabaci | 44.11 | SEQ_NO_2 | A0A9N9ZZ81_BEMTA | BEMITA_LOCUS712 |
| Bemisia tabaci | 43.89 | SEQ_NO_2 | A0A9P0A0R5_BEMTA | BEMITA_LOCUS1220 |
| Bemisia tabaci | 45.66 | SEQ_NO_2 | XP_018913080 | LOC109041261 |
| Bemisia tabaci | 42.86 | SEQ_NO_2 | XP_018916839 | LOC109043928 |
| Bemisia tabaci | 42.86 | SEQ_NO_2 | A0A9P0AAZ4_BEMTA | BEMITA_LOCUS5870 |
| Bemisia tabaci | 45.45 | SEQ_NO_2 | A0A9N9ZZE3_BEMTA | BEMITA_LOCUS711 |
| Bemisia tabaci | 42.5 | SEQ_NO_2 | A0A9P0ACD7_BEMTA | BEMITA_LOCUS9987 |
| Bemisia tabaci | 44.53 | SEQ_NO_2 | XP_018909634 | LOC109038863 |
| Bemisia tabaci | 45.44 | SEQ_NO_2 | XP_018910999 | LOC109039790 |
| Bemisia tabaci | 45.44 | SEQ_NO_2 | A0A9P0C7P2_BEMTA | BEMITA_LOCUS508 |
| Bemisia tabaci | 42.29 | SEQ_NO_2 | A0A9P0EYF1_BEMTA | BEMITA_LOCUS741 |
| Bemisia tabaci | 44.18 | SEQ_NO_2 | A0A9P0EVX9_BEMTA | BEMITA_LOCUS715 |
| Bemisia tabaci | 41.9 | SEQ_NO_2 | XP_018911743 | LOC109040305 |
| Bemisia tabaci | 41.9 | SEQ_NO_2 | XP_018911752 | LOC109040305 |
| Bemisia tabaci | 41.9 | SEQ_NO_2 | XP_018911760 | LOC109040305 |
| Bemisia tabaci | 41.9 | SEQ_NO_2 | A0A9N9ZZ93_BEMTA | BEMITA_LOCUS563 |
| Bemisia tabaci | 41.9 | SEQ_NO_2 | XP_018912951 | LOC109041173 |
| Bemisia tabaci | 41.87 | SEQ_NO_2 | XP_018911771 | LOC109040323 |
| Bemisia tabaci | 42.28 | SEQ_NO_2 | XP_018909683 | LOC109038886 |
| Bemisia tabaci | 42.28 | SEQ_NO_2 | A0A9P0EWC8_BEMTA | BEMITA_LOCUS1215 |
| Bemisia tabaci | 43.78 | SEQ_NO_2 | XP_018913229 | LOC109041347 |
| Bemisia tabaci | 43.78 | SEQ_NO_2 | XP_018913231 | LOC109041347 |
| Bemisia tabaci | 43.06 | SEQ_NO_2 | XP_018901769 | LOC109033552 |
| Bemisia tabaci | 43.06 | SEQ_NO_2 | XP_018901774 | LOC109033552 |
| Bemisia tabaci | 43.57 | SEQ_NO_2 | XP_018913232 | LOC109041347 |
| Bemisia tabaci | 42.86 | SEQ_NO_2 | XP_018909560 | LOC109038816 |
| Bemisia tabaci | 41.93 | SEQ_NO_2 | XP_018909635 | LOC109038864 |
| Bemisia tabaci | 41.93 | SEQ_NO_2 | A0A9P0EXZ6_BEMTA | BEMITA_LOCUS1217 |
| Bemisia tabaci | 42.15 | SEQ_NO_2 | A0A9P0G2P9_BEMTA | BEMITA_LOCUS562 |
| Bemisia tabaci | 42.32 | SEQ_NO_2 | A0A9P0EYV1_BEMTA | BEMITA_LOCUS1218 |
| Bemisia tabaci | 46.27 | SEQ_NO_2 | A0A9N9ZZI1_BEMTA | BEMITA_LOCUS719 |
| Bemisia tabaci | 42.54 | SEQ_NO_2 | XP_018909688 | LOC109038888 |
| Bemisia tabaci | 42.54 | SEQ_NO_2 | XP_018909689 | LOC109038888 |
| Bemisia tabaci | 42.34 | SEQ_NO_2 | A0A9P0EW39_BEMTA | BEMITA_LOCUS1214 |
| Bemisia tabaci | 42 | SEQ_NO_2 | A0A9N9ZZG6_BEMTA | BEMITA_LOCUS509 |
| Bemisia tabaci | 42.51 | SEQ_NO_2 | XP_018906323 | LOC109036509 |
| Bemisia tabaci | 40.54 | SEQ_NO_2 | CAH0383210 | empty |
| Bemisia tabaci | 40.54 | SEQ_NO_2 | A0A9P0A0H8_BEMTA | BEMITA_LOCUS2674 |
| Bemisia tabaci | 41.72 | SEQ_NO_2 | XP_018906325 | LOC109036510 |
| Bemisia tabaci | 41.72 | SEQ_NO_2 | XP_018906326 | LOC109036510 |
| Bemisia tabaci | 41.72 | SEQ_NO_2 | XP_018906327 | LOC109036510 |
| Bemisia tabaci | 41.72 | SEQ_NO_2 | A0A9P0AI47_BEMTA | BEMITA_LOCUS10849 |
| Bemisia tabaci | 42.22 | SEQ_NO_2 | A0A9P0AI57_BEMTA | BEMITA_LOCUS10850 |
| Bemisia tabaci | 41.03 | SEQ_NO_2 | A0A9P0EZ74_BEMTA | BEMITA_LOCUS1216 |
| Bemisia tabaci | 42.19 | SEQ_NO_2 | XP_018913096 | LOC109041272 |
| Bemisia tabaci | 41.6 | SEQ_NO_2 | XP_018910980 | LOC109039775 |
| Bemisia tabaci | 41.78 | SEQ_NO_2 | A0A9P0EVP0_BEMTA | BEMITA_LOCUS754 |
| Bemisia tabaci | 40.43 | SEQ_NO_2 | XP_018909732 | LOC109038916 |
| Bemisia tabaci | 43.5 | SEQ_NO_2 | XP_018911009 | LOC109039790 |
| Bemisia tabaci | 43.5 | SEQ_NO_2 | XP_018911017 | LOC109039790 |
| Bemisia tabaci | 43.5 | SEQ_NO_2 | XP_018911026 | LOC109039790 |
| Bemisia tabaci | 39.26 | SEQ_NO_2 | A0A9P0A0P0_BEMTA | BEMITA_LOCUS1219 |
| Bemisia tabaci | 37.24 | SEQ_NO_2 | XP_018912954 | LOC109041177 |
| Bemisia tabaci | 37.24 | SEQ_NO_2 | XP_018912955 | LOC109041177 |
| Bemisia tabaci | 37.24 | SEQ_NO_2 | XP_018912956 | LOC109041177 |
| Bemisia tabaci | 37.67 | SEQ_NO_2 | A0A9P0EYV7_BEMTA | BEMITA_LOCUS833 |
| Bemisia tabaci | 37.48 | SEQ_NO_2 | XP_018912975 | LOC109041190 |
| Bemisia tabaci | 37.48 | SEQ_NO_2 | XP_018912974 | LOC109041190 |
| Bemisia tabaci | 37.26 | SEQ_NO_2 | CAH0770735 | empty |
| Bemisia tabaci | 37.26 | SEQ_NO_2 | XP_018906603 | LOC109036701 |
| Bemisia tabaci | 37.26 | SEQ_NO_2 | A0A9P0CFJ1_BEMTA | BEMITA_LOCUS7574 |
| Bemisia tabaci | 38.63 | SEQ_NO_2 | XP_018906609 | LOC109036701 |
| Bemisia tabaci | 38.63 | SEQ_NO_2 | XP_018906616 | LOC109036701 |
| Bemisia tabaci | 34.82 | SEQ_NO_2 | A0A9P0EVN6_BEMTA | BEMITA_LOCUS737 |
| Bemisia tabaci | 35.12 | SEQ_NO_2 | XP_018915851 | LOC109043187 |
| Bemisia tabaci | 35.19 | SEQ_NO_2 | A0A9P0A0P2_BEMTA | BEMITA_LOCUS1393 |
| Bemisia tabaci | 41.07 | SEQ_NO_2 | XP_018910988 | LOC109039775 |
| Bemisia tabaci | 34.72 | SEQ_NO_2 | A0A9P0A9R2_BEMTA | BEMITA_LOCUS6185 |
| Bemisia tabaci | 34.52 | SEQ_NO_2 | XP_018900030 | LOC109032390 |
| Bemisia tabaci | 34.52 | SEQ_NO_2 | XP_018900031 | LOC109032390 |
| Bemisia tabaci | 34.52 | SEQ_NO_2 | XP_018900033 | LOC109032390 |
| Bemisia tabaci | 33.67 | SEQ_NO_2 | XP_018896687 | LOC109030266 |
| Bemisia tabaci | 33.67 | SEQ_NO_2 | XP_018896688 | LOC109030266 |
| Bemisia tabaci | 33.67 | SEQ_NO_2 | XP_018896689 | LOC109030266 |
| Bemisia tabaci | 33.67 | SEQ_NO_2 | XP_018896685 | LOC109030266 |
| Bemisia tabaci | 33.67 | SEQ_NO_2 | XP_018896686 | LOC109030266 |
| Bemisia tabaci | 37.98 | SEQ_NO_2 | XP_018912957 | LOC109041177 |
| Bemisia tabaci | 39.57 | SEQ_NO_2 | A0A9P0G2T1_BEMTA | BEMITA_LOCUS1217 |
| Bemisia tabaci | 53.75 | SEQ_NO_2 | A0A9P0F2R0_BEMTA | BEMITA_LOCUS6269 |
| Bemisia tabaci | 33.4 | SEQ_NO_2 | CAH0394487 | empty |
| Bemisia tabaci | 33.4 | SEQ_NO_2 | A0A9P0F9Z7_BEMTA | BEMITA_LOCUS12780 |
| Bemisia tabaci | 33 | SEQ_NO_2 | XP_018900402 | LOC109032625 |
| Bemisia tabaci | 33 | SEQ_NO_2 | XP_018900403 | LOC109032625 |
| Bemisia tabaci | 33 | SEQ_NO_2 | XP_018900401 | LOC109032625 |
| Bemisia tabaci | 32.53 | SEQ_NO_2 | XP_018915857 | LOC109043191 |
| Bemisia tabaci | 32.53 | SEQ_NO_2 | A0A9P0FZM8_BEMTA | BEMITA_LOCUS1392 |
| Bemisia tabaci | 30.74 | SEQ_NO_2 | XP_018908021 | LOC109037691 |
| Bemisia tabaci | 30.74 | SEQ_NO_2 | XP_018908019 | LOC109037691 |
| Bemisia tabaci | 30.74 | SEQ_NO_2 | XP_018908020 | LOC109037691 |
| Bemisia tabaci | 31.76 | SEQ_NO_2 | A0A9P0A0K2_BEMTA | BEMITA_LOCUS1394 |
| Bemisia tabaci | 31.36 | SEQ_NO_2 | XP_018915815 | LOC109043164 |
| Bemisia tabaci | 31.16 | SEQ_NO_2 | CAH0383098 | empty |
| Bemisia tabaci | 31.16 | SEQ_NO_2 | A0A9P0F0F5_BEMTA | BEMITA_LOCUS2575 |
| Bemisia tabaci | 30.89 | SEQ_NO_2 | A0A9P0EVZ2_BEMTA | BEMITA_LOCUS755 |
| Bemisia tabaci | 43.77 | SEQ_NO_2 | XP_018906862 | LOC109036898 |
| Bemisia tabaci | 34.65 | SEQ_NO_2 | XP_018915852 | LOC109043187 |
| Bemisia tabaci | 56.06 | SEQ_NO_2 | A0A9P0F3D5_BEMTA | BEMITA_LOCUS6270 |
| Bemisia tabaci | 31.14 | SEQ_NO_2 | XP_018913094 | LOC109041271 |
| Bemisia tabaci | 32.17 | SEQ_NO_2 | XP_018915816 | LOC109043164 |
| Bemisia tabaci | 26.94 | SEQ_NO_2 | CAH0388120 | empty |
| Bemisia tabaci | 26.94 | SEQ_NO_2 | XP_018901708 | LOC109033507 |
| Bemisia tabaci | 26.94 | SEQ_NO_2 | A0A9P0F1M4_BEMTA | BEMITA_LOCUS7060 |
| Bemisia tabaci | 39.34 | SEQ_NO_2 | A0A9P0AIR7_BEMTA | BEMITA_LOCUS9987 |
| Bemisia tabaci | 26.76 | SEQ_NO_2 | CAH0388121 | empty |
| Bemisia tabaci | 26.76 | SEQ_NO_2 | XP_018901709 | LOC109033507 |
| Bemisia tabaci | 26.76 | SEQ_NO_2 | A0A9P0F4U1_BEMTA | BEMITA_LOCUS7060 |
| Bemisia tabaci | 33.02 | SEQ_NO_2 | XP_018915859 | LOC109043191 |
| Bemisia tabaci | 26.81 | SEQ_NO_2 | XP_018910831 | LOC109039686 |
| Bemisia tabaci | 26.81 | SEQ_NO_2 | XP_018910832 | LOC109039686 |
| Bemisia tabaci | 26 | SEQ_NO_2 | CAH0388939 | empty |
| Bemisia tabaci | 26 | SEQ_NO_2 | XP_018898680 | LOC109031556 |
| Bemisia tabaci | 26 | SEQ_NO_2 | A0A9P0F2B0_BEMTA | BEMITA_LOCUS7819 |
| Bemisia tabaci | 25.92 | SEQ_NO_2 | CAH0389626 | empty |
| Bemisia tabaci | 25.92 | SEQ_NO_2 | XP_018909262 | LOC109038613 |
| Bemisia tabaci | 25.92 | SEQ_NO_2 | A0A9P0ABG5_BEMTA | BEMITA_LOCUS8436 |
| Bemisia tabaci | 24.9 | SEQ_NO_2 | A0A9P0AGI2_BEMTA | BEMITA_LOCUS10891 |
| Bemisia tabaci | 24.95 | SEQ_NO_2 | XP_018915405 | LOC109042890 |
| Bemisia tabaci | 24.95 | SEQ_NO_2 | XP_018915406 | LOC109042890 |
| Bemisia tabaci | 27.31 | SEQ_NO_2 | A0A9P0EW98_BEMTA | BEMITA_LOCUS1396 |
| Bemisia tabaci | 27.66 | SEQ_NO_2 | XP_018915817 | LOC109043165 |
| Bemisia tabaci | 25.39 | SEQ_NO_2 | XP_018906977 | LOC109036976 |
| Bemisia tabaci | 28.6 | SEQ_NO_2 | XP_018918218 | LOC109044767 |
| Bemisia tabaci | 28.6 | SEQ_NO_2 | XP_018918219 | LOC109044767 |
| Bemisia tabaci | 28.6 | SEQ_NO_2 | XP_018918220 | LOC109044767 |
| Bemisia tabaci | 28.6 | SEQ_NO_2 | XP_018918217 | LOC109044767 |
| Bemisia tabaci | 28.6 | SEQ_NO_2 | XP_018918216 | LOC109044767 |
| Bemisia tabaci | 52.67 | SEQ_NO_2 | A0A9N9ZZQ5_BEMTA | BEMITA_LOCUS717 |
| Bemisia tabaci | 23.69 | SEQ_NO_2 | A0A9P0AHE1_BEMTA | BEMITA_LOCUS11376 |
| Bemisia tabaci | 25.15 | SEQ_NO_2 | A0A9P0F6T8_BEMTA | BEMITA_LOCUS10705 |
| Bemisia tabaci | 28.22 | SEQ_NO_2 | A0A9P0C0C4_BEMTA | BEMITA_LOCUS1348 |
| Bemisia tabaci | 23.65 | SEQ_NO_2 | XP_018904527 | LOC109035374 |
| Bemisia tabaci | 23.65 | SEQ_NO_2 | XP_018904526 | LOC109035374 |
| Bemisia tabaci | 33.87 | SEQ_NO_2 | XP_018900034 | LOC109032390 |
| Bemisia tabaci | 25.77 | SEQ_NO_2 | XP_018904504 | LOC109035359 |
| Bemisia tabaci | 25.56 | SEQ_NO_2 | A0A9P0ALC7_BEMTA | BEMITA_LOCUS11377 |
| Bemisia tabaci | 24.17 | SEQ_NO_2 | ARX98207 | empty |
| Bemisia tabaci | 24.17 | SEQ_NO_2 | XP_018897624 | LOC109030883 |
| Bemisia tabaci | 24.17 | SEQ_NO_2 | A0A1Z1XG35_BEMTA | empty |
| Bemisia tabaci | 27.46 | SEQ_NO_2 | A0A9P0F887_BEMTA | BEMITA_LOCUS12206 |
| Bemisia tabaci | 32.19 | SEQ_NO_2 | CAH0394489 | empty |
| Bemisia tabaci | 32.19 | SEQ_NO_2 | A0A9P0F926_BEMTA | BEMITA_LOCUS12780 |
| Bemisia tabaci | 32.19 | SEQ_NO_2 | CAH0394488 | empty |
| Bemisia tabaci | 32.19 | SEQ_NO_2 | A0A9P0F8R1_BEMTA | BEMITA_LOCUS12780 |
| Bemisia tabaci | 24.8 | SEQ_NO_2 | XP_018909924 | LOC109039048 |
| Bemisia tabaci | 23 | SEQ_NO_2 | XP_018913137 | LOC109041294 |
| Bemisia tabaci | 24.49 | SEQ_NO_2 | A0A9P0AC69_BEMTA | BEMITA_LOCUS6675 |
| Bemisia tabaci | 24.14 | SEQ_NO_2 | XP_018897433 | LOC109030767 |
| Bemisia tabaci | 29.13 | SEQ_NO_2 | XP_018913095 | LOC109041271 |
| Bemisia tabaci | 23.84 | SEQ_NO_2 | A0A9P0FYC7_BEMTA | BEMITA_LOCUS1640 |
| Bemisia tabaci | 23 | SEQ_NO_2 | A0A9P0A4W1_BEMTA | BEMITA_LOCUS3474 |
| Bemisia tabaci | 21.07 | SEQ_NO_2 | XP_018899440 | LOC109032015 |
| Bemisia tabaci | 24.63 | SEQ_NO_2 | A0A9P0CF03_BEMTA | BEMITA_LOCUS6098 |
| Bemisia tabaci | 24.63 | SEQ_NO_2 | XP_018897978 | LOC109031099 |
| Bemisia tabaci | 26.22 | SEQ_NO_2 | XP_018918255 | LOC109044786 |
| Bemisia tabaci | 26.22 | SEQ_NO_2 | A0A9P0CAM0_BEMTA | BEMITA_LOCUS1346 |
| Bemisia tabaci | 22.63 | SEQ_NO_2 | XP_018913138 | LOC109041294 |
| Bemisia tabaci | 22.63 | SEQ_NO_2 | A0A9P0EYC6_BEMTA | BEMITA_LOCUS664 |
| Bemisia tabaci | 26.12 | SEQ_NO_2 | XP_018916631 | LOC109043774 |
| Bemisia tabaci | 26.12 | SEQ_NO_2 | XP_018916630 | LOC109043774 |
| Bemisia tabaci | 23.58 | SEQ_NO_2 | XP_018903363 | LOC109034588 |
| Bemisia tabaci | 26.48 | SEQ_NO_2 | XP_018906978 | LOC109036976 |
| Bemisia tabaci | 24.68 | SEQ_NO_2 | XP_018897977 | LOC109031099 |
| Bemisia tabaci | 24.68 | SEQ_NO_2 | A0A9P0AAM2_BEMTA | BEMITA_LOCUS5680 |
| Bemisia tabaci | 25.89 | SEQ_NO_2 | CAH0771318 | empty |
| Bemisia tabaci | 25.89 | SEQ_NO_2 | A0A9P0CAK4_BEMTA | BEMITA_LOCUS8079 |
| Bemisia tabaci | 25.73 | SEQ_NO_2 | XP_018916629 | LOC109043772 |
| Bemisia tabaci | 23.86 | SEQ_NO_2 | A0A9P0AID5_BEMTA | BEMITA_LOCUS10756 |
| Bemisia tabaci | 25.56 | SEQ_NO_2 | XP_018901760 | LOC109033544 |
| Bemisia tabaci | 24.01 | SEQ_NO_2 | XP_018906910 | LOC109036938 |
| Bemisia tabaci | 25.28 | SEQ_NO_2 | A0A9P0G4K7_BEMTA | BEMITA_LOCUS10755 |
| Bemisia tabaci | 26.15 | SEQ_NO_2 | A0A9P0CDF0_BEMTA | BEMITA_LOCUS12171 |
| Bemisia tabaci | 26.15 | SEQ_NO_2 | XP_018905130 | LOC109035808 |
| Bemisia tabaci | 26.15 | SEQ_NO_2 | XP_018905131 | LOC109035808 |
| Bemisia tabaci | 26.15 | SEQ_NO_2 | XP_018905132 | LOC109035808 |
| Bemisia tabaci | 26.15 | SEQ_NO_2 | A0A9P0C6R4_BEMTA | BEMITA_LOCUS12171 |
| Bemisia tabaci | 26.15 | SEQ_NO_2 | XP_018905129 | LOC109035808 |
| Bemisia tabaci | 26.15 | SEQ_NO_2 | XP_018905128 | LOC109035808 |
| Bemisia tabaci | 26.15 | SEQ_NO_2 | A0A9P0CH78_BEMTA | BEMITA_LOCUS12171 |
| Bemisia tabaci | 26.15 | SEQ_NO_2 | ARX98212 | empty |
| Bemisia tabaci | 26.15 | SEQ_NO_2 | XP_018905127 | LOC109035808 |
| Bemisia tabaci | 26.15 | SEQ_NO_2 | A0A1Z1XG30_BEMTA | empty |
| Bemisia tabaci | 26.15 | SEQ_NO_2 | XP_018905126 | LOC109035808 |
| Bemisia tabaci | 26.15 | SEQ_NO_2 | A0A9P0CDP4_BEMTA | BEMITA_LOCUS12171 |
| Bemisia tabaci | 21.92 | SEQ_NO_2 | A0A9P0EYI8_BEMTA | BEMITA_LOCUS405 |
| Bemisia tabaci | 21.73 | SEQ_NO_2 | XP_018910394 | LOC109039379 |
| Bemisia tabaci | 22.87 | SEQ_NO_2 | XP_018905607 | LOC109036110 |
| Bemisia tabaci | 23.11 | SEQ_NO_2 | XP_018901924 | LOC109033670 |
| Bemisia tabaci | 25.17 | SEQ_NO_2 | XP_018906911 | LOC109036939 |
| Bemisia tabaci | 25.17 | SEQ_NO_2 | A0A9P0AJS7_BEMTA | BEMITA_LOCUS10749 |
| Bemisia tabaci | 25.74 | SEQ_NO_2 | XP_018903297 | LOC109034537 |
| Bemisia tabaci | 24.89 | SEQ_NO_2 | A0A9P0AB17_BEMTA | BEMITA_LOCUS5900 |
| Bemisia tabaci | 25.81 | SEQ_NO_2 | ARX98206 | empty |
| Bemisia tabaci | 25.81 | SEQ_NO_2 | A0A1Z1XG37_BEMTA | empty |
| Bemisia tabaci | 25.81 | SEQ_NO_2 | A0A9P0FAJ7_BEMTA | BEMITA_LOCUS13573 |
| Bemisia tabaci | 24.72 | SEQ_NO_2 | XP_018916871 | LOC109043946 |
| Bemisia tabaci | 22.81 | SEQ_NO_2 | XP_018914125 | LOC109042030 |
| Bemisia tabaci | 22.81 | SEQ_NO_2 | A0A9P0AKD5_BEMTA | BEMITA_LOCUS11059 |
| Bemisia tabaci | 23.48 | SEQ_NO_2 | CAH0388118 | empty |
| Bemisia tabaci | 23.48 | SEQ_NO_2 | A0A9P0ACX9_BEMTA | BEMITA_LOCUS7058 |
| Bemisia tabaci | 23.09 | SEQ_NO_2 | CAH0389139 | empty |
| Bemisia tabaci | 23.09 | SEQ_NO_2 | A0A9P0AD18_BEMTA | BEMITA_LOCUS8000 |
| Bemisia tabaci | 22.67 | SEQ_NO_2 | XP_018906907 | LOC109036935 |
| Bemisia tabaci | 23.27 | SEQ_NO_2 | XP_018901750 | LOC109033536 |
| Bemisia tabaci | 23.37 | SEQ_NO_2 | XP_018901751 | LOC109033536 |
| Bemisia tabaci | 25.28 | SEQ_NO_2 | A0A9P0G5J5_BEMTA | BEMITA_LOCUS10004 |
| Bemisia tabaci | 22.84 | SEQ_NO_2 | A0A9P0C9R0_BEMTA | BEMITA_LOCUS10706 |
| Bemisia tabaci | 22.84 | SEQ_NO_2 | XP_018906975 | LOC109036975 |
| Bemisia tabaci | 25.58 | SEQ_NO_2 | XP_018897621 | LOC109030881 |
| Bemisia tabaci | 25.58 | SEQ_NO_2 | XP_018897618 | LOC109030881 |
| Bemisia tabaci | 25.58 | SEQ_NO_2 | XP_018897620 | LOC109030881 |
| Bemisia tabaci | 24.83 | SEQ_NO_2 | XP_018916605 | LOC109043754 |
| Bemisia tabaci | 24.83 | SEQ_NO_2 | XP_018916604 | LOC109043754 |
| Bemisia tabaci | 24.24 | SEQ_NO_2 | CAH0383301 | empty |
| Bemisia tabaci | 24.24 | SEQ_NO_2 | A0A9P0A3J4_BEMTA | BEMITA_LOCUS2761 |
| Bemisia tabaci | 24.89 | SEQ_NO_2 | A0A9P0F8D9_BEMTA | BEMITA_LOCUS10752 |
| Bemisia tabaci | 20.72 | SEQ_NO_2 | XP_018899442 | LOC109032015 |
| Bemisia tabaci | 20.72 | SEQ_NO_2 | A0A9P0CAV5_BEMTA | BEMITA_LOCUS4980 |
| Bemisia tabaci | 25.17 | SEQ_NO_2 | XP_018897625 | LOC109030883 |
| Bemisia tabaci | 24.61 | SEQ_NO_2 | CAH0389226 | empty |
| Bemisia tabaci | 24.61 | SEQ_NO_2 | A0A9P0AD54_BEMTA | BEMITA_LOCUS8077 |
| Bemisia tabaci | 21.44 | SEQ_NO_2 | CAH0389136 | empty |
| Bemisia tabaci | 21.44 | SEQ_NO_2 | A0A9P0ABQ5_BEMTA | BEMITA_LOCUS7998 |
| Bemisia tabaci | 22.68 | SEQ_NO_2 | XP_018908558 | LOC109038077 |
| Bemisia tabaci | 24.34 | SEQ_NO_2 | CAH0772341 | empty |
| Bemisia tabaci | 24.34 | SEQ_NO_2 | XP_018906439 | LOC109036578 |
| Bemisia tabaci | 24.34 | SEQ_NO_2 | A0A9P0G5E3_BEMTA | BEMITA_LOCUS8956 |
| Bemisia tabaci | 24.44 | SEQ_NO_2 | A0A9P0F157_BEMTA | BEMITA_LOCUS3824 |
| Bemisia tabaci | 24.44 | SEQ_NO_2 | ARX98211 | empty |
| Bemisia tabaci | 24.44 | SEQ_NO_2 | XP_018895971 | LOC109029784 |
| Bemisia tabaci | 24.44 | SEQ_NO_2 | A0A1Z1XG24_BEMTA | empty |
| Bemisia tabaci | 24.44 | SEQ_NO_2 | XP_018895964 | LOC109029784 |
| Bemisia tabaci | 23.24 | SEQ_NO_2 | XP_018908925 | LOC109038346 |
| Bemisia tabaci | 23.24 | SEQ_NO_2 | CAH0777229 | empty |
| Bemisia tabaci | 23.24 | SEQ_NO_2 | XP_018908924 | LOC109038346 |
| Bemisia tabaci | 23.24 | SEQ_NO_2 | A0A9P0G1G9_BEMTA | BEMITA_LOCUS13217 |
| Bemisia tabaci | 22.52 | SEQ_NO_2 | XP_018915407 | LOC109042891 |
| Bemisia tabaci | 22.52 | SEQ_NO_2 | A0A9P0AK32_BEMTA | BEMITA_LOCUS10899 |
| Bemisia tabaci | 21.25 | SEQ_NO_2 | XP_018908586 | LOC109038097 |
| Bemisia tabaci | 24.16 | SEQ_NO_2 | XP_018897468 | LOC109030788 |
| Bemisia tabaci | 24.16 | SEQ_NO_2 | XP_018897467 | LOC109030788 |
| Bemisia tabaci | 24.16 | SEQ_NO_2 | XP_018916621 | LOC109043765 |
| Bemisia tabaci | 24.16 | SEQ_NO_2 | XP_018916620 | LOC109043765 |
| Bemisia tabaci | 24.22 | SEQ_NO_2 | XP_018914907 | LOC109042553 |
| Bemisia tabaci | 25.41 | SEQ_NO_2 | XP_018901761 | LOC109033544 |
| Bemisia tabaci | 25.41 | SEQ_NO_2 | XP_018901762 | LOC109033544 |
| Bemisia tabaci | 24.04 | SEQ_NO_2 | XP_018901754 | LOC109033538 |
| Bemisia tabaci | 23.94 | SEQ_NO_2 | CAH0389224 | empty |
| Bemisia tabaci | 23.94 | SEQ_NO_2 | A0A9P0AAS3_BEMTA | BEMITA_LOCUS8075 |
| Bemisia tabaci | 21.79 | SEQ_NO_2 | XP_018902173 | LOC109033825 |
| Bemisia tabaci | 20.98 | SEQ_NO_2 | CAH0394926 | empty |
| Bemisia tabaci | 20.98 | SEQ_NO_2 | XP_018902179 | LOC109033828 |
| Bemisia tabaci | 20.98 | SEQ_NO_2 | A0A9P0F776_BEMTA | BEMITA_LOCUS13173 |
| Bemisia tabaci | 23.73 | SEQ_NO_2 | ARX98213 | empty |
| Bemisia tabaci | 23.73 | SEQ_NO_2 | XP_018901427 | LOC109033310 |
| Bemisia tabaci | 23.73 | SEQ_NO_2 | A0A1Z1XG31_BEMTA | empty |
| Bemisia tabaci | 23.99 | SEQ_NO_2 | XP_018907714 | LOC109037475 |
| Bemisia tabaci | 24.1 | SEQ_NO_2 | CAH0394501 | empty |
| Bemisia tabaci | 24.1 | SEQ_NO_2 | A0A9P0F6V5_BEMTA | BEMITA_LOCUS12792 |
| Bemisia tabaci | 23.66 | SEQ_NO_2 | CAH0770634 | empty |
| Bemisia tabaci | 23.66 | SEQ_NO_2 | A0A9P0G547_BEMTA | BEMITA_LOCUS7484 |
| Bemisia tabaci | 23.66 | SEQ_NO_2 | XP_018916201 | LOC109043457 |
| Bemisia tabaci | 23.87 | SEQ_NO_2 | CAH0382991 | empty |
| Bemisia tabaci | 23.87 | SEQ_NO_2 | XP_018918090 | LOC109044710 |
| Bemisia tabaci | 23.87 | SEQ_NO_2 | A0A9P0A048_BEMTA | BEMITA_LOCUS2479 |
| Bemisia tabaci | 22.84 | SEQ_NO_2 | XP_018903760 | LOC109034842 |
| Bemisia tabaci | 21.59 | SEQ_NO_2 | CAH0394924 | empty |
| Bemisia tabaci | 21.59 | SEQ_NO_2 | A0A9P0AME5_BEMTA | BEMITA_LOCUS13171 |
| Bemisia tabaci | 23.71 | SEQ_NO_2 | XP_018916627 | LOC109043770 |
| Bemisia tabaci | 23.28 | SEQ_NO_2 | XP_018917470 | LOC109044283 |
| Bemisia tabaci | 23.28 | SEQ_NO_2 | XP_018917469 | LOC109044283 |
| Bemisia tabaci | 22.39 | SEQ_NO_2 | XP_018912545 | LOC109040896 |
| Bemisia tabaci | 23.54 | SEQ_NO_2 | XP_018897434 | LOC109030768 |
| Bemisia tabaci | 21.74 | SEQ_NO_2 | XP_018902172 | LOC109033825 |
| Bemisia tabaci | 22.58 | SEQ_NO_2 | A0A9P0AQA1_BEMTA | BEMITA_LOCUS13791 |
| Bemisia tabaci | 22.88 | SEQ_NO_2 | XP_018905208 | LOC109035853 |
| Bemisia tabaci | 22.88 | SEQ_NO_2 | A0A9P0F680_BEMTA | BEMITA_LOCUS12088 |
| Bemisia tabaci | 23.76 | SEQ_NO_2 | XP_018908719 | LOC109038195 |
| Bemisia tabaci | 23.76 | SEQ_NO_2 | XP_018908720 | LOC109038195 |
| Bemisia tabaci | 23.76 | SEQ_NO_2 | XP_018908721 | LOC109038195 |
| Bemisia tabaci | 23.06 | SEQ_NO_2 | A0A9P0F223_BEMTA | BEMITA_LOCUS5499 |
| Bemisia tabaci | 23.27 | SEQ_NO_2 | XP_018906443 | LOC109036579 |
| Bemisia tabaci | 22.91 | SEQ_NO_2 | A0A9P0CAY8_BEMTA | BEMITA_LOCUS10706 |
| Bemisia tabaci | 23.16 | SEQ_NO_2 | XP_018903308 | LOC109034545 |
| Bemisia tabaci | 24.82 | SEQ_NO_2 | XP_018899443 | LOC109032015 |
| Bemisia tabaci | 24.82 | SEQ_NO_2 | XP_018899441 | LOC109032015 |
| Bemisia tabaci | 24.82 | SEQ_NO_2 | A0A9P0F278_BEMTA | BEMITA_LOCUS4980 |
| Bemisia tabaci | 23.32 | SEQ_NO_2 | A0A9P0AAL6_BEMTA | BEMITA_LOCUS6675 |
| Bemisia tabaci | 21.01 | SEQ_NO_2 | XP_018902136 | LOC109033813 |
| Bemisia tabaci | 23.37 | SEQ_NO_2 | CAH0388119 | empty |
| Bemisia tabaci | 23.37 | SEQ_NO_2 | A0A9P0F1N7_BEMTA | BEMITA_LOCUS7059 |
| Bemisia tabaci | 21.58 | SEQ_NO_2 | XP_018901931 | LOC109033672 |
| Bemisia tabaci | 21.58 | SEQ_NO_2 | XP_018901932 | LOC109033672 |
| Bemisia tabaci | 21.58 | SEQ_NO_2 | XP_018901933 | LOC109033672 |
| Bemisia tabaci | 21.58 | SEQ_NO_2 | XP_018901934 | LOC109033672 |
| Bemisia tabaci | 22.37 | SEQ_NO_2 | CAH0389480 | empty |
| Bemisia tabaci | 22.37 | SEQ_NO_2 | A0A9P0AF98_BEMTA | BEMITA_LOCUS8301 |
| Bemisia tabaci | 23.53 | SEQ_NO_2 | A0A9P0F7P8_BEMTA | BEMITA_LOCUS13696 |
| Bemisia tabaci | 23.85 | SEQ_NO_2 | CAH0389469 | empty |
| Bemisia tabaci | 23.85 | SEQ_NO_2 | A0A9P0F586_BEMTA | BEMITA_LOCUS8295 |
| Bemisia tabaci | 23.85 | SEQ_NO_2 | ARX98209 | empty |
| Bemisia tabaci | 23.85 | SEQ_NO_2 | XP_018902338 | LOC109033933 |
| Bemisia tabaci | 23.85 | SEQ_NO_2 | XP_018902339 | LOC109033933 |
| Bemisia tabaci | 23.85 | SEQ_NO_2 | XP_018902340 | LOC109033933 |
| Bemisia tabaci | 23.85 | SEQ_NO_2 | A0A1Z1XG23_BEMTA | empty |
| Bemisia tabaci | 23.85 | SEQ_NO_2 | XP_018902337 | LOC109033933 |
| Bemisia tabaci | 23.85 | SEQ_NO_2 | XP_018902336 | LOC109033933 |
| Bemisia tabaci | 23.85 | SEQ_NO_2 | CAH0394705 | empty |
| Bemisia tabaci | 23.85 | SEQ_NO_2 | A0A9P0AM39_BEMTA | BEMITA_LOCUS12972 |
| Bemisia tabaci | 23.96 | SEQ_NO_2 | CAH0770635 | empty |
| Bemisia tabaci | 23.96 | SEQ_NO_2 | A0A9P0G3Y9_BEMTA | BEMITA_LOCUS7485 |
| Bemisia tabaci | 21.37 | SEQ_NO_2 | A0A9P0CA77_BEMTA | BEMITA_LOCUS3471 |
| Bemisia tabaci | 23.73 | SEQ_NO_2 | XP_018916224 | LOC109043474 |
| Bemisia tabaci | 23.73 | SEQ_NO_2 | XP_018916225 | LOC109043474 |
| Bemisia tabaci | 23.62 | SEQ_NO_2 | XP_018915002 | LOC109042602 |
| Bemisia tabaci | 23.62 | SEQ_NO_2 | XP_018915003 | LOC109042602 |
| Bemisia tabaci | 23.62 | SEQ_NO_2 | XP_018914998 | LOC109042602 |
| Bemisia tabaci | 23.62 | SEQ_NO_2 | XP_018914999 | LOC109042602 |
| Bemisia tabaci | 23.62 | SEQ_NO_2 | XP_018915000 | LOC109042602 |
| Bemisia tabaci | 23.62 | SEQ_NO_2 | XP_018915001 | LOC109042602 |
| Bemisia tabaci | 23.04 | SEQ_NO_2 | CAH0388812 | empty |
| Bemisia tabaci | 23.04 | SEQ_NO_2 | A0A9P0A8F5_BEMTA | BEMITA_LOCUS7699 |
| Bemisia tabaci | 23.09 | SEQ_NO_2 | XP_018906909 | LOC109036937 |
| Bemisia tabaci | 23.09 | SEQ_NO_2 | XP_018897438 | LOC109030772 |
| Bemisia tabaci | 23.09 | SEQ_NO_2 | XP_018897440 | LOC109030772 |
| Bemisia tabaci | 23.09 | SEQ_NO_2 | XP_018897441 | LOC109030772 |
| Bemisia tabaci | 23.09 | SEQ_NO_2 | XP_018897442 | LOC109030772 |
| Bemisia tabaci | 23.09 | SEQ_NO_2 | XP_018897443 | LOC109030772 |
| Bemisia tabaci | 23.09 | SEQ_NO_2 | XP_018897444 | LOC109030772 |
| Bemisia tabaci | 23.09 | SEQ_NO_2 | XP_018897445 | LOC109030772 |
| Bemisia tabaci | 23.09 | SEQ_NO_2 | XP_018897446 | LOC109030772 |
| Bemisia tabaci | 23.09 | SEQ_NO_2 | XP_018897447 | LOC109030772 |
| Bemisia tabaci | 23.09 | SEQ_NO_2 | A0A9P0ACI5_BEMTA | BEMITA_LOCUS6670 |
| Bemisia tabaci | 20.77 | SEQ_NO_2 | A0A9P0A9J9_BEMTA | BEMITA_LOCUS6668 |
| Bemisia tabaci | 21.89 | SEQ_NO_2 | XP_018903285 | LOC109034531 |
| Bemisia tabaci | 21.89 | SEQ_NO_2 | A0A9P0AHL2_BEMTA | BEMITA_LOCUS10003 |
| Bemisia tabaci | 21.94 | SEQ_NO_2 | XP_018915065 | LOC109042643 |
| Bemisia tabaci | 21.94 | SEQ_NO_2 | XP_018915064 | LOC109042643 |
| Bemisia tabaci | 21.94 | SEQ_NO_2 | XP_018915063 | LOC109042643 |
| Bemisia tabaci | 22.87 | SEQ_NO_2 | A0A9P0F7D5_BEMTA | BEMITA_LOCUS10754 |
| Bemisia tabaci | 22.97 | SEQ_NO_2 | CAH0389468 | empty |
| Bemisia tabaci | 22.97 | SEQ_NO_2 | A0A9P0F4L7_BEMTA | BEMITA_LOCUS8294 |
| Bemisia tabaci | 22.97 | SEQ_NO_2 | XP_018900261 | LOC109032534 |
| Bemisia tabaci | 22.42 | SEQ_NO_2 | CAH0394912 | empty |
| Bemisia tabaci | 22.42 | SEQ_NO_2 | XP_018902113 | LOC109033794 |
| Bemisia tabaci | 22.42 | SEQ_NO_2 | A0A9P0F921_BEMTA | BEMITA_LOCUS13159 |
| Bemisia tabaci | 23.33 | SEQ_NO_2 | XP_018914126 | LOC109042030 |
| Bemisia tabaci | 22.8 | SEQ_NO_2 | XP_018916868 | LOC109043945 |
| Bemisia tabaci | 20.74 | SEQ_NO_2 | XP_018901923 | LOC109033669 |
| Bemisia tabaci | 22.75 | SEQ_NO_2 | XP_018903364 | LOC109034589 |
| Bemisia tabaci | 22.75 | SEQ_NO_2 | A0A9P0A2S3_BEMTA | BEMITA_LOCUS1641 |
| Bemisia tabaci | 22.7 | SEQ_NO_2 | XP_018906908 | LOC109036936 |
| Bemisia tabaci | 22.7 | SEQ_NO_2 | A0A9P0F6V6_BEMTA | BEMITA_LOCUS10753 |
| Bemisia tabaci | 23.04 | SEQ_NO_2 | A0A9P0F3C3_BEMTA | BEMITA_LOCUS5596 |
| Bemisia tabaci | 22.68 | SEQ_NO_2 | A0A9P0CBH2_BEMTA | BEMITA_LOCUS6669 |
| Bemisia tabaci | 20.62 | SEQ_NO_2 | A0A9P0F0V1_BEMTA | BEMITA_LOCUS3473 |
| Bemisia tabaci | 22.78 | SEQ_NO_2 | A0A9P0CCX9_BEMTA | BEMITA_LOCUS10706 |
| Bemisia tabaci | 23.87 | SEQ_NO_2 | XP_018908722 | LOC109038195 |
| Bemisia tabaci | 22.32 | SEQ_NO_2 | XP_018897397 | LOC109030744 |
| Bemisia tabaci | 22.45 | SEQ_NO_2 | XP_018897435 | LOC109030769 |
| Bemisia tabaci | 22.81 | SEQ_NO_2 | ARX98208 | empty |
| Bemisia tabaci | 22.81 | SEQ_NO_2 | XP_018896237 | LOC109029964 |
| Bemisia tabaci | 22.81 | SEQ_NO_2 | XP_018896239 | LOC109029964 |
| Bemisia tabaci | 22.81 | SEQ_NO_2 | XP_018896240 | LOC109029964 |
| Bemisia tabaci | 22.81 | SEQ_NO_2 | XP_018896241 | LOC109029964 |
| Bemisia tabaci | 22.81 | SEQ_NO_2 | XP_018896242 | LOC109029964 |
| Bemisia tabaci | 22.81 | SEQ_NO_2 | XP_018896243 | LOC109029964 |
| Bemisia tabaci | 22.81 | SEQ_NO_2 | XP_018896244 | LOC109029964 |
| Bemisia tabaci | 22.81 | SEQ_NO_2 | A0A1Z1XG36_BEMTA | empty |
| Bemisia tabaci | 22.6 | SEQ_NO_2 | CAH0394925 | empty |
| Bemisia tabaci | 22.6 | SEQ_NO_2 | XP_018902174 | LOC109033825 |
| Bemisia tabaci | 22.6 | SEQ_NO_2 | A0A9P0AP25_BEMTA | BEMITA_LOCUS13172 |
| Bemisia tabaci | 20.21 | SEQ_NO_2 | XP_018901925 | LOC109033671 |
| Bemisia tabaci | 20.21 | SEQ_NO_2 | XP_018901926 | LOC109033671 |
| Bemisia tabaci | 20.21 | SEQ_NO_2 | XP_018901927 | LOC109033671 |
| Bemisia tabaci | 20.21 | SEQ_NO_2 | XP_018901929 | LOC109033671 |
| Bemisia tabaci | 20.21 | SEQ_NO_2 | XP_018901930 | LOC109033671 |
| Bemisia tabaci | 21.97 | SEQ_NO_2 | A0A9P0EVH0_BEMTA | BEMITA_LOCUS481 |
| Bemisia tabaci | 21.97 | SEQ_NO_2 | A0A9P0A8B3_BEMTA | BEMITA_LOCUS5899 |
| Bemisia tabaci | 21.26 | SEQ_NO_2 | XP_018907730 | LOC109037470 |
| Bemisia tabaci | 30.25 | SEQ_NO_2 | CAH0394845 | empty |
| Bemisia tabaci | 30.25 | SEQ_NO_2 | A0A9P0ANY2_BEMTA | BEMITA_LOCUS13097 |
| Bemisia tabaci | 24.87 | SEQ_NO_2 | A0A9P0F9E5_BEMTA | BEMITA_LOCUS13573 |
| Bemisia tabaci | 23.17 | SEQ_NO_2 | XP_018916869 | LOC109043945 |
| Bemisia tabaci | 22.9 | SEQ_NO_2 | A0A9P0F4U7_BEMTA | BEMITA_LOCUS10751 |
| Bemisia tabaci | 23.56 | SEQ_NO_2 | CAH0394169 | empty |
| Bemisia tabaci | 23.56 | SEQ_NO_2 | ARX98205 | empty |
| Bemisia tabaci | 23.56 | SEQ_NO_2 | XP_018909446 | LOC109038730 |
| Bemisia tabaci | 23.56 | SEQ_NO_2 | XP_018909447 | LOC109038730 |
| Bemisia tabaci | 23.56 | SEQ_NO_2 | A0A9P0AL36_BEMTA | BEMITA_LOCUS12498 |
| Bemisia tabaci | 23.56 | SEQ_NO_2 | A0A1Z1XG25_BEMTA | empty |
| Bemisia tabaci | 22.2 | SEQ_NO_2 | XP_018915465 | LOC109042933 |
| Bemisia tabaci | 22.2 | SEQ_NO_2 | XP_018915470 | LOC109042933 |
| Bemisia tabaci | 22.2 | SEQ_NO_2 | XP_018915474 | LOC109042933 |
| Bemisia tabaci | 22.2 | SEQ_NO_2 | A0A9P0CFZ4_BEMTA | BEMITA_LOCUS11787 |
| Bemisia tabaci | 22.15 | SEQ_NO_2 | XP_018915482 | LOC109042933 |
| Bemisia tabaci | 24.37 | SEQ_NO_2 | XP_018905206 | LOC109035851 |
| Bemisia tabaci | 21.57 | SEQ_NO_2 | XP_018910315 | LOC109039334 |
| Bemisia tabaci | 24.12 | SEQ_NO_2 | A0A9P0F877_BEMTA | BEMITA_LOCUS12174 |
| Bemisia tabaci | 20.6 | SEQ_NO_2 | CAH0762940 | empty |
| Bemisia tabaci | 20.6 | SEQ_NO_2 | A0A9P0C7R5_BEMTA | BEMITA_LOCUS3015 |
| Bemisia tabaci | 20.6 | SEQ_NO_2 | XP_018907712 | LOC109037470 |
| Bemisia tabaci | 21.52 | SEQ_NO_2 | XP_018909895 | LOC109039026 |
| Bemisia tabaci | 20.73 | SEQ_NO_2 | ARX98210 | empty |
| Bemisia tabaci | 20.73 | SEQ_NO_2 | A0A1Z1XG22_BEMTA | empty |
| Bemisia tabaci | 20.73 | SEQ_NO_2 | XP_018907720 | LOC109037470 |
| Bemisia tabaci | 23.5 | SEQ_NO_2 | A0A9P0AAL9_BEMTA | BEMITA_LOCUS6674 |
| Bemisia tabaci | 22.27 | SEQ_NO_2 | XP_018897257 | LOC109030646 |
| Bemisia tabaci | 22.27 | SEQ_NO_2 | XP_018897258 | LOC109030646 |
| Bemisia tabaci | 22.27 | SEQ_NO_2 | A0A9P0CBG5_BEMTA | BEMITA_LOCUS6287 |
| Bemisia tabaci | 20.7 | SEQ_NO_2 | A0A9P0F4Y8_BEMTA | BEMITA_LOCUS10750 |
| Bemisia tabaci | 21.41 | SEQ_NO_2 | CAH0389225 | empty |
| Bemisia tabaci | 21.41 | SEQ_NO_2 | A0A9P0AD46_BEMTA | BEMITA_LOCUS8075 |
| Bemisia tabaci | 20.35 | SEQ_NO_2 | A0A9P0A7L3_BEMTA | BEMITA_LOCUS4923 |
| Bemisia tabaci | 22.04 | SEQ_NO_2 | CAH0389223 | empty |
| Bemisia tabaci | 22.04 | SEQ_NO_2 | A0A9P0AD71_BEMTA | BEMITA_LOCUS8075 |
| Bemisia tabaci | 22.14 | SEQ_NO_2 | XP_018903761 | LOC109034842 |
| Bemisia tabaci | 20.96 | SEQ_NO_2 | XP_018901036 | LOC109033067 |
| Bemisia tabaci | 20.96 | SEQ_NO_2 | XP_018901037 | LOC109033067 |
| Bemisia tabaci | 20.77 | SEQ_NO_2 | XP_018913794 | LOC109041818 |
| Bemisia tabaci | 21.8 | SEQ_NO_2 | XP_018916628 | LOC109043770 |
| Bemisia tabaci | 24.13 | SEQ_NO_2 | CAH0383300 | empty |
| Bemisia tabaci | 24.13 | SEQ_NO_2 | A0A9P0F080_BEMTA | BEMITA_LOCUS2761 |
| Bemisia tabaci | 23.47 | SEQ_NO_2 | A0A9P0C8H8_BEMTA | BEMITA_LOCUS6674 |
| Bemisia tabaci | 24.23 | SEQ_NO_2 | XP_018897469 | LOC109030788 |
| Bemisia tabaci | 22.19 | SEQ_NO_2 | A0A9P0ALX2_BEMTA | BEMITA_LOCUS14196 |
| Bemisia tabaci | 20.72 | SEQ_NO_2 | XP_018915067 | LOC109042643 |
| Bemisia tabaci | 23.46 | SEQ_NO_2 | XP_018897369 | LOC109030728 |
| Bemisia tabaci | 22.16 | SEQ_NO_2 | A0A9P0CED7_BEMTA | BEMITA_LOCUS10706 |
| Bemisia tabaci | 22.16 | SEQ_NO_2 | XP_018906976 | LOC109036975 |
| Bemisia tabaci | 22.88 | SEQ_NO_2 | A0A9P0AN16_BEMTA | BEMITA_LOCUS13520 |
| Bemisia tabaci | 20.9 | SEQ_NO_2 | XP_018915066 | LOC109042643 |
| Bemisia tabaci | 46.47 | SEQ_NO_2 | XP_018906867 | LOC109036902 |
| Bemisia tabaci | 22.32 | SEQ_NO_2 | XP_018905122 | LOC109035805 |
| Bemisia tabaci | 21.37 | SEQ_NO_2 | CAH0388664 | empty |
| Bemisia tabaci | 21.37 | SEQ_NO_2 | XP_018906730 | LOC109036802 |
| Bemisia tabaci | 21.37 | SEQ_NO_2 | A0A9P0AE61_BEMTA | BEMITA_LOCUS7567 |
| Bemisia tabaci | 21.37 | SEQ_NO_2 | XP_018906722 | LOC109036802 |
| Bemisia tabaci | 22.54 | SEQ_NO_2 | CAH0394104 | empty |
| Bemisia tabaci | 22.54 | SEQ_NO_2 | A0A9P0F695_BEMTA | BEMITA_LOCUS12441 |
| Bemisia tabaci | 21.02 | SEQ_NO_2 | A0A9P0AH05_BEMTA | BEMITA_LOCUS10002 |
| Bemisia tabaci | 25 | SEQ_NO_2 | XP_018904514 | LOC109035367 |
| Bemisia tabaci | 25 | SEQ_NO_2 | XP_018904515 | LOC109035367 |
| Bemisia tabaci | 25 | SEQ_NO_2 | XP_018904516 | LOC109035367 |
| Bemisia tabaci | 26.04 | SEQ_NO_2 | A0A9P0AET3_BEMTA | BEMITA_LOCUS11378 |
| Bemisia tabaci | 21.85 | SEQ_NO_2 | A0A9P0AM64_BEMTA | BEMITA_LOCUS12087 |
| Bemisia tabaci | 50.4 | SEQ_NO_2 | XP_018906865 | LOC109036900 |
| Candidatus | 28.16 | SEQ_NO_2 | ASX26719 | empty |
| Hamiltonella defensa | | | | |
| (Bemisia tabaci) | | | | |
| (Bemisia tabaci) | | | | |
| Bemisia tabaci | 29.17 | SEQ_NO_2 | CAH0390415 | empty |
| Bemisia tabaci | 29.17 | SEQ_NO_2 | A0A9P0AAW1_BEMTA | BEMITA_LOCUS9140 |
| Bemisia tabaci | 26.7 | SEQ_NO_2 | XP_018904517 | LOC109035367 |
| Bemisia tabaci | 26.7 | SEQ_NO_2 | XP_018904518 | LOC109035367 |
| Bemisia tabaci | 22.36 | SEQ_NO_6 | XP_018912317 | LOC109040740 |
| Bemisia tabaci | 22.16 | SEQ_NO_6 | CAH0388666 | empty |
| Bemisia tabaci | 22.16 | SEQ_NO_6 | A0A9P0AC80_BEMTA | BEMITA_LOCUS7569 |
| Bemisia tabaci | 20.35 | SEQ_NO_6 | A0A9P0AAD4_BEMTA | BEMITA_LOCUS6419 |
| Bemisia tabaci | 20.35 | SEQ_NO_6 | XP_018897322 | LOC109030689 |
| Bemisia tabaci | 21.92 | SEQ_NO_6 | XP_018906948 | LOC109036952 |
| Bemisia tabaci | 21.92 | SEQ_NO_6 | CAH0770726 | empty |
| Bemisia tabaci | 21.92 | SEQ_NO_6 | A0A9P0G3Z6_BEMTA | BEMITA_LOCUS7569 |
| Bemisia tabaci | 21.92 | SEQ_NO_6 | XP_018906940 | LOC109036952 |
| Bemisia tabaci | 21.92 | SEQ_NO_6 | CAH0388667 | empty |
| Bemisia tabaci | 21.92 | SEQ_NO_6 | A0A9P0A9U3_BEMTA | BEMITA_LOCUS7569 |
| Bemisia tabaci | 100 | SEQ_NO_22 | XP_018908582 | LOC109038094 |
| Bemisia tabaci | 99.93 | SEQ_NO_22 | XP_018908583 | LOC109038094 |
| Bemisia tabaci | 99.93 | SEQ_NO_22 | CAH0389150 | empty |
| Bemisia tabaci | 99.93 | SEQ_NO_22 | A0A9P0ADT3_BEMTA | BEMITA_LOCUS8010 |
| Bemisia tabaci | 58.58 | SEQ_NO_22 | XP_018907576 | LOC109037395 |
| Bemisia tabaci | 58.31 | SEQ_NO_22 | CAH0390278 | empty |
| Bemisia tabaci | 58.31 | SEQ_NO_22 | XP_018907575 | LOC109037395 |
| Bemisia tabaci | 58.31 | SEQ_NO_22 | A0A9P0F3D8_BEMTA | BEMITA_LOCUS9019 |
| Bemisia tabaci | 58.25 | SEQ_NO_22 | XP_018907571 | LOC109037395 |
| Bemisia tabaci | 58.18 | SEQ_NO_22 | XP_018907573 | LOC109037395 |
| Bemisia tabaci | 58.05 | SEQ_NO_22 | CAH0390277 | empty |
| Bemisia tabaci | 58.05 | SEQ_NO_22 | A0A9P0AGJ4_BEMTA | BEMITA_LOCUS9019 |
| Bemisia tabaci | 57.92 | SEQ_NO_22 | XP_018907572 | LOC109037395 |
| Bemisia tabaci | 54.33 | SEQ_NO_22 | CAH0390280 | empty |
| Bemisia tabaci | 54.33 | SEQ_NO_22 | A0A9P0F6R0_BEMTA | BEMITA_LOCUS9019 |
| Bemisia tabaci | 52.87 | SEQ_NO_22 | CAH0390279 | empty |
| Bemisia tabaci | 52.87 | SEQ_NO_22 | A0A9P0F3E2_BEMTA | BEMITA_LOCUS9019 |
| Bemisia tabaci | 34.01 | SEQ_NO_22 | XP_018899809 | LOC109032241 |
| Bemisia tabaci | 33.94 | SEQ_NO_22 | A0A9P0F935_BEMTA | BEMITA_LOCUS11622 |
| Bemisia tabaci | 33.99 | SEQ_NO_22 | A0A9P0F7Q5_BEMTA | BEMITA_LOCUS11622 |
| Bemisia tabaci | 35.32 | SEQ_NO_22 | XP_018897575 | LOC109030846 |
| Bemisia tabaci | 35.24 | SEQ_NO_22 | CAH0390818 | empty |
| Bemisia tabaci | 35.24 | SEQ_NO_22 | A0A9P0AGR3_BEMTA | BEMITA_LOCUS9509 |
| Bemisia tabaci | 35.54 | SEQ_NO_22 | A0A9P0AB69_BEMTA | BEMITA_LOCUS6635 |
| Bemisia tabaci | 35.52 | SEQ_NO_22 | XP_018902692 | LOC109034150 |
| Bemisia tabaci | 34.92 | SEQ_NO_22 | XP_018907669 | LOC109037449 |
| Bemisia tabaci | 34.92 | SEQ_NO_22 | CAH0390503 | empty |
| Bemisia tabaci | 34.92 | SEQ_NO_22 | A0A9P0ADN3_BEMTA | BEMITA_LOCUS9222 |
| Bemisia tabaci | 100 | SEQ_NO_34 | XP_018903291 | LOC109034533 |
| Bemisia tabaci | 96.24 | SEQ_NO_34 | CAH0389903 | empty |
| Bemisia tabaci | 96.24 | SEQ_NO_34 | A0A9P0F5J9_BEMTA | BEMITA_LOCUS8680 |
| Bemisia tabaci | 96.08 | SEQ_NO_34 | CAH0772022 | empty |
| Bemisia tabaci | 96.08 | SEQ_NO_34 | A0A9P0G2D3_BEMTA | BEMITA_LOCUS8680 |
| Bemisia tabaci | 96.08 | SEQ_NO_34 | CAH0772023 | empty |
| Bemisia tabaci | 96.08 | SEQ_NO_34 | A0A9P0GOR9_BEMTA | BEMITA_LOCUS8680 |
| Bemisia tabaci | 58.82 | SEQ_NO_34 | XP_018903294 | LOC109034536 |
| Bemisia tabaci | 58.63 | SEQ_NO_34 | CAH0389901 | empty |
| Bemisia tabaci | 58.63 | SEQ_NO_34 | A0A9P0F4Z1_BEMTA | BEMITA_LOCUS8678 |
| Trialeurodes | 58.25 | SEQ_NO_34 | QPA18374 | UGT352P4 |
| vaporariorum | | | | |
| (greenhouse | | | | |
| whitefly) | | | | |
| Trialeurodes | 58.25 | SEQ_NO_34 | A0A873P525_TRIVP | UGT352P4 |
| vaporariorum | | | | |
| Trialeurodes | 57.25 | SEQ_NO_34 | QPA18388 | UGT352P3 |
| vaporariorum | | | | |
| (greenhouse | | | | |
| whitefly) | | | | |
| Trialeurodes | 57.25 | SEQ_NO_34 | A0A873P529_TRIVP | UGT352P3 |
| vaporariorum | | | | |
| Trialeurodes | 56.76 | SEQ_NO_34 | QPA18408 | UGT352P2 |
| vaporariorum | | | | |
| (greenhouse | | | | |
| whitefly) | | | | |
| Trialeurodes | 56.76 | SEQ_NO_34 | A0A873P545_TRIVP | UGT352P2 |
| vaporariorum | | | | |
| Bemisia tabaci | 56.55 | SEQ_NO_34 | CAH0389900 | empty |
| Bemisia tabaci | 56.55 | SEQ_NO_34 | XP_018903292 | LOC109034534 |
| Bemisia tabaci | 56.55 | SEQ_NO_34 | A0A9P0F6E6_BEMTA | BEMITA_LOCUS8677 |
| Bemisia tabaci | 55.64 | SEQ_NO_34 | A0A9P0AMH4_BEMTA | BEMITA_LOCUS14101 |
| Bemisia tabaci | 56.3 | SEQ_NO_34 | XP_018907501 | LOC109037352 |
| Bemisia tabaci | 56.21 | SEQ_NO_34 | CAH0389711 | empty |
| Bemisia tabaci | 56.21 | SEQ_NO_34 | A0A9P0ADW9_BEMTA | BEMITA_LOCUS8515 |
| Bemisia tabaci | 56.07 | SEQ_NO_34 | XP_018896850 | LOC109030371 |
| Trialeurodes | 55.05 | SEQ_NO_34 | QPA18376 | UGT352P8 |
| vaporariorum | | | | |
| (greenhouse | | | | |
| whitefly) | | | | |
| Trialeurodes | 55.05 | SEQ_NO_34 | A0A873P522_TRIVP | UGT352P8 |
| vaporariorum | | | | |
| Bemisia tabaci | 54.63 | SEQ_NO_34 | XP_018901073 | LOC109033094 |
| Bemisia tabaci | 54.44 | SEQ_NO_34 | A0A9P0A386_BEMTA | BEMITA_LOCUS1841 |
| Bemisia tabaci | 54.67 | SEQ_NO_34 | XP_018900942 | LOC109033002 |
| Bemisia tabaci | 54.67 | SEQ_NO_34 | XP_018900943 | LOC109033002 |
| Bemisia tabaci | 54.68 | SEQ_NO_34 | XP_018895878 | LOC109029739 |
| Bemisia tabaci | 54.68 | SEQ_NO_34 | XP_018895879 | LOC109029739 |
| Bemisia tabaci | 54.68 | SEQ_NO_34 | A0A9P0F3L6_BEMTA | BEMITA_LOCUS5848 |
| Trialeurodes | 52.96 | SEQ_NO_34 | QPA18373 | UGT352P6 |
| vaporariorum | | | | |
| (greenhouse | | | | |
| whitefly) | | | | |
| Trialeurodes | 52.96 | SEQ_NO_34 | A0A873P505_TRIVP | UGT352P6 |
| vaporariorum | | | | |
| Bemisia tabaci | 53.36 | SEQ_NO_34 | A0A9P0A8B2_BEMTA | BEMITA_LOCUS5429 |
| Trialeurodes | 53.57 | SEQ_NO_34 | QPA18401 | UGT352S1 |
| vaporariorum | | | | |
| (greenhouse | | | | |
| whitefly) | | | | |
| Trialeurodes | 53.57 | SEQ_NO_34 | A0A873P542_TRIVP | UGT352S1 |
| vaporariorum | | | | |
| Bemisia tabaci | 53.88 | SEQ_NO_34 | A0A9P0ALJ1_BEMTA | BEMITA_LOCUS13576 |
| Bemisia tabaci | 54.3 | SEQ_NO_34 | A0A9P0F653_BEMTA | BEMITA_LOCUS12011 |
| Bemisia tabaci | 53.5 | SEQ_NO_34 | XP_018897569 | LOC109030843 |
| Bemisia tabaci | 53.6 | SEQ_NO_34 | XP_018911798 | LOC109040322 |
| Bemisia tabaci | 53.4 | SEQ_NO_34 | CAH0389712 | empty |
| Bemisia tabaci | 53.4 | SEQ_NO_34 | A0A9P0ADX4_BEMTA | BEMITA_LOCUS8516 |
| Bemisia tabaci | 54.21 | SEQ_NO_34 | XP_018907463 | LOC109037316 |
| Bemisia tabaci | 54.76 | SEQ_NO_34 | XP_018903293 | LOC109034535 |
| Bemisia tabaci | 53.31 | SEQ_NO_34 | XP_018909894 | LOC109039029 |
| Bemisia tabaci | 53.92 | SEQ_NO_34 | XP_018896856 | LOC109030375 |
| Bemisia tabaci | 51.93 | SEQ_NO_34 | XP_018912485 | LOC109040852 |
| Bemisia tabaci | 53.52 | SEQ_NO_34 | XP_018896849 | LOC109030370 |
| Bemisia tabaci | 53.02 | SEQ_NO_34 | A0A9P0F7S7_BEMTA | BEMITA_LOCUS14129 |
| Bemisia tabaci | 53.22 | SEQ_NO_34 | A0A9P0API1_BEMTA | BEMITA_LOCUS13456 |
| Bemisia tabaci | 53.12 | SEQ_NO_34 | XP_018914530 | LOC109042315 |
| Bemisia tabaci | 53.03 | SEQ_NO_34 | A0A9P0FAE0_BEMTA | BEMITA_LOCUS13342 |
| Bemisia tabaci | 52.53 | SEQ_NO_34 | CAH0772019 | empty |
| Bemisia tabaci | 52.53 | SEQ_NO_34 | A0A9P0CAB0_BEMTA | BEMITA_LOCUS8679 |
| Bemisia tabaci | 53.85 | SEQ_NO_34 | XP_018896848 | LOC109030369 |
| Bemisia tabaci | 53.13 | SEQ_NO_34 | A0A9P0AQR1_BEMTA | BEMITA_LOCUS14102 |
| Trialeurodes | 52.64 | SEQ_NO_34 | QPA18379 | UGT352T1 |
| vaporariorum | | | | |
| (greenhouse | | | | |
| whitefly) | | | | |
| Trialeurodes | 52.64 | SEQ_NO_34 | A0A873P519_TRIVP | UGT352T1 |
| vaporariorum | | | | |
| Bemisia tabaci | 53.14 | SEQ_NO_34 | A0A9P0AJL4_BEMTA | BEMITA_LOCUS14103 |
| Bemisia tabaci | 52.84 | SEQ_NO_34 | XP_018909893 | LOC109039028 |
| Bemisia tabaci | 52.16 | SEQ_NO_34 | XP_018897720 | LOC109030936 |
| Trialeurodes | 52.16 | SEQ_NO_34 | QPA18380 | UGT352P7 |
| vaporariorum | | | | |
| (greenhouse | | | | |
| whitefly) | | | | |
| Trialeurodes | 52.16 | SEQ_NO_34 | A0A873P514_TRIVP | UGT352P7 |
| vaporariorum | | | | |
| Bemisia tabaci | 53.46 | SEQ_NO_34 | A0A9P0FA46_BEMTA | BEMITA_LOCUS14098 |
| Trialeurodes | 52.65 | SEQ_NO_34 | QPA18381 | UGT352P5 |
| vaporariorum | | | | |
| (greenhouse | | | | |
| whitefly) | | | | |
| Trialeurodes | 52.65 | SEQ_NO_34 | A0A873P518_TRIVP | UGT352P5 |
| vaporariorum | | | | |
| Bemisia tabaci | 52.75 | SEQ_NO_34 | A0A9P0AP04_BEMTA | BEMITA_LOCUS14104 |
| Bemisia tabaci | 52.26 | SEQ_NO_34 | CAH0389259 | empty |
| Bemisia tabaci | 52.26 | SEQ_NO_34 | A0A9P0A948_BEMTA | BEMITA_LOCUS8106 |
| Bemisia tabaci | 52.07 | SEQ_NO_34 | XP_018916669 | LOC109043808 |
| Bemisia tabaci | 52.56 | SEQ_NO_34 | XP_018896847 | LOC109030368 |
| Bemisia tabaci | 52.57 | SEQ_NO_34 | XP_018904060 | LOC109035046 |
| Bemisia tabaci | 51.98 | SEQ_NO_34 | XP_018896827 | LOC109030357 |
| Bemisia tabaci | 51.98 | SEQ_NO_34 | XP_018896828 | LOC109030357 |
| Bemisia tabaci | 51.79 | SEQ_NO_34 | XP_018896855 | LOC109030374 |
| Bemisia tabaci | 51.41 | SEQ_NO_34 | A0A9P0ALR6_BEMTA | BEMITA_LOCUS14105 |
| Bemisia tabaci | 50.75 | SEQ_NO_34 | XP_018912232 | LOC109040684 |
| Trialeurodes | 51.13 | SEQ_NO_34 | QPA18378 | UGT352R1 |
| vaporariorum | | | | |
| (greenhouse | | | | |
| whitefly) | | | | |
| Trialeurodes | 51.13 | SEQ_NO_34 | A0A873P521_TRIVP | UGT352R1 |
| vaporariorum | | | | |
| Bemisia tabaci | 50.56 | SEQ_NO_34 | CAH0772768 | empty |
| Bemisia tabaci | 50.56 | SEQ_NO_34 | A0A9P0CB14_BEMTA | BEMITA_LOCUS9337 |
| Bemisia tabaci | 52.54 | SEQ_NO_34 | A0A9P0F651_BEMTA | BEMITA_LOCUS9953 |
| Bemisia tabaci | 52.96 | SEQ_NO_34 | A0A9P0ANU3_BEMTA | BEMITA_LOCUS13575 |
| Bemisia tabaci | 51.05 | SEQ_NO_34 | A0A9P0AP15_BEMTA | BEMITA_LOCUS14107 |
| Bemisia tabaci | 50.09 | SEQ_NO_34 | XP_018896115 | LOC109029876 |
| Bemisia tabaci | 50.66 | SEQ_NO_34 | CAH0390832 | empty |
| Bemisia tabaci | 50.66 | SEQ_NO_34 | A0A9P0F5R1_BEMTA | BEMITA_LOCUS9523 |
| Trialeurodes | 50.57 | SEQ_NO_34 | QPA18383 | UGT352U1 |
| vaporariorum | | | | |
| (greenhouse | | | | |
| whitefly) | | | | |
| Trialeurodes | 50.57 | SEQ_NO_34 | A0A873P517_TRIVP | UGT352U1 |
| vaporariorum | | | | |
| Bemisia tabaci | 49.72 | SEQ_NO_34 | CAH0382890 | empty |
| Bemisia tabaci | 49.72 | SEQ_NO_34 | A0A9P0EZV9_BEMTA | BEMITA_LOCUS2385 |
| Bemisia tabaci | 49.63 | SEQ_NO_34 | XP_018912467 | LOC109040838 |
| Bemisia tabaci | 53.43 | SEQ_NO_34 | CAH0383324 | empty |
| Bemisia tabaci | 53.43 | SEQ_NO_34 | A0A9P0A0Q0_BEMTA | BEMITA_LOCUS2784 |
| Bemisia tabaci | 49.72 | SEQ_NO_34 | XP_018901673 | LOC109033483 |
| Bemisia tabaci | 50.09 | SEQ_NO_34 | XP_018900337 | LOC109032577 |
| Bemisia tabaci | 50.87 | SEQ_NO_34 | XP_018896852 | LOC109030373 |
| Bemisia tabaci | 50.87 | SEQ_NO_34 | XP_018896854 | LOC109030373 |
| Bemisia tabaci | 49.53 | SEQ_NO_34 | A0A9P0F6P6_BEMTA | BEMITA_LOCUS9954 |
| Bemisia tabaci | 50.87 | SEQ_NO_34 | A0A9P0AQI0_BEMTA | BEMITA_LOCUS14108 |
| Bemisia tabaci | 49.34 | SEQ_NO_34 | XP_018916276 | LOC109043524 |
| Bemisia tabaci | 49.06 | SEQ_NO_34 | A0A9P0C602_BEMTA | BEMITA_LOCUS9973 |
| Bemisia tabaci | 50.78 | SEQ_NO_34 | XP_018896851 | LOC109030372 |
| Bemisia tabaci | 49.23 | SEQ_NO_34 | XP_018916283 | LOC109043524 |
| Bemisia tabaci | 49.23 | SEQ_NO_34 | XP_018916291 | LOC109043524 |
| Bemisia tabaci | 50.7 | SEQ_NO_34 | XP_018912233 | LOC109040684 |
| Bemisia tabaci | 53.94 | SEQ_NO_34 | XP_018909896 | LOC109039029 |
| Bemisia tabaci | 53.28 | SEQ_NO_34 | XP_018914531 | LOC109042315 |
| Bemisia tabaci | 55.18 | SEQ_NO_34 | XP_018900944 | LOC109033002 |
| Trialeurodes | 56.58 | SEQ_NO_34 | QPA18393 | UGT352P1 |
| vaporariorum | | | | |
| (greenhouse | | | | |
| whitefly) | | | | |
| Trialeurodes | 56.58 | SEQ_NO_34 | A0A873P531_TRIVP | UGT352P1 |
| vaporariorum | | | | |
| Bemisia tabaci | 46.78 | SEQ_NO_34 | XP_018913220 | LOC109041340 |
| Trialeurodes | 50.64 | SEQ_NO_34 | QPA18391 | UGT352V1 |
| vaporariorum | | | | |
| (greenhouse | | | | |
| whitefly) | | | | |
| Trialeurodes | 50.64 | SEQ_NO_34 | A0A873P532_TRIVP | UGT352V1 |
| vaporariorum | | | | |
| Bemisia tabaci | 50.21 | SEQ_NO_34 | A0A9P0CD96_BEMTA | BEMITA_LOCUS11747 |
| Bemisia tabaci | 46 | SEQ_NO_34 | A0A9P0A0M7_BEMTA | BEMITA_LOCUS713 |
| Bemisia tabaci | 55.14 | SEQ_NO_34 | XP_018901369 | LOC109033275 |
| Bemisia tabaci | 55.14 | SEQ_NO_34 | XP_018901370 | LOC109033275 |
| Bemisia tabaci | 56.49 | SEQ_NO_34 | CAH0772020 | empty |
| Bemisia tabaci | 56.49 | SEQ_NO_34 | A0A9P0CEX0_BEMTA | BEMITA_LOCUS8679 |
| Bemisia tabaci | 49.68 | SEQ_NO_34 | XP_018903778 | LOC109034860 |
| Bemisia tabaci | 53.05 | SEQ_NO_34 | XP_018896116 | LOC109029876 |
| Bemisia tabaci | 50.46 | SEQ_NO_34 | A0A9P0F839_BEMTA | BEMITA_LOCUS14108 |
| Trialeurodes | 59.46 | SEQ_NO_34 | QPA18375 | UGT352Q1 |
| vaporariorum | | | | |
| (greenhouse | | | | |
| whitefly) | | | | |
| Trialeurodes | 59.46 | SEQ_NO_34 | A0A873P524_TRIVP | UGT352Q1 |
| vaporariorum | | | | |
| Bemisia tabaci | 50.11 | SEQ_NO_34 | A0A9P0F5P4_BEMTA | BEMITA_LOCUS11747 |
| Trialeurodes | 41.92 | SEQ_NO_34 | A0A873P516_TRIVP | UGT358B1 |
| vaporariorum | | | | |
| Bemisia tabaci | 41.67 | SEQ_NO_34 | A0A9P0CFL4_BEMTA | BEMITA_LOCUS13814 |
| Bemisia tabaci | 42.07 | SEQ_NO_34 | XP_018903756 | LOC109034840 |
| Bemisia tabaci | 42.07 | SEQ_NO_34 | XP_018903757 | LOC109034840 |
| Bemisia tabaci | 58.74 | SEQ_NO_34 | CAH0389713 | empty |
| Bemisia tabaci | 58.74 | SEQ_NO_34 | A0A9P0AFM1_BEMTA | BEMITA_LOCUS8516 |
| Bemisia tabaci | 42.04 | SEQ_NO_34 | A0A9P0ANF8_BEMTA | BEMITA_LOCUS13814 |
| Bemisia tabaci | 54.71 | SEQ_NO_34 | A0A9P0CFK9_BEMTA | BEMITA_LOCUS14105 |
| Bemisia tabaci | 40 | SEQ_NO_34 | A0A9P0ANQ3_BEMTA | BEMITA_LOCUS13940 |
| Bemisia tabaci | 40.04 | SEQ_NO_34 | XP_018910145 | LOC109039208 |
| Trialeurodes | 35.59 | SEQ_NO_34 | QPA18409 | UGT387A1 |
| vaporariorum | | | | |
| (greenhouse | | | | |
| whitefly) | | | | |
| Trialeurodes | 35.59 | SEQ_NO_34 | A0A873P549_TRIVP | UGT387A1 |
| vaporariorum | | | | |
| Bemisia tabaci | 39.59 | SEQ_NO_34 | A0A9P0ALI6_BEMTA | BEMITA_LOCUS13940 |
| Bemisia tabaci | 32.2 | SEQ_NO_34 | XP_018907189 | LOC109037135 |
| Bemisia tabaci | 32.38 | SEQ_NO_34 | XP_018900076 | LOC109032412 |
| Bemisia tabaci | 31.58 | SEQ_NO_34 | CAH0762836 | empty |
| Bemisia tabaci | 31.58 | SEQ_NO_34 | A0A9P0C7Q1_BEMTA | BEMITA_LOCUS2923 |
| Bemisia tabaci | 31.52 | SEQ_NO_34 | XP_018899270 | LOC109031899 |
| Bemisia tabaci | 31.52 | SEQ_NO_34 | XP_018899272 | LOC109031899 |
| Bemisia tabaci | 30.87 | SEQ_NO_34 | XP_018910301 | LOC109039327 |
| Bemisia tabaci | 30.87 | SEQ_NO_34 | XP_018910303 | LOC109039327 |
| Trialeurodes | 30.9 | SEQ_NO_34 | QPA18405 | UGT355E1 |
| vaporariorum | | | | |
| (greenhouse | | | | |
| whitefly) | | | | |
| Trialeurodes | 30.9 | SEQ_NO_34 | A0A873P558_TRIVP | UGT355E1 |
| vaporariorum | | | | |
| Bemisia tabaci | 29.98 | SEQ_NO_34 | CAH0389551 | empty |
| Bemisia tabaci | 29.98 | SEQ_NO_34 | A0A9P0ACB7_BEMTA | BEMITA_LOCUS8366 |
| Bemisia tabaci | 30.81 | SEQ_NO_34 | XP_018900049 | LOC109032402 |
| Bemisia tabaci | 29.29 | SEQ_NO_34 | CAH0387889 | empty |
| Bemisia tabaci | 29.29 | SEQ_NO_34 | A0A9P0A9U5_BEMTA | BEMITA_LOCUS6849 |
| Bemisia tabaci | 29.51 | SEQ_NO_34 | XP_018914989 | LOC109042596 |
| Bemisia tabaci | 29.98 | SEQ_NO_34 | A0A9P0AF77_BEMTA | BEMITA_LOCUS10122 |
| Bemisia tabaci | 30.96 | SEQ_NO_34 | XP_018900057 | LOC109032402 |
| Bemisia tabaci | 30.96 | SEQ_NO_34 | XP_018900058 | LOC109032402 |
| Bemisia tabaci | 31.02 | SEQ_NO_34 | CAH0388827 | empty |
| Bemisia tabaci | 31.02 | SEQ_NO_34 | A0A9P0A8G3_BEMTA | BEMITA_LOCUS7713 |
| Bemisia tabaci | 29.52 | SEQ_NO_34 | XP_018904950 | LOC109035695 |
| Trialeurodes | 31.56 | SEQ_NO_34 | QPA18407 | UGT354D5 |
| vaporariorum | | | | |
| (greenhouse | | | | |
| whitefly) | | | | |
| Trialeurodes | 31.56 | SEQ_NO_34 | A0A873P535_TRIVP | UGT354D5 |
| vaporariorum | | | | |
| Bemisia tabaci | 30.89 | SEQ_NO_34 | XP_018911268 | LOC109039954 |
| Bemisia tabaci | 29.8 | SEQ_NO_34 | CAH0383478 | empty |
| Bemisia tabaci | 29.8 | SEQ_NO_34 | A0A9P0A0Z1_BEMTA | BEMITA_LOCUS2922 |
| Trialeurodes | 30.08 | SEQ_NO_34 | QPA18392 | UGT354D2 |
| vaporariorum | | | | |
| (greenhouse | | | | |
| whitefly) | | | | |
| Trialeurodes | 30.08 | SEQ_NO_34 | A0A873P530_TRIVP | UGT354D2 |
| vaporariorum | | | | |
| Bemisia tabaci | 30.13 | SEQ_NO_34 | XP_018915403 | LOC109042889 |
| Bemisia tabaci | 30.13 | SEQ_NO_34 | XP_018915404 | LOC109042889 |
| Trialeurodes | 30.13 | SEQ_NO_34 | QPA18398 | UGT359B1 |
| vaporariorum | | | | |
| (greenhouse | | | | |
| whitefly) | | | | |
| Trialeurodes | 30.13 | SEQ_NO_34 | A0A873P539_TRIVP | UGT359B1 |
| vaporariorum | | | | |
| Trialeurodes | 31.75 | SEQ_NO_34 | QPA18403 | UGT354D3 |
| vaporariorum | | | | |
| (greenhouse | | | | |
| whitefly) | | | | |
| Trialeurodes | 31.75 | SEQ_NO_34 | A0A873P541_TRIVP | UGT354D3 |
| vaporariorum | | | | |
| Bemisia tabaci | 30 | SEQ_NO_34 | XP_018900056 | LOC109032402 |
| Bemisia tabaci | 47.45 | SEQ_NO_34 | XP_018912243 | LOC109040694 |
| Bemisia tabaci | 28.83 | SEQ_NO_34 | CAH0770868 | empty |
| Bemisia tabaci | 28.83 | SEQ_NO_34 | A0A9P0CDH6_BEMTA | BEMITA_LOCUS7687 |
| Bemisia tabaci | 29.59 | SEQ_NO_34 | XP_018910401 | LOC109039385 |
| Bemisia tabaci | 29.59 | SEQ_NO_34 | A0A9P0G304_BEMTA | BEMITA_LOCUS12283 |
| Bemisia tabaci | 29.59 | SEQ_NO_34 | XP_018909188 | LOC109038550 |
| Bemisia tabaci | 29.15 | SEQ_NO_34 | CAH0383687 | empty |
| Bemisia tabaci | 29.15 | SEQ_NO_34 | A0A9P0A1B5_BEMTA | BEMITA_LOCUS3112 |
| Trialeurodes | 30.49 | SEQ_NO_34 | QPA18377 | UGT360A5 |
| vaporariorum | | | | |
| (greenhouse | | | | |
| whitefly) | | | | |
| Trialeurodes | 30.49 | SEQ_NO_34 | A0A873P511_TRIVP | UGT360A5 |
| vaporariorum | | | | |
| Bemisia tabaci | 29.57 | SEQ_NO_34 | A0A9P0AI87_BEMTA | BEMITA_LOCUS10897 |
| Bemisia tabaci | 29.18 | SEQ_NO_34 | CAH0762742 | empty |
| Bemisia tabaci | 29.18 | SEQ_NO_34 | A0A9P0G2Q8_BEMTA | BEMITA_LOCUS2841 |
| Bemisia tabaci | 29.16 | SEQ_NO_34 | A0A9N9ZZ79_BEMTA | BEMITA_LOCUS425 |
| Bemisia tabaci | 28.57 | SEQ_NO_34 | CAH0388801 | empty |
| Bemisia tabaci | 28.57 | SEQ_NO_34 | XP_018905881 | LOC109036264 |
| Bemisia tabaci | 28.57 | SEQ_NO_34 | A0A9P0F271_BEMTA | BEMITA_LOCUS7688 |
| Bemisia tabaci | 28.94 | SEQ_NO_34 | CAH0383688 | empty |
| Bemisia tabaci | 28.94 | SEQ_NO_34 | XP_018917940 | LOC109044583 |
| Bemisia tabaci | 28.94 | SEQ_NO_34 | A0A9P0A3V6_BEMTA | BEMITA_LOCUS3113 |
| Bemisia tabaci | 28.97 | SEQ_NO_34 | XP_018896916 | LOC109030420 |
| Bemisia tabaci | 28.97 | SEQ_NO_34 | XP_018910400 | LOC109039385 |
| Bemisia tabaci | 28.97 | SEQ_NO_34 | A0A9P0CG61_BEMTA | BEMITA_LOCUS12283 |
| Trialeurodes | 29.92 | SEQ_NO_34 | QPA18371 | UGT356F1 |
| vaporariorum | | | | |
| (greenhouse | | | | |
| whitefly) | | | | |
| Trialeurodes | 29.92 | SEQ_NO_34 | A0A873P507_TRIVP | UGT356F1 |
| vaporariorum | | | | |
| Bemisia tabaci | 28.95 | SEQ_NO_34 | XP_018907955 | LOC109037643 |
| Bemisia tabaci | 29.22 | SEQ_NO_34 | CAH0383496 | empty |
| Bemisia tabaci | 29.22 | SEQ_NO_34 | A0A9P0A3P3_BEMTA | BEMITA_LOCUS2940 |
| Bemisia tabaci | 29 | SEQ_NO_34 | A0A9P0APB5_BEMTA | BEMITA_LOCUS14051 |
| Bemisia tabaci | 28.62 | SEQ_NO_34 | A0A9P0F9U0_BEMTA | BEMITA_LOCUS14050 |
| Bemisia tabaci | 29.56 | SEQ_NO_34 | XP_018896975 | LOC109030459 |
| Bemisia tabaci | 29.56 | SEQ_NO_34 | XP_018896976 | LOC109030459 |
| Trialeurodes | 31.42 | SEQ_NO_34 | QPA18370 | UGT356E1 |
| vaporariorum | | | | |
| (greenhouse | | | | |
| whitefly) | | | | |
| Trialeurodes | 31.42 | SEQ_NO_34 | A0A873P509_TRIVP | UGT356E1 |
| vaporariorum | | | | |
| Trialeurodes | 29.2 | SEQ_NO_34 | QPA18396 | UGT356D1 |
| vaporariorum | | | | |
| (greenhouse | | | | |
| whitefly) | | | | |
| Trialeurodes | 29.2 | SEQ_NO_34 | A0A873P543_TRIVP | UGT356D1 |
| vaporariorum | | | | |
| Bemisia tabaci | 28.84 | SEQ_NO_34 | XP_018917928 | LOC109044573 |
| Bemisia tabaci | 28.57 | SEQ_NO_34 | XP_018900051 | LOC109032402 |
| Bemisia tabaci | 28.57 | SEQ_NO_34 | XP_018900052 | LOC109032402 |
| Bemisia tabaci | 28.57 | SEQ_NO_34 | XP_018900053 | LOC109032402 |
| Bemisia tabaci | 28.57 | SEQ_NO_34 | XP_018900054 | LOC109032402 |
| Bemisia tabaci | 29.15 | SEQ_NO_34 | XP_018896358 | LOC109030053 |
| Bemisia tabaci | 28.82 | SEQ_NO_34 | A0A9P0A5Z5_BEMTA | BEMITA_LOCUS4253 |
| Bemisia tabaci | 29.09 | SEQ_NO_34 | XP_018898301 | LOC109031307 |
| Trialeurodes | 28.28 | SEQ_NO_34 | QPA18397 | UGT386L1 |
| vaporariorum | | | | |
| (greenhouse | | | | |
| whitefly) | | | | |
| Trialeurodes | 28.28 | SEQ_NO_34 | A0A873P527_TRIVP | UGT386L1 |
| vaporariorum | | | | |
| Bemisia tabaci | 28.6 | SEQ_NO_34 | XP_018897564 | LOC109030838 |
| Bemisia tabaci | 29.76 | SEQ_NO_34 | A0A9P0F097_BEMTA | BEMITA_LOCUS3616 |
| Trialeurodes | 27.62 | SEQ_NO_34 | QPA18386 | UGT360B2 |
| vaporariorum | | | | |
| (greenhouse | | | | |
| whitefly) | | | | |
| Trialeurodes | 27.62 | SEQ_NO_34 | A0A873P533_TRIVP | UGT360B2 |
| vaporariorum | | | | |
| Bemisia tabaci | 28.96 | SEQ_NO_34 | CAH0383689 | empty |
| Bemisia tabaci | 28.96 | SEQ_NO_34 | A0A9P0A3U2_BEMTA | BEMITA_LOCUS3114 |
| Bemisia tabaci | 29.07 | SEQ_NO_34 | CAH0388138 | empty |
| Bemisia tabaci | 29.07 | SEQ_NO_34 | XP_018901567 | LOC109033421 |
| Bemisia tabaci | 29.07 | SEQ_NO_34 | A0A9P0F1M9_BEMTA | BEMITA_LOCUS7076 |
| Bemisia tabaci | 29.41 | SEQ_NO_34 | XP_018917997 | LOC109044625 |
| Bemisia tabaci | 28.36 | SEQ_NO_34 | XP_018900050 | LOC109032402 |
| Bemisia tabaci | 27.9 | SEQ_NO_34 | XP_018905658 | LOC109036144 |
| Bemisia tabaci | 27.9 | SEQ_NO_34 | XP_018905659 | LOC109036144 |
| Bemisia tabaci | 27.9 | SEQ_NO_34 | XP_018905660 | LOC109036144 |
| Bemisia tabaci | 27.85 | SEQ_NO_34 | XP_018898507 | LOC109031455 |
| Bemisia tabaci | 28.99 | SEQ_NO_34 | XP_018905849 | LOC109036250 |
| Bemisia tabaci | 28.99 | SEQ_NO_34 | XP_018905858 | LOC109036250 |
| Bemisia tabaci | 28.99 | SEQ_NO_34 | XP_018905867 | LOC109036250 |
| Trialeurodes | 27.75 | SEQ_NO_34 | QPA18384 | UGT356G1 |
| vaporariorum | | | | |
| (greenhouse | | | | |
| whitefly) | | | | |
| Trialeurodes | 27.75 | SEQ_NO_34 | A0A873P537_TRIVP | UGT356G1 |
| vaporariorum | | | | |
| Bemisia tabaci | 28.22 | SEQ_NO_34 | XP_018905835 | LOC109036246 |
| Bemisia tabaci | 29.19 | SEQ_NO_34 | A0A9P0CBE9_BEMTA | BEMITA_LOCUS12283 |
| Bemisia tabaci | 28.46 | SEQ_NO_34 | XP_018899263 | LOC109031897 |
| Bemisia tabaci | 28.24 | SEQ_NO_34 | XP_018911460 | LOC109040109 |
| Bemisia tabaci | 28.02 | SEQ_NO_34 | CAH0388428 | empty |
| Bemisia tabaci | 28.02 | SEQ_NO_34 | A0A9P0AC41_BEMTA | BEMITA_LOCUS7342 |
| Trialeurodes | 28.13 | SEQ_NO_34 | QPA18395 | UGT355G1 |
| vaporariorum | | | | |
| (greenhouse | | | | |
| whitefly) | | | | |
| Trialeurodes | 28.13 | SEQ_NO_34 | A0A873P544_TRIVP | UGT355G1 |
| vaporariorum | | | | |
| Bemisia tabaci | 28.46 | SEQ_NO_34 | XP_018899264 | LOC109031897 |
| Bemisia tabaci | 28.46 | SEQ_NO_34 | XP_018899265 | LOC109031897 |
| Bemisia tabaci | 28.46 | SEQ_NO_34 | XP_018899266 | LOC109031897 |
| Bemisia tabaci | 28.46 | SEQ_NO_34 | XP_018899267 | LOC109031897 |
| Bemisia tabaci | 28.46 | SEQ_NO_34 | XP_018899268 | LOC109031897 |
| Bemisia tabaci | 28.46 | SEQ_NO_34 | XP_018899269 | LOC109031897 |
| Bemisia tabaci | 28.46 | SEQ_NO_34 | XP_018899262 | LOC109031897 |
| Trialeurodes | 28.52 | SEQ_NO_34 | A0A873P546_TRIVP | UGT355F1 |
| vaporariorum | | | | |
| Bemisia tabaci | 28.27 | SEQ_NO_34 | CAH0383819 | empty |
| Bemisia tabaci | 28.27 | SEQ_NO_34 | A0A9P0A518_BEMTA | BEMITA_LOCUS3228 |
| Bemisia tabaci | 28.05 | SEQ_NO_34 | A0A9P0EVM8_BEMTA | BEMITA_LOCUS360 |
| Bemisia tabaci | 27.85 | SEQ_NO_34 | XP_018896780 | LOC109030336 |
| Trialeurodes | 27.43 | SEQ_NO_34 | QPA18385 | UGT360B1 |
| vaporariorum | | | | |
| (greenhouse | | | | |
| whitefly) | | | | |
| Trialeurodes | 27.43 | SEQ_NO_34 | A0A873P534_TRIVP | UGT360B1 |
| vaporariorum | | | | |
| Bemisia tabaci | 29.39 | SEQ_NO_34 | A0A9P0CED8_BEMTA | BEMITA_LOCUS10122 |
| Trialeurodes | 29.98 | SEQ_NO_34 | QPA18399 | UGT359C1 |
| vaporariorum | | | | |
| (greenhouse | | | | |
| whitefly) | | | | |
| Trialeurodes | 29.98 | SEQ_NO_34 | A0A873P536_TRIVP | UGT359C1 |
| vaporariorum | | | | |
| Bemisia tabaci | 28.95 | SEQ_NO_34 | XP_018900059 | LOC109032402 |
| Bemisia tabaci | 27.45 | SEQ_NO_34 | XP_018904599 | LOC109035425 |
| Bemisia tabaci | 27.45 | SEQ_NO_34 | XP_018904600 | LOC109035425 |
| Bemisia tabaci | 27.41 | SEQ_NO_34 | CAH0388912 | empty |
| Bemisia tabaci | 27.41 | SEQ_NO_34 | A0A9P0ABC6_BEMTA | BEMITA_LOCUS7796 |
| Bemisia tabaci | 27.41 | SEQ_NO_34 | CAH0770866 | empty |
| Bemisia tabaci | 27.41 | SEQ_NO_34 | A0A9P0G534_BEMTA | BEMITA_LOCUS7686 |
| Trialeurodes | 27.63 | SEQ_NO_34 | QPA18404 | UGT360B6 |
| vaporariorum | | | | |
| (greenhouse | | | | |
| whitefly) | | | | |
| Trialeurodes | 27.63 | SEQ_NO_34 | A0A873P555_TRIVP | UGT360B6 |
| vaporariorum | | | | |
| Bemisia tabaci | 27.63 | SEQ_NO_34 | CAH0388802 | empty |
| Bemisia tabaci | 27.63 | SEQ_NO_34 | A0A9P0F269_BEMTA | BEMITA_LOCUS7689 |
| Trialeurodes | 28.23 | SEQ_NO_34 | QPA18389 | UGT380B4 |
| vaporariorum | | | | |
| (greenhouse | | | | |
| whitefly) | | | | |
| Trialeurodes | 28.23 | SEQ_NO_34 | A0A873P528_TRIVP | UGT380B4 |
| vaporariorum | | | | |
| Bemisia tabaci | 27.13 | SEQ_NO_34 | XP_018907242 | LOC109037151 |
| Bemisia tabaci | 27.13 | SEQ_NO_34 | XP_018907209 | LOC109037151 |
| Bemisia tabaci | 27.13 | SEQ_NO_34 | XP_018907216 | LOC109037151 |
| Bemisia tabaci | 27.13 | SEQ_NO_34 | XP_018907223 | LOC109037151 |
| Bemisia tabaci | 27.13 | SEQ_NO_34 | XP_018907232 | LOC109037151 |
| Bemisia tabaci | 27.31 | SEQ_NO_34 | XP_018907262 | LOC109037151 |
| Trialeurodes | 28.08 | SEQ_NO_34 | QPA18387 | UGT360B3 |
| vaporariorum | | | | |
| (greenhouse | | | | |
| whitefly) | | | | |
| Trialeurodes | 28.08 | SEQ_NO_34 | A0A873P515_TRIVP | UGT360B3 |
| vaporariorum | | | | |
| Trialeurodes | 31.74 | SEQ_NO_34 | QPA18400 | UGT357A2 |
| vaporariorum | | | | |
| (greenhouse | | | | |
| whitefly) | | | | |
| Trialeurodes | 31.74 | SEQ_NO_34 | A0A873P538_TRIVP | UGT357A2 |
| vaporariorum | | | | |
| Bemisia tabaci | 27.27 | SEQ_NO_34 | CAH0770867 | empty |
| Bemisia tabaci | 27.27 | SEQ_NO_34 | A0A9P0CAE3_BEMTA | BEMITA_LOCUS7686 |
| Bemisia tabaci | 26.59 | SEQ_NO_34 | XP_018907253 | LOC109037151 |
| Trialeurodes | 26.56 | SEQ_NO_34 | QPA18368 | UGT355D1 |
| vaporariorum | | | | |
| (greenhouse | | | | |
| whitefly) | | | | |
| Trialeurodes | 26.56 | SEQ_NO_34 | A0A873P513_TRIVP | UGT355D1 |
| vaporariorum | | | | |
| Bemisia tabaci | 27.02 | SEQ_NO_34 | XP_018906826 | LOC109036871 |
| Bemisia tabaci | 27.02 | SEQ_NO_34 | XP_018906827 | LOC109036871 |
| Bemisia tabaci | 27.02 | SEQ_NO_34 | A0A9P0APF5_BEMTA | BEMITA_LOCUS14329 |
| Bemisia tabaci | 26.23 | SEQ_NO_34 | A0A9P0A482_BEMTA | BEMITA_LOCUS5198 |
| Bemisia tabaci | 61.97 | SEQ_NO_34 | A0A9P0APP5_BEMTA | BEMITA_LOCUS14100 |
| Bemisia tabaci | 26.04 | SEQ_NO_34 | XP_018897454 | LOC109030778 |
| Bemisia tabaci | 35.05 | SEQ_NO_34 | XP_018900077 | LOC109032412 |
| Bemisia tabaci | 34.31 | SEQ_NO_34 | CAH0762837 | empty |
| Bemisia tabaci | 34.31 | SEQ_NO_34 | A0A9P0CBZ6_BEMTA | BEMITA_LOCUS2923 |
| Bemisia tabaci | 31.14 | SEQ_NO_34 | CAH0763060 | empty |
| Bemisia tabaci | 31.14 | SEQ_NO_34 | A0A9P0C9G1_BEMTA | BEMITA_LOCUS3114 |
| Bemisia tabaci | 33.6 | SEQ_NO_34 | CAH0763197 | empty |
| Bemisia tabaci | 33.6 | SEQ_NO_34 | A0A9P0C6Q3_BEMTA | BEMITA_LOCUS3229 |
| Bemisia tabaci | 33.6 | SEQ_NO_34 | XP_018899273 | LOC109031899 |
| Bemisia tabaci | 30.68 | SEQ_NO_34 | XP_018897565 | LOC109030838 |
| Bemisia tabaci | 28.44 | SEQ_NO_34 | A0A9P0CCZ4_BEMTA | BEMITA_LOCUS10122 |
| Trialeurodes | 36.47 | SEQ_NO_34 | A0A873P552_TRIVP | UGT354D4 |
| vaporariorum | | | | |
| Bemisia tabaci | 31.85 | SEQ_NO_34 | XP_018900061 | LOC109032402 |
| Bemisia tabaci | 31.85 | SEQ_NO_34 | XP_018900060 | LOC109032402 |
| Trialeurodes | 29.9 | SEQ_NO_34 | QPA18390 | UGT354D1 |
| vaporariorum | | | | |
| (greenhouse | | | | |
| whitefly) | | | | |
| Trialeurodes | 29.9 | SEQ_NO_34 | A0A873P526_TRIVP | UGT354D1 |
| vaporariorum | | | | |
| Bemisia tabaci | 30.25 | SEQ_NO_34 | XP_018917944 | LOC109044588 |
| Trialeurodes | 30.05 | SEQ_NO_34 | QPA18402 | UGT360B5 |
| vaporariorum | | | | |
| (greenhouse | | | | |
| whitefly) | | | | |
| Trialeurodes | 30.05 | SEQ_NO_34 | A0A873P540_TRIVP | UGT360B5 |
| vaporariorum | | | | |
| Bemisia tabaci | 41.61 | SEQ_NO_34 | A0A9P0ANY8_BEMTA | BEMITA_LOCUS14107 |
| Bemisia tabaci | 30.29 | SEQ_NO_34 | A0A9P0FA32_BEMTA | BEMITA_LOCUS14050 |
| Bemisia tabaci | 30.48 | SEQ_NO_34 | XP_018910403 | LOC109039385 |
| Bemisia tabaci | 30.48 | SEQ_NO_34 | XP_018910402 | LOC109039385 |
| Bemisia tabaci | 32.46 | SEQ_NO_34 | XP_018911461 | LOC109040109 |
| Trialeurodes | 28.14 | SEQ_NO_34 | QPA18372 | UGT356F2 |
| vaporariorum | | | | |
| (greenhouse | | | | |
| whitefly) | | | | |
| Trialeurodes | 28.14 | SEQ_NO_34 | A0A873P506_TRIVP | UGT356F2 |
| vaporariorum | | | | |
| Trialeurodes | 30.25 | SEQ_NO_34 | QPA18369 | UGT353G1 |
| vaporariorum | | | | |
| (greenhouse | | | | |
| whitefly) | | | | |
| Trialeurodes | 30.25 | SEQ_NO_34 | A0A873P502_TRIVP | UGT353G1 |
| vaporariorum | | | | |
| Bemisia tabaci | 24.04 | SEQ_NO_34 | CAH0388444 | empty |
| Bemisia tabaci | 24.04 | SEQ_NO_34 | A0A9P0F1X2_BEMTA | BEMITA_LOCUS7356 |
| Bemisia tabaci | 26.08 | SEQ_NO_34 | A0A9P0CC41_BEMTA | BEMITA_LOCUS4253 |
| Bemisia tabaci | 27.3 | SEQ_NO_34 | XP_018907126 | LOC109037087 |
| Bemisia tabaci | 35.85 | SEQ_NO_34 | CAH0383690 | empty |
| Bemisia tabaci | 35.85 | SEQ_NO_34 | A0A9P0A5B3_BEMTA | BEMITA_LOCUS3115 |
| Bemisia tabaci | 36.43 | SEQ_NO_34 | CAH0383389 | empty |
| Bemisia tabaci | 36.43 | SEQ_NO_34 | A0A9P0F0A5_BEMTA | BEMITA_LOCUS2843 |
| Bemisia tabaci | 33.21 | SEQ_NO_34 | CAH0383112 | empty |
| Bemisia tabaci | 33.21 | SEQ_NO_34 | A0A9P0A4B0_BEMTA | BEMITA_LOCUS2588 |
| Bemisia tabaci | 31.32 | SEQ_NO_34 | CAH0383691 | empty |
| Bemisia tabaci | 31.32 | SEQ_NO_34 | A0A9P0EXU5_BEMTA | BEMITA_LOCUS3115 |
| Bemisia tabaci | 34.51 | SEQ_NO_34 | XP_018918008 | LOC109044638 |
| Bemisia tabaci | 67.37 | SEQ_NO_34 | XP_018896857 | LOC109030376 |
| Bemisia tabaci | 31.35 | SEQ_NO_34 | XP_018912138 | LOC109040611 |
| Bemisia tabaci | 39.42 | SEQ_NO_34 | XP_018911252 | LOC109039965 |
| Bemisia tabaci | 22.82 | SEQ_NO_34 | XP_018918007 | LOC109044638 |
| Bemisia tabaci | 43.3 | SEQ_NO_34 | CAH0389264 | empty |
| Bemisia tabaci | 43.3 | SEQ_NO_34 | A0A9P0AEX2_BEMTA | BEMITA_LOCUS8111 |
| Bemisia tabaci | 38.32 | SEQ_NO_34 | A0A9P0APD8_BEMTA | BEMITA_LOCUS14099 |
| Bemisia tabaci | 28.89 | SEQ_NO_34 | XP_018905481 | LOC109036030 |
| Bemisia tabaci | 30.61 | SEQ_NO_34 | CAH0383821 | empty |
| Bemisia tabaci | 30.61 | SEQ_NO_34 | A0A9P0EXY2_BEMTA | BEMITA_LOCUS3229 |
| Bemisia tabaci | 29.85 | SEQ_NO_34 | XP_018896426 | LOC109030082 |
| Bemisia tabaci | 29.85 | SEQ_NO_34 | XP_018896418 | LOC109030082 |
| Bemisia tabaci | 29.85 | SEQ_NO_34 | XP_018896408 | LOC109030082 |
| Bemisia tabaci | 24.62 | SEQ_NO_38 | CAH0383692 | empty |
| Bemisia tabaci | 24.62 | SEQ_NO_38 | A0A9P0EY03_BEMTA | BEMITA_LOCUS3115 |
| Bemisia tabaci | 100 | SEQ_NO_14 | XP_018909595 | LOC109038565 |
| Bemisia tabaci | 100 | SEQ_NO_14 | A0A9P0EZ48_BEMTA | BEMITA_LOCUS4387 |
| Bemisia tabaci | 42.59 | SEQ_NO_14 | XP_018910638 | LOC109039568 |
| Bemisia tabaci | 42.36 | SEQ_NO_14 | XP_018910640 | LOC109039568 |
| Bemisia tabaci | 42.36 | SEQ_NO_14 | A0A7S5HGM8_BEMTA | empty |
| Bemisia tabaci | 42.36 | SEQ_NO_14 | XP_018910639 | LOC109039568 |
| Bemisia tabaci | 42.36 | SEQ_NO_14 | XP_018910637 | LOC109039568 |
| Bemisia tabaci | 42.36 | SEQ_NO_14 | A0A9P0F1Z4_BEMTA | BEMITA_LOCUS4742 |
| Bemisia tabaci | 100 | SEQ_NO_18 | XP_018899455 | LOC109032020 |
| Bemisia tabaci | 100 | SEQ_NO_18 | A0A9P0F270_BEMTA | BEMITA_LOCUS4952 |
| Bemisia tabaci | 97.18 | SEQ_NO_18 | XP_018899457 | LOC109032020 |
| Bemisia tabaci | 97.86 | SEQ_NO_18 | QHB15685 | empty |
| Bemisia tabaci | 97.86 | SEQ_NO_18 | XP_018899456 | LOC109032020 |
| Bemisia tabaci | 97.86 | SEQ_NO_18 | A0A7S5LK73_BEMTA | empty |
| Bemisia tabaci | 99.8 | SEQ_NO_18 | XP_018899458 | LOC109032020 |
| Bemisia tabaci | 99.8 | SEQ_NO_18 | XP_018899454 | LOC109032020 |
| Bemisia tabaci | 19.32 | SEQ_NO_18 | XP_018902053 | LOC109033749 |
| Bemisia tabaci | 19.32 | SEQ_NO_18 | A0A9P0A1C1_BEMTA | BEMITA_LOCUS3550 |
| Bemisia tabaci | 99.94 | SEQ_NO_26 | XP_018898272 | LOC109031284 |
| Bemisia tabaci | 98.67 | SEQ_NO_26 | CAH0387825 | empty |
| Bemisia tabaci | 98.67 | SEQ_NO_26 | A0A9P0A6Y6_BEMTA | BEMITA_LOCUS6793 |
| Bemisia tabaci | 98.15 | SEQ_NO_26 | CAH0387826 | empty |
| Bemisia tabaci | 98.15 | SEQ_NO_26 | A0A9P0AAW0_BEMTA | BEMITA_LOCUS6793 |
| Bemisia tabaci | 63.56 | SEQ_NO_26 | XP_018898232 | LOC109031257 |
| Bemisia tabaci | 63.56 | SEQ_NO_26 | XP_018898233 | LOC109031257 |
| Bemisia tabaci | 63.56 | SEQ_NO_26 | XP_018898234 | LOC109031257 |
| Bemisia tabaci | 63.46 | SEQ_NO_26 | CAH0387824 | empty |
| Bemisia tabaci | 63.46 | SEQ_NO_26 | A0A9P0ACR5_BEMTA | BEMITA_LOCUS6792 |
| Bemisia tabaci | 55.95 | SEQ_NO_26 | A0A9P0F494_BEMTA | BEMITA_LOCUS10084 |
| Bemisia tabaci | 56.63 | SEQ_NO_26 | XP_018909305 | LOC109038638 |
| Bemisia tabaci | 55.89 | SEQ_NO_26 | XP_018909304 | LOC109038638 |
| Bemisia tabaci | 55.89 | SEQ_NO_26 | XP_018909303 | LOC109038638 |
| Bemisia tabaci | 55.86 | SEQ_NO_26 | XP_018909306 | LOC109038638 |
| Bemisia tabaci | 55.86 | SEQ_NO_26 | XP_018909307 | LOC109038638 |
| Bemisia tabaci | 37.01 | SEQ_NO_26 | XP_018909312 | LOC109038644 |
| Bemisia tabaci | 36.56 | SEQ_NO_26 | XP_018909313 | LOC109038644 |
| Bemisia tabaci | 38.92 | SEQ_NO_26 | XP_018909314 | LOC109038644 |
| Bemisia tabaci | 28.56 | SEQ_NO_26 | CAH0394171 | empty |
| Bemisia tabaci | 28.56 | SEQ_NO_26 | A0A9P0AMW0_BEMTA | BEMITA_LOCUS12499 |
| Bemisia tabaci | 28.44 | SEQ_NO_26 | XP_018909445 | LOC109038729 |
| Bemisia tabaci | 22.1 | SEQ_NO_8 | CAH0777181 | empty |

1. A recombinant polynucleotide molecule comprising at least one polynucleotide sequence having at least about 85% sequence identity to at least 18 contiguous nucleotides of a target nucleotide sequence, wherein said target nucleotide sequence:
a) encodes a polypeptide involved in the trehalose biosynthesis pathway or sugar transporters, optionally, a hexokinase (Hx), trehalose 6-phosphate synthase (TPS), or UTP-glucose-1-phosphate uridylyltransferase (UGP1), ST1, or ST2; and/or
b) encodes a polypeptide involved in detoxification of plant toxins, optionally, UDP-glucosyltransferase (e.g., UDP-GT1, UDP-GT2, UDP-GT3) or ABC transporters (e.g., ABC1);
wherein said recombinant polynucleotide molecule disrupts the activity of said one or more polypeptides when provided in the diet of an invertebrate pest.

2. The recombinant polynucleotide molecule of claim 1, wherein said target nucleotide sequence:
a) encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40; and/or
b) has a sequence selected from the group consisting of SEQ ID NO:1, 3, 5, 7, 9, 11, 13, 15, 17, 19, 21, 23, 25, 27, 29, 31, 33, 35, 37, 39, 134, and 135;
wherein said recombinant polynucleotide molecule disrupts the activity of said one or more polypeptides when provided in the diet of an invertebrate pest.

3. The recombinant polynucleotide molecule of claim 1, further comprising at least one polynucleotide sequence having at least about 85% sequence identity to at least 18 contiguous nucleotides of a second target nucleotide sequence, wherein said second target nucleotide sequence:
a) encodes a polypeptide of an osmoregulation gene in an invertebrate pest optionally, a water-carrying aquaporin, optionally, AQP1; or an α-glucosidase gene, optionally, SUC1, SUC2, SUC3, SUC4, SUC5, SUC7, SUC12; and/or
b) encodes a polypeptide of an essential symbiosis gene, optionally an aspartate aminotransferase (AAT); Arginosuccinate lyase (argH); a diaminopimelate decarboxylase (LysA); a branched-chain-amino-acid aminotransferase gene (BCAT); a 4-hydroxy-tetrahydrodipicolinate reductase (dapB); and/or Chorismate mutase (CM); Diaminopimelate epimerase (DapF); Biotin synthase (e.g., BioA, BioB, bioD).

4. The recombinant polynucleotide molecule of claim 1, further comprising at least one polynucleotide sequence having at least about 85% sequence identity to at least 18 contiguous nucleotides of a second target nucleotide sequence, wherein said second target nucleotide sequence:
a) encodes a polypeptide having a sequence selected from the group consisting of SEQ ID NO:41, 43, 45, 47, 49, 51, 53, 98, 100, 102, 104, and 106; and/or
b) has a sequence of any of SEQ ID NO:42, 44, 46, 48, 50, 52, 54, 97, 99, 101, 103, 105, 107-115, 136, and 137.

5. The recombinant polynucleotide molecule of claim 1:
a) further comprising at least two polynucleotides, at least three polynucleotides, at least four polynucleotides, or at least five polynucleotides, optionally singular, stacked, linear, repeat, multimer, or inverted repeat;
b) wherein said polynucleotide is a first polynucleotide sequence operably linked to a heterologous promoter;
c) wherein said polynucleotide is a first polynucleotide sequence encoding an interfering RNA molecule;
d) wherein said polynucleotide is a first polynucleotide sequence encoding a ssRNA molecule or a dsRNA molecule;
e) wherein said polynucleotide is a first polynucleotide sequence that has at least about 90%, at least about 95%, or 100% sequence identity to at least 18 contiguous nucleotides of said target sequence; and/or
f) wherein said polynucleotide is a first polynucleotide sequence that has at least about 85% sequence identity to at least 19 contiguous nucleotides, at least 20 contiguous nucleotides, or at least 21 contiguous nucleotides of said target sequence.

6-10. (canceled)

11. The recombinant polynucleotide molecule of claim 1,
wherein the at least one polynucleotide sequence:
a) disrupts trehalose biosynthesis activity, sugar transporter activity, or detoxification activity; and/or
b) disrupts osmoregulation or biosynthesis of nutrients.

12. (canceled)

13. The recombinant polynucleotide molecule of claim 1, wherein the invertebrate pest is a pest of the order Hemiptera.

14. The recombinant polynucleotide molecule of claim 13, wherein the pest of the order Hemiptera is a whitefly, optionally, a Bemisia species pest.

15. A plant, plant part, plant cell, seed, or commodity product comprising the recombinant polynucleotide molecule of claim 1.

16. (canceled)

17. A composition comprising the recombinant polynucleotide molecule of claim 1, wherein said target nucleotide sequence:
a) encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40; and/or
b) has a sequence selected from the group consisting of SEQ ID NO:1, 3, 5, 7, 9, 11, 13, 15, 17, 19, 21, 23, 25, 27, 29, 31, 33, 35, 37, 39, 134, and 135;
wherein said composition disrupts the activity of said polypeptide when provided in the diet of an invertebrate pest.

18. The composition of claim 17, wherein the polynucleotide molecule is an interfering RNA molecule or encodes an interfering RNA molecule.

19. The composition of claim 18, wherein the polynucleotide molecule is a ssRNA molecule or a dsRNA molecule or encodes a ssRNA molecule or a dsRNA molecule.

20. A method for controlling invertebrate pest infestation, the method comprising providing a polynucleotide molecule comprising at least one polynucleotide sequence having at least 85% sequence identity to at least 18 contiguous nucleotides of a target nucleotide sequence in the diet of an invertebrate pest, wherein said polynucleotide molecule disrupts the activity of a polypeptide encoded by said target nucleotide sequence, wherein said target nucleotide sequence:
a) encodes a polypeptide of a sugar transport and storage gene in an invertebrate pest optionally, a hexokinase (Hx), trehalose 6-phosphate synthase (TPS), or UTP-glucose-1-phosphate uridylyltransferase (UGP1), ST1, or ST2; and/or
b) encodes a polypeptide of phytotoxin detoxification gene in an invertebrate pest optionally, UDP-glucosyltransferase (e.g., UDP-GT1, UDP-GT2, UDP-GT3) or ABC transporters (e.g., ABC1).

21. The method of claim 20, wherein said polynucleotide molecule further comprises providing at least a second polynucleotide sequence having at least about 85% sequence identity to at least 18 contiguous nucleotides of a second target nucleotide sequence, wherein said second target nucleotide sequence:
a) encodes a polypeptide of an osmoregulation gene in an invertebrate pest optionally, a water-carrying aquaporin, optionally, AQP1; or an α-glucosidase gene, optionally, SUC1, SUC2, SUC3, SUC4, SUC5, SUC7, SUC12; and/or
b) encodes a polypeptide of an essential symbiosis gene, optionally an aspartate aminotransferase (AAT); Arginosuccinate lyase (argH); a diaminopimelate decarboxylase (LysA); a branched-chain-amino-acid aminotransferase gene (BCAT); a 4-hydroxy-tetrahydrodipicolinate reductase (dapB); and/or Chorismate mutase (CM); Diaminopimelate epimerase (DapF); Biotin synthase (e.g., BioA, BioB, bioD).

22. A method for controlling invertebrate pest infestation, the method comprising providing a polynucleotide molecule comprising a first polynucleotide sequence having at least 85% sequence identity to at least 18 contiguous nucleotides of a target nucleotide sequence in the diet of an invertebrate pest, wherein said polynucleotide molecule disrupts the activity of a polypeptide encoded by said target nucleotide sequence, wherein said target nucleotide sequence:
a) encodes a polypeptide having a sequence selected from the group consisting of SEQ ID NO:2, 4, 6, 8, 10, 12, 14, 16, 18, 20, 22, 24, 26, 28, 30, 32, 34, 36, 38, and 40; or
b) has a sequence selected from the group consisting of SEQ ID NO:1, 3, 5, 7, 9, 11, 13, 15, 17, 19, 21, 23, 25, 27, 29, 31, 33, 35, 37, 39, 134, and 135.

23. The method of claim 22, further comprising providing a second polynucleotide sequence having at least about 85% sequence identity to at least 18 contiguous nucleotides of a second target nucleotide sequence, wherein said second target nucleotide sequence:
a) encodes a polypeptide having a sequence selected from the group consisting of SEQ ID NO:41, 43, 45, 47, 49, 51, 53, 98, 100, 102, 104, and 106; and/or
b) has a sequence of any of SEQ ID NO:42, 44, 46, 48, 50, 52, 54, 97, 99, 101, 103, 105, 107-115, 136, and 137.

24. The method of claim 22, wherein the polynucleotide molecule is:
a) an interfering RNA molecule or encodes an interfering RNA molecule; and/or
b) is a ssRNA molecule or a dsRNA molecule or encodes a ssRNA molecule or a dsRNA molecule.

25. (canceled)

26. The method of claim 22, wherein providing the polynucleotide molecule comprises providing a plant, plant part, plant cell, seed, or composition comprising said polynucleotide molecule in the diet of the invertebrate pest.

27. The method of claim 22, wherein the invertebrate pest is a pest of the order Hemiptera.

28. The method of claim 27, wherein the pest of the order Hemiptera is a whitefly, optionally, a Bemisia species pest.
